SEQUENCE LISTING (268 sequences)

SEQ ID NO: 1 - protein, 14 aa, Artificial Sequence
  Synthetic construct BT1
GRCTKSIPPICFPD

SEQ ID NO: 2 - protein, 16 aa, Artificial Sequence
  Synthetic construct BT2
CGAKGTKSIPPICFPD

SEQ ID NO: 3 - protein, 16 aa, Artificial Sequence
  Synthetic construct BT3
CGAKATKSIPPICFPD

SEQ ID NO: 4 - protein, 16 aa, Artificial Sequence
  Synthetic construct BT4
CGAKGTKSIPPIGFCD

SEQ ID NO: 5 - protein, 15 aa, Artificial Sequence
  Synthetic construct BT5
CGRATKSIPPICFPD

SEQ ID NO: 6 - protein, 15 aa, Artificial Sequence
  Synthetic construct BT6
CGSATKSIPPICFPD

SEQ ID NO: 7 - protein, 15 aa, Artificial Sequence
  Synthetic construct BT7
CGAATKSIPPICFPD

SEQ ID NO: 8 - protein, 12 aa, Artificial Sequence
  Synthetic construct BT8
CGRATKSIPPIC

SEQ ID NO: 9 - protein, 13 aa, Artificial Sequence
  Synthetic construct BT9
CGRATKSIPPICF

SEQ ID NO: 10 - protein, 13 aa, Artificial Sequence
  Synthetic construct BT10
CGRATKSIPAICF

SEQ ID NO: 11 - protein, 14 aa, Artificial Sequence
  Synthetic construct BT11
CGRATKSIPPIAFC

SEQ ID NO: 12 - protein, 13 aa, Artificial Sequence
  Synthetic construct BT12
CGRATKSIPPQCY

SEQ ID NO: 13 - protein, 13 aa, Artificial Sequence
  Synthetic construct BT13
CGRATKSIPPIAC

SEQ ID NO: 14 - protein, 13 aa, Artificial Sequence
  Synthetic construct BT14
CGRATKSLPPACF

SEQ ID NO: 15 - protein, 13 aa, Artificial Sequence
  Synthetic construct BT15
CGRATRSIPPACF

SEQ ID NO: 16 - protein, 13 aa, Artificial Sequence
  Synthetic construct BT16
CGRATKSLPAICF

SEQ ID NO: 17 - protein, 13 aa, Artificial Sequence
  Synthetic construct BT17
CGRATRSIPPICF

SEQ ID NO: 18 - protein, 13 aa, Artificial Sequence
  Synthetic construct BT18
CGRATRSIPPICY

SEQ ID NO: 19 - protein, 13 aa, Artificial Sequence
  Synthetic construct BT19
CGRATRSIPPICA

SEQ ID NO: 20 - protein, 13 aa, Artificial Sequence
  Synthetic construct BT20
CGRATRSIAPICF

SEQ ID NO: 21 - protein, 13 aa, Artificial Sequence
  Synthetic construct BT21
CGRATRSAPPICF

SEQ ID NO: 22 - protein, 13 aa, Artificial Sequence
  Synthetic construct BT22
CGRATRAIPPICF

SEQ ID NO: 23 - protein, 14 aa, Artificial Sequence
  Synthetic construct BT23
GTCTRSIPPICNPN

SEQ ID NO: 24 - protein, 13 aa, Artificial Sequence
  Synthetic construct BT24
CGTATKSIPPICN

SEQ ID NO: 25 - protein, 13 aa, Artificial Sequence
  Synthetic construct BT25
CGTATKSIPPICF

SEQ ID NO: 26 - protein, 13 aa, Artificial Sequence
  Synthetic construct BT26
CGRAAKSIPPICF

SEQ ID NO: 27 - protein, 13 aa, Artificial Sequence
  Synthtic construct BT27
CGRATKAIPPICF

SEQ ID NO: 28 - protein, 13 aa, Artificial Sequence
  Synthetic construct BT28
  MOD_RES (8)..(8): Nle
CGRATKSXPPICF

SEQ ID NO: 29 - protein, 14 aa, Artificial Sequence
  Synthetic construct BT29
  MOD_RES (1)..(1): homocysteine
  MOD_RES (14)..(14): homocysteine
XGRATKSIPPIAFX

SEQ ID NO: 30 - protein, 13 aa, Artificial Sequence
  Synthetic construct BT30
CGRATKSIPPIFC

SEQ ID NO: 31 - protein, 13 aa, Artificial Sequence
  Synthetic construct BT31
CGRATKSIPPAFC

SEQ ID NO: 32 - protein, 15 aa, Artificial Sequence
  Synthetic construct BT32
CGRATKSIPPIAFGC

SEQ ID NO: 33 - protein, 15 aa, Artificial Sequence
  Synthetic construct BT33
  MOD_RES (1)..(1): homocysteine
  MOD_RES (15)..(15): homocysteine
XGRATKSIPPIAFGX

SEQ ID NO: 34 - protein, 14 aa, Artificial Sequence
  Synthetic construct BT34
CGRATKSIPPQARC

SEQ ID NO: 35 - protein, 13 aa, Artificial Sequence
  Synthetic construct BT35
  MOD_RES (4)..(4): Abu
CGRXTKSIPPICF

SEQ ID NO: 36 - protein, 11 aa, Artificial Sequence
  Synthetic construct BT36
CWTKSIPPKPC

SEQ ID NO: 37 - protein, 12 aa, Artificial Sequence
  Synthetic construct BT37
CGWTKSIPPKPC

SEQ ID NO: 38 - protein, 13 aa, Artificial Sequence
  Synthetic construct BT38
CGRWTKSIPPKPC

SEQ ID NO: 39 - protein, 13 aa, Artificial Sequence
  Synthetic construct BT39
CGRWTKSIPPAFC

SEQ ID NO: 40 - protein, 13 aa, Artificial Sequence
  Synthetic construct BT40
CGRWTKSIPPIFC

SEQ ID NO: 41 - protein, 11 aa, Artificial Sequence
  Synthetic construct BT41
GRCPKILKKCF

SEQ ID NO: 42 - protein, 12 aa, Artificial Sequence
  Synthetic construct BT42
CGRAPKILKKCF

SEQ ID NO: 43 - protein, 11 aa, Artificial Sequence
  Synthetic construct BT43
GRCPKILQRCF

SEQ ID NO: 44 - protein, 12 aa, Artificial Sequence
  Synthetic construct BT44
CGRAPKILQRCF

SEQ ID NO: 45 - protein, 11 aa, Artificial Sequence
  Synthetic construct BT45
RCTKSIPPICF

SEQ ID NO: 46 - protein, 13 aa, Artificial Sequence
  Synthetic construct BT46
  MOD_RES (2)..(2): 4Hyp
CXRATKSIPPICF

SEQ ID NO: 47 - protein, 13 aa, Artificial Sequence
  Synthetic construct BT47
CGRSTKSIPPICF

SEQ ID NO: 48 - protein, 13 aa, Artificial Sequence
  Synthetic construct BT48
CGRLTKSIPPICF

SEQ ID NO: 49 - protein, 13 aa, Artificial Sequence
  Synthetic construct BT49
CGRITKSIPPICF

SEQ ID NO: 50 - protein, 13 aa, Artificial Sequence
  Synthetic construct BT50
  MOD_RES (4)..(4): Nle
CGRXTKSIPPICF

SEQ ID NO: 51 - protein, 13 aa, Artificial Sequence
  Synthetic construct BT51
CGRVTKSIPPICF

SEQ ID NO: 52 - protein, 13 aa, Artificial Sequence
  Synthetic construct BT52
CGRFTKSIPPICF

SEQ ID NO: 53 - protein, 13 aa, Artificial Sequence
  Synthetic construct BT53
CGRYTKSIPPICF

SEQ ID NO: 54 - protein, 13 aa, Artificial Sequence
  Synthetic construct BT54
CGRQTKSIPPICF

SEQ ID NO: 55 - protein, 13 aa, Artificial Sequence
  Synthetic construct BT55
CGRNTKSIPPICF

SEQ ID NO: 56 - protein, 13 aa, Artificial Sequence
  Synthetic construct BT56
CGRHTKSIPPICF

SEQ ID NO: 57 - protein, 13 aa, Artificial Sequence
  Synthetic construct BT57
CGRWTKSIPPICF

SEQ ID NO: 58 - protein, 13 aa, Artificial Sequence
  Synthetic construct BT58
CGRETKSIPPICF

SEQ ID NO: 59 - protein, 13 aa, Artificial Sequence
  Synthetic construct BT59
CGRPTKSIPPICF

SEQ ID NO: 60 - protein, 13 aa, Artificial Sequence
  Synthetic construct BT60
CGRGTKSIPPICF

SEQ ID NO: 61 - protein, 11 aa, Artificial Sequence
  Synthetic construct BT61
RCTRSIPPHCW

SEQ ID NO: 62 - protein, 11 aa, Artificial Sequence
  Synthetic construct BT62
RCTKSIPPHCF

SEQ ID NO: 63 - protein, 11 aa, Artificial Sequence
  Synthetic construct BT63
RCTKSIPPQCH

SEQ ID NO: 64 - protein, 11 aa, Artificial Sequence
  Synthetic construct BT64
RCTKSNPPQCQ

SEQ ID NO: 65 - protein, 11 aa, Artificial Sequence
  Synthetic construct BT65
RCTKSLPPQCS

SEQ ID NO: 66 - protein, 13 aa, Artificial Sequence
  Synthetic construct BT66
CPRATKSIPPICF

SEQ ID NO: 67 - protein, 13 aa, Artificial Sequence
  Synthetic construct BT67
CARATKSIPPICF

SEQ ID NO: 68 - protein, 11 aa, Artificial Sequence
  Synthetic construct BT68
ACTKSNPPQCR

SEQ ID NO: 69 - protein, 13 aa, Artificial Sequence
  Synthetic construct BT69
  MOD_RES (9)..(9): 4Hyp
CARATKSIXPICF

SEQ ID NO: 70 - protein, 13 aa, Artificial Sequence
  Synthetic construct BT70
  MOD_RES (10)..(10): 4Hyp
CARATKSIPXICF

SEQ ID NO: 71 - protein, 13 aa, Artificial Sequence
  Synthetic construct BT71
CVRATKSIPPICF

SEQ ID NO: 72 - protein, 13 aa, Artificial Sequence
  Synthetic construct BT72
CLRATKSIPPICF

SEQ ID NO: 73 - protein, 13 aa, Artificial Sequence
  Synthetic construct BT73
CIRATKSIPPICF

SEQ ID NO: 74 - protein, 13 aa, Artificial Sequence
  Synthetic construct BT74
  MOD_RES (2)..(2): Abu
CXRATKSIPPICF

SEQ ID NO: 75 - protein, 13 aa, Artificial Sequence
  Synthetic construct BT75
CSRATKSIPPICF

SEQ ID NO: 76 - protein, 13 aa, Artificial Sequence
  Synthetic construct BT76
CRRATKSIPPICF

SEQ ID NO: 77 - protein, 13 aa, Artificial Sequence
  Synthetic construct BT77
CKRATKSIPPICF

SEQ ID NO: 78 - protein, 13 aa, Artificial Sequence
  Synthetic construct BT78
CERATKSIPPICF

SEQ ID NO: 79 - protein, 13 aa, Artificial Sequence
  Synthetic construct BT79
CQRATKSIPPICF

SEQ ID NO: 80 - protein, 13 aa, Artificial Sequence
  Synthetic construct BT80
  MOD_RES (2)..(2): Nle
CXRATKSIPPICF

SEQ ID NO: 81 - protein, 12 aa, Artificial Sequence
  Synthetic construct CH1
LCTFSIPPQCYG

SEQ ID NO: 82 - protein, 12 aa, Artificial Sequence
  Synthetic construct CH2
CLAFSIPPQCYG

SEQ ID NO: 83 - protein, 13 aa, Artificial Sequence
  Synthetic construct CH3
CGLAFSIPPQCYG

SEQ ID NO: 84 - protein, 12 aa, Artificial Sequence
  Synthetic construct CH4
SCTYSIPPQCYG

SEQ ID NO: 85 - protein, 12 aa, Artificial Sequence
  Synthetic construct CH5
FCTFSIPPQCYG

SEQ ID NO: 86 - protein, 14 aa, Artificial Sequence
  Synthetic construct CH6
CGSGTYSIPPQCYG

SEQ ID NO: 87 - protein, 14 aa, Artificial Sequence
  Synthetic construct CH7
CGFGTFSIPPQCYG

SEQ ID NO: 88 - protein, 12 aa, Artificial Sequence
  Synthetic construct CH8
CSATYSIPPQCY

SEQ ID NO: 89 - protein, 13 aa, Artificial Sequence
  Synthetic construct CH9
CGSATYSIPPQCY

SEQ ID NO: 90 - protein, 12 aa, Artificial Sequence
  Synthetic construct CH10
FCTYSIPPQCYG

SEQ ID NO: 91 - protein, 12 aa, Artificial Sequence
  Synthetic construct CH11
SCTFSIPPQCYG

SEQ ID NO: 92 - protein, 14 aa, Artificial Sequence
  Synthetic construct CH12
CGFATFSIPPQCYG

SEQ ID NO: 93 - protein, 14 aa, Artificial Sequence
  Synthetic construct CH13
CGSATFSIPPQCYG

SEQ ID NO: 94 - protein, 14 aa, Artificial Sequence
  Synthetic construct CH14
CGFATYSIPPQCYG

SEQ ID NO: 95 - protein, 14 aa, Artificial Sequence
  Synthetic construct CH15
CGSATYSIPPQCYG

SEQ ID NO: 96 - protein, 13 aa, Artificial Sequence
  Synthetic construct CH16
CSATYSIPPQCYG

SEQ ID NO: 97 - protein, 11 aa, Artificial Sequence
  Synthetic construct CH17
ICTFSIPAQCV

SEQ ID NO: 98 - protein, 11 aa, Artificial Sequence
  Synthetic construct CH18
ACTYSIPAKCF

SEQ ID NO: 99 - protein, 12 aa, Artificial Sequence
  Synthetic construct CH19
FCTLSIPPQCYG

SEQ ID NO: 100 - protein, 14 aa, Artificial Sequence
  Synthetic construct CH20
GKCLYSIPPICFPN

SEQ ID NO: 101 - protein, 14 aa, Artificial Sequence
  Synthetic construct CH21
CGNATYSIPPQCYG

SEQ ID NO: 102 - protein, 14 aa, Artificial Sequence
  Synthetic construct CH22
CGTATYSIPPQCYG

SEQ ID NO: 103 - protein, 14 aa, Artificial Sequence
  Synthetic construct CH23
CGSATYSIPAQCVG

SEQ ID NO: 104 - protein, 14 aa, Artificial Sequence
  Synthetic construct CH24
CGSATYSIPAKCFG

SEQ ID NO: 105 - protein, 14 aa, Artificial Sequence
  Synthetic construct CH25
GTCTFSIPPICNPN

SEQ ID NO: 106 - protein, 12 aa, Artificial Sequence
  Synthetic construct CH26
GTCTFSIPPICN

SEQ ID NO: 107 - protein, 13 aa, Artificial Sequence
  Synthetic construct CH27
CGTATFSIPPICN

SEQ ID NO: 108 - protein, 13 aa, Artificial Sequence
  Synthetic construct CH28
CPGEAMAYIRSCF

SEQ ID NO: 109 - protein, 14 aa, Artificial Sequence
  Synthetic construct CH29
CGGSATYSIPPQCY

SEQ ID NO: 110 - protein, 14 aa, Artificial Sequence
  Synthetic construct CH30
CGYATYSIPPQCYG

SEQ ID NO: 111 - protein, 13 aa, Artificial Sequence
  Synthetic construct CH31
CGAATYSIPAKCF

SEQ ID NO: 112 - protein, 14 aa, Artificial Sequence
  Synthetic construct CH32
CGGAATYSIPAKCF

SEQ ID NO: 113 - protein, 11 aa, Artificial Sequence
  Synthetic construct CH33
FCTYSIPPQCY

SEQ ID NO: 114 - protein, 12 aa, Artificial Sequence
  Synthetic construct CH34
FCTYSIPPQCYA

SEQ ID NO: 115 - protein, 11 aa, Artificial Sequence
  Synthetic construct CH35
FCTYSIPPQCR

SEQ ID NO: 116 - protein, 11 aa, Artificial Sequence
  Synthetic construct CH36
FCTYSIPAKCY

SEQ ID NO: 117 - protein, 11 aa, Artificial Sequence
  Synthetic construct CH37
FCTYSIPAQCY

SEQ ID NO: 118 - protein, 11 aa, Artificial Sequence
  Synthetic construct CH38
ICTFSIPAQCI

SEQ ID NO: 119 - protein, 11 aa, Artificial Sequence
  Synthetic construct CH39
ICTFSIPAQCV

SEQ ID NO: 120 - protein, 11 aa, Artificial Sequence
  Synthetic construct CH40
ICTFSIPAQCF

SEQ ID NO: 121 - protein, 11 aa, Artificial Sequence
  Synthetic construct CH41
FCTYSMPPHCV

SEQ ID NO: 122 - protein, 11 aa, Artificial Sequence
  Synthetic construct CH42
RCDFSWPPRCL

SEQ ID NO: 123 - protein, 11 aa, Artificial Sequence
  Synthetic construct CH43
FCAYSNPPQCQ

SEQ ID NO: 124 - protein, 11 aa, Artificial Sequence
  Synthetic construct CH44
FCAYSNPPKCQ

SEQ ID NO: 125 - protein, 11 aa, Artificial Sequence
  Synthetic construct CH45
FCAYSYPPKCQ

SEQ ID NO: 126 - protein, 11 aa, Artificial Sequence
  Synthetic construct CH46
FCNYSNPPQCQ

SEQ ID NO: 127 - protein, 11 aa, Artificial Sequence
  Synthetic construct CH47
ICTYSIPAQCI

SEQ ID NO: 128 - protein, 11 aa, Artificial Sequence
  Synthetic construct CH48
VCTFSNPAMCH

SEQ ID NO: 129 - protein, 11 aa, Artificial Sequence
  Synthetic construct CH49
MCTFSHPAKCV

SEQ ID NO: 130 - protein, 11 aa, Artificial Sequence
  Synthetic construct CH50
MCTFSDPGMCS

SEQ ID NO: 131 - protein, 11 aa, Artificial Sequence
  Synthetic construct CH51
PCTYSIPPQCY

SEQ ID NO: 132 - protein, 12 aa, Artificial Sequence
  Synthetic construct CH52
  MOD_RES (8)..(8): 4Hyp
FCTYSIPXQCYG

SEQ ID NO: 133 - protein, 12 aa, Artificial Sequence
  Synthetic construct CH53
  MOD_RES (7)..(7): 4Hyp
FCTYSIXPQCYG

SEQ ID NO: 134 - protein, 11 aa, Artificial Sequence
  Synthetic construct EC1
LCTASIPPQCY

SEQ ID NO: 135 - protein, 11 aa, Artificial Sequence
  Synthetic construct EC2
LCTLSIPPQCY

SEQ ID NO: 136 - protein, 13 aa, Artificial Sequence
  Synthetic construct EC3
CGLATASIPPQCY

SEQ ID NO: 137 - protein, 13 aa, Artificial Sequence
  Synthetic construct EC4
CGLGTASIPPQCY

SEQ ID NO: 138 - protein, 12 aa, Artificial Sequence
  Synthetic construct EC5
CLATASIPPQCY

SEQ ID NO: 139 - protein, 13 aa, Artificial Sequence
  Synthetic construct EC6
CGLATASIPPICQ

SEQ ID NO: 140 - protein, 13 aa, Artificial Sequence
  Synthetic construct EC7
CGLATLSIPPICQ

SEQ ID NO: 141 - protein, 13 aa, Artificial Sequence
  Synthetic construct EC8
CGRETASIPPICQ

SEQ ID NO: 142 - protein, 13 aa, Artificial Sequence
  Synthetic construct EC9
CGRETASIPPQCK

SEQ ID NO: 143 - protein, 13 aa, Artificial Sequence
  Synthetic construct EC10
CGRETASIPPQKC

SEQ ID NO: 144 - protein, 14 aa, Artificial Sequence
  Synthetic construct EC11
CGRETASIPPQKGC

SEQ ID NO: 145 - protein, 11 aa, Artificial Sequence
  Synthetic construct EC12
LCTASIPPICQ

SEQ ID NO: 146 - protein, 11 aa, Artificial Sequence
  Synthetic construct EC13
ICTLSIPAQCV

SEQ ID NO: 147 - protein, 11 aa, Artificial Sequence
  Synthetic construct EC14
YCTASIPPQCY

SEQ ID NO: 148 - protein, 13 aa, Artificial Sequence
  Synthetic construct EC15
CGYATASIPPQCY

SEQ ID NO: 149 - protein, 14 aa, Artificial Sequence
  Synthetic construct EC16
CGGLATASIPPICQ

SEQ ID NO: 150 - protein, 14 aa, Artificial Sequence
  Synthetic construct EC17
CGGLATLSIPPICQ

SEQ ID NO: 151 - protein, 11 aa, Artificial Sequence
  Synthetic construct EC18
LCTASIPPQCQ

SEQ ID NO: 152 - protein, 11 aa, Artificial Sequence
  Synthetic construct EC19
ICTASIPPQCQ

SEQ ID NO: 153 - protein, 11 aa, Artificial Sequence
  Synthetic construct EC20
LCTASIPPQCR

SEQ ID NO: 154 - protein, 11 aa, Artificial Sequence
  Synthetic construct EC21
LCTASIPPICR

SEQ ID NO: 155 - protein, 11 aa, Artificial Sequence
  Synthetic construct EC22
VCTASIPPICQ

SEQ ID NO: 156 - protein, 11 aa, Artificial Sequence
  Synthetic construct EC23
ICTASIPPICQ

SEQ ID NO: 157 - protein, 11 aa, Artificial Sequence
  Synthetic construct EC24
FCTASIPPICQ

SEQ ID NO: 158 - protein, 11 aa, Artificial Sequence
  Synthetic construct EC25
LCTASNPPICQ

SEQ ID NO: 159 - protein, 11 aa, Artificial Sequence
  Synthetic construct EC26
MCTASMPPQCH

SEQ ID NO: 160 - protein, 11 aa, Artificial Sequence
  Synthetic construct EC27
ICTASYPPQCR

SEQ ID NO: 161 - protein, 11 aa, Artificial Sequence
  Synthetic construct EC28
LCTASNPPTCR

SEQ ID NO: 162 - protein, 11 aa, Artificial Sequence
  Synthetic construct EC29
YCTASIPPICQ

SEQ ID NO: 163 - protein, 13 aa, Artificial Sequence
  Synthetic construct EC30
CGIATASIPPICQ

SEQ ID NO: 164 - protein, 13 aa, Artificial Sequence
  Synthetic construct EC31
  MOD_RES (4)..(4): Abu
CGIXTASIPPICQ

SEQ ID NO: 165 - protein, 13 aa, Artificial Sequence
  Synthetic construct EC32
  MOD_RES (4)..(4): Nle
CGIXTASIPPICQ

SEQ ID NO: 166 - protein, 13 aa, Artificial Sequence
  Synthetic construct EC33
CGILTASIPPICQ

SEQ ID NO: 167 - protein, 13 aa, Artificial Sequence
  Synthetic construct EC34
CGISTASIPPICQ

SEQ ID NO: 168 - protein, 13 aa, Artificial Sequence
  Synthetic construct EC35
CGITTASIPPICQ

SEQ ID NO: 169 - protein, 13 aa, Artificial Sequence
  Synthetic construct EC36
CGIFTASIPPICQ

SEQ ID NO: 170 - protein, 13 aa, Artificial Sequence
  Synthetic construct EC37
CGIYTASIPPICQ

SEQ ID NO: 171 - protein, 13 aa, Artificial Sequence
  Synthetic construct EC38
CGINTASIPPICQ

SEQ ID NO: 172 - protein, 13 aa, Artificial Sequence
  Synthetic construct EC39
CGIQTASIPPICQ

SEQ ID NO: 173 - protein, 13 aa, Artificial Sequence
  Synthetic construct EC40
CGIHTASIPPICQ

SEQ ID NO: 174 - protein, 13 aa, Artificial Sequence
  Synthetic construct EC41
CGIRTASIPPICQ

SEQ ID NO: 175 - protein, 13 aa, Artificial Sequence
  Synthetic construct EC42
CGIKTASIPPICQ

SEQ ID NO: 176 - protein, 13 aa, Artificial Sequence
  Synthetic construct EC43
CGIWTASIPPICQ

SEQ ID NO: 177 - protein, 13 aa, Artificial Sequence
  Synthetic construct EC44
CPIATASIPPICQ

SEQ ID NO: 178 - protein, 13 aa, Artificial Sequence
  Synthetic construct EC45
CAIATASIPPICQ

SEQ ID NO: 179 - protein, 13 aa, Artificial Sequence
  Synthetic construct EC46
  MOD_RES (2)..(2): 4Hyp
CXIATASIPPICQ

SEQ ID NO: 180 - protein, 11 aa, Artificial Sequence
  Synthetic construct EC47
  MOD_RES (7)..(7): 4Hyp
ICTASIXPICQ

SEQ ID NO: 181 - protein, 11 aa, Artificial Sequence
  Synthetic construct EC48
  MOD_RES (8)..(8): 4Hyp
ICTASIPXICQ

SEQ ID NO: 182 - protein, 21 aa, Artificial Sequence
  Synthetic construct EC49
QGADTPPVGGLCTASIPPQCY

SEQ ID NO: 183 - protein, 21 aa, Artificial Sequence
  Synthetic construct EC50
SNGNAVEDGGLCTASIPPQCY

SEQ ID NO: 184 - protein, 31 aa, Artificial Sequence
  Synthetic construct GLP-1 (7-37)
HAEGTFTSDVSSYLEGQAAKEFIAWLVKGRG

SEQ ID NO: 185 - protein, 31 aa, Artificial Sequence
  Synthetic construct GLP-1(G8)
HGEGTFTSDVSSYLEGQAAKEFIAWLVKGRG

SEQ ID NO: 186 - protein, 38 aa, Artificial Sequence
  Synthetic construct G7-GLP-1
GGGGGGGHAEGTFTSDVSSYLEGQAAKEFIAWLVKGRG

SEQ ID NO: 187 - protein, 40 aa, Artificial Sequence
  Synthetic construct D-GLP-1
GGIPIGGHAEGTFTSDVSSYLEGQAAKEFIAWLVKGRGGK

SEQ ID NO: 188 - protein, 42 aa, Artificial Sequence
  Synthetic construct N-GLP-1
GGQRFSRGGHAEGTFTSDVSSYLEGQAAKEFIAWLVKGRGGK

SEQ ID NO: 189 - protein, 45 aa, Artificial Sequence
  Synthetic construct T-GLP-1
GGCPKILQRCGGHAEGTFTSDVSSYLEGQAAKEFIAWLVKGRGGK

SEQ ID NO: 190 - protein, 50 aa, Artificial Sequence
  Synthetic construct DT-GLP-1
GGCPKILQRCGGIPIGGHAEGTFTSDVSSYLEGQAAKEFIAWLVKGRGGK

SEQ ID NO: 191 - protein, 52 aa, Artificial Sequence
  Synthetic construct NT-GLP-1
GGCPKILQRCGGQRFSRGGHAEGTFTSDVSSYLEGQAAKEFIAWLVKGRGGK

SEQ ID NO: 192 - protein, 47 aa, Artificial Sequence
  Synthetic construct DN-GLP-1
GGQRFSRGGIPIGGHAEGTFTSDVSSYLEGQAAKEFIAWLVKGRGGK

SEQ ID NO: 193 - protein, 55 aa, Artificial Sequence
  Synthetic construct DNT-GLP-1
CPKILQRCGGQRFSRGGIPIGGHAEGTFTSDVSSYLEGQAAKEFIAWLVKGRGGK

SEQ ID NO: 194 - protein, 53 aa, Artificial Sequence
  Synthetic construct BT1-D-GLP-1
GRCTKSIPPICFPGGIPIGGHAEGTFTSDVSSYLEGQAAKEFIAWLVKGRGGK

SEQ ID NO: 195 - protein, 55 aa, Artificial Sequence
  Synthetic construct BT1-N-GLP-1
GRCTKSIPPICFPGGQRFSRGGHAEGTFTSDVSSYLEGQAAKEFIAWLVKGRGGK

SEQ ID NO: 196 - protein, 52 aa, Artificial Sequence
  Synthetic construct D-GLP-1-BT1
GIPIGGHAEGTFTSDVSSYLEGQAAKEFIAWLVKGRGGKGRCTKSIPPICFP

SEQ ID NO: 197 - protein, 54 aa, Artificial Sequence
  Synthetic construct N-GLP-1-BT1
GQRFSRGGHAEGTFTSDVSSYLEGQAAKEFIAWLVKGRGGKGRCTKSIPPICFP

SEQ ID NO: 198 - protein, 53 aa, Artificial Sequence
  Synthetic construct BT9-D-GLP-1
CGRATKSIPPICFGGIPIGGHAEGTFTSDVSSYLEGQAAKEFIAWLVKGRGGK

SEQ ID NO: 199 - protein, 55 aa, Artificial Sequence
  Synthetic construct BT9-N-GLP-1
CGRATKSIPPICFGGQRFSRGGHAEGTFTSDVSSYLEGQAAKEFIAWLVKGRGGK

SEQ ID NO: 200 - protein, 53 aa, Artificial Sequence
  Synthetic construct D-GLP-1-BT9
GIPIGGHAEGTFTSDVSSYLEGQAAKEFIAWLVKGRGGKGCGRATKSIPPICF

SEQ ID NO: 201 - protein, 55 aa, Artificial Sequence
  Synthetic construct N-GLP-1-BT9
GQRFSRGGHAEGTFTSDVSSYLEGQAAKEFIAWLVKGRGGKGCGRATKSIPPICF

SEQ ID NO: 202 - protein, 51 aa, Artificial Sequence
  Synthetic construct CH4-D-GLP-1
SCTYSIPPQCYGGIPIGGHAEGTFTSDVSSYLEGQAAKEFIAWLVKGRGGK

SEQ ID NO: 203 - protein, 52 aa, Artificial Sequence
  Synthetic construct D-GLP-1-CH4
GIPIGGHAEGTFTSDVSSYLEGQAAKEFIAWLVKGRGGKGSCTYSIPPQCYG

SEQ ID NO: 204 - protein, 51 aa, Artificial Sequence
  Synthetic construct CH10-D-GLP-1
FCTYSIPPQCYGGIPIGGHAEGTFTSDVSSYLEGQAAKEFIAWLVKGRGGK

SEQ ID NO: 205 - protein, 52 aa, Artificial Sequence
  Synthetic construct D-GLP-1-CH10
GIPIGGHAEGTFTSDVSSYLEGQAAKEFIAWLVKGRGGKGFCTYSIPPQCYG

SEQ ID NO: 206 - protein, 51 aa, Artificial Sequence
  Synthetic construct EC1-D-GLP-1
LCTASIPPQCYGGIPIGGHAEGTFTSDVSSYLEGQAAKEFIAWLVKGRGGK

SEQ ID NO: 207 - protein, 51 aa, Artificial Sequence
  Synthetic construct D-GLP-1-EC1
GIPIGGHAEGTFTSDVSSYLEGQAAKEFIAWLVKGRGGKGLCTASIPPQCY

SEQ ID NO: 208 - protein, 51 aa, Artificial Sequence
  Synthetic construct EC12-D-GLP-1
LCTASIPPICQGGIPIGGHAEGTFTSDVSSYLEGQAAKEFIAWLVKGRGGK

SEQ ID NO: 209 - protein, 51 aa, Artificial Sequence
  Synthetic construct D-GLP-1-EC12
GIPIGGHAEGTFTSDVSSYLEGQAAKEFIAWLVKGRGGKGLCTASIPPICQ

SEQ ID NO: 210 - protein, 13 aa, Artificial Sequence
  Synthetic construct PCSK9_1
TVFTSWEEYLDWV

SEQ ID NO: 211 - protein, 26 aa, Artificial Sequence
  Synthetic construct PCSK9_2
TVFTSWEEALDWVCGRATKSIPPICF

SEQ ID NO: 212 - protein, 27 aa, Artificial Sequence
  Synthetic construct PCSK9_3
CGRATKSIPPICFGTVFTSWEEALDWV

SEQ ID NO: 213 - protein, 26 aa, Artificial Sequence
  Synthetic construct PCSK9_4
WEEALDWVCGRATKSIPPICFTVFTS

SEQ ID NO: 214 - protein, 27 aa, Artificial Sequence
  Synthetic construct PCSK9_5
WEEALDWVCGRATKSIPPICFGTVFTS

SEQ ID NO: 215 - protein, 27 aa, Artificial Sequence
  Synthetic construct PCSK9_6
WEEYLDYVCGRATKSIPPICFGTVFTS

SEQ ID NO: 216 - protein, 27 aa, Artificial Sequence
  Synthetic construct PCSK9_7
TVFTSGCGRATKSIPPICFWEEYLDWV

SEQ ID NO: 217 - protein, 28 aa, Artificial Sequence
  Synthetic construct PCSK9_8
TVFTSGCGRATKSIPPICFSWEEYLDWV

SEQ ID NO: 218 - protein, 25 aa, Artificial Sequence
  Synthetic construct PCSK9_9
TVFTSGRCTKSIPPICFWEEYLDWV

SEQ ID NO: 219 - protein, 30 aa, Artificial Sequence
  Synthetic construct PCSK9_10
GTNEALDNNCGRATKSIPPICFYVANDLKI

SEQ ID NO: 220 - protein, 28 aa, Artificial Sequence
  Synthetic construct PCSK9_11
GTNEALDNNCGRATKSIPPICFVANDLK

SEQ ID NO: 221 - protein, 30 aa, Artificial Sequence
  Synthetic construct PCSK9_12
WEEALDWVCGRATKSIPPICFGYVANDLKI

SEQ ID NO: 222 - protein, 27 aa, Artificial Sequence
  Synthetic construct PCSK9_13
GTNEALDNNCGRATKSIPPICFTVFTS

SEQ ID NO: 223 - protein, 28 aa, Artificial Sequence
  Synthetic construct PCSK9_14
GTNEALDNNCGRATKSIPPICFGTVFTS

SEQ ID NO: 224 - protein, 26 aa, Artificial Sequence
  Synthetic construct PCSK9_2CH
TVFTSWEEALDWVGFCTYSIPPQCYG

SEQ ID NO: 225 - protein, 25 aa, Artificial Sequence
  Synthetic construct PCSK9_2EC
TVFTSWEEALDWVGICTASIPPICQ

SEQ ID NO: 226 - protein, 26 aa, Artificial Sequence
  Synthetic construct PCSK9_3CH
FCTYSIPPQCYGGTVFTSWEEALDWV

SEQ ID NO: 227 - protein, 25 aa, Artificial Sequence
  Synthetic construct PCSK9_3EC
ICTASIPPICQGTVFTSWEEALDWV

SEQ ID NO: 228 - protein, 26 aa, Artificial Sequence
  Synthetic construct PCSK9_5CH
WEEALDWVGFCTYSIPPQCYGTVFTS

SEQ ID NO: 229 - protein, 26 aa, Artificial Sequence
  Synthetic construct PCSK9_5EC
WEEALDWVGICTASIPPICQGTVFTS

SEQ ID NO: 230 - protein, 26 aa, Artificial Sequence
  Synthetic construct PCSK9_6CH
WEEYLDYVGFCTYSIPPQCYGTVFTS

SEQ ID NO: 231 - protein, 26 aa, Artificial Sequence
  Synthetic construct PCSK9_6EC
WEEYLDYVGICTASIPPICQGTVFTS

SEQ ID NO: 232 - protein, 26 aa, Artificial Sequence
  Synthetic construct PCSK9_9CH
TVFTSGFCTYSIPPQCYGWEEYLDWV

SEQ ID NO: 233 - protein, 25 aa, Artificial Sequence
  Synthetic construct PCSK9_9EC
TVFTSGICTASIPPICQWEEYLDWV

SEQ ID NO: 234 - protein, 32 aa, Artificial Sequence
  Synthetic construct Calcitonin
CSNLSTCGLGKLSQEAHKLQTYPRTNTGSGTP

SEQ ID NO: 235 - protein, 46 aa, Artificial Sequence
  Synthetic construct Cal-BT
CSNLSTCGLGKLSQEAHKLQTYPRTNTGSGTPGCARATKSIPPICF

SEQ ID NO: 236 - protein, 45 aa, Artificial Sequence
  Synthetic construct Cal-CH
CSNLSTCGLGKLSQEAHKLQTYPRTNTGSGTPGFCTYSIPPQCYG

SEQ ID NO: 237 - protein, 44 aa, Artificial Sequence
  Synthetic construct Cal-EC
CSNLSTCGLGKLSQEAHKLQTYPRTNTGSGTPGICTASIPPICQ

SEQ ID NO: 238 - protein, 14 aa, Artificial Sequence
  Synthetic construct 17A
IHVTIPADLWDWIN

SEQ ID NO: 239 - protein, 28 aa, Artificial Sequence
  Synthetic construct 17A-BT
IHVTIPADLWDWINGCGRATKSIPPICF

SEQ ID NO: 240 - protein, 27 aa, Artificial Sequence
  Synthetic construct 17A-CH
IHVTIPADLWDWINGFCTYSIPPQCYG

SEQ ID NO: 241 - protein, 26 aa, Artificial Sequence
  Synthetic construct 17A-EC
IHVTIPADLWDWINGICTASIPPICQ

SEQ ID NO: 242 - protein, 37 aa, Artificial Sequence
  Synthetic construct LL-37
LLGDFFRKSKEKIGKEFKRIVQRIKDFLRNLVPRTES

SEQ ID NO: 243 - protein, 24 aa, Artificial Sequence
  Synthetic construct Histatin 5
DSHAKRHHGYKRKFHEKHHSHRGY

SEQ ID NO: 244 - protein, 13 aa, Artificial Sequence
  Synthetic construct indolicidin
ILPWKWPWWPWRR

SEQ ID NO: 245 - protein, 22 aa, Artificial Sequence
  Synthetic construct Pexiganan
GIGKFLKKAKKFGKAFVKILKK

SEQ ID NO: 246 - protein, 27 aa, Artificial Sequence
  Synthetic construct MAF-1A
KKFKETADKLIESALQQLESSLAKEMK

SEQ ID NO: 247 - protein, 36 aa, Artificial Sequence
  Synthetic construct Sifuvirtide
SWETWEREIENYTRQIYRILEESQEQQDRNERDLLE

SEQ ID NO: 248 - protein, 36 aa, Artificial Sequence
  Synthetic construct Enfuvirtide
YTSLIHSLIEESQNQQEKNEQELLELDKWASLWNWF

SEQ ID NO: 249 - protein, 20 aa, Artificial Sequence
  Synthetic construct C1-1
SFYSVLFLWGTCGGFSHSWY

SEQ ID NO: 250 - protein, 14 aa, Artificial Sequence
  Synthetic construct p14
RRGRTGRGRRGIYR

SEQ ID NO: 251 - protein, 17 aa, Artificial Sequence
  Synthetic construct E2-550
SWFGCTWMNSTGFTKTC

SEQ ID NO: 252 - protein, 18 aa, Artificial Sequence
  Synthetic construct C5A
SWLRDIWDWICEVLSDFK

SEQ ID NO: 253 - protein, 18 aa, Artificial Sequence
  Synthetic construct cagL-cagL
KNKNFIKGIRKLMLAHNK

SEQ ID NO: 254 - protein, 20 aa, Artificial Sequence
  Synthetic construct CagA-ASPP2
GPNIQKLLYQRTTIAAMETI

SEQ ID NO: 255 - protein, 21 aa, Artificial Sequence
  Synthetic construct P1
TGTLLLILSDVNDNAPIPEPR

SEQ ID NO: 256 - protein, 24 aa, Artificial Sequence
  Synthetic construct DiaPep 277
VLGGGCALLRCIPALDSLTPANED

SEQ ID NO: 257 - protein, 39 aa, Artificial Sequence
  Synthetic construct exendin-4
HGEGTFTSDLSKQMEEEAVRLFIEWLKNGGPSSGAPPPS

SEQ ID NO: 258 - protein, 42 aa, Artificial Sequence
  Synthetic construct EGF-A1
GTNECLDNNGGCSHVCNDLKIGYECLCPDGFQLVAQRRCEDI

SEQ ID NO: 259 - protein, 26 aa, Artificial Sequence
  Synthetic construct EGF-A5
GTNECLDNNGGCSHVCNDLKIGYECL

SEQ ID NO: 260 - protein, 11 aa, Artificial Sequence
  Synthetic construct BMS-962476
PYKHSGYYHRP

SEQ ID NO: 261 - protein, 21 aa, Artificial Sequence
  Synthetic construct Tag7
ALRSNYVLKGHRDVQRTLSPG

SEQ ID NO: 262 - protein, 20 aa, Artificial Sequence
  Synthetic construct ZDC
FNMQQRFYLHPNENAKKSRD

SEQ ID NO: 263 - protein, 30 aa, Artificial Sequence
  Synthetic construct endostatin
CPAASARDFQPVLHLVALCSPLSGGMRGIR

SEQ ID NO: 264 - protein, 36 aa, Artificial Sequence
  Synthetic construct HIF-1??
GLPQLTSYDCEVNAPIQGSRNLLQGEELLRALDQVN

SEQ ID NO: 265 - protein, 23 aa, Artificial Sequence
  Synthetic construct Bcl-2 BH3
EDIIRNIARHLAQVGDSNDRSIW

SEQ ID NO: 266 - protein, 26 aa, Artificial Sequence
  Synthetic construct HVEM
ECCPKCSPGYRVKEACGELTGTVCEP

SEQ ID NO: 267 - protein, 12 aa, Artificial Sequence
  Synthetic construct pDI
LTFEHYWAQLTS

SEQ ID NO: 268 - protein, 34 aa, Artificial Sequence
  Synthetic construct PPKID4
GPSQPTYPGDDAPVRRLSFFYILLDLYLDAPGVC

(19) United States
(12) Patent Application Publication
(10) Pub. No.: US 2023/0416329 A1
(43) Pub. Date: Dec. 28, 2023

(54) A PEPTIDE WITH DISULFIDE BONDS AND INHIBITORY ACTIVITY AGAINST SERINE PROTEASES, DERIVED HYBRID PEPTIDES THEREOF, AND USES THEREOF

(71) Applicant: INSTITUTE OF MATERIA MEDICA , CHINESE ACADEMY OF MEDICAL SCICENCES, BEIJING (CN)

(72) Inventors: Wei WANG, BEIJING (CN); Zhufang SHEN, BEIJING (CN); Minzhi LIU, BEIJING (CN); Caina LI, BEIJING (CN); Sujuan SUN, BEIJING (CN); Ying MA, BEIJING (CN); Hui CAO, BEIJING (CN); Haijing ZHANG, BEIJING (CN); Yan YANG, BEIJING (CN); Lianqiu WU, BEIJING (CN)

(21) Appl. No.: 18/039,218

(22) PCT Filed: Nov. 29, 2021
(86) PCT No.: PCT/CN2021/134179

§ 371 Date: May 26, 2023

(30) Foreign Application Priority Data
Nov. 30, 2020 (CN) 202011396904.2

(51) Int. Cl. C07K 14/605; C07K 7/08; C07K 19/00; C07K 14/585; A61P 29/00; A61K 9/48

(52) U.S. Cl. CPC C07K 14/605; C07K 7/08; C07K 19/00; C07K 14/585; A61P 29/00; A61K 9/4891; A61K 38/00

(57) ABSTRACT

Provided are a polypeptide containing disulfide bonds and capable of inhibiting activity of serine protease, and a use thereof, relating to three types of linear polypeptide molecules, respectively capable of inhibiting the activity of small intestine protein metabolic enzymes such as trypsin, chymotrypsin, and elastase. Said polypeptide molecules may be broadly fused to another polypeptide or protein drug capable of treating a disease, so as to form a hybrid peptide. The hybrid peptide may inhibit the degradation of metabolic enzymes to improve the stability of a peptide or protein drug for treating a disease, such that the curative effect of direct injection administration is improved, while also facilitating direct administration absorption of the polypeptide or protein drug in the small intestine, and implementing oral administration of the protein polypeptide drug.

### FIELD OF THE INVENTION

The present invention belongs to the field of biological medicine technology, and relates to a peptide with the inhibitory activity against metabolic serine hydrolases (e.g., trypsin, chymotrypsin and elastase), or its analog having N-terminal, C-terminal, or side chain modified by PEGylation, phosphorylation, amidation or acylation, or a pharmaceutically acceptable salt thereof. Particularly, the present invention also relates to an application of active peptides with inhibitory activity against serine proteases. These peptides, their pegylated, phosphorylated, amideated or acylated analogues or pharmaceutically acceptable salts are fused with proteins, peptides or glycoproteins with therapeutic activities. They form hybrid peptides by N- or C-terminal fusion or insertion into those proteins or peptides. The hybrid polypeptides still maintain the activities of inhibiting serine proteases, thus improving the stability and efficacy of in vivo administration the of therapeutic proteins and peptides.

### BACKGROUND OF THE INVENTION

Bioactive proteins and peptides have been widely used to treat a variety of chronic and potentially life-threatening diseases such as cancer, inflammatory diseases and diabetes. The bindings between proteins and peptides with their targets are specific, namely they are of highly specific interactions with target molecules, and low specificity for non-target molecules. Long-term administration of proteins and peptides can also show low accumulation in tissues, thus reducing the side effects of the drugs. In addition, peptides are metabolized into constituent amino acids in vivo, thus reducing the risk of complications caused by toxic metabolic intermediates. At present, due to the low bioavailability caused by the stability of protein and peptide in the gastrointestinal tract and the absorption barrier related to molecular size, the subcutaneous or intravenous administration of protein and peptide drugs is still the most widely used route of administration. Although the widely used and convenient oral administration is particularly attractive to patients, there are two major obstacles that gastrointestinal the hydrolysis of gastrointestinal digestive enzymes and the low permeability of intestinal epithelial cells need to be overcome^{1,2}.

To solve the challenges related to the oral delivery of proteins and peptides, such as the stability in the gastrointestinal tract and the low-permeability absorption across the epithelial cell layer of the small intestine, many pharmaceutical technologies of oral formulations have been developed including absorption enhancers, protease inhibitors and degradable carrier materials, etc. They are helpful for overcoming the problems of protease degradation and osmotic absorption barrier in combine with enteric coating and nanoparticle technology.

The intestinal villus absorption surface of an adult is nearly 200 m², which is responsible for the absorption and transport of up to 90% nutrients in the body. Therefore, the microanatomy structure and physiological function of the small intestine indicate that it is the most ideal release position for oral delivery of protein and peptide drugs. The use of enteric-coated drug delivery system can avoid enzymatic degradation of biological drugs when they pass through the stomach and directly reach the small intestine for absorption. There is high concentration of proteolytic enzymes secreted by the pancreas or small intestinal mucosal cells in the lumen of the small intestine, which is another problem of oral administration of biological drugs. The key to obtain drugs with appropriate oral activity is to protect therapeutic proteins and peptides from proteolytic hydrolysis in the lumen of the small intestine.

In recent research reports, the application of many trypsin and chymotrypsin inhibitors, such as soybean trypsin inhibitor, pancreatic protease inhibitor and aprotinin, reduces the degradation effect of these enzymes and improves the oral bioavailability of insulin³.

Due to the low toxicity and strong inhibitory activity against peptide protease inhibitors, polypeptide protease inhibitors have so far been used to the highest extent as auxiliary agents to overcome the enzymatic barrier of perorally administered therapeutic peptides and proteins. Among these peptide protease inhibitors, a Bowman-Birk inhibitor (BBI) inhibitor of soybean trypsin inhibitor family with two inhibitory loops, is known to inhibit human trypsin as well as chymotrypsin. Moreover, these protease inhibitors of BBI family also showed inhibitory activity against elastase. This circumstance of their multiple functions is completely accord with multiple proteolytic events of the pancreatic enzymes. Therefore, these protease inhibitors have been widely used as inhibitors of therapeutic proteins and peptides against protease hydrolysis, which were publicly described in Patent Cooperation Treaty (PCT) patents WO2014191545, WO2019239405 and WO2017161184.

Compared with BBI inhibitor, sunflower trypsin inhibitor 1 (SFTI-1) is a cyclic peptide isolated from sunflower seeds that contains only 14 amino acid residues. It can be used as a protease inhibitor, an oral drug component for the treatment of diabetes, as described in PCT WO2020023386. SFTI-1 is head-tail cyclized to form a rigid structure, including two short D-folding, one intramolecular disulfide bond. These structural characteristics help to stabilize the protease inhibitory active loop of SFTI-1, which forms the molecular structure basis of its extremely strong inhibitory activity against trypsin (Kᵢ<0.1 nM)⁴. SFTI-1 has been successfully used to engineer inhibitors for an increasing number of protease therapeutic targets, including cancer-related protease inhibitors such as matriptase^{5, 6}, metorypsin⁷ and kallikrein associated protease 4 (KLK4)^{8, 9}. SFTI-1 has also been used to engineer the protease inhibitor related to skin diseases, including KLK5^{10, 11, 12, 13} and KLK7¹⁴. In addition, SFTI-1 mutants have been designed as protease inhibitors towards matriptase-2 involving in iron overload disorders¹⁵, subtilisin-like protease furin¹⁶, cathepsin G implicated in chronic inflammation^{17,18}, specific neutrophil-like elastase-like protease 3¹⁹, plasmin implicated in fibrinolysis²⁰ and chymase²¹. Besides these, the very small size and high proteolytic stability of SFTI-1 have made it an excellent scaffold for protein engineering in which peptide fragments with completely novel function can be grafted into the SFTI-1 framework, for engineering radiotherapeutics²², pro-angiogenic compounds²³, bradykinin B1 receptor antagonist²⁴, Melanocortin receptor agonists²⁵, and other peptide segments derived from annexin A1, α-fibrinogen epitopes and CD2 adhesion domain can be grafted into SFTI-1 scaffold for the treatment of inflammatory bowel diseases (IBDs)²⁶ and rheumatoid arthritis^{27,28}. However, the peptide length of these engineered protease inhibitory loops or grafted active epitopes is limited to less than 10 amino acid residues. SFTI-1 framework can't tolerate the grafting of length peptide of more than 10 amino acid residues (e.g. glucagon-like peptide-1, 30 aa) or proteins (e.g. antibodies).

The polypeptide protease inhibitors and biopharmaceuticals can be encapsulated into nanoparticle system at the same time, which can effectively protect them from enzymatic degradation and improve the intestinal absorption of polypeptide proteins and peptides. However, one of the major disadvantages of these inhibitors is that they have high toxicity, especially in the long-term use of the drug. And inhibition of protease inhibitors in the gastrointestinal tract may interfere with normal digestion and absorption of protein, causing reversible or even irreversible structural and functional damage to it. What's more, polypeptide protease inhibitors are specific and only play a role at a certain time and at certain sites. And biopharmaceuticals and polypeptide protease inhibitors must simultaneously pass through the metabolic sites. Moreover, the use of polypeptide protease inhibitors may increase the number of the intact drug at the absorption site but will not help passing through biological membranes. The presence of polypeptide protease inhibitors will affect the normal absorption of nutrition in the gastrointestinal tract, and may even generate feedback regulation to stimulate excessive secretion and expression of metabolic enzymes. Long-term treatment will lead to splenic hypertrophy and hyperplasia.

### SUMMARY OF THE INVENTION

It is accordingly an object of the present invention to provide the disulfide-constrained peptide with inhibitory activities against serine proteases, via simplifying and optimizing the inhibitory loops of polypeptide protease inhibitors of the soybean Bowman-Birk Inhibitor (BBI), sunflower trypsin inhibitor-1 (SFTI-1). The present invention also presents an application of the peptide inhibitors, or its analog having N-terminal, C-terminal, or side chain modified by PEGylation, phosphorylation, amidation, or acylation, or a pharmaceutically acceptable salt thereof, against serine proteases such as trypsin, chymotrypsin or elastase. These peptide inhibitors can be formed hybrid polypeptides by fusion with the therapeutic proteins and peptides. The formed hybrid peptides still maintain the inhibitory activity against trypsin, chymotrypsin or elastase and their tolerance to other metabolic enzymes is also enhanced, and their pharmacological activity in vivo are improved.

In a first aspect, the present invention provides a peptide, or its analog having N-terminal, C-terminal, or side chain modified by PEGylation, phosphorylation, amidation, or acylation, or a pharmaceutically acceptable salt thereof, wherein the peptide has a general formula (M):

Xaa6-Xaa5-Xaa4-Xaa3-Xaa2-Xaa1-Xaa1′-Xaa2′-Xaa3′-Xaa4′-Xaa5′-Cys6′-Xaa7′-Xaa8′ (M);
wherein:
Xaa1 is selected from the group consisting of Lys, Arg, Tyr, Phe, Ala, and Leu;
Xaa2 is selected from the group consisting of Thr and Ala;
Xaa3 is selected from the group consisting of Ala, Abu, Tyr, Nle, Ser, Gln, Leu, Ile, Val, Phe, Asn, His, Trp, Glu, Pro, Hyp, Gly, Thr, Arg, Cys, and Hcy;
Xaa4 is selected from the group consisting of Arg, Lys, Ser, Ala, Thr, Tyr, Leu, Ile, Val, Met or Arg;
Xaa5 is selected from the group consisting of Gly, Pro, Ala, Hyp, Val, Leu, Ile, Abu, Ser, Arg, Lys, Glu, Qln, and Nle, or absent;
Xaa6 is selected from the group consisting of Cys and Hcy, or absent;
Xaa1′ is selected from the group consisting of Ser and Ala;
Xaa2′ is selected from the group consisting of Ile, Leu, Nle, Arg, Phe, Tyr, Asn, Val, Met, Thr, His, Lys, Ser, Ala, Met, Asp, Trp, and Glu;
Xaa3′ is selected from the group consisting of Pro and Hyp;
Xaa4′ is selected from the group consisting of Pro, Ala, Gly, and Hyp;
Xaa5′ is selected from the group consisting of Ile, Leu, Ala, Gln, Met, Phe, Asp, Glu, His, Tyr, Ser, Thr, Val, Asn, Lys, Arg, Gly, and Trp;
Cys6′ is selected from the group consisting of Cys and Hcy;
Xaa7′ is selected from the group consisting of Phe, Tyr, Asn, Ala, Trp, His, Gln, Ser, Hyp, Val, Arg, and Ile;
Xaa8′ is selected from the group consisting of Gly and Ala, or absent;
wherein, one and only one of Xaa3 and Xaa6 must be Cys, or Hcy,
when Xaa3 is Cys or Hcy, both Xaa5 and Xaa6 are absent, and the peptide is cyclized via a disulfide bond between Xaa3 and Cys6′;
when Xaa6 is Cys or Hcy, the peptide is cyclized via a disulfide bond between Xaa6 and Cys6′.

In an embodiment, the present invention provides a peptide, or its analog having N-terminal, C-terminal or side chain modified by PEGylation, phosphorylation, amidation, or acylation, or a pharmaceutically acceptable salt thereof, wherein the peptide with inhibitory activities of serine proteases has a general formula (I):

Cys6-Xaa5-Xaa4-Xaa3-Xaa2-Xaa1-Xaa1′-Xaa2′-Xaa3′-Xaa4′-Xaa5′-Cys6′-Xaa7′ (I);
wherein, Cys6 and Cys6′ are independently selected from Cys or Hcy; the peptide is cyclized via a disulfide bond between Cys6 and Cys6′;
wherein with the proviso that
if Xaa1 is Lys; or Arg, then
Xaa2 is selected from the group consisting of Thr and Ala;
Xaa3 is selected from the group consisting of Ala, Abu, Tyr, Nle, Ser, Gln, Leu, Ile, Val, Phe, Asn, His, Trp, Glu, Pro, Hyp, and Gly;
Xaa4 is selected from the group consisting of Arg, Lys, Ser, Ala, and Thr;
Xaa5 is selected from the group consisting of Gly, Pro, Ala, Hyp, Val, Leu, Ile, Abu, Ser, Arg, Lys, Glu, Qln, and Nle;
Xaa1′ is selected from the group consisting of Ser and Ala;
Xaa2′ is selected from the group consisting of Ile, Leu, Nle, Arg, Phe, Tyr, Asn, Val, Met, Thr, His, Lys, Ser, Ala, and Met;
Xaa3′ is selected from the group consisting of Pro and Hyp;
Xaa4′ is selected from the group consisting of Pro, Ala, and Hyp;
Xaa5′ is selected from the group consisting of Ile, Leu, Ala, Gln, Met, Phe, Asp, Glu, His, Tyr, Ser, Thr, Val, Asn, Lys, Arg, and Gly;
Xaa7′ is selected from the group consisting of Phe, Tyr, Asn, Ala, Trp, His, Gln, Ser, and Hyp; wherein with the proviso that
if Xaa1 is Tyr, or Phe, then
Xaa2 is selected from the group consisting of Thr and Ala;
Xaa3 is selected from the group consisting of Ala, Abu, Gly, Tyr, Nle, Ser, Gln, Leu, Ile, Val, Phe, Asn, His, Trp, Glu, Pro, and Arg;
Xaa4 is selected from the group consisting of Ser, Ala, Phe, Thr, Lys, Tyr, Leu, Ile, Val, Met, and Arg;
Xaa5 is selected from the group consisting of Gly, Pro, Hyp, and Ala;
Xaa1′ is selected from the group consisting of Ser and Ala;
Xaa2′ is selected from the group consisting of Ile, Phe, Leu, Ala, Met, Asn, His, Asp, Tyr, Trp, and Glu;
Xaa3′ is selected from the group consisting of Pro and Hyp;
Xaa4′ is selected from the group consisting of Pro, Ala, Gly and Hyp;
Xaa5′ is selected from the group consisting of Ile, Leu, Gln, Met, Arg, Phe, His, Lys, Arg, Trp, Tyr, Ala, Ser, Thr, Val, Asp, Asn, Glu, and Gly;
Xaa7′ is selected from the group consisting of Tyr, Phe, Asn, Val, Arg, Ile, Gln, Ser, and His; wherein with the proviso that
if Xaa1 is Ala, or Leu, then
Xaa2 is selected from the group consisting of Thr and Ala;
Xaa3 is selected from the group consisting of Ala, Abu, Gly, Tyr, Nle, Ser, Gln, Leu, Ile, Val, Phe, Asn, His, Trp, Glu, Pro, and Arg;
Xaa4 is selected from the group consisting of Ile, Leu, Val, Ala, and Tyr;
Xaa5 is selected from the group consisting of Gly, Pro, Hyp, and Ala;
Xaa1′ is selected from the group consisting of Ser and Ala;
Xaa2′ is selected from the group consisting of Ile, Asn, Tyr, and Ala;
Xaa3′ is selected from the group consisting of Pro and Hyp;
Xaa4′ is selected from the group consisting of Pro, Hyp and Ala;
Xaa5′ is selected from the group consisting of Ile and Gln;
Xaa7′ is selected from the group consisting of Gln, Tyr, Arg, His and Asn;
   wherein peptides in the claims does not include the peptide SEQ ID NO: 1.

Hereinafter the amino acid or its residue listed in Table 1 is provided and suitable for the present invention. The abbreviations used herein follow the naming conventions suggested by the IUPAC Commission on the Nomenclature of Organic Chemistry and the IUPAC-IUB Commission on Biochemical Nomenclature.

TABLE 1
Nomenclature of Amino Acids

| | Abbreviation | Abbreviation |
|---|---|---|
| Definition | (one letter) | (three letters) |
| L-Alanine | A | Ala |
| L-Arginine | R | Arg |
| L-Asparagine | N | Asn |
| L-Aspartic acid | D | Asp |
| L-Cysteine | C | Cys |
| L-Glutamine | Q | Gln |
| L-Glutamic acid | E | Glu |
| Glycine | G | Gly |
| L-Histidine | H | His |
| L-Isoleucine | I | Ile |
| L-Leucine | L | Leu |
| L-Lysine | K | Lys |
| L-Methionine | M | Met |
| L-Phenylalanine | F | Phe |
| L-Proline | P | Pro |
| L-Serine | S | Ser |
| L-Threonine | T | Thr |
| L-Tryptophan | W | Trp |
| L-Tyrosine | Y | Tyr |
| L-Valine | V | Val |
| L-Homocysteine | | Hcy |
| L-2-Aminobutyric acid | | Abu |
| L-Norleucine | | Nle |
| L-4-Hydroxyproline | | Hyp |

In a particular embodiment, the present invention provides a peptide, or its analog having N-terminal, C-terminal or side chain modified by PEGylation, phosphorylation, amidation, or acylation, or a pharmaceutically acceptable salt thereof, wherein the peptide is preferably with anti-trypsin activity among its inhibitory activities of serine proteases.
wherein:
Xaa1 is selected from the group consisting of Lys and Arg;
Xaa2 is selected from the group consisting of Thr and Ala;
Xaa3 is selected from the group consisting of Ala, Abu, Tyr, Gly, Nle, Ser, Thr, and Gln;
Xaa4 is selected from the group consisting of Arg, Lys, Ser, Ala, and Thr;
Xaa5 is selected from the group consisting of Ala, Gly, and Pro;
Xaa1′ is selected from the group consisting of Ser and Ala;
Xaa2′ is selected from the group consisting of Ile, Leu, Nle and Ala;
Xaa3′ is selected from the group consisting of Pro and Hyp;
Xaa4′ is selected from the group consisting of Pro and Ala;
Xaa5′ is selected from the group consisting of Ile, Ala, and Gln; and
Xaa7′ is selected from the group consisting of Phe and Tyr.

In another particularly preferred embodiment of the present invention, the anti-trypsin peptide or its analog having N-terminal, C-terminal or side chain modified by PEGylation, phosphorylation, amidation, or acylation, or a pharmaceutically acceptable salt thereof, is selected from the group consisting of the following sequences: SEQ ID NO: 9, SEQ ID NO: 10, SEQ ID NO: 16, SEQ ID NO: 17, SEQ ID NO: 25, SEQ ID NO: 27, SEQ ID NO: 28, SEQ ID NO: 35, SEQ ID NO: 46, SEQ ID NO: 47, SEQ ID NO: 49, SEQ ID NO: 50, SEQ ID NO: 51, SEQ ID NO: 53, SEQ ID NO: 54, SEQ ID NO: 55, SEQ ID NO: 57, SEQ ID NO: 60, SEQ ID NO: SEQ ID NO: 67, SEQ ID NO: 69, SEQ ID NO: 70, SEQ ID NO: 71, SEQ ID NO: 74, SEQ ID NO: 75, SEQ ID NO: 76, SEQ ID NO: 77, SEQ ID NO: 78, and SEQ ID NO: 79.

In another more particularly preferred embodiment of the present invention, the anti-trypsin peptide or its analog having N-terminal, C-terminal or side chain modified by PEGylation, phosphorylation, amidation, or acylation, or a pharmaceutically acceptable salt thereof, is selected from the group consisting of the following sequences: SEQ ID NO: 9, SEQ ID NO: 35, SEQ ID NO: 47, SEQ ID NO: 50, SEQ ID NO: 53, SEQ ID NO: 54, SEQ ID NO: 67, SEQ ID NO: 75, SEQ ID NO: 76, SEQ ID NO: 77, SEQ ID NO: 78 and SEQ ID NO: 79.

In another particular embodiment, the present invention provides a peptide, or its analog having N-terminal, C-terminal or side chain modified by PEGylation, phosphorylation, amidation, or acylation, or a pharmaceutically acceptable salt thereof, wherein the peptide is preferably with anti-chymotrypsin among its inhibitory activities of serine proteases.
wherein
Xaa1 is selected from the group consisting of Tyr and Phe;
Xaa2 is selected from the group consisting of Thr and Ala;
Xaa3 is selected from the group consisting of Ala and Abu;
Xaa4 is selected from the group consisting of Ser, Ala, Phe, and Thr;
Xaa5 is selected from the group consisting of Ala, Gly, and Pro;
Xaa1′ is Ser;
Xaa2′ is selected from the group consisting of Ile, Ala, and Asn;
Xaa3′ is selected from the group consisting of Pro and Hyp;
Xaa4′ is selected from the group consisting of Pro, Ala, and Hyp;
Xaa5′ is selected from the group consisting of Ile and Gln;
Xaa7′ is selected from the group consisting of Tyr, Phe, Asn, Gln, and His; and
Xaa8′ is selected from the group consisting of Gly and Ala, or absent.

In another more particularly preferred embodiment of the present invention, the anti-chymotrypsin peptide or its analog having N-terminal, C-terminal or side chain modified by PEGylation, phosphorylation, amidation, or acylation, or a pharmaceutically acceptable salt thereof, is selected from the group consisting of the following sequences: SEQ ID NO: 93, SEQ ID NO: 95, SEQ ID NO: 103, SEQ ID NO: 104, SEQ ID NO: 107, SEQ ID NO: 111 and SEQ ID NO: 112.

In another particular embodiment, the present invention provides a peptide, or its analog having N-terminal, C-terminal or side chain modified by PEGylation, phosphorylation, amidation, or acylation, or a pharmaceutically acceptable salt thereof, wherein the peptide is preferably with inhibitory activity against chymotrypsin-like elastase among its inhibitory activities of serine proteases.
wherein
Xaa1 is selected from the group consisting of Ala and Leu;
Xaa2 is selected from the group consisting of Thr and Ala;
Xaa3 is selected from the group consisting of Ala, Abu, Gly, Tyr, Nle, Ser, Gln, Leu, Ile, Val, Phe, Asn, His, Trp, Glu, Pro, and Arg;
Xaa4 is selected from the group consisting of Ile, Leu, Val, Ala, and Tyr;
Xaa5 is selected from the group consisting of Gly, Pro, Ala, and Hyp;
Xaa1′ is selected from the group consisting of Ser and Ala;
Xaa2′ is selected from the group consisting of Ile and Asn;
Xaa3′ is selected from the group consisting of Pro and Hyp;
Xaa4′ is selected from the group consisting of Pro and Hyp;
Xaa5′ is selected from the group consisting of Ile and Gln; and
Xaa7′ is selected from the group consisting of Gln and Tyr.

In another more particularly preferred embodiment, the present invention provides a peptide, or its analog having N-terminal, C-terminal or side chain modified by PEGylation, phosphorylation, amidation, or acylation, or a pharmaceutically acceptable salt thereof, wherein the peptide with inhibitory activity against elastase is selected from the group consisting of the following sequences: SEQ ID NO: 140 and SEQ ID NO: 165.

In another embodiment, the present invention provides a peptide, or its analog having N-terminal, C-terminal or side chain modified by PEGylation, phosphorylation, amidation, or acylation, or a pharmaceutically acceptable salt thereof, wherein the peptide with inhibitory activities of serine proteases has a general formula (II):

Xaa4-Cys3-Xaa2-Xaa1-Xaa1′-Xaa2′-Xaa3′-Xaa4′-Xaa5′-Cys6′-Xaa7′-Xaa8′ (II);
wherein, Cys3 or Cys6′ are independently selected from Cys or Hcy; the peptide is cyclized via a disulfide bond between Cys3 and Cys6′;
wherein with the proviso that
if Xaa1 is Lys; or Arg, then
Xaa2 is selected from the group consisting of Thr and Ala;
Xaa4 is selected from the group consisting of Arg, Lys, Ser, Ala, and Thr;
Xaa1′ is selected from the group consisting of Ser and Ala;
Xaa2′ is selected from the group consisting of Ile, Leu, Nle, Arg, Phe, Tyr, Asn, Val, Met, Thr, His, Lys, Ser, Ala and Met;
Xaa3′ is selected from the group consisting of Pro and Hyp;
Xaa4′ is selected from the group consisting of Pro, Ala and Hyp;
Xaa5′ is selected from the group consisting of Ile, Leu, Ala, Gln, Met, Phe, Asp, Glu, His, Tyr, Ser, Thr, Val, Asn, Lys, Arg and Gly;
Xaa7′ is selected from the group consisting of Phe, Tyr, Asn, Ala, Trp, His, Gln, Ser and Hyp;
Xaa8′ is absent;
wherein with the proviso that
if Xaa1 is Tyr, or Phe, then
Xaa2 is selected from the group consisting of Thr and Ala;
Xaa4 is selected from the group consisting of Ser, Ala, Phe, Thr, Lys, Tyr, Leu, Ile, Val, Met and Arg;
Xaa1′ is selected from the group consisting of Ser and Ala;
Xaa2′ is selected from the group consisting of Ile, Phe, Leu, Ala, Met, Asn, His, Asp, Tyr, Trp and Glu;
Xaa3′ is selected from the group consisting of Pro and Hyp;
Xaa4′ is selected from the group consisting of Pro, Ala, Gly and Hyp;
Xaa5′ is selected from the group consisting of Ile, Leu, Gln, Met, Arg, Phe, His, Lys, Arg, Trp, Tyr, Ala, Ser, Thr, Val, Asp, Asn, Glu and Gly;
Xaa7′ is selected from the group consisting of Tyr, Phe, Asn, Val, Arg, Ile, Gln, Ser and His;
Xaa8′ is selected from the group consisting of Gly and Ala, or absent;
Wherein with the proviso that
If Xaa1 is Ala, or Leu, then
Xaa2 is selected from the group consisting of Thr, or Ala;
Xaa4 is selected from the group consisting of Ile, Leu, Val, Ala, or Tyr;
Xaa1′ is selected from the group consisting of Ser and Ala;
Xaa2′ is selected from the group consisting of Ile, Asn, Tyr and Ala;
Xaa3′ is selected from the group consisting of Pro and Hyp;
Xaa4′ is selected from the group consisting of Pro, Hyp and Ala;
Xaa5′ is selected from the group consisting of Ile and Gln;
Xaa7′ is selected from the group consisting of Gln, Tyr, Arg, His and Asn;
Xaa8′ is absent;
wherein peptides in the claims does not include the peptide SEQ ID NO: 1.

In a particular embodiment, the present invention provides a peptide, or its analog having N-terminal, C-terminal or side chain modified by PEGylation, phosphorylation, amidation, or acylation, or a pharmaceutically acceptable salt thereof, wherein the peptide is preferably with anti-trypsin activity among its inhibitory activities of serine proteases.
wherein
Xaa1 is selected from the group consisting of Lys and Arg;
Xaa2 is selected from the group consisting of Thr and Ala;
Xaa4 is selected from the group consisting of Arg, Lys, Ser, Ala and Thr;
Xaa1′ is selected from the group consisting of Ser and Ala;
Xaa2′ is selected from the group consisting of Ile, Leu, Nle and Ala;
Xaa3′ is selected from the group consisting of Pro and Hyp;
Xaa4′ is selected from the group consisting of Pro and Ala;
Xaa5′ is selected from the group consisting of Ile, Ala and Gln;
Xaa7′ is selected from the group consisting of Phe and Tyr; and
Xaa8′ is absent.

In another particularly preferred embodiment of the present invention, the anti-trypsin peptide, or its analog having N-terminal, C-terminal or side chain modified by PEGylation, phosphorylation, amidation, or acylation, or a pharmaceutically acceptable salt thereof, is selected from the group consisting of the following sequences: SEQ ID NO: 45, SEQ ID NO: 65 and SEQ ID NO: 66.

In another particular embodiment, the present invention provides a peptide, or its analog having N-terminal, C-terminal or side chain modified by PEGylation, phosphorylation, amidation, or acylation, or a pharmaceutically acceptable salt thereof, wherein the peptide is preferably with anti-chymotrypsin activity among its inhibitory activities of serine proteases.
wherein
Xaa1 is selected from the group consisting of Tyr and Phe;
Xaa2 is selected from the group consisting of Thr and Ala;
Xaa4 is selected from the group consisting of Ser, Ala, Phe and Thr;
Xaa1′ is Ser;
Xaa2′ is selected from the group consisting of Ile, Ala and Asn;
Xaa3′ is selected from the group consisting of Pro and Hyp;
Xaa4′ is selected from the group consisting of Pro, Ala and Hyp;
Xaa5′ is selected from the group consisting of Ile and Gln;
Xaa7′ is selected from the group consisting of Tyr, Phe, Asn, Gln and His; and
Xaa8′ is Gly, or absent.

In another particularly preferred embodiment of the present invention, the anti-chymotrypsin peptide or its analog having N-terminal, C-terminal or side chain modified by PEGylation, phosphorylation, amidation, or acylation, or a pharmaceutically acceptable salt thereof, is selected from the group consisting of the following sequences: SEQ ID NO: 85, SEQ ID NO: 90, SEQ ID NO: 91, SEQ ID NO: 98, SEQ ID NO: 105, SEQ ID NO: 106, SEQ ID NO: 113, SEQ ID NO: 114, SEQ ID NO: 115, SEQ ID NO: 131, SEQ ID NO: 132 and SEQ ID NO: 133.

In another more particularly preferred embodiment of the present invention, the anti-chymotrypsin peptide, or its analog having N-terminal, C-terminal or side chain modified by PEGylation, phosphorylation, amidation, or acylation, or a pharmaceutically acceptable salt thereof, is selected from the following the group consisting of sequences: SEQ ID NO: 85 and SEQ ID NO: 90.

In another particular embodiment, the present invention provides a peptide, or its analog having N-terminal, C-terminal or side chain modified by PEGylation, phosphorylation, amidation, or acylation, or a pharmaceutically acceptable salt thereof, wherein the peptide is preferably with inhibitory activity against chymotrypsin-like elastase among its inhibitory activities of serine proteases.
wherein
Xaa1 is selected from the group consisting of Ala and Leu;
Xaa2 is selected from the group consisting of Thr and Ala;
Xaa4 is selected from the group consisting of Ile, Leu, Val, Ala and Tyr;
Xaa1′ is selected from the group consisting of Ser and Ala;
Xaa2′ is selected from the group consisting of Ile and Asn;
Xaa3′ is selected from the group consisting of Pro and Hyp;
Xaa4′ is selected from the group consisting of Pro and Hyp;
Xaa5′ is selected from the group consisting of Ile and Gln;
Xaa7′ is selected from the group consisting of Gln and Tyr; and
Xaa8′ is absent.

In another particularly preferred embodiment, the present invention provides a peptide, or its analog having N-terminal, C-terminal or side chain modified by PEGylation, phosphorylation, amidation, or acylation, or a pharmaceutically acceptable salt thereof, wherein the peptide with inhibitory activity against elastase is selected from the group consisting of the following sequences: SEQ ID NO: 134, SEQ ID NO: 145, SEQ ID NO: 151, SEQ ID NO: 155, SEQ ID NO: 156, SEQ ID NO: 158 and SEQ ID NO: 162.

In another more particularly preferred embodiment, the present invention provides a peptide, or its analog having N-terminal, C-terminal or side chain modified by PEGylation, phosphorylation, amidation, or acylation, or a pharmaceutically acceptable salt thereof, wherein the peptide with inhibitory activity against elastase is selected from the group consisting of the following sequences: SEQ ID NO: 145, SEQ ID NO: 155 and SEQ ID NO: 156.

In a particular embodiment, the present invention provides peptide inhibitors against serine proteases, in which trypsin, chymotrypsin and elastase are preferable.

The present invention also provides a hybrid peptide, which comprises the peptide inhibiting serine proteases. The peptide, or its analog having N-terminal, C-terminal or side chain modified by PEGylation, phosphorylation, amidation, or acylation, or a pharmaceutically acceptable salt thereof, was fused to N-terminus or C-terminus of a therapeutical protein or peptide, or inserted into an intramolecular location of a therapeutical protein or peptide to form a hybrid peptide. The hybrid peptide has a general formula selected from the group, consisting of:

B-L-A (III);

A-L-B (IV);

A1-L1-B-L2-A2 (V);
wherein
the molecular mass of the hybrid peptide is 1.5 kDa to 30 kDa;
B is a disulfide-constrained peptide with inhibitory activity against serine proteases, or its analog having N-terminal, C-terminal or side chain modified by PEGylation, phosphorylation, amidation, or acylation, or a pharmaceutically acceptable salt thereof;
L is a linker which optionally has 1, 2, 3, 4 or 5 glycine or proline residues;
A is a bioactive oligopeptide;
A1 and A2 are peptide segments of N-terminal and C-terminal of bioactive oligopeptide A, respectively;
L1 and L2 are linkers which optionally have 1, 2, 3, 4 or 5 glycine or proline residues, or absent.

In one aspect, the present invention provides a method for the application of therapeutic glucagon like peptide-1 (GLP-1), or its analog containing N-terminal, C-terminal or side chain modified by PEGylation, phosphorylation, amidation, or acylation, or a pharmaceutically acceptable salt thereof, which is attached with peptide inhibitors against serine proteases. The hybrid peptide formed with serine protease inhibitor described above, is selected from the group consisting of the following sequences: SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208 and SEQ ID NO: 209. The hybrid peptide is applied to treat type II diabetes and/or obesity.

In another aspect, the present invention provides a method for the use of a therapeutically active peptide SEQ ID NO: 210, or its analog having N-terminal, C-terminal or side chain modified by PEGylation, phosphorylation, amidation, or acylation, or a pharmaceutically acceptable salt thereof, which is attached with peptide inhibitors against serine proteases. The active peptide has the ability of inhibiting the protein-protein interaction between proprotein convertase subtilisin/kexin type 9 kexin preprotein converting enzyme (PCSK9) and low-density lipoprotein receptor (LDLR) protein. The hybrid peptide formed with protease inhibitor described above is selected from the group consisting of the following sequences: SEQ ID NO: 211, SEQ ID NO: 212, SEQ ID NO: 214, SEQ ID NO: 215, SEQ ID NO: 216, SEQ ID NO: 217, SEQ ID NO: 218, SEQ ID NO:224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232 and SEQ ID NO: 233. The hybrid peptide is applied to treat familial hypercholesterolemia.

In another aspect, the present invention provides a method for the use of a therapeutically active peptide salmon calcitonin (SEQ ID NO: 234), or its analog having N-terminal, C-terminal or side chain modified by PEGylation, phosphorylation, amidation, or acylation, or a pharmaceutically acceptable salt thereof, which is attached with peptide inhibitors against serine proteases. The hybrid peptide formed with protease inhibitor described above is selected from the group consisting of the following sequences SEQ ID NO: 235, SEQ ID NO: 236, and SEQ ID NO: 237. The hybrid peptide is applied to treat bone related diseases and calcium disorders such as osteoporosis and/or osteoarthritis.

In another aspect, the present invention provides a method for the use of a therapeutically active peptide (SEQ ID NO: 238), or its analog having N-terminal, C-terminal or side chain modified by PEGylation, phosphorylation, amidation, or acylation, or a pharmaceutically acceptable salt thereof, which is attached with peptide inhibitors against serine proteases. The active peptide has the ability of inhibiting the interaction between IL-17A and IL-17RA, and its hybrid peptide formed with protease inhibitor described above is selected from the group consisting of the following sequences SEQ ID NO: 239, SEQ ID NO: 240, and SEQ ID NO: 241. The hybrid peptide is applied to treat inflammatory diseases, including inflammatory lung disease, asthma, chronic obstructive pulmonary disease, inflammatory bowel disease, arthritis, autoimmune diseases, rheumatoid arthritis, psoriasis, and systemic sclerosis.

The present invention also provides a peptide composition that can contain at least one, two, or three peptides or analogues thereof having the structure shown in Formula (I) or (II), or a pharmaceutically acceptable salt thereof, as well as one or more hybrid peptides, or analogues thereof, or a pharmaceutically acceptable salt thereof.

In a particular embodiment, a hybrid peptide composition comprises of a hybrid therapeutic glucagon-like peptide-1 (GLP-1), or its analog having N-terminal, C-terminal or side chain modified by PEGylation, phosphorylation, amidation, or acylation, or a pharmaceutically acceptable salt thereof, which is formed with protease inhibitor described above. Wherein the hybrid peptide is selected from the group consisting of the following sequences: SEQ ID NO: 200, SEQ ID NO: 204, and SEQ ID NO: 208.

In a particular embodiment, a hybrid peptide composition comprises of a hybrid therapeutic active peptide (SEQ ID NO: 210), or its analog having N-terminal, C-terminal or side chain modified by PEGylation, phosphorylation, amidation, or acylation, or a pharmaceutically acceptable salt thereof, which is formed with protease inhibitor described above. Wherein the hybrid peptide is selected from the group consisting of the following sequences: SEQ ID NO: 211, SEQ ID NO: 212, SEQ ID NOs: 214-216, SEQ ID NO: 218 and SEQ ID NOs: 224-233.

In a particular embodiment, a hybrid peptide composition comprises of a hybrid therapeutic active peptide salmon calcitonin (SEQ ID NO: 234), or its analog having N-terminal, C-terminal or side chain modified by PEGylation, phosphorylation, amidation, or acylation, or a pharmaceutically acceptable salt thereof, which is formed with protease inhibitor described above. Wherein the hybrid peptide is selected from the group consisting of the following sequences: SEQ ID NOs: 235-237.

In another particular embodiment, a hybrid peptide composition comprises of a hybrid therapeutic active peptide (SEQ ID NO: 238), or its analog having N-terminal, C-terminal or side chain modified by PEGylation, phosphorylation, amidation, or acylation, or a pharmaceutically acceptable salt thereof, which is formed with protease inhibitor described above. Wherein the hybrid peptide is selected from the group consisting of the following sequences: SEQ ID NOs: 239-241.

In an aspect, the present invention provides pharmaceutical excipients that can be co administered, further comprising pharmaceutically acceptable carriers, diluents, dispersants, promoters, and/or excipients, to promote the permeation and absorption of biologically active hybrid peptides or a pharmaceutically acceptable salt through the intestinal epithelium.

In another aspect, the present invention provides a method of administration of biologically active hybrid peptides or a pharmaceutically acceptable salt, suitable for injection and/or oral administration.

In an embodiment, the present invention provides a protectively pharmaceutical delivery tool including enteric coated capsules, microcapsules, or particles that effectively transport bioactive hybrid peptides or biological therapeutic agents to the intestinal absorption site, blocking the contact and degradation of bioactive hybrid peptides or a pharmaceutically acceptable salt with pepsin.

In another embodiment, the protease inhibitors, therapeutic oligopeptides, and hybrid peptides in the present invention, SEQ ID NOs: 1-241, as described above, can be obtained using well-known peptide synthesis techniques such as classical solid phase or liquid phase synthesis or synthesized using recombinant DNA technology.

Beneficial technical effects: the invention can improve the stability of bioactive peptides for treating various diseases in vivo, promote the realization of oral administration, improve patient compliance with medication, and reduce side effects, with beneficial economic value.

In order that the invention may be more readily understood and put into practice, one or more preferred embodiments thereof will now be described, only by way of example, with reference to the accompanying drawings.

### BRIEF DESCRIPTION OF THE DRAWINGS

The various features of the present invention have particularities in the claims. Referring to the following detailed description, a better understanding of the features and advantages of the invention will be obtained, utilizing the principles of the invention in the illustrative embodiment, the figures include:

FIG. 1. Determination of the Michaelis constant Kₘ of trypsin. The Michaelis constant Kₘ of trypsin hydrolysis substrate BApNA can be obtained by plotting the initial velocity V₀ with the concentration of BApNA using Prism software. The experiment was performed with three replicates, and data were expressed as “mean±standard deviation”.

FIG. 2. Determination of the inhibitory activities of peptides against trypsin. By adding different concentrations of trypsin inhibitory peptides (BT1, BT2, BT3, and BT45), their inhibitory effects on trypsin were tested, and their concentrations of 50% inhibition of enzyme activity (IC₅₀ value) were measured. The experiment was performed with three replicates, and data were expressed as “mean±standard deviation”.

FIG. 3. Determination of the inhibitory activities of peptides against trypsin. By adding different concentrations of trypsin inhibitory peptides (BT1, BT5, BT6, and BT7), their inhibitory effects on trypsin were tested, and their concentrations of 50% inhibition of enzyme activity (IC₅₀ value) were measured. The experiment was performed with three replicates, and data were expressed as “mean±standard deviation”.

FIG. 4. Determination of the inhibitory activities of peptides against trypsin. By adding different concentrations of trypsin inhibitory peptides (BT45, BT9, BT10, BT11, BT15, BT16, BT17, BT27, and BT28), their inhibitory effects on trypsin were tested, and their concentrations of 50% inhibition of enzyme activity (IC₅₀ value) were measured. The experiment was performed with three replicates, and data were expressed as “mean±standard deviation”.

FIG. 5. Determination of the inhibitory activities of peptides against trypsin. By adding different concentrations of trypsin inhibitory peptides (BT9, BT25, BT26, BT35, BT47, BT50, BT53, and BT54), their inhibitory effects on trypsin were tested, and their concentrations of 50% inhibition of enzyme activity (IC₅₀ value) were measured. The experiment was performed with three replicates, and data were expressed as “mean±standard deviation”.

FIG. 6. Determination of the inhibitory activities of peptides against trypsin. By adding different concentrations of trypsin inhibitory peptides (BT9, BT25, BT26, BT66 and BT67), their inhibitory effects on trypsin were tested, and their concentrations of 50% inhibition of enzyme activity (IC₅₀ value) were measured. The experiment was performed with three replicates, and data were expressed as “mean±standard deviation”.

FIG. 7. Determination of the Michaelis constant Kₘ of chymotrypsin. The Michaelis constant Kₘ of chymotrypsin hydrolysis substrate AAPFpNA can be obtained by plotting the initial velocity V₀ with the concentration of AAPFpNA using Prism software. The experiment was performed with three replicates, and data were expressed as “mean±standard deviation”.

FIG. 8. Determination of the inhibitory activities of peptides against chymotrypsin. By adding different concentrations of trypsin inhibitory peptides (CH1, CH4, CH5 and CH7), their inhibitory effects on chymotrypsin were tested, and their concentrations of 50% inhibition of enzyme activity (IC₅₀ value) were measured. The experiment was performed with three replicates, and data were expressed as “mean±standard deviation”.

FIG. 9. Determination of the inhibitory activities of peptides against chymotrypsin. By adding different concentrations of trypsin inhibitory peptides (CH5, CH10, CH11, CH13, CH17, CH18, CH19, CH23 and CH24), their inhibitory effects on chymotrypsin were tested, and their concentrations of 50% inhibition of enzyme activity (IC₅₀ value) were measured. The experiment was performed with three replicates, and data were expressed as “mean±standard deviation”.

FIG. 10. Determination of the inhibitory activities of peptides against chymotrypsin. By adding different concentrations of trypsin inhibitory peptides (CH10, CH26, CH27, CH31, CH32, CH33, CH34 and CH35), their inhibitory effects on chymotrypsin were tested, and their concentrations of 50% inhibition of enzyme activity (IC₅₀ value) were measured. The experiment was performed with three replicates, and data were expressed as “mean±standard deviation”.

FIG. 11. Determination of the inhibitory activities of peptides against chymotrypsin. By adding different concentrations of trypsin inhibitory peptides (CH10, CH47, CH49, CH51, CH52 and CH53), their inhibitory effects on chymotrypsin were tested, and their concentrations of 50% inhibition of enzyme activity (IC₅₀ value) were measured. The experiment was performed with three replicates, and data were expressed as “mean±standard deviation”.

FIG. 12. Determination of the Michaelis constant Kₘ of elastase. The Michaelis constant Kₘ, of elastase hydrolysis substrate AAAPFpNA can be obtained by plotting the initial velocity V₀ with the concentration of AAAPFpNA using Prism software. The experiment was performed with three replicates, and data were expressed as “mean±standard deviation”.

FIG. 13. Determination of the inhibitory activities of peptides against elastase. By adding different concentrations of elastase inhibitory peptides (EC1, EC2, EC7 and EC12), their inhibitory effects on elastase were tested, and their concentrations of 50% inhibition of enzyme activity (IC₅₀ value) were measured. The experiment was performed with three replicates, and data were expressed as “mean±standard deviation”.

FIG. 14. Determination of the inhibitory activities of peptides against elastase. By adding different concentrations of elastase inhibitory peptides (EC12, EC18, EC19, EC22, EC23 and EC29), their inhibitory effects on elastase were tested, and their concentrations of 50% inhibition of enzyme activity (IC₅₀ value) were measured. The experiment was performed with three replicates, and data were expressed as “mean±standard deviation”.

FIG. 15. Analyses of enzymatic degradation of GLP-1 and its analogues by DPP-IV. 25 μM of GLP-1 and its analogues were incubated with 0.5 ng/μL of DPP-IV in 100 mM Tris-HCl buffer (pH 8.0) at 37° C. for 12 h. The amount of the prototype peptide at 0 h was taken as 100%. At different time point, 50 μL aliquots was taken out, and 10% (v/v) TFA was added to terminate the reaction. The remaining percentage (%) of the peptide relative to the prototype peptide at that time point was analyzed by reverse phase high performance liquid chromatography. The experiment was performed with three replicates, and data were expressed as “mean±standard deviation”. A, SEQ ID NO: 186-190, SEQ ID NO: 192, SEQ ID NO: 193; B, SEQ ID NO: 194-201; C, SEQ ID NO: 202-205; D, SEQ ID NO: 206-209.

FIG. 16. Analyses of enzymatic degradation of GLP-1 and its analogues by NEP24.11. 30 μM of GLP-1 and its analogues were incubated with 1.0 ng/μL of NEP24.11 in 50 mM HEPES, 50 mM NaCl buffer (pH 7.4) at 37° C. for 8 h. The amount of the prototype peptide at 0 h was taken as 100%. At different time point, 50 μL aliquots was taken out, and 10% (v/v) TFA was added to terminate the reaction. The remaining percentage (%) of the peptide relative to the prototype peptide at that time point was analyzed by reverse phase high performance liquid chromatography. The experiment was performed with three replicates, and data were expressed as “mean±standard deviation”. A, SEQ ID NO: 186-193; B, SEQ ID NO: 194-201.

FIG. 17. Analyses of enzymatic degradation of GLP-1 and its analogues by trypsin. 60 μM of GLP-1 and its analogues were incubated with 2.0 ng/μL of trypsin in 50 mM Tris, 20 mM CaCl₂ buffer (pH 7.8) at 37° C. for 9 min or 60 min. The amount of the prototype peptide at 0 h was taken as 100%. At different time point, 25 μL aliquots was taken out, and 10% (v/v) TFA was added to terminate the reaction. The remaining percentage (%) of the peptide relative to the prototype peptide at that time point was analyzed by reverse phase high performance liquid chromatography. The experiment was performed with three replicates, and data were expressed as “mean±standard deviation”. A, SEQ ID NO: 186-193; B, SEQ ID NO: 194, SEQ ID NO: 196, SEQ ID NO: 198, SEQ ID NO: 200; C, SEQ ID NO: 195, SEQ ID NO: 197, SEQ ID NO: 199, SEQ ID NO: 201.

FIG. 18. Analyses of enzymatic degradation of GLP-1 and its analogues by chymotrypsin. 60 μM of GLP-1 and its analogues were incubated with 1.0 ng/μL of chymotrypsin in 50 mM Tris, 20 mM CaCl₂ buffer (pH 7.8) at 37° C. for 9 min or 60 min. The amount of the prototype peptide at 0 h was taken as 100%. At different time point, 25 μL aliquots was taken out, and 10% (v/v) TFA was added to terminate the reaction. The remaining percentage (%) of the peptide relative to the prototype peptide at that time point was analyzed by reverse phase high performance liquid chromatography. The experiment was performed with three replicates, and data were expressed as “mean±standard deviation”. A, SEQ ID NO: 186-193; B, SEQ ID NO: 194-201; C, SEQ ID NO: 202-205.

FIG. 19. Analyses of enzymatic degradation of GLP-1 and its analogues by elastase. 60 μM of GLP-1 and its analogues (SEQ ID NO: 206-209) were incubated with 10 ng/μL of elastase in 50 mM Tris buffer (pH 8.0) at 37° C. for 60 min. The amount of the prototype peptide at 0 h was taken as 100%. At different time point, 25 μL aliquots was taken out, and 10% (v/v) TFA was added to terminate the reaction. The remaining percentage (%) of the peptide relative to the prototype peptide at that time point was analyzed by reverse phase high performance liquid chromatography. The experiment was performed with three replicates, and data were expressed as “mean±standard deviation”.

FIG. 20. Analyses of enzymatic degradation of GLP-1 and its analogues by human serum. 30 μM of GLP-1 and its analogues were incubated with 25% (v/v) of human serum in 50 mM Tris buffer (pH 7.0) at 37° C. for 12 h. The amount of the prototype peptide at 0 h was taken as 100%. At different time point, 100 μL aliquots was taken out, and 300 μL precooled anhydrous methanol was added to terminate the reaction. The remaining percentage (%) of the peptide relative to the prototype peptide at that time point was analyzed by reverse phase high performance liquid chromatography. The experiment was performed with three replicates, and data were expressed as “mean±standard deviation”. The sample was subjected to high-speed centrifugation, supernatant extraction, and freeze drying, followed by dissolution in 50% (v/v) methanol/water solution, and analyzed by reverse phase high performance liquid chromatography. The experiment was performed with three replicates, and data were expressed as “mean±standard deviation”. A, SEQ ID NO: 194, SEQ ID NO: 196, SEQ ID NO: 198, SEQ ID NO: 200; B, SEQ ID NO: 202-205; C, SEQ ID NO: 206-209.

FIG. 21. In vivo hypoglycemic activities of GLP-1 analogues by subcutaneous administration. Normal ICR mice were subcutaneously injected with GLP-1 and its analogues or corresponding volumes of saline (1.0 μmol/kg, n=10). After 30 min, glucose solution (2 g/kg) was administered by gavage, and blood was collected from the tail tip at 30, 60, and 120 min after glucose administration. Blood glucose was measured using glucose oxidase method, and blood glucose values and area under the blood glucose curve (AUC) were calculated at each timepoint. Data are expressed as “mean±standard error”, and p<0.05 is considered to be statistically significant. A, SEQ ID NO: 194, SEQ ID NO: 196, SEQ ID NO: 198, SEQ ID NO: 200; B, SEQ ID NO: 195, SEQ ID NO: 197, SEQ ID NO: 199, SEQ ID NO: 201; C, SEQ ID NO: 202-205; D, SEQ ID NO: 206-209.

FIG. 22. In vivo hypoglycemic activities of GLP-1 analogues by duodenal administration. Normal ICR mice were anesthetized with inhalation of ether, and the duodenum was surgically removed and injected with GLP-1 and its analogues or a corresponding volume of saline (10.0 mol/kg, n=9-11), and finally the incision was sewed. After 15 min, glucose solution (2 g/kg) was administered by gavage, and blood was collected from the tail tips at 15, 30, and 60 min after glucose administration. Blood glucose was measured using glucose oxidase method, and blood glucose values and area under the blood glucose curve (AUC) were calculated at each timepoint. Data are expressed as “mean±standard error”, and p<0.05 is considered to be statistically significant. A, SEQ ID NO: 194, SEQ ID NO: 196, SEQ ID NO: 198, SEQ ID NO: 200; B, SEQ ID NO: 202-205; C, SEQ ID NO: 206-209.

FIG. 23. In vivo hypoglycemic activities and dose-effect relationship of GLP-1 analogues by duodenal administration. Normal ICR mice were anesthetized with inhalation of ether, and the duodenum was surgically removed and injected with different doses (2.5, 5.0, 10.0 μmol/kg, n=9-11) or a combination of different proportions (5.0+5.0 μmol/kg, 5.0+5.0+5.0 μmol/kg, n=14-15) of GLP-1 analogues or corresponding volume of saline, and finally the incision was sewed. After 15 min, glucose solution (2 g/kg) was administered by gavage, and blood was collected from the tail tips at 15, 30, and 60 min after glucose administration. Blood glucose was measured using glucose oxidase method, and blood glucose values and area under the blood glucose curve (AUC) were calculated at each timepoint. Data are expressed as “mean±standard error”, and p<0.05 is considered to be statistically significant. The dose-effect relationships of A. SEQ ID NO: 200; B. SEQ ID NO: 204; C. compositions (SEQ ID NO: 200 and SEQ ID NO: 204) and (SEQ ID NO: 200, SEQ ID NO: 204 and SEQ ID NO: 208).

FIG. 24. The blood calcium concentration percentage-time curve of rat. Compared with the normal control group (Con), the serum calcium concentration of rats in the commercially available salmon calcitonin (sCat) group significantly decreased at the 3, 4, 6, 8, 12 and 24 h after administration, with a statistically significant difference (**p<0.01). The synthetic calcitonin analogue (CalM) group significantly decreased at the 3 h after administration, with a statistically significant difference (**p<0.01). However, the encapsulated Cal-BT group did not effectively reduce the blood calcium concentration of rats within 24 h after administration, with no statistically significant difference.

### DETAILED DESCRIPTION OF ILLUSTRATIVE EMBODIMENTS

In order to simplify the structure of natural protease inhibitor SFTI-1, and improve its specificity of active loop and inhibitory activities against serine protease, three series of peptides having intramolecular disulfide bonds were screened and identified using a rational design method. They inhibited the enzymatic activities of trypsin, chymotrypsin, and elastase, respectively, secreted by the pancreas. The activities of these three proteases are the main limiting factor for the absorption of therapeutic peptides and proteins into the blood circulation in the small intestine epithelium. Therefore, four biologically active peptides selected as candidates in the present invention are used to verify whether these three types of peptides with different protease inhibitory activities can be used as a universal molecular scaffold to form a fusion hybrid peptide with therapeutic peptides, whether they can improve the stability of the therapeutic peptides in the hybrid peptide to tolerate metabolic enzyme hydrolysis, and whether they can promote the absorption of the formed hybrid peptide in the intestinal epithelium and the pharmacological activities in vivo. The experimental results confirm that these three types of peptide molecular scaffolds with different protease inhibitory activities can be widely used to improve the stabilities and efficacies of therapeutic peptides and proteins in vivo.

Using the measurement method of inhibiting enzyme activity in vitro, at the first step a truncated monocyclic SFTI-1 mutant BT45 (SEQ ID NO: 45) containing only a disulfide bond was designed and synthesized. The experimental results indicated that its inhibition constant (Kᵢ) was the same as that of monocyclic SFTI-1 (BT1, SEQ ID NO: 1) only containing a disulfide bond (6.4 nM). The results confirmed that the truncated mutant BT45 was the most core peptide (molecular scaffold) for inhibiting trypsin. In order to explore whether the mutation at P3 site will seriously affect the trypsin inhibitory activity of the core scaffold, Cys was mutated to Gly or Ala with the addition of amino acid residues between disulfide bonds, that is, the extended loop between disulfide bonds. The research results confirmed that the molecular scaffold with trypsin inhibitory activity can be changed, then BT2 (SEQ ID NO: 2) and BT3 (SEQ ID NO: 3) were obtained. Besides, in another particular embodiment, the molecular scaffolds including SEQ ID NO: 5, SEQ ID NO: 6, and SEQ ID NO: 7 with enhanced trypsin inhibitory activity was obtained. Combining the truncated core scaffold described above and the extension strategy of the peptide segment between the disulfide bond, a series of amino acid site mutations are performed, and the optimized molecular scaffolds with excellent trypsin inhibitory activities such as SEQ ID NO: 9, SEQ ID NO: 10, SEQ ID NO: 16, SEQ ID NO: 17, SEQ ID NO: 25, SEQ ID NO: 27, SEQ ID NO: 28, SEQ ID NO: 35, SEQ ID NO: 46, SEQ ID NO: 47, SEQ ID NO: 49, SEQ ID NO: 50, SEQ ID NO: 51, SEQ ID NO: 53, SEQ ID NO: 54, SEQ ID NO: 55, SEQ ID NO: 57, SEQ ID NO: 60, SEQ ID NO: 65, SEQ ID NO: 66, SEQ ID NO: 67, SEQ ID NO: 69, SEQ ID NO: 70, SEQ ID NO: 71, SEQ ID NO: 74, SEQ ID NO: 75, SEQ ID NO: 76, SEQ ID NO: 77, SEQ ID NO: 78 and SEQ ID NO: 79, were obtained.

The P1 site of a serine protease inhibitory peptide determines the specificity of different serine proteases, along with the P1 sites of chymotrypsin being Tyr and Phe, and the P1 sites of elastase being Ala and Leu. Only a few literatures have reported the molecular scaffolds of active peptide against pancreatic chymotrypsin^{29,30,31} and elastase³², but their inhibitory activities of them are weak. Based on the core scaffold of anti-trypsin peptide, in the present invention the protease specificity of the molecular scaffold by replacing the P1 site was changed, and then the inhibition activities of peptides with different recognition sites were evaluated. After a series of optimization experiments, the inhibitory scaffold peptides against chymotrypsin were obtained as follows: SEQ ID NO: 85, SEQ ID NO: 90, SEQ ID NO: 91, SEQ ID NO: 98, SEQ ID NO: 105, SEQ ID NO: 106, SEQ ID NO: 113 SEQ ID NO: 114, SEQ ID NO: 115, SEQ ID NO: 131, SEQ ID NO: 132, and SEQ ID NO: 133; and the inhibitory scaffold against porcine pancreatic elastase were obtained as follows: SEQ ID NO: 134, SEQ ID NO: 145, SEQ ID NO: 151, SEQ ID NO: 155, SEQ ID NO: 156, SEQ ID NO: 158, and SEQ ID NO: 162.

### Definition

Unless otherwise defined herein, all terms used in this application shall have the meanings that are commonly understood by those of ordinary skill in the art. The following definitions provided below were used to illustrate and clarify the description and claims of the present invention.

The singular forms “a”, “an”, and “the” include the plural, unless the context clearly indicates otherwise.

The term “including” is used to mean “including but not limited to”. “Including” and “including but not limited to” are interchangeable.

The term “amino acid” or “any amino acid” as used herein refers to any and all amino acids, including naturally occurring amino acids (e.g α-amino acids), unnatural amino acids, and non-natural amino acids. It includes D-amino acids and L-amino acids. Natural amino acids include those naturally founded in nature, such as, e.g., 20 amino acids that are combined into peptide chains to form structural units of a large number of proteins, and these amino acids are mainly L-stereoisomers. “Unnatural” or “non-natural” amino acids are non-proteinogenic amino acids (i.e., those that are not naturally encoded or do not exist in genetic codons) that are naturally occurring or chemically synthesized.

These “unnatural” or “non-natural” amino acids have the same basic chemical structure as natural amino acids, i.e., compounds that bind to a hydrogen bound carbon, carboxyl, amino, and R-group, such as homocysteine, n-leucine, hydroxyproline, and 2-aminobutyric acid, and retain the same basic chemical structure as natural amino acids when participating in intramolecular peptide bonds.

As is clear to the skilled artisan, the peptide sequence disclosed herein is shown from left to right, wherein the left end of the sequence is the N-terminus of the peptide, and the right end of the sequence being the C-terminal of the peptide.

The terms “protein” and “peptide” are used interchangeably herein and broadly referred to a sequence of two or more amino acids linked together via peptide bonds. It should be understood that the two terms do not imply a specific length of amino acid polymer, nor are they intended to imply or distinguish whether peptides are produced using recombinant techniques, chemical synthesis, or enzymatic synthesis, or whether they are naturally occurring.

The term “a pharmaceutically acceptable salt” as used herein represents the salt or zwitterionic form of the peptide or compound of the present invention, which is water-soluble or oil-soluble or dispersible and suitable for the treatment of diseases without excessive toxicity, irritation, and allergic reactions. They are commensurate with a reasonable benefit/risk ratio and are effective for their intended use. The salt can be prepared during the final separation and purification of the compound, or separately by reacting the amino group with a suitable acid. Representative salts by acid addition reaction include acetate, hydrochloride, lactate, citrate, phosphate, and tartrate.

As used herein, the term “loop” in the present invention refers to the reaction loop, following the nomenclature of Schecter and Berger³³. In general formulas (I) and (II), the “loop” has intramolecular disulfide bonds and covers substrate-protease interaction sites. The P1 site corresponding to the Xaa1 residue in General Formulas (I) and (II) is the main determinant of protease specificity.

As used herein, the term “molecular scaffold” refers to and is used interchangeably used with “inhibitory ring”, which has different protease specificity determined by the Xaa1 residue in General Formulas (I) and (II). In some embodiments, the molecular scaffold is a mutant scaffold that contains modifications e.g. substitutions for natural or non-natural amino acids.

The term “linker” used in the present invention broadly refers to a peptide segment rich in glycine or proline that promotes the formation of turn structures, capable of connecting two peptides together and forming a chemical structure.

As is clear to a person skilled in the art, peptides with multiple cysteine residues often form disulfide bonds between two cysteine residues. All such peptides shown in the present invention are defined as optionally comprising one or more of these disulfide bonds.

The term “protease inhibitor” or “enzyme inhibitor” as used in the present invention refers to peptide molecules that inhibit the function of proteases. In one aspect, protease inhibitors (serine protease inhibitors) inhibit protease class from the serine proteases. In another aspect, protease inhibitors inhibit trypsin found in the gastrointestinal tract of mammals.

### Therapeutic Peptides

Glucagon like peptide-1 (GLP-1) is an endogenous hormone with anti-diabetes activity. GLP-1 is inactivated by the exopeptidase dipeptidyl peptidase IV (DPP-IV) and neutral endopeptidase 24.11 (NEP). The half-life of fully active GLP-I in vivo is approximately 90 s. In order to improve its stability in blood circulation, an inhibitory peptide, diprotin A (IPI)³⁴ and/or Opiorphin (QRFSR)³⁵, is connected to the N-terminal of GLP-1 through linkers such as “GG” (two glycine residues). The candidate GLP-1 analogue is further fused with the peptide inhibitor (molecular skeleton) disclosed in the present invention, and its hypoglycemic effect is tested through oral administration. In one embodiment, GLP-1 analogues SEQ ID NO: 184, SEQ ID NOS: 186-209 have been confirmed to have hypoglycemic activity by subcutaneous injection. In another embodiment, SEQ ID NO: 200, SEQ ID NO: 202, SEQ ID NO: 204, and SEQ ID NO: 205 have been confirmed to be absorbed into the blood circulation and have hypoglycemic activity by duodenal administration. The hypoglycemic effect of GLP-1 analogues administered orally can be achieved through enteric coated capsules. In another embodiment, hybrid GLP-1 analogues containing different protease inhibitory peptides are provided with combined effects.

Bacillus subtilisin/type 9 kexin preprotein converting enzyme (PCSK9) regulates low density lipoprotein cholesterol (LDL-C) levels by mediating LDL receptor (LDLR) protein degradation. Since PCSK9 is an important target for controlling plasma LDL-C levels by inhibiting protein protein-protein interaction (PPI) of PCSK9-LDLR, the main strategy for inhibiting PCSK9 binding to LDLR is to effectively reduce LDL-C levels by using LDLR binding sites that antagonize PCSK9³⁶. Although these monoclonal antibodies represent successful strategies for suppressing PCSK9, they cannot meet the compliance of patients with long-term treatment. In order to improve patient compliance, the inhibitory peptide Pep2-8³⁷ has been identified, but only in vitro biochemical analysis and cell level activity studies have been confirmed. The analogue of Pep2-8 (SEQ ID NO: 210, PCSK9_1) was selected as a candidate therapeutic peptide to further fuse with the peptide serine protease inhibitor (molecular scaffold) disclosed in the present invention, and its therapeutic efficacy in treating hypercholesterolemia was tested by direct duodenal administration. In one embodiment, the inhibition experiment of PCSK9-LDLR molecules confirmed that SEQ ID NO: 211, SEQ ID NO: 212, SEQ ID NO: 214, SEQ ID NO: 215, SEQ ID NO: 216, SEQ ID NO: 217, SEQ ID NO: 218, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231 SEQ ID NO: 232 and SEQ ID NO: 233 have good inhibitory effects in vitro. In another embodiment, a hyperlipidemia model was used to evaluate the effects of subcutaneous injection of SEQ ID NO: 214, SEQ ID NO: 215, SEQ ID NO: 218, SEQ ID NO: 229, SEQ ID NO: 230, and SEQ ID NO: 231. These peptides have excellent lowering lipid (total cholesterol) activities in vivo.

Human calcitonin (hCT) is a peptide hormone that contains 32 amino acid residues and is mainly produced by parafollicular cells of the thyroid gland. Many calcitonin homologues have been isolated, such as salmon calcitonin (sCT), eel calcitonin, porcine calcitonin, and chicken calcitonin. Among them, sCT is more effective and durable than hCT, and has been widely used in the treatment of osteoporosis, bone metastasis, paget disease, hypercalcemia shock, and chronic pain in advanced cancer. Calcitonin is currently only available in solution form and can be administered through intravenous, intramuscular, subcutaneous, or intranasal administration. However, the administration of these calcitonin drugs is significantly less convenient than oral administration, and causes more patient discomfort. Usually, this inconvenience or discomfort can lead to serious non-compliance with the treatment plan. In order to overcome these limitations and provide a better tolerable form of treatment, the sCT analogue is used as a candidate therapeutic peptide, which is further fused with the peptide serine protease inhibitor (molecular scaffold) disclosed in the present invention. Through oral administration, it is confirmed to be effective for treating osteoporosis or osteoarthritis.

Interleukin-17A (IL-17A) is a cytokine secreted by activated Th17 cells, CD8⁺ T cells, y6 T cells, and NK cells. It can regulate the production of mediators such as antimicrobial peptides (defensins). Various cell types of proinflammatory cytokines and chemokines, such as fibroblasts and synovial cells, are involved in neutrophil biology, inflammation, organ damage, and host defense. IL-17A mediates its action by interacting with interleukin-17 receptor A (IL-17RA) and receptor C (IL-17RC). The inappropriate or excessive production of IL-17A is related to various diseases and relative pathology, including rheumatoid arthritis, airway allergy (including allergic airway diseases such as asthma), skin allergy (including atopic dermatitis), systemic sclerosis, inflammatory bowel diseases including ulcerative colitis and Crohn's disease, and lung diseases including chronic obstructive pulmonary disease. Anti IL-17A's antibodies such as Secukizumab, Ixekizumab, and Bimekizumab have been used to treat IL-17A-mediated inflammatory disorders and diseases. Since the pharmacokinetics, efficacy, and safety of antibody therapy will depend on specific components, there is a need to improve antibody drugs suitable for the treatment of IL-17A mediated diseases. It is difficult to develop small molecule compounds targeting protein interactions due to the large and shallow interaction interfaces of IL-17A/IL-17RA interactions in structure. The fusion of a peptide antagonist with high affinity for IL-17A and anti-IL-22 antibody to form a bispecific fusion body was studied. Unfortunately, these findings reveal the problem of poor stability of inhibitory peptides against IL-17Ain cell cultures^{38, 39}. An analogue of IL-17A peptide antagonist (SEQ ID NO: 238) was selected as a candidate therapeutic peptide and further combined with the peptide inhibitor (molecular scaffold) against serine protease disclosed in the present invention to test the anti-inflammatory activity in vivo through duodenal administration. In one embodiment, the anti-inflammatory activity of SEQ ID NO: 239 and SEQ ID NO: 240 was evaluated by subcutaneous injection using an ear swelling model. In another embodiment, direct duodenal administration was performed, and the results confirmed that SEQ ID NO: 239 and SEQ ID NO: 240 which was absorbed into the blood circulation through the intestinal epithelium had anti-inflammatory activities.

The peptide protease inhibitor obtained in the present invention can be widely used to improve the stability of therapeutic peptides or proteins against digestive enzymes. Among them, therapeutic peptides or proteins are not limited to the peptides disclosed in the present invention and selected as examples. The therapeutic peptide or protein can be selected from the following sequences: LL-37 (SEQ ID NO: 242, LLGDFFRKSKEKEGKEFKRIVQRIKDFLRNLVPRTES) and its analogues with antibacterial, antiviral, and immunomodulatory activities; positively charged cationic antibacterial peptides Histatin 5 (SEQ ID NO: 243, DSHAKRHHGYKRKFHEKHSHSHRGY), indolicin (SEQ ID NO: 244, ILPWKWPWRR), and Pexiganan (SEQ ID NO: 245, GIGKFLKKKKKFGKAFVKILKK) and their analogues; antifungal peptide MAF-1A (SEQ ID NO: 246, KKFKETADKLIESALQQLESSSLAKEMK); anti-HIV Sifuviritide (SEQ ID NO: 247, SWETWEREIENYTRQIYRILEESQEQQDRNERDLLE) and Enfuviritide (SEQ ID NO: 248, YTSLIHSLIEESQNQEQEKNEQELLELDKWASLWWF) and their analogues; anti-HBV peptide Cl-1 (SEQ ID NO: 249, SFYSVLFLWGTCGGFSHSWY) and its analogues; anti-HCV active polypeptides p14 (SEQ ID NO: 250, RRGRTGRGRRGIYR), E2-550 (SEQ ID NO: 251, SWFGCTWMNSTGFTKTC), and C5A (SEQ ID NO: 252, SWLRDIWDWICEVLSDFK) and their analogues; anti-helicobacter pylon active peptides cagL-cagL (SEQ ID NO: 253, KNKNNFIKGIRKLMLAHNK), CagA-ASP2 (SEQ ID NO: 254, GPNIQKLLYQRTTIAAMETI), P1 (SEQ ID NO: 255, TGTLLLILSDVNDNAPIPEPR) and their analogues; DiaPep 277 (SEQ ID NO: 256, VLGGGALLRCIPALDSLTPANEL) and its analogues for treatment of type I diabetes; exendin-4, SEQ ID NO: 257, HGEGTFTSDLSKQMEEEAVRLFIEWLKNGGPSSGAPPPS and its analogues for the treatment of type II diabetes; EGF-A1 (SEQ ID NO: 258, GTNECLDNGGCSHVCNDLKIGYECCPDGFQLVAQRRCEDI), EGF-A5 (SEQ ID NO: 259, GTNECLDNGGCSHVCNDLKIGYECL), and BMS-962476 (SEQ ID NO: 260, PYKHSGYYHRP) and their analogues, which are active peptides for lowering blood lipids; anti-inflammatory active peptides Tag7 (SEQ ID NO: 261, ALRSNYVLKGHRDVQRTLSPG) and ZDC (SEQ ID NO: 262, FNMQQRFYLHPNENAKKSRD) and their analogues; Active peptides that inhibit tumor genesis or development, such as tumor angiogenesis inhibitor endothelin (SEQ ID NO: 263, CPAASARDFQPVLVALCSPLSGGMGRGIR), hypoxia inducible factor 1 α (hypoxia-inducible factor 1 α, HIF-1 α) Inhibitory peptides (SEQ ID NO: 264, GLPQLTSYDCEVNAPIQGSRNLLQGEELLALDQVN), Bcl-2 BH3 (SEQ ID NO: 265, EDIIRNIRHLAQVGDSNDRSIW), human virus entry mediator (HVEM) (SEQ ID NO: 266, ECCPKCSPGYRVKEACGELTGTVCEP), antagonistic peptides such as pDI (SEQ ID NO: 267, LTFEHYWAQLTS) PPKID4 (SEQ ID NO: 268, GPSQPTYPGDDAPVRRLSFFYILLDLYLDAPGVC) and its analogues that antagonize Bak/Bcl-2, and so on. The hybrid peptides formed by these therapeutically active peptides and the peptide protease inhibitors obtained in the present invention may not be limited to subcutaneous injection or oral administration or topical use.

### Peptide Synthesis

The peptide in the present invention can be prepared by various methods. For example, peptides can be synthesized through commonly used solid-phase synthesis methods, such as α-amino group t-BOC or FMOC protection method well known in the art. Here, amino acids are sequentially added to a growing chain of amino acids. The solid-phase synthesis method is particularly suitable for synthesizing peptides or relatively short peptides in large-scale production, e.g., peptides with a length of up to about 70 amino acids.

### Enzyme Inhibitory Activity Test

The inhibition constants of various synthetic active peptide inhibitors (molecular scaffolds) against seine proteases were measured. The substrates N-succinyl-Ala-Ala-Pro-Phe-p-nitroaniline (AAPFpNA), N_{α}—Benzoyl-L-arginine-4-nitroaniline hydrochloride (BApNA) and N-succinyl-Ala-Ala-p-nitroaniline (AAApNA) are used in competitive binding reaction to determine the inhibitory activities against α-chymotrypsin, bovine trypsin, and porcine pancreatic elastase, respectively. Experiments for α-chymotrypsin and bovine trypsin inhibition were measured in 20 mM CaCl₂, 50 mM Tris-HCl buffer (pH 7.8), and that for porcine elastase inhibition was measured in 50 mM Tris-HCl buffer (pH 8.0). The concentration of the peptide was measured using optical density (OD) at 280 nm. The Michaelis constant (Kₘ) of the enzyme hydrolysis substrate is calculated from the initial rate of substrate hydrolysis at 405 nm. The absorbance value of the substrate was measured at 405 nm after complete hydrolysis. All data were processed using nonlinear regression.

### Enteric Coated Capsule

The solid oral pharmaceutical composition in the present invention includes a dosage form, which is an enteric coated capsule. Such capsules are not limited to relatively stable shells used to encapsulate pharmaceutical preparations for oral administration. The two main types of capsules are capsules with hard and soft shells, which are typically used for dry, powdered ingredients, micro pellets, or mini tablets, primarily for oils and active ingredients dissolved or suspended in oils. Both capsules with hard and soft shells can be made from aqueous solutions of gelling agents, e.g., animal proteins, or gelatin, or plant polysaccharides or their derivatives, e.g., carrageenan, and modified forms of starch and cellulose. Other components can be added to the gelling agent solution, such as plasticizers, glycerol, and/or sorbitol, to reduce the hardness of the capsule, colorants, preservatives, disintegrants, lubricants, and surface treatment agents. The capsules in the present invention are coated with polymethacrylic acid/acrylate to form enteric coated capsules. Wherein the capsule packaging material targeting the duodenum and small intestine is selected from Eudragit L100 or L100-55. The packaging material for targeting the colon is selected from Eudragit S100 and can be prepared according to methods well known in the art, e.g., enteric coating or modified enteric coating.

### Method for Preparing Solid Oral Pharmaceutical Compositions

The solid oral pharmaceutical composition in the present invention can be prepared as known in the art. The solid oral pharmaceutical composition can be prepared as described in the embodiments herein.

### REFERENCES

1. Drucker D J. Advances in oral peptide therapeutics. Nat Rev Drug Discov, 2020, 19, 277-289.
2. Ibrahim Y H Y, Regdon G Jr, Hamedelniel E I, Sovány T. Review of recently used techniques and materials to improve the efficiency of orally administered proteins/peptides. Daru, 2020, 28, 403-416.
3. Renukuntla J, Vadlapudi A D, Patel A, Boddu S H S, Mitra A K. Approaches for enhancing oral bioavailability of peptides and proteins. Int J Pharm, 2013, 447, 75-93.
4. Luckett S, Garcia R S, Barker J J, Konarev A V, Shewry P R, Clarke A R, Brady R L. High-resolution structure of a potent, cyclic proteinase inhibitor from sunflower seeds. J Mol Biol, 1999, 290, 525-533.
5. Fittler H, Avrutina O, Glotzbach B, Empting M, Kolmar H. Combinatorial tuning of peptidic drug candidates: high-affinity matriptase inhibitors through incremental structure-guided optimization. Org Biomol Chem, 2013, 11, 1848-1857.
6. Quimbar P, Malik U, Sommerhoff C P, Kaas Q, Chan L Y, Huang Y H, Grundhuber M, Dunse K, Craik D J, Anderson M A, Daly N L. High-affinity cyclic peptide matriptase inhibitors. J Biol Chem, 2013, 288, 13885-13896.
7. de Veer S J, Li C Y, Swedberg J E, Schroeder C I, Craik D J. Engineering potent mesotrypsin inhibitors based on the plant-derived cyclic peptide, sunflower trypsin inhibitor-1. Eur J Med Chem. 2018, 155, 695-704.
8. Swedberg J E, Nigon L V, Reid J C, de Veer S J, Walpole C M, Stephens C R, Walsh T P, Takayama T K, Hooper J D, Clements J A, Buckle A M, Harris J M. Substrate-guided design of a potent and selective kallikrein-related peptidase inhibitor for kallikrein 4. Chem Biol, 2009, 16, 633-643.
9. Swedberg J E, de Veer S J, Sit K C, Reboul C F, Buckle A M, Harris J M. Mastering the canonical loop of serine protease inhibitors: enhancing potency by optimising the internal hydrogen bond network. PLoS One, 2011, 6, e19302.
10. de Veer S J, Swedberg J E, Akcan M, Rosengren K J, Brattsand M, Craik D J, Harris J M. Engineered protease inhibitors based on sunflower trypsin inhibitor-1 (SFTI-1) provide insights into the role of sequence and conformation in Laskowski mechanism inhibition. Biochem J, 2015a, 469, 243-253.
11. de Veer S J, Swedberg J E, Brattsand M, Clements J A, Harris J M. Exploring the active site binding specificity of kallikrein-related peptidase 5 (KLK5) guides the design of new peptide substrates and inhibitors. Biol Chem, 2016, 397, 1237-1249.
12. Shariff L, Zhu Y, Cowper B, Di W, Macmillan D. Sunflower trypsin inhibitor (SFTI-1) analogues of synthetic and biological origin via N→S acyl transfer: potential inhibitors of human Kallikrein-5 (KLK5). Tetrahedron, 2014, 70, 7675-7680.
13. Li C Y, de Veer S J, White A M, Chen X, Harris J M, Swedberg J E, Craik D J. Amino Acid Scanning at P5′ within the Bowman-Birk Inhibitory Loop Reveals Specificity Trends for Diverse Serine Proteases. J Med Chem, 2019, 62, 3696-3706.
14. de Veer S J, Furio L, Swedberg J E, Munro C A, Brattsand M, Clements J A, Hovnanian A, Harris J M. Selective substrates and inhibitors for kallikrein-related peptidase 7 (KLK7) shed light on KLK proteolytic activity in the stratum corneum. J Invest Dermatol, 2017, 137, 430-439.
15. Gitlin A, Debowski D, Kama N, Legowska A, Stimberg M, Gutschow M, Rolka K. Inhibitors of matriptase-2 based on the trypsin inhibitor SFTI-1. Chembiochem, 2015, 16, 1601-1607.
16. Fittler H, Depp A, Avrutina O, Dahms S O, Than M E, Empting M, Kolmar H. Engineering a constrained peptidic scaffold towards potent and selective furin inhibitors. Chembiochem, 2015, 16, 2441-2444.
17. Swedberg J E, Li C Y, de Veer S J, Wang C K, Craik D J. Design of Potent and Selective Cathepsin G Inhibitors Based on the Sunflower Trypsin Inhibitor-1 Scaffold. J Med Chem, 2017, 60, 658-667.
18. Legowska A, Debowski D, Lesner A, Wysocka M, Rolka K. Introduction of non-natural amino acid residues into the substrate-specific P1 position of trypsin inhibitor SFTI-1 yields potent chymotrypsin and cathepsin G inhibitors. Bioorg Med Chem, 2009, 17, 3302-3307.
19. Tian S, Swedberg J E, Li C Y, Craik D J, de Veer S J. Iterative optimization of the cyclic peptide SFTI-1 yields potent inhibitors of neutrophil proteinase 3. ACS Med Chem Lett, 2019, 10, 1234-1239.
20. Swedberg J E, Wu G, Mahatmanto T, Durek T, Caradoc-Davies T T, Whisstock J C, Law RHP, Craik D J. Highly potent and selective plasmin inhibitors based on the sunflower trypsin inhibitor-1 scaffold attenuate fibrinolysis in plasma. J Med Chem, 2019, 62, 552-560.
21. Choi Yi Li, Kuok Yap, Joakim E Swedberg, David J Craik, Simon J de Veer. Binding Loop Substitutions in the Cyclic Peptide SFTI-1 Generate Potent and Selective Chymase Inhibitors. J Med Chem, 2020, 63, 816-826.
22. Boy R G, Mier W, Nothelfer E M, Altmann A, Eisenhut M, Kolmar H, Tomaszowski M, Kramer S, Haberkorn U. Sunflower trypsin inhibitor 1 derivatives as molecular scaffolds for the development of novel peptidic radiopharmaceuticals. Mol Imaging Biol, 2010, 12, 377-385.
23. Chan L Y, Gunasekera S, Henriques S T, Worth N F, Le S J, Clark R J, Campbell J H, Craik D J, Daly N L. Engineering Pro-Angiogenic Peptides Using Stable, Disulfide-Rich Cyclic Scaffolds. Blood, 2011, 118, 6709-6717.
24. Qiu Y B, Taichi M, Wei N, Yang H, Luo K Q, Tam J P. An Orally Active Bradykinin B1 Receptor Antagonist Engineered as a Bifunctional Chimera of Sunflower Trypsin Inhibitor. J Med Chem, 2017, 60, 504-510.
25. Durek T, Cromm P M, White A M, Schroeder C I, Kaas Q, Weidmann J, Fuaad A A, Cheneval O, Harvey P J, Daly N L, Zhou Y, Dellsen D, Osterlund T, Larsson N, Knerr L, Bauer U, Kessler H, Cai M, Hruby V J, Plowright A T, Craik D J. Development of Novel Melanocortin Receptor Agonists Based on the Cyclic Peptide Framework of Sunflower Trypsin Inhibitor-1. J Med Chem, 2018, 61, 3674-3684.
26. Cobos Caceres C, Bansal P S, Navarro S, Wilson D, Don L, Giacomin P, Loukas A, Daly N L. An engineered cyclic peptide alleviates symptoms of inflammation in a murine model of inflammatory bowel disease. J Biol Chem, 2017, 292, 10288-10294.
27. Sable R, Durek T, Taneja V, Craik D J, Pallerla S, Gauthier T, Jois S. Constrained Cyclic Peptides as Immunomodulatory Inhibitors of the CD2:CD58 Protein-Protein Interaction. ACS Chem Biol, 2016, 11, 2366-2374.
28. Gunasekera S, Fernandes-Cerqueira C, Wennmalm S, Wahamaa H, Sommarin Y, Catrina A I, Jakobsson P J, Goransson U. Stabilized Cyclic Peptides as Scavengers of Autoantibodies: Neutralization of Anticitrullinated Protein/Peptide Antibodies in Rheumatoid Arthritis. ACS Chem Biol, 2018, 13, 1525-1535.
29. McBride J D, Brauer A B, Nievo M, Leatherbarrow R J. The role of threonine in the P2 position of Bowman-Birk proteinase inhibitors: studies on P2 variation in cyclic peptides encompassing the reactive site loop. J Mol Biol, 1998, 282: 447-458.
30. McBride J D, Freeman N, Domingo G J, Leatherbarrow R J. Selection of chymotrypsin inhibitors from a conformationally-constrained combinatorial peptide library. J Mol Biol, 1996, 259: 819-827.
31. McBride J D, Freeman H N, Leatherbarrow R J. Identification of chymotrypsin inhibitors from a second-generation template assisted combinatorial peptide library. J Pept Sci, 2000, 6: 446-452.
32. McBride J D, Freeman H N, Leatherbarrow R J. Selection of human elastase inhibitors from a conformationally constrained combinatorial peptide library. Eur J Biochem, 1999, 266: 403-412.
33. Schechter I, Berger A. On the size of the active site in proteases. I. Papain. Biochem Biophys Res Commun, 1967, 27: 157-162.
34. Umezawa H, Aoyagi T, Ogawa K, Naganawa H, Hamada M, Takeuchi T. Diprotins A and B, inhibitors of dipeptidyl aminopeptidase IV, produced by bacteria. J Antibiot (Tokyo), 1984, 37: 422-425.
35. Wisner A, Dufour E, Messaoudi M, Nejdi A, Marcel A, Ungeheuer M N, et al. Human Opiorphin, a natural antinociceptive modulator of opioid-dependent pathways. Proc Natl Acad Sci USA, 2006, 103: 17979-17984.
36. Lammi C, Sgrignani J, Arnoldi A, Grazioso G. Biological Characterization of Computationally Designed Analogs of peptide TVFTSWEEYLDWV (Pep2-8) with Increased PCSK9 Antagonistic Activity. Sci Rep, 2019, 9: 2343.
37. Zhang Y, Eigenbrot C, Zhou L, Shia S, Li W, Quan C, et al. Identification of a small peptide that inhibits PCSK9 protein binding to the low density lipoprotein receptor. J Biol Chem, 2014, 289: 942-955.
38. Vugmeyster Y, Zhang Y E, Zhong X, Wright J, Leung S S. Pharmacokinetics of anti-IL17A and anti-IL22 peptide-antibody bispecific genetic fusions in mice. Int Immunopharmacol, 2014, 18: 225-227.
39. Liu S, Desharnais J, Sahasrabudhe P V, Jin P, Li W, Oates B D, et al. Inhibiting complex IL-17A and IL-17RA interactions with a linear peptide. Sci Rep, 2016, 6: 26071.

The example below is provided to illustrate the embodiments in the present invention and is intended only for a better understanding of the present invention, but is not be interpreted as limiting the scope or spirit of the invention.

### EXAMPLES

### Example 1 Solid-Phase Synthesis of Peptides

According to the sequence of the amino acid residues of each polypeptide, the polypeptide is synthesized from C-terminal to N-terminal one by one through the solid-phase chemical synthesis method using Fmoc (9-fluorenylmethoxycarbonyl) amino protective agents; when the synthesis of the linear peptide protected by the side chain of the amino acid is completed, the linear peptide is cut from the resin, the protective group of amino acid residues in the linear peptide is removed, then the intramolecular sulfhydryl group is oxidized to form the disulfide bonds, finally, the target polypeptide is obtained by the purification of HPLC reversed-phase C18 column chromatography.

### I. Materials

1. Resins: Fmoc-Ala-Wang resin, Fmoc-Arg(Pbf)-Wang resin, Fmoc-Asn(Trt)-Wang resin, Fmoc-Asp(OtBu)-Wang resin, Fmoc-Gln(Trt)-Wang resin, Fmoc-Gly-Wang resin, Fmoc-Lys(Boc)-Wang resin, Fmoc-Phe-Wang resin, Fmoc-Pro-Wang resin, Fmoc-Ser(tBu)-Wang resin, Fmoc-Tyr(tBu)-Wang resin, Fmoc-Val-Wang resin, Fmoc-homoCys(Trt)-2-Cl-Trt resin, Fmoc-Pro-2-Cl-Trt resin, Fmoc-Lys(Boc)-Rink Amide AM resin, Fmoc-Pro-Rink Amide-AM Resin, Fmoc-Phe Rink Amide-AM Resin.
2. Amino acids: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Cys(Acm)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-homoCys(Trt)-OH, Fmoc-Abu-OH, Fmoc-Hyp(Trt)-OH, Fmoc-Nle-OH.
3. Reagents: piperidine, DMF, DCM, 4-Picoline, DIEA, HATU, HOBT, TBTU, DIC, TFA, EDT, TIPS, TA, phenol, diethyl ether, DMSO, distilled water.

### II. Synthesis Method 1

### SEQ ID NO: 9 (Cys-Gly-Arg-Ala-Thr-Lys-Ser-Ile-Pro-Pro-Ile-Cys-Phe)

1. Fmoc-Phe-Wang resin is used as the starting material, and the scale of synthesis is 0.1 mmol. The peptide is synthesized from C-terminal to N-terminal, the N-terminal Fmoc protective group is removed by piperidine/DMF (1:3, v/v) firstly to make the N-terminal a free amino group, 4-fold equivalent Fmoc-Cys(Trt)-OH is dissolved into HOBt/DIC to graft with the resin, the second amino acid residue of C-terminal (Cys) is introduced to obtain Fmoc-Cys(Trt)-Phe-Wang resin. As mentioned above, deprotect firstly and then repeatedly connect each amino acid residue of the polypeptide sequence successively, finally the peptide segment with protective groups is obtained, namely Fmoc-Cys(Trt)-Gly-Arg(Pbf)-Ala-Thr(tBu)-Lys(Boc)-Ser(tBu)-Ile-Pro-Pro-Ile-Cys(Trt)-Phe-Wang resin. DMF and DCM are necessary to be used to wash the resin alternately for 6 times after each step of the reaction above, taking the resin for Kaiser Test reaction. If the condensation reaction of any one of the amino acid residues was incomplete, the condensation should be repeated once, until the desired target peptide segment is obtained.
2. The peptide was removed of Fmoc and cleaved from the resin by treatment with cleavage reagent (TFA, EDT, TA, phenol, distilled water, TIPS mixed in certain proportion) at 30° C. for 3 h. After cleavage of the protecting group, the filtrate was added into a large amount of cold ether to precipitate the peptide, and then centrifuged. Washed with ether for several times and lyophilized, the crude peptide was obtained, namely Cys-Gly-Arg-Ala-Thr-Lys-Ser-Ile-Pro-Pro-Ile-Cys-Phe.
3. Dissolve the above-mentioned crude polypeptide into DMSO/H₂O (1:4, v/v) solution at the concentration of 4 mg/mL. Take the reaction solution and tracked by HPLC after 24 h, if the oxidation reaction was complete, then perform purification directly, if the oxidation reaction was incomplete, then the reaction time should be extended until the reaction is complete.
4. The target polypeptide is obtained through the purification of HPLC reversed-phase C18 column chromatography, those chemical structure is characterized by MALDI-TOF mass spectrometry, and the measured molecular weight of SEQ ID NO: 9 is 1391.06 Da ([M+H]⁺).

### SEQ ID NO: 1

### (Gly-Arg-Cys-Thr-Lys-Ser-Ile-Pro-Pro-Ile-Cys-Phe-Pro-Asp)

Fmoc-Asp(OtBu)-Wang resin was selected as the starting material of peptide SEQ ID NO: 1, which was synthesized according to the method described in chemical synthesis of S peptide EQ ID NO: 9. At first, the amino acids were added successively to synthesize the peptide segment with the protecting groups, namely Fmoc-Gly-Arg(Pbf)-Cys(Trt)-Thr(tBu)-Lys(Boc)-Ser(tBu)-Ile-Pro-Pro-Ile-Cys(Trt)-Phe-Pro-Asp(OtBu)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 1532.31 Da ([M+H]⁺).

### SEQ ID NO: 10

### (Cys-Gly-Arg-Ala-Thr-Lys-Ser-Ile-Pro-Ala-Ile-Cys-Phe)

Peptide SEQ ID NO: 10 was synthesized according to the method described in chemical synthesis of peptide SEQ ID NO: 9. At first, the amino acids were added successively to synthesize the peptide segment with the protecting groups, namely Fmoc-Cys(Trt)-Gly-Arg(Pbf)-Ala-Thr(tBu)-Lys(Boc)-Ser(tBu)-Ile-Pro-Ala-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 1364.72 Da ([M+H]⁺).

### SEQ ID NO: 211

### (Thr-Val-Phe-Thr-Ser-Trp-Glu-Glu-Ala-Leu-Asp-Trp-Val-Cys-Gly-Arg-Ala-Thr-Lys-Ser-Ile-Pro-Pro-Ile-Cys-Phe)

Peptide SEQ ID NO: 211 was synthesized according to the method described in chemical synthesis of peptide SEQ ID NO: 9. At first, the amino acids were added successively to synthesize the peptide segment with the protecting groups, namely Fmoc-Thr(tBu)-Val-Phe-Thr(tBu)-Ser(tBu)-Trp(Boc)-Glu(OtBu)-Glu(OtBu)-Ala-Leu-Asp(OtBu)-Trp(Boc)-Val-Cys(Trt)-Gly-Arg(Pbf)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2956.82 Da ([M+H]⁺).

### SEQ ID NO: 212

### (Cys-Gly-Arg-Ala-Thr-Lys-Ser-Ile-Pro-Pro-Ile-Cys-Phe-Gly-Thr-Val-Phe-Thr-Ser-Trp-Glu-Glu-Ala-Leu-Asp-Trp-Val)

Fmoc-Val-Wang resin was selected as the starting material for chemical synthesis of peptide SEQ ID NO: 212 synthesized according to the method described in chemical synthesis of peptide SEQ ID NO: 9. At first, the amino acids were added successively to synthesize the peptide segment with the protecting groups, namely Fmoc-Cys(Trt)-Gly-Arg(Pbf)-Ala-Thr(tBu)-Lys(Boc)-Ser(tBu)-Ile-Pro-Pro-Ile-Cys(Trt)-Phe-Gly-Thr(tBu)-Val-Phe-Thr(tBu)-Ser(tBu)-Trp(Boc)-Glu(OtBu)-Glu(OtBu)-Ala-Leu-Asp(OtBu)-Trp(Boc)-Val-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 3013.20 Da ([M+H]⁺).

### SEQ ID NO: 214

### (Trp-Glu-Glu-Ala-Leu-Asp-Trp-Val-Cys-Gly-Arg-Ala-Thr-Lys-Ser-Ile-Pro-Pro-Ile-Cys-Phe-Gly-Thr-Val-Phe-Thr-Ser)

Fmoc-Ser(tBu)-Wang resin was selected as the starting material for chemical synthesis of peptide SEQ ID NO: 214 synthesized according to the method described in chemical synthesis of peptide SEQ ID NO: 9. At first, the amino acids were added successively to synthesize the peptide segment with the protecting groups, namely Fmoc-Trp(Boc)-Glu(OtBu)-Glu(OtBu)-Ala-Leu-Asp(OtBu)-Trp(Boc)-Val-Cys(Trt)-Gly-Arg(Pbf)-Ala-Thr(tBu)-Lys(Boc)-Ser(tBu)-Ile-Pro-Pro-Ile-Cys(Trt)-Phe-Gly-Thr(tBu)-Val-Phe-Thr(tBu)-Ser(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 3012.71 Da ([M+H]⁺).

### SEQ ID NO: 215

### (Trp-Glu-Glu-Tyr-Leu-Asp-Tyr-Val-Cys-Gly-Arg-Ala-Thr-Lys-Ser-Ile-Pro-Pro-Ile-Cys-Phe-Gly-Thr-Val-Phe-Thr-Ser)

Fmoc-Ser(tBu)-Wang resin was selected as the starting material of chemical synthesis of peptide SEQ ID NO: 215 synthesized according to the method described in chemical synthesis of peptide SEQ ID NO: 9. At first, the amino acids were added successively to synthesize the peptide segment with the protecting groups, namely Fmoc-Trp(Boc)-Glu(OtBu)-Glu(OtBu)-Tyr(tBu)-Leu-Asp(OtBu)-Tyr(tBu)-Val-Cys(Trt)-Gly-Arg(Pbf)-Ala-Thr(tBu)-Lys(Boc)-Ser(tBu)-Ile-Pro-Pro-Ile-Cys(Trt)-Phe-Gly-Thr(tBu)-Val-Phe-Thr(tBu)-Ser(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 3082.43 Da ([M+H]⁺).

### SEQ ID NO: 216

### (Thr-Val-Phe-Thr-Ser-Gly-Cys-Gly-Arg-Ala-Thr-Lys-Ser-Ile-Pro-Pro-Ile-Cys-Phe-Trp-Glu-Glu-Tyr-Leu-Asp-Trp-Val)

Fmoc-Val-Wang resin was selected as the starting material of chemical synthesis of peptide SEQ ID NO: 216, which was synthesized according to the method described in chemical synthesis of peptide SEQ ID NO: 9. At first, the amino acids were added successively to synthesize the peptide segment with the protecting groups, namely Fmoc-Thr(tBu)-Val-Phe-Thr(tBu)-Ser(tBu)-Gly-Cys(Trt)-Gly-Arg(Pbf)-Ala-Thr(tBu)-Lys(Boc)-Ser(tBu)-Ile-Pro-Pro-Ile-Cys(Trt)-Phe-Trp(Boc)-Glu(OtBu)-Glu(OtBu)-Tyr(tBu)-Leu-Asp(OtBu)-Trp(Boc)-Val- Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 3105.15 Da ([M+H]⁺).

### SEQ ID NO: 218

### (Thr-Val-Phe-Thr-Ser-Gly-Arg-Cys-Thr-Lys-Ser-Ile-Pro-Pro-Ile-Cys-Phe-Trp-Glu-Glu-Tyr-Leu-Asp-Trp-Val)

Fmoc-Val-Wang resin was selected as the starting material of chemical synthesis of peptide SEQ ID NO: 218, which was synthesized according to the method described in chemical synthesis of peptide SEQ ID NO: 9. At first, the amino acids were added successively to synthesize the peptide segment with the protecting groups, namely Fmoc-Thr(tBu)-Val-Phe-Thr(tBu)-Ser(tBu)-Gly-Arg(Pbf)-Cys(Trt)-Thr(tBu)-Lys(Boc)-Ser(tBu)-Ile-Pro-Pro-Ile-Cys(Trt)-Phe-Trp(Boc)-Glu(OtBu)-Glu(OtBu)-Tyr(tBu)-Leu-Asp(OtBu)-Trp(Boc)-Val-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2977.09 Da ([M+H]⁺).

### SEQ ID NO: 224

### (Thr-Val-Phe-Thr-Ser-Trp-Glu-Glu-Ala-Leu-Asp-Trp-Val-Gly-Phe-Cys-Thr-Tyr-Ser-Ile-Pro-Pro-Gln-Cys-Tyr-Gly)

Fmoc-Gly-Wang resin was selected as the starting material of chemical synthesis of peptide SEQ ID NO: 224, which was synthesized according to the method described in chemical synthesis of peptide SEQ ID NO: 9. At first, the amino acids were added successively to synthesize the peptide segment with the protecting groups, namely Fmoc-Thr(tBu)-Val-Phe-Thr(tBu)-Ser(tBu)-Trp(Boc)-Glu(OtBu)-Glu(OtBu)-Ala-Leu-Asp(OtBu)-Trp(Boc)-Val-Gly-Phe-Cys(Trt)-Thr(tBu)-Tyr(tBu)-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2999.77 Da ([M+H]⁺).

### SEQ ID NO: 225

### (Thr-Val-Phe-Thr-Ser-Trp-Glu-Glu-Ala-Leu-Asp-Trp-Val-Gly-Ile-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 225, which was synthesized according to the method described in chemical synthesis of peptide SEQ ID NO: 9. At first, the amino acids were added successively to synthesize the peptide segment with the protecting groups, namely Fmoc-Thr(tBu)-Val-Phe-Thr(tBu)-Ser(tBu)-Trp(Boc)-Glu(OtBu)-Glu(OtBu)-Ala-Leu-Asp(OtBu)-Trp(Boc)-Val-Gly-Ile-Cys(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2766.11 Da ([M+H]⁺).

### SEQ ID NO: 226

### (Phe-Cys-Thr-Tyr-Ser-Ile-Pro-Pro-Gln-Cys-Tyr-Gly-Gly-Thr-Val-Phe-Thr-Ser-Trp-Glu-Glu-Ala-Leu-Asp-Trp-Val)

Fmoc-Val-Wang resin was selected as the starting material of chemical synthesis of peptide SEQ ID NO: 226, which was synthesized according to the method described in chemical synthesis of peptide SEQ ID NO: 9. At first, the amino acids were added successively to synthesize the peptide segment with the protecting groups, namely Fmoc-Phe-Cys(Trt)-Thr(tBu)-Tyr(tBu)-Ser(tBu)-Ile-Pro-Pro-Gln(Trt)-Cys(Trt)-Tyr(tBu)-Gly-Gly-Thr(tBu)-Val-Phe-Thr(tBu)-Ser(tBu)-Trp(Boc)-Glu(OtBu)-Glu(OtBu)-Ala-Leu-Asp(OtBu)-Trp(Boc)-Val-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2999.12 Da ([M+H]⁺).

### SEQ ID NO: 227

### (Ile-Cys-Thr-Ala-Ser-Ile-Pro-Pro-Ile-Cys-Gln-Gly-Thr-Val-Phe-Thr-Ser-Trp-Glu-Glu-Ala-Leu-Asp-Trp-Val)

Fmoc-Val-Wang resin was selected as the starting material of chemical synthesis of peptide SEQ ID NO: 227, which was synthesized according to the method described in chemical synthesis of peptide SEQ ID NO: 9. At first, the amino acids were added successively to synthesize the peptide segment with the protecting groups, namely Fmoc-Ile-Cys(Trt)-Thr(tBu)-Ala-Ser(tBu)-Ile-Pro-Pro-Ile-Cys(Trt)-Gln(Trt)-Gly-Thr(tBu)-Val-Phe-Thr(tBu)-Ser(tBu)-Trp(Boc)-Glu(OtBu)-Glu(OtBu)-Ala-Leu-Asp(OtBu)-Trp(Boc)-Val-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2766.78 Da ([M+H]⁺).

### SEQ ID NO: 228

### (Trp-Glu-Glu-Ala-Leu-Asp-Trp-Val-Gly-Phe-Cys-Thr-Tyr-Ser-Ile-Pro-Pro-Gln-Cys-Tyr-Gly-Thr-Val-Phe-Thr-Ser)

Fmoc-Ser(tBu)-Wang resin was selected as the starting material of chemical synthesis of peptide SEQ ID NO: 228, which was synthesized according to the method described in chemical synthesis of peptide SEQ ID NO: 9. At first, the amino acids were added successively to synthesize the peptide segment with the protecting groups, namely Fmoc-Trp(Boc)-Glu(OtBu)-Glu(OtBu)-Ala-Leu-Asp(OtBu)-Trp(Boc)-Val-Gly-Phe-Cys(Trt)-Thr(tBu)-Tyr(tBu)-Ser(tBu)-Ile-Pro-Pro-Gln(Trt)-Cys(Trt)-Tyr(tBu)-Gly-Thr(tBu)-Val-Phe-Thr(tBu)- Ser(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2999.34 Da ([M+H]⁺).

### SEQ ID NO: 229

### (Trp-Glu-Glu-Ala-Leu-Asp-Trp-Val-Gly-Ile-Cys-Thr-Ala-Ser-Ile-Pro-Pro-Ile-Cys-Gln-Gly-Thr-Val-Phe-Thr-Ser)

Fmoc-Ser(tBu)-Wang resin was selected as the starting material of chemical synthesis of peptide SEQ ID NO: 229, which was synthesized according to the method described in chemical synthesis of peptide SEQ ID NO: 9. At first, the amino acids were added successively to synthesize the peptide segment with the protecting groups, namely Fmoc-Trp(Boc)-Glu(OtBu)-Glu(OtBu)-Ala-Leu-Asp(OtBu)-Trp(Boc)-Val-Gly-Ile-Cys(Trt)-Thr(tBu)-Ala-Ser(tBu)-Ile-Pro-Pro-Ile-Cys(Trt)-Gln(Trt)-Gly-Thr(tBu)-Val-Phe-Thr(tBu)-Ser(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2822.72 Da ([M+H]⁺).

### SEQ ID NO: 230

### (Trp-Glu-Glu-Tyr-Leu-Asp-Tyr-Val-Gly-Phe-Cys-Thr-Tyr-Ser-Ile-Pro-Pro-Gln-Cys-Tyr-Gly-Thr-Val-Phe-Thr-Ser)

Fmoc-Ser(tBu)-Wang resin was selected as the starting material of chemical synthesis of peptide SEQ ID NO: 230, which was synthesized according to the method described in chemical synthesis of peptide SEQ ID NO: 9. At first, the amino acids were added successively to synthesize the peptide segment with the protecting groups, namely Fmoc-Trp(Boc)-Glu(OtBu)-Glu(OtBu)-Tyr(tBu)-Leu-Asp(OtBu)-Tyr(tBu)-Val-Gly-Phe-Cys(Trt)-Thr (tBu)-Tyr(tBu)-Ser(tBu)-Ile-Pro-Pro-Gln(Trt)-Cys(Trt)-Tyr(tBu)-Gly-Thr(tBu)-Val-Phe-Thr(tBu)-Ser(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 1021.6 Da ([M−H]³⁻).

### SEQ ID NO: 231

### (Trp-Glu-Glu-Tyr-Leu-Asp-Tyr-Val-Gly-Ile-Cys-Thr-Ala-Ser-Ile-Pro-Pro-Ile-Cys-Gln-Gly-Thr-Val-Phe-Thr-Ser)

Fmoc-Ser(tBu)-Wang resin was selected as the starting material of chemical synthesis of peptide SEQ ID NO: 230, which was synthesized according to the method described in chemical synthesis of peptide SEQ ID NO: 9. At first, the amino acids were added successively to synthesize the peptide segment with the protecting groups, namely Fmoc-Trp(Boc)-Glu(OtBu)-Glu(OtBu)-Tyr(tBu)-Leu-Asp(OtBu)-Tyr(tBu)-Val-Gly-Ile-Cys(Trt)-Thr (tBu)-Ala-Ser(tBu)-Ile-Pro-Pro-Ile-Cys(Trt)-Gln(Trt)-Gly-Thr(tBu)-Val-Phe-Thr(tBu)-Ser(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2891.97 Da ([M+H]⁺).

### SEQ ID NO: 232

### (Thr-Val-Phe-Thr-Ser-Gly-Phe-Cys-Thr-Tyr-Ser-Ile-Pro-Pro-Gln-Cys-Tyr-Gly-Trp-Glu-Glu-Tyr-Leu-Asp-Trp-Val)

Fmoc-Val-Wang resin was selected as the starting material of chemical synthesis of peptide SEQ ID NO: 232, which was synthesized according to the method described in chemical synthesis of peptide SEQ ID NO: 9. At first, the amino acids were added successively to synthesize the peptide segment with the protecting groups, namely Fmoc-Thr(tBu)-Val-Phe-Thr(tBu)-Ser(tBu)-Gly-Phe-Cys(Trt)-Thr(tBu)-Tyr(tBu)-Ser(tBu)-Ile-Pro-Pro-Gln(Trt)-Cys(Trt)-Tyr(tBu)-Gly-Trp(Boc)-Glu(OtBu)-Glu(OtBu)-Tyr(tBu)-Leu-Asp(OtBu)-Trp(Boc)-Val- Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 3091.42 Da ([M+H]⁺).

### SEQ ID NO: 233

### (Thr-Val-Phe-Thr-Ser-Gly-Ile-Cys-Thr-Ala-Ser-Ile-Pro-Pro-Ile-Cys-Gln-Trp-Glu-Glu-Tyr-Leu-Asp-Trp-Val)

Fmoc-Val-Wang resin was selected as the starting material of chemical synthesis of peptide SEQ ID NO: 233, which was synthesized according to the method described in chemical synthesis of peptide SEQ ID NO: 9. At first, the amino acids were added successively to synthesize the peptide segment with the protecting groups, namely Fmoc-Thr(tBu)-Val-Phe-Thr(tBu)-Ser(tBu)-Gly-Ile-Cys(Trt)-Thr(tBu)-Ala-Ser(tBu)-Ile-Pro-Pro-Ile-Cys(Trt)-Gln(Trt)-Trp(Boc)-Glu(OtBu)-Glu(OtBu)-Tyr(tBu)-Leu-Asp(OtBu)-Trp(Boc)-Val-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 2858.21 Da ([M+H]⁺).

### III. Synthesis Method 2

### SEQ ID NO: 45 (Arg-Cys-Thr-Lys-Ser-Ile-Pro-Pro-Ile-Cys-Phe)

1. Weighed Fmoc-Phe-Wang resin into the glass reaction column and added DCM to swell for 30 min, and then the DCM was removed by vacuum filtration.
2. Washed the resin with DMF for three times, added piperidine/DMF (1:4, v/v) solution to react for 20 min to remove the protecting group Fmoc. The solution was removed by vacuum filtration, then washed the resin with DMF for six times.
3. Weighed Fmoc-Cys(Trt)-OH and TBTU, and added them into the resin and dissolved by DMF. Added DIEA to react for 30 min, and took out of the resin to perform Kaiser Test. It was proved that the reaction was completed when the solution became bright yellow and the resin became yellow. The solvent could be removed by vacuum filtration.
4. Repeated steps 2 and 3 and then obtained the peptide segment with the protecting group, namely Fmoc-Arg(Pbf)-Cys(Trt)-Thr(tBu)-Lys(Boc)-Ser(tBu)-Ile-Pro-Pro-Ile-Cys(Trt)-Phe-Wang resin. Removed the Fmoc, washed the resin with DMF, DCM and methanol for three times, respectively, and then drained the resin by vacuum filtration.
5. The resin and the side-chain protecting group were removed by treatment with cleavage reagent (TFA, EDT, TA, phenol and distilled water mixed in certain proportion). Filtered with gravel core, added ether into the filtrate for precipitation, centrifuged and washed the solid for three times, drained by vacuum filtration.
6. Dissolved with H₂O/acetonitrile (9:1, v/v), and the volume was increased to 100 mL. Added dilute ammonia solution to adjust pH to basic (pH≈8) and the sample was taken out to test the activity of the thiol group. It indicated the presence of the thiol group when the solution turned yellow. The oxidation was completed (more than 90%) when the solution became clear after addition of 2-3 drops of hydrogen peroxide to react for 5-10 min. Added glacial acetic acid to adjust pH to acidic (pH≈6), and the chemical structure of the peptide was characterized by mass spectrometry. The target peptide of the correct molecular weight was obtained by purification using reversed-phase HPLC on a C18 column.
7. The measured molecular weight of peptide SEQ ID NO: 45 is 1262.40 Da ([M+3H]³+=421.80).

### SEQ ID NO: 16

### (Cys-Gly-Arg-Ala-Thr-Lys-Ser-Leu-Pro-Ala-Ile-Cys-Phe)

Peptide SEQ ID NO: 16 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ala-Thr(tBu)-Lys(Boc)-Ser(tBu)-Leu-Pro-Ala-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1365.09 Da ([M+H]⁺).

### SEQ ID NO: 17

### (Cys-Gly-Arg-Ala-Thr-Arg-Ser-Ile-Pro-Pro-Ile-Cys-Phe)

Peptide SEQ ID NO: 17 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ala-Thr(tBu)-Arg(Pbf)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1418.88 Da ([M+2H]²⁺=710.44).

### SEQ ID NO: 25

### (Cys-Gly-Thr-Ala-Thr-Lys-Ser-Ile-Pro-Pro-Ile-Cys-Phe)

Peptide SEQ ID NO: 25 was synthesized according to the method described in chemical synthesis of peptide SEQ ID NO: 45. At first, the amino acids were added successively to synthesize the peptide segment with the protecting groups, namely Fmoc-Cys(Trt)-Gly-Thr(tBu)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1335.00 Da ([M+2H]²⁺=668.50).

### SEQ ID NO: 27

### (Cys-Gly-Arg-Ala-Thr-Lys-Ala-Ile-Pro-Pro-Ile-Cys-Phe)

Peptide SEQ ID NO: 27 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ala-Thr(tBu)-Lys(Boc)-Ala-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1374.80 Da ([M+2H]²⁺=688.40).

### SEQ ID NO: 28

### (Cys-Gly-Arg-Ala-Thr-Lys-Ser-Nle-Pro-Pro-Ile-Cys-Phe)

Peptide SEQ ID NO: 28 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ala-Thr(tBu)-Lys(Boc)-Ser(tBu)-Nle-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1390.00 Da ([M+2H]²⁺=696.00).

### SEQ ID NO: 35

### (Cys-Gly-Arg-Abu-Thr-Lys-Ser-Ile-Pro-Pro-Ile-Cys-Phe)

Peptide SEQ ID NO: 35 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Abu-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1404.50 Da ([M+2H]²⁺=703.25).

### SEQ ID NO: 46

### (Cys-Hyp-Arg-Ala-Thr-Lys-Ser-Ile-Pro-Pro-Ile-Cys-Phe)

Peptide SEQ ID NO: 46 was synthesized according to the method described in chemical synthesis of peptide SEQ ID NO: 45. At first, the amino acids were added successively to synthesize the peptide segment with the protecting groups, namely Fmoc-Cys(Trt)-Hyp(Trt)-Arg(Pbf)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1446.60 Da ([M+3H]³⁺=483.20).

### SEQ ID NO: 47

### (Cys-Gly-Arg-Ser-Thr-Lys-Ser-Ile-Pro-Pro-Ile-Cys-Phe)

Peptide SEQ ID NO: 47 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ser(tBu)-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1407.00 Da ([M+3H]³⁺=470.00).

### SEQ ID NO: 49

### (Cys-Gly-Arg-Ile-Thr-Lys-Ser-Ile-Pro-Pro-Ile-Cys-Phe)

Peptide SEQ ID NO: 49 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ile-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1432.50 Da ([M+3H]³⁺=478.50).

### SEQ ID NO: 50

### (Cys-Gly-Arg-Nle-Thr-Lys-Ser-Ile-Pro-Pro-Ile-Cys-Phe)

Peptide SEQ ID NO: 50 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Nle-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1432.50 Da ([M+3H]³+=478.50).

### SEQ ID NO: 51

### (Cys-Gly-Arg-Val-Thr-Lys-Ser-Ile-Pro-Pro-Ile-Cys-Phe)

Peptide SEQ ID NO: 51 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Val-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1418.40 Da ([M+3H]³⁺=473.80).

### SEQ ID NO: 53

### (Cys-Gly-Arg-Tyr-Thr-Lys-Ser-Ile-Pro-Pro-Ile-Cys-Phe)

Peptide SEQ ID NO: 53 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Tyr(tBu)-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1482.90 Da ([M+3H]³⁺=495.30).

### SEQ ID NO: 54

### (Cys-Gly-Arg-Gln-Thr-Lys-Ser-Ile-Pro-Pro-Ile-Cys-Phe)

Peptide SEQ ID NO: 54 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Gln(Trt)-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1447.50 Da ([M+3H]³⁺=483.50).

### SEQ ID NO: 55

### (Cys-Gly-Arg-Asn-Thr-Lys-Ser-Ile-Pro-Pro-Ile-Cys-Phe)

Peptide SEQ ID NO: 55 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Asn(Trt)-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1433.40 Da ([M+3H]³⁺=478.80).

### SEQ ID NO: 57

### (Cys-Gly-Arg-Trp-Thr-Lys-Ser-Ile-Pro-Pro-Ile-Cys-Phe)

Peptide SEQ ID NO: 57 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Trp(Boc)-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1505.70 Da ([M+3H]³⁺=502.90).

### SEQ ID NO: 60

### (Cys-Gly-Arg-Gly-Thr-Lys-Ser-Ile-Pro-Pro-Ile-Cys-Phe)

Peptide SEQ ID NO: 60 was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Gly-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1376.20 Da ([M+2H]²⁺=689.10).

### SEQ ID NO: 65

### (Arg-Cys-Thr-Lys-Ser-Leu-Pro-Pro-Gln-Cys-Ser)

Fmoc-Ser(tBu)-Wang resin was selected as the starting material of chemical synthesis of peptide SEQ ID NO: 65, which was synthesized according to the method described in chemical synthesis of peptide SEQ ID NO: 45. At first, the amino acids were added successively to synthesize the peptide segment with the protecting groups, namely Fmoc-Arg(Pbf)-Cys(Trt)-Thr(tBu)-Lys(Boc)-Ser(tBu)-Leu-Pro-Pro-Gln(Trt)-Cys(Trt)-Ser(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1216.80 Da ([M+3H]³⁺=406.60).

### SEQ ID NO: 66

### (Cys-Pro-Arg-Ala-Thr-Lys-Ser-Ile-Pro-Pro-Ile-Cys-Phe)

Peptide SEQ ID NO: 66 was synthesized according to the method described in chemical synthesis of peptide SEQ ID NO: 45. At first, the amino acids were added successively to synthesize the peptide segment with the protecting groups, namely Fmoc-Cys(Trt)-Pro-Arg(Pbf)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1430.10 Da ([M+3H]³⁺=477.70).

### SEQ ID NO: 67

### (Cys-Ala-Arg-Ala-Thr-Lys-Ser-Ile-Pro-Pro-Ile-Cys-Phe)

Peptide SEQ ID NO: 67 was synthesized according to the method described in chemical synthesis of peptide SEQ ID NO: 45. At first, the amino acids were added successively to synthesize the peptide segment with the protecting groups, namely Fmoc-Cys(Trt)-Ala-Arg(Pbf)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1404.30 Da ([M+3H]³+=469.10).

### SEQ ID NO: 69

### (Cys-Ala-Arg-Ala-Thr-Lys-Ser-Ile-Hyp-Pro-Ile-Cys-Phe)

Peptide SEQ ID NO: 69 was synthesized according to the method described in chemical synthesis of peptide SEQ ID NO: 45. At first, the amino acids were added successively to synthesize the peptide segment with the protecting groups, namely Fmoc-Cys(Trt)-Ala-Arg(Pbf)-Ala-Thr(tBu)-Lys(Boc)-Ser(tBu)-Ile-Hyp(Trt)-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1420.80 Da ([M+3H]³⁺=474.60).

### SEQ ID NO: 70

### (Cys-Ala-Arg-Ala-Thr-Lys-Ser-Ile-Pro-Hyp-Ile-Cys-Phe)

Peptide SEQ ID NO: 70 was synthesized according to the method described in chemical synthesis of peptide SEQ ID NO: 45. At first, the amino acids were added successively to synthesize the peptide segment with the protecting groups, namely Fmoc-Cys(Trt)-Ala-Arg(Pbf)-Ala-Thr(tBu)-Lys(Boc)-Ser(tBu)-Ile-Pro-Hyp(Trt)-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1420.80 Da ([M+3H]³⁺=474.60).

### SEQ ID NO: 85

### (Phe-Cys-Thr-Phe-Ser-Ile-Pro-Pro-Gln-Cys-Tyr-Gly)

Fmoc-Gly-Wang resin was selected as the starting material of chemical synthesis of peptide SEQ ID NO: 85, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr (tBu)-Phe-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1360.02 Da ([M+K+H]²⁺=700.01).

### SEQ ID NO: 90

### (Phe-Cys-Thr-Tyr-Ser-Ile-Pro-Pro-Gln-Cys-Tyr-Gly)

Fmoc-Gly-Wang resin was selected as the starting material of chemical synthesis of peptide SEQ ID NO: 90, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr(tBu)-Tyr(tBu)-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1375.55 Da ([M+Na]=1398.55).

### SEQ ID NO: 91

### (Ser-Cys-Thr-Phe-Ser-Ile-Pro-Pro-Gln-Cys-Tyr-Gly)

Fmoc-Gly-Wang resin was selected as the starting material of chemical synthesis of peptide SEQ ID NO: 91, which was synthesized according to the method described in chemical synthesis of peptide SEQ ID NO: 45. At first, the amino acids were added successively to synthesize the peptide segment with the protecting groups, namely Fmoc-Ser(tBu)-Cys(Trt)-Thr(tBu)-Phe-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1300.55 Da ([M+H]⁺).

### SEQ ID NO: 98

### (Ala-Cys-Thr-Tyr-Ser-Ile-Pro-Ala-Lys-Cys-Phe)

Peptide SEQ ID NO: 98 was synthesized according to the method described in chemical synthesis of peptide SEQ ID NO: 45. At first, the amino acids were added successively to synthesize the peptide segment with the protecting groups, namely Fmoc-Ala-Cys(Trt)-Thr(tBu)-Tyr(tBu)-Ser(tBu)-Ile-Pro-Ala-Lys(Boc)-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1200.80 Da ([M+2H]²⁺=601.40).

### SEQ ID NO: 105

### (Gly-Thr-Cys-Thr-Phe-Ser-Ile-Pro-Pro-Ile-Cys-Asn-Pro-Asn)

Fmoc-Asn(Trt)-Wang resin was selected as the starting material of chemical synthesis of peptide SEQ ID NO: 105, which was synthesized according to the method described in chemical synthesis of peptide SEQ ID NO: 45. At first, the amino acids were added successively to synthesize the peptide segment with the protecting groups, namely Fmoc-Gly-Thr(tBu)-Cys(Trt)-Thr(tBu)-Phe-Ser(tBu)-Ile-Pro-Pro-Ile-Cys(Trt)-Asn(Trt)-Pro-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1461.00 Da ([M+2H]=731.50).

### SEQ ID NO: 106

### (Gly-Thr-Cys-Thr-Phe-Ser-Ile-Pro-Pro-Ile-Cys-Asn)

Fmoc-Asn(Trt)-Wang resin was selected as the starting material of chemical synthesis of peptide SEQ ID NO: 106, which was synthesized according to the method described in chemical synthesis of peptide SEQ ID NO: 45. At first, the amino acids were added successively to synthesize the peptide segment with the protecting groups, namely Fmoc-Gly-Thr(tBu)-Cys(Trt)-Thr(tBu)-Phe-Ser(tBu)-Ile-Pro-Pro-Ile-Cys(Trt)-Asn(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1249.50 Da ([M+Na]^{m}=1272.50).

### SEQ ID NO: 113

### (Phe-Cys-Thr-Tyr-Ser-Ile-Pro-Pro-Gln-Cys-Tyr)

Fmoc-Tyr(tBu)-Wang resin was selected as the starting material of chemical synthesis of peptide SEQ ID NO: 113, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr(tBu)-Tyr(tBu)-Ser(tBu)-Ile-Pro-Pro-Gln(Trt)-Cys(Trt)-Tyr(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1318.80 Da ([M+2H]²⁺=660.40).

### SEQ ID NO: 114

### (Phe-Cys-Thr-Tyr-Ser-Ile-Pro-Pro-Gln-Cys-Tyr-Ala)

Fmoc-Ala-Wang resin was selected as the starting material of chemical synthesis of peptide SEQ ID NO: 114, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr(tBu)-Tyr(tBu)-Ser(tBu)-Ile-Pro-Pro-Gln(Trt)-Cys(Trt)-Tyr(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1390.80 Da ([M+2H]²⁺=696.40).

### SEQ ID NO: 115

### (Phe-Cys-Thr-Tyr-Ser-Ile-Pro-Pro-Gln-Cys-Arg)

Fmoc-Arg(Pbf)-Wang resin was selected as the starting material of chemical synthesis of peptide SEQ ID NO: 115, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr(tBu)-Tyr(tBu)-Ser(tBu)-Ile-Pro-Pro-Gln(Trt)-Cys(Trt)-Arg(Pbf)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1312.20 Da ([M+2H]²⁺=657.10).

### SEQ ID NO: 131

### (Pro-Cys-Thr-Tyr-Ser-Ile-Pro-Pro-Gln-Cys-Tyr)

Fmoc-Tyr(tBu)-Wang resin was selected as the starting material of chemical synthesis of peptide SEQ ID NO: 131, which was synthesized according to the method described in chemical synthesis of peptide SEQ ID NO: 45. At first, the amino acids were added successively to synthesize the peptide segment with the protecting groups, namely Fmoc-Pro-Cys(Trt)-Thr(tBu)-Tyr(tBu)-Ser(tBu)-Ile-Pro-Pro-Gln(Trt)-Cys(Trt)-Tyr(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1268.80 Da ([M+2H]²⁺=635.40).

### SEQ ID NO: 132

### (Phe-Cys-Thr-Tyr-Ser-Ile-Pro-Hyp-Gln-Cys-Tyr-Gly)

Fmoc-Gly-Wang resin was selected as the starting material of chemical synthesis of peptide SEQ ID NO: 132, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr(tBu)-Tyr(tBu)-Ser(tBu)-Ile-Pro-Hyp(Trt)-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1392.40 Da ([M+2H]²⁺=697.20).

### SEQ ID NO: 133

### (Phe-Cys-Thr-Tyr-Ser-Ile-Hyp-Pro-Gln-Cys-Tyr-Gly)

Fmoc-Gly-Wang resin was selected as the starting material of chemical synthesis of peptide SEQ ID NO: 133, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr(tBu)-Tyr(tBu)-Ser(tBu)-Ile-Hyp(Trt)-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1392.00 Da ([M+2H]²+=697.00).

### SEQ ID NO: 134

### (Leu-Cys-Thr-Ala-Ser-Ile-Pro-Pro-Gln-Cys-Tyr)

Fmoc-Tyr(tBu)-Wang resin was selected as the starting material of chemical synthesis of peptide SEQ ID NO: 134, which was synthesized according to the method described in chemical synthesis of peptide SEQ ID NO: 45. At first, the amino acids were added successively to synthesize the peptide segment with the protecting groups, namely Fmoc-Leu-Cys(Trt)-Thr(tBu)-Ala-Ser(tBu)-Ile-Pro-Pro-Gln(Trt)-Cys(Trt)-Tyr(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1193.20 Da ([M+2H]²=597.60).

### SEQ ID NO: 145

### (Leu-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 145, which was synthesized according to the method described in chemical synthesis of peptide SEQ ID NO: 45. At first, the amino acids were added successively to synthesize the peptide segment with the protecting groups, namely Fmoc-Leu-Cys(Trt)-Thr (tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1143.50 Da ([M+H]⁺).

### SEQ ID NO: 151

### (Leu-Cys-Thr-Ala-Ser-Ile-Pro-Pro-Gln-Cys-Gln)

Fmoc-Gln(Trt)-Wang resin was selected as the starting material of chemical synthesis of peptide SEQ ID NO: 151, which was synthesized according to the method described in chemical synthesis of peptide SEQ ID NO: 45. At first, the amino acids were added successively to synthesize the peptide segment with the protecting groups, namely Fmoc-Leu-Cys(Trt)-Thr(tBu)-Ala-Ser(tBu)-Ile-Pro-Pro-Gln(Trt)-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1157.6 Da ([M+2H]²=579.80).

### SEQ ID NO: 155

### (Val-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 155, which was synthesized according to the method described in chemical synthesis of peptide SEQ ID NO: 45. At first, the amino acids were added successively to synthesize the peptide segment with the protecting groups, namely Fmoc-Leu-Cys(Trt)-Thr(tBu)-Ala-Ser(tBu)-Ile-Pro-Pro-Gln(Trt)-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1129.10 Da ([M+2H]²⁺=565.55).

### SEQ ID NO: 156

### (Ile-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 156, which was synthesized according to the method described in chemical synthesis of peptide SEQ ID NO: 45. At first, the amino acids were added successively to synthesize the peptide segment with the protecting groups, namely Fmoc-Ile-Cys(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1143.15 Da ([M+H]⁺).

### SEQ ID NO: 158

### (Leu-Cys-Thr-Ala-Ser-Asn-Pro-Pro-Ile-Cys-Gln)

Fmoc-Gln(Trt)-Wang resin was selected as the starting material of chemical synthesis of peptide SEQ ID NO: 158, which was synthesized according to the method described in chemical synthesis of peptide SEQ ID NO: 45. At first, the amino acids were added successively to synthesize the peptide segment with the protecting groups, namely Fmoc-Leu-Cys(Trt)-Thr(tBu)-Ala-Ser(tBu)-Asn(Trt)-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1143.80 Da ([M+2H]²+=572.90).

### SEQ ID NO: 162

### (Tyr-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 162, which was synthesized according to the method described in chemical synthesis of peptide SEQ ID NO: 45. At first, the amino acids were added successively to synthesize the peptide segment with the protecting groups, namely Fmoc-Tyr(tBu)-Cys(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1193.30 Da ([M−H]⁻=1192.30).

### SEQ ID NO: 163

### (Cys-Gly-Ile-Ala-Thr-Ala-Ser-Ile-Pro-Pro-Ile-Cys-Gln)

Fmoc-Gln(Trt)-Wang resin was selected as the starting material of chemical synthesis of peptide SEQ ID NO: 163, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Ala-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1270.80 Da ([M+2H]²⁺=636.40).

### SEQ ID NO: 164

### (Cys-Gly-Ile-Abu-Thr-Ala-Ser-Ile-Pro-Pro-Ile-Cys-Gln)

Fmoc-Gln(Trt)-Wang resin was selected as the starting material of chemical synthesis of peptide SEQ ID NO: 164, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Abu-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1285.70 Da ([M+H]=1285.70).

### SEQ ID NO: 165

### (Cys-Gly-Ile-Nle-Thr-Ala-Ser-Ile-Pro-Pro-Ile-Cys-Gln)

Fmoc-Gln(Trt)-Wang resin was selected as the starting material of chemical synthesis of peptide SEQ ID NO: 165, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Nle-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1312.80 Da ([M+2H]²⁺=657.40).

### SEQ ID NO: 166

### (Cys-Gly-Ile-Leu-Thr-Ala-Ser-Ile-Pro-Pro-Ile-Cys-Gln)

Fmoc-Gln(Trt)-Wang resin was selected as the starting material of chemical synthesis of peptide SEQ ID NO: 166, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Leu-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1313.00 Da ([M+2H]²⁺=657.50).

### SEQ ID NO: 167

### (Cys-Gly-Ile-Ser-Thr-Ala-Ser-Ile-Pro-Pro-Ile-Cys-Gln)

Fmoc-Gln(Trt)-Wang resin was selected as the starting material of chemical synthesis of peptide SEQ ID NO: 167, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Ser(tBu)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1287.00 Da ([M+2H]²=644.50).

### SEQ ID NO: 168

### (Cys-Gly-Ile-Thr-Thr-Ala-Ser-Ile-Pro-Pro-Ile-Cys-Gln)

Fmoc-Gln(Trt)-Wang resin was selected as the starting material of chemical synthesis of peptide SEQ ID NO: 168, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Thr(tBu)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1301.95 Da ([M+H]⁺).

### SEQ ID NO: 169

### (Cys-Gly-Ile-Phe-Thr-Ala-Ser-Ile-Pro-Pro-Ile-Cys-Gln)

Fmoc-Gln(Trt)-Wang resin was selected as the starting material of chemical synthesis of peptide SEQ ID NO: 169, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Phe-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1346.80 Da ([M+2H]²⁺=674.40).

### SEQ ID NO: 170

### (Cys-Gly-Ile-Tyr-Thr-Ala-Ser-Ile-Pro-Pro-Ile-Cys-Gln)

Fmoc-Gln(Trt)-Wang resin was selected as the starting material of chemical synthesis of peptide SEQ ID NO: 170, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Tyr(tBu)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1363.23 Da ([M+H]⁺).

### SEQ ID NO: 171

### (Cys-Gly-Ile-Asn-Thr-Ala-Ser-Ile-Pro-Pro-Ile-Cys-Gln)

Fmoc-Gln(Trt)-Wang resin was selected as the starting material of chemical synthesis of peptide SEQ ID NO: 171, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Asn(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1314.27 Da ([M+H]⁺).

### SEQ ID NO: 172

### (Cys-Gly-Ile-Gln-Thr-Ala-Ser-Ile-Pro-Pro-Ile-Cys-Gln)

Fmoc-Gln(Trt)-Wang resin was selected as the starting material of chemical synthesis of peptide SEQ ID NO: 172, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Gln(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1327.80 Da ([M+2H]²⁺=664.90).

### SEQ ID NO: 173

### (Cys-Gly-Ile-His-Thr-Ala-Ser-Ile-Pro-Pro-Ile-Cys-Gln)

Fmoc-Gln(Trt)-Wang resin was selected as the starting material of chemical synthesis of peptide SEQ ID NO: 173, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-His(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1337.00 Da ([M+2H]²⁺=669.50).

### SEQ ID NO: 174

### (Cys-Gly-Ile-Arg-Thr-Ala-Ser-Ile-Pro-Pro-Ile-Cys-Gln)

Fmoc-Gln(Trt)-Wang resin was selected as the starting material of chemical synthesis of peptide SEQ ID NO: 174, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Arg(Pbf)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1356.58 Da ([M+H]⁺).

### SEQ ID NO: 175

### (Cys-Gly-Ile-Lys-Thr-Ala-Ser-Ile-Pro-Pro-Ile-Cys-Gln)

Fmoc-Gln(Trt)-Wang resin was selected as the starting material of chemical synthesis of peptide SEQ ID NO: 175, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Lys(Boc)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1328.00 Da ([M+2H]²⁺=665.00).

### SEQ ID NO: 176

### (Cys-Gly-Ile-Trp-Thr-Ala-Ser-Ile-Pro-Pro-Ile-Cys-Gln)

Fmoc-Gln(Trt)-Wang resin was selected as the starting material of chemical synthesis of peptide SEQ ID NO: 176, which was synthesized according to the method described in chemical synthesis of peptide SEQ ID NO: 45. At first, the amino acids were added successively to synthesize the peptide segment with the protecting groups, namely Fmoc-Cys(Trt)-Gly-Ile-Trp(Boc)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1386.33 Da ([M+H]⁺).

### SEQ ID NO: 177

### (Cys-Pro-Ile-Ala-Thr-Ala-Ser-Ile-Pro-Pro-Ile-Cys-Gln)

Fmoc-Gln(Trt)-Wang resin was selected as the starting material of chemical synthesis of peptide SEQ ID NO: 177, which was synthesized according to the method described in chemical synthesis of peptide SEQ ID NO: 45. At first, the amino acids were added successively to synthesize the peptide segment with the protecting groups, namely Fmoc-Cys(Trt)-Pro-Ile-Ala-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1311.70 Da ([M+H]=1311.70).

### SEQ ID NO: 178

### (Cys-Ala-Ile-Ala-Thr-Ala-Ser-Ile-Pro-Pro-Ile-Cys-Gln)

Fmoc-Gln(Trt)-Wang resin was selected as the starting material of chemical synthesis of peptide SEQ ID NO: 178, which was synthesized according to the method described in chemical synthesis of peptide SEQ ID NO: 45. At first, the amino acids were added successively to synthesize the peptide segment with the protecting groups, namely Fmoc-Cys(Trt)-Ala-Ile-Ala-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1285.40 Da ([M+2H]²⁺=643.70).

### SEQ ID NO: 179

### (Cys-Hyp-Ile-Ala-Thr-Ala-Ser-Ile-Pro-Pro-Ile-Cys-Gln)

Fmoc-Gln(Trt)-Wang resin was selected as the starting material of chemical synthesis of peptide SEQ ID NO: 179, which was synthesized according to the method described in chemical synthesis of peptide SEQ ID NO: 45. At first, the amino acids were added successively to synthesize the peptide segment with the protecting groups, namely Fmoc-Cys(Trt)-Hyp(Trt)-Ile-Ala-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1327.20 Da ([M+2H]²⁺=664.60).

### SEQ ID NO: 180

### (Ile-Cys-Thr-Ala-Ser-Ile-Hyp-Pro-Ile-Cys-Gln)

Fmoc-Gln(Trt)-Wang resin was selected as the starting material of chemical synthesis of peptide SEQ ID NO: 180, which was synthesized according to the method described in chemical synthesis of peptide SEQ ID NO: 45. At first, the amino acids were added successively to synthesize the peptide segment with the protecting groups, namely Fmoc-Ile-Cys(Trt)-Thr(tBu)-Ala-Ser(tBu)-Ile-Hyp(Trt)-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1159.20 Da ([M+2H]²⁺=580.60).

### SEQ ID NO: 181

### (Ile-Cys-Thr-Ala-Ser-Ile-Pro-Hyp-Ile-Cys-Gln)

Fmoc-Gln(Trt)-Wang resin was selected as the starting material of chemical synthesis of peptide SEQ ID NO: 181, which was synthesized according to the method described in chemical synthesis of peptide SEQ ID NO: 45. At first, the amino acids were added successively to synthesize the peptide segment with the protecting groups, namely Fmoc-Ile-Cys(Trt)-Thr(tBu)-Ala-Ser(tBu)-Ile-Pro-Hyp(Trt)-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 1158.60 Da ([M−H]⁻=1157.60).

### SEQ ID NO: 194

### (Gly-Arg-Cys-Thr-Lys-Ser-Ile-Pro-Pro-Ile-Cys-Phe-Pro-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 194, which was synthesized according to the method described in chemical synthesis of peptide SEQ ID NO: 45. At first, the amino acids were added successively to synthesize the peptide segment with the protecting groups, namely Fmoc-Gly-Arg(Pbf)-Cys(Trt)-Thr(tBu)-Lys(Boc)-Ser(tBu)-Ile-Pro-Pro-Ile-Cys(Trt)-Phe-Pro-Gly-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)- Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5492.00 Da ([M+8H]⁸⁺=687.50).

### SEQ ID NO: 195

### (Gly-Arg-Cys-Thr-Lys-Ser-Ile-Pro-Pro-Ile-Cys-Phe-Pro-Gly-Gly-Gln-Arg-Phe-Ser-Arg-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 195, which was synthesized according to the method described in chemical synthesis of peptide SEQ ID NO: 45. At first, the amino acids were added successively to synthesize the peptide segment with the protecting groups, namely Fmoc-Gly-Arg(Pbf)-Cys(Trt)-Thr(tBu)-Lys(Boc)-Ser(tBu)-Ile-Pro-Pro-Ile-Cys(Trt)-Phe-Pro-Gly-Gly-Gln (Trt)-Arg(Pbf)-Phe-Ser(tBu)-Arg(Pbf)-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val- Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5842.40 Da ([M+8H]⁸⁺=731.30).

### SEQ ID NO: 196

### (Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Arg-Cys-Thr-Lys-Ser-Ile-Pro-Pro-Ile-Cys-Phe-Pro)

Fmoc-Pro-Wang resin was selected as the starting material of chemical synthesis of peptide SEQ ID NO: 196, which was synthesized according to the method described in chemical synthesis of peptide SEQ ID NO: 45. At first, the amino acids were added successively to synthesize the peptide segment with the protecting groups, namely Fmoc-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala- Trp(Boc)-Leu-Val-Lys(Boc)-Gly-Arg(Pbf)-Gly-Gly-Lys(Boc)-Gly-Arg(Pbf)-Cys(Trt)-Thr(tBu)-Lys(Boc)-Ser(tBu)-Ile-Pro-Pro-Ile-Cys(Trt)-Phe-Pro-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5437.75 Da ([M+5H]⁵+=1088.55).

### SEQ ID NO: 197

### (Gly-Gln-Arg-Phe-Ser-Arg-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Arg-Cys-Thr-Lys-Ser-Ile-Pro-Pro-Ile-Cys-Phe-Pro)

Fmoc-Pro-Wang resin was selected as the starting material of chemical synthesis of peptide SEQ ID NO: 197, which was synthesized according to the method described in chemical synthesis of peptide SEQ ID NO: 45. At first, the amino acids were added successively to synthesize the peptide segment with the protecting groups, namely Fmoc-Gly-Gln(Trt)-Arg(Pbf)-Phe-Ser(tBu)-Arg(Pbf)-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala- Lys(Boc)-Glu(OtBu)-Phe-Ile-Ala-Trp(Boc)-Leu-Val-Lys(Boc)-Gly-Arg(Pbf)-Gly-Gly-Lys(Boc)-Gly-Arg(Pbf)-Cys(Trt)-Thr(tBu)-Lys(Boc)-Ser(tBu)-Ile-Pro-Pro-Ile-Cys(Trt)-Phe-Pro-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5789.70 Da ([M+6H]⁶+=965.95).

### SEQ ID NO: 198

### (Cys-Gly-Arg-Ala-Thr-Lys-Ser-Ile-Pro-Pro-Ile-Cys-Phe-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 198, which was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ala-Thr(tBu)-Lys(Boc)-Ser(tBu)-Ile-Pro-Pro-Ile-Cys(Trt)-Phe-Gly-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)- Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5465.85 Da ([M+5H]⁵+=1094.17).

### SEQ ID NO: 199

### (Cys-Gly-Arg-Ala-Thr-Lys-Ser-Ile-Pro-Pro-Ile-Cys-Phe-Gly-Gly-Gln-Arg-Phe-Ser-Arg-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 199, which was synthesized according to the method described in chemical synthesis of peptide SEQ ID NO: 45. At first, the amino acids were added successively to synthesize the peptide segment with the protecting groups, namely Fmoc-Cys(Trt)-Gly-Arg(Pbf)-Ala-Thr(tBu)-Lys(Boc)-Ser(tBu)-Ile-Pro-Pro-Ile-Cys(Trt)-Phe-Gly-Gly-Gln(Trt)-Arg(Pbf)-Phe-Ser(tBu)-Arg(Pbf)-Gly-Gly-His(Trt)-Ala-Glu(OtBu)-Gly-Thr(tBu)-Phe- Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5815.56 Da ([M+6H]⁶⁺=970.26).

### SEQ ID NO: 200

### (Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Cys-Gly-Arg-Ala-Thr-Lys-Ser-Ile-Pro-Pro-Ile-Cys-Phe)

Fmoc-Phe-Wang resin was selected as the starting material of chemical synthesis of peptide SEQ ID NO: 200, which was synthesized according to the method described in chemical synthesis of peptide SEQ ID NO: 45. At first, the amino acids were added successively to synthesize the peptide segment with the protecting groups, namely Fmoc-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala- Trp(Boc)-Leu-Val-Lys(Boc)-Gly-Arg(Pbf)-Gly-Gly-Lys(Boc)-Gly-Cys(Trt)-Gly-Arg(Pbf)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5465.60 Da ([M+7H]⁸⁺=781.80).

### SEQ ID NO: 201

### (Gly-Gln-Arg-Phe-Ser-Arg-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Cys-Gly-Arg-Ala-Thr-Lys-Ser-Ile-Pro-Pro-Ile-Cys-Phe)

Fmoc-Phe-Wang resin was selected as the starting material of chemical synthesis of peptide SEQ ID NO: 201, which was synthesized according to the method described in chemical synthesis of peptide SEQ ID NO: 45. At first, the amino acids were added successively to synthesize the peptide segment with the protecting groups, namely Fmoc-Gly-Gln(Trt)-Arg(Pbf)-Phe-Ser(tBu)-Arg(Pbf)-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala- Lys(Boc)-Glu(OtBu)-Phe-Ile-Ala-Trp(Boc)-Leu-Val-Lys(Boc)-Gly-Arg(Pbf)-Gly-Gly-Lys(Boc)-Gly-Cys(Trt)-Gly-Arg(Pbf)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5816.70 Da ([M+6H]⁶+=970.45).

### SEQ ID NO: 202

### (Ser-Cys-Thr-Tyr-Ser-Ile-Pro-Pro-Gln-Cys-Tyr-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 202, which was synthesized according to the method described in chemical synthesis of peptide SEQ ID NO: 45. At first, the amino acids were added successively to synthesize the peptide segment with the protecting groups, namely Fmoc-Ser(tBu)-Cys(Trt)-Thr(tBu)-Tyr(tBu)-Ser(tBu)-Ile-Pro-Pro-Gln(Trt)-Cys(Trt)-Tyr(tBu)-Gly-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)- Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5333.10 Da ([M−3H]³⁻=1776.70).

### SEQ ID NO: 203

### (Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Ser-Cys-Thr-Tyr-Ser-Ile-Pro-Pro-Gln-Cys-Tyr-Gly)

Fmoc-Gly-Wang resin was selected as the starting material of chemical synthesis of peptide SEQ ID NO: 203, which was synthesized according to the method described in chemical synthesis of peptide SEQ ID NO: 45. At first, the amino acids were added successively to synthesize the peptide segment with the protecting groups, namely Fmoc-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala- Trp(Boc)-Leu-Val-Lys(Boc)-Gly-Arg(Pbf)-Gly-Gly-Lys(Boc)-Gly-Ser(tBu)-Cys(Trt)-Thr(tBu)-Tyr(tBu)-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5391.00 Da ([M+5H]⁵+=1079.20).

### SEQ ID NO: 204

### (Phe-Cys-Thr-Tyr-Ser-Ile-Pro-Pro-Gln-Cys-Tyr-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 204, which was synthesized according to the method described in chemical synthesis of peptide SEQ ID NO: 45. At first, the amino acids were added successively to synthesize the peptide segment with the protecting groups, namely Fmoc-Phe-Cys(Trt)-Thr(tBu)-Tyr(tBu)-Ser(tBu)-Ile-Pro-Pro-Gln(Trt)-Cys(Trt)-Tyr(tBu)-Gly-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val- Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5395.20 Da ([M−3H]³⁻=1797.40).

### SEQ ID NO: 205

### (Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Phe-Cys-Thr-Tyr-Ser-Ile-Pro-Pro-Gln-Cys-Tyr-Gly)

Fmoc-Gly-Wang resin was selected as the starting material of chemical synthesis of peptide SEQ ID NO: 205, which was synthesized according to the method described in chemical synthesis of peptide SEQ ID NO: 45. At first, the amino acids were added successively to synthesize the peptide segment with the protecting groups, namely Fmoc-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala- Trp(Boc)-Leu-Val-Lys(Boc)-Gly-Arg(Pbf)-Gly-Gly-Lys(Boc)-Gly-Phe-Cys(Trt)-Thr(tBu)-Tyr(tBu)-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5450.50 Da ([M+5H]⁵+=1091.10).

### SEQ ID NO: 206

### (Leu-Cys-Thr-Ala-Ser-Ile-Pro-Pro-Gln-Cys-Tyr-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 206, which was synthesized according to the method described in chemical synthesis of peptide SEQ ID NO: 45. At first, the amino acids were added successively to synthesize the peptide segment with the protecting groups, namely Fmoc-Leu-Cys(Trt)-Thr(tBu)-Ala-Ser(tBu)-Ile-Pro-Pro-Gln(Trt)-Cys(Trt)-Tyr(tBu)-Gly-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val- Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5268.50 Da ([M+5H]⁵⁺=1054.70).

### SEQ ID NO: 207

### (Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Leu-Cys-Thr-Ala-Ser-Ile-Pro-Pro-Gln-Cys-Tyr)

Fmoc-Tyr(tBu)-Wang resin was selected as the starting material of chemical synthesis of peptide SEQ ID NO: 207, which was synthesized according to the method described in chemical synthesis of peptide SEQ ID NO: 45. At first, the amino acids were added successively to synthesize the peptide segment with the protecting groups, namely Fmoc-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe- Ile-Ala-Trp(Boc)-Leu-Val-Lys(Boc)-Gly-Arg(Pbf)-Gly-Gly-Lys(Boc)-Gly-Leu-Cys(Trt)-Thr(tBu)-Ala-Ser(tBu)-Ile-Pro-Pro-Gln(Trt)-Cys(Trt)-Tyr(tBu)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5267.00 Da ([M+5H]⁵+=1054.40).

### SEQ ID NO: 208

### (Leu-Cys-Thr-Ala-Ser-Ile-Pro-Pro-Ile-Cys-Gln-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 208, which was synthesized according to the method described in chemical synthesis of peptide SEQ ID NO: 45. At first, the amino acids were added successively to synthesize the peptide segment with the protecting groups, namely Fmoc-Leu-Cys(Trt)-Thr(tBu)-Ala-Ser(tBu)-Ile-Pro-Pro-Ile-Cys(Trt)-Gln(Trt)-Gly-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)- Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5218.00 Da ([M+5H]⁵⁺=1044.60).

### SEQ ID NO: 209

### (Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Leu-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 209, which was synthesized according to the method described in chemical synthesis of peptide SEQ ID NO: 45. At first, the amino acids were added successively to synthesize the peptide segment with the protecting groups, namely Fmoc-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe- Ile-Ala-Trp(Boc)-Leu-Val-Lys(Boc)-Gly-Arg(Pbf)-Gly-Gly-Lys(Boc)-Gly-Leu-Cys(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 5218.00 Da ([M+5H]⁵+=1044.60).

### SEQ ID NO: 239

### (Ile-His-Val-Thr-Ile-Pro-Ala-Asp-Leu-Trp-Asp-Trp-Ile-Asn-Gly-Cys-Gly-Arg-Ala-Thr-Lys-Ser-Ile-Pro-Pro-Ile-Cys-Phe)

Fmoc-Phe-Wang resin was selected as the starting material of chemical synthesis of peptide SEQ ID NO: 239, which was synthesized according to the method described in chemical synthesis of peptide SEQ ID NO: 45. At first, the amino acids were added successively to synthesize the peptide segment with the protecting groups, namely Fmoc-Ile-His(Trt)-Val-Thr(tBu)-Ile-Pro-Ala-Asp(OtBu)-Leu-Trp(Boc)-Asp(OtBu)-Trp(Boc)-Ile-Asn(Trt)-Gly-Cys(Trt)-Gly-Arg(Pbf)-Ala-Thr(tBu)-Lys(Boc)-Ser(tBu)-Ile-Pro-Pro-Ile-Cys(Trt)-Phe-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 3122.40 Da ([M+4H]⁴+=781.60).

### SEQ ID NO: 240

### (Ile-His-Val-Thr-Ile-Pro-Ala-Asp-Leu-Trp-Asp-Trp-Ile-Asn-Gly-Phe-Cys-Thr-Tyr-Ser-Ile-Pro-Pro-Gln-Cys-Tyr-Gly)

Fmoc-Gly-Wang resin was selected as the starting material of chemical synthesis of peptide SEQ ID NO: 240, which was synthesized according to the method described in chemical synthesis of peptide SEQ ID NO: 45. At first, the amino acids were added successively to synthesize the peptide segment with the protecting groups, namely Fmoc-Ile-His(Trt)-Val-Thr(tBu)-Ile-Pro-Ala-Asp(OtBu)-Leu-Trp(Boc)-Asp(OtBu)-Trp(Boc)-Ile-Asn(Trt)-Gly-Phe-Cys(Trt)-Thr(tBu)-Tyr(tBu)-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 3108.00 Da ([M+3H]³⁺=1037.00).

### SEQ ID NO: 241

### (Ile-His-Val-Thr-Ile-Pro-Ala-Asp-Leu-Trp-Asp-Trp-Ile-Asn-Gly-Ile-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 240, which was synthesized according to the method described in chemical synthesis of peptide SEQ ID NO: 45. At first, the amino acids were added successively to synthesize the peptide segment with the protecting groups, namely Fmoc-Ile-His(Trt)-Val-Thr(tBu)-Ile-Pro-Ala-Asp(OtBu)-Leu-Trp(Boc)-Asp(OtBu)-Trp(Boc)-Ile-Asn(Trt)-Gly-Ile-Cys(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of the cleavage buffer, followed by formation of the disulfide bond via oxidation. Finally, the target peptide segment was obtained by separation and purification with the measured molecular weight of 2874.90 Da ([M+3H]³+=959.30).

### IV. Synthesis Method 3

### SEQ ID NO: 29 (Hcy-Gly-Arg-Ala-Thr-Lys-Ser-Ile-Pro-Pro-Ile-Ala-Phe-Hcy)

1. Weighed Fmoc-homoCys(Trt)-2-Cl-Trt resin into the glass reaction column and added DCM to swell for 30 min, the DCM was removed by vacuum filtration.
2. Washed the resin with DMF for three times, added piperidine/DMF (1:4, v/v) solution to react for 20 min to remove the protecting group Fmoc. The solution was removed by vacuum filtration, then washed the resin with DMF for six times.
3. Weighed Fmoc-Phe-OH and TBTU. Added them into the resin and dissolved by DMF. Added DIEA to react for 30 min, and took out of the resin to perform Kaiser Test. It was proved that the reaction was complete when the solution became bright yellow and the resin became yellow. The solvent could be removed by vacuum filtration.
4. Repeated steps 2 and 3 could obtain the peptide segment with the protecting group, namely Fmoc-Hcy(Trt)-Gly-Arg(Pbf)-Ala-Thr(tBu)-Lys(Boc)-Ser(tBu)-Ile-Pro-Pro-Ile-Ala-Phe-homoCys(Trt)-2-Cl-Trt resin. Removed the Fmoc, washed the resin with DMF, DCM and methanol for three times each, and then drained the resin by vacuum filtration.
5. The resin and the side-chain protecting group were removed by treatment with cleavage reagent (TFA, EDT, TA, phenol and distilled water mixed in certain proportion). Filtered with gravel core, added ether into the filtrate for precipitation, centrifuged and washed the solid for three times, drained by vacuum filtration.
6. Dissolved with H₂O/acetonitrile (9:1, v/v), and the volume was increased to 100 mL. Added dilute ammonia solution to adjust pH to basic (pH≈8) and the sample was taken to test the activity of the thiol group. It indicated the presence of the thiol group when the solution turned yellow. The oxidation was complete (more than 90%) when the solution became clear after addition of 2-3 drops of hydrogen peroxide to react for 5-10 min. Added glacial acetic acid to adjust pH to acidic (pH≈6), and the chemical structure of the peptide was characterized by mass spectrometry. The target peptide of the correct molecular weight was obtained by purification using reversed-phase HPLC on a C18 column.
7. The measured molecular weight of peptide SEQ ID NO: 29 is 1489.00 Da ([M+2H]²+=745.50).

### SEQ ID NO: 33

### (Hcy-Gly-Arg-Ala-Thr-Lys-Ser-Ile-Pro-Pro-Ile-Ala-Phe-Gly-Hcy)

Peptide SEQ ID NO: 33 was synthesized according to the method described in chemical synthesis of peptide SEQ ID NO: 29. At first, the amino acids were added successively to synthesize the peptide segment with the protecting groups, namely Fmoc-homoCys(Trt)-Gly-Arg(Pbf)-Ala-Thr(tBu)-Lys(Boc)-Ser(tBu)-Ile-Pro-Pro-Ile-Ala-Phe-Gly-homoCys(Trt)-2-Cl-Trt resin. Then the Fmoc group was removed, and the resin and the side-chain protecting groups were also removed by addition of the cleavage buffer, followed by formation of the disulfide bond via oxidation. Finally, the target peptide segment was obtained with the measured molecular weight of 1546.60 Da ([[M+2H]²⁺=774.30).

### V. Synthesis Method 4

### SEQ ID NO: 234

### (Cys-Ser-Asn-Leu-Ser-Thr-Cys-Gly-Leu-Gly-Lys-Leu-Ser-Gln-Glu-Ala-His-Lys-Leu-Gln-Thr-Tyr-Pro-Arg-Thr-Asn-Thr-Gly-Ser-Gly-Thr-Pro)

1. Weighed Fmoc-Pro-2-Cl-Trt resin into the glass reaction column and added DCM to swell for 30 min, the DCM was removed by vacuum filtration.
2. Washed the resin with DMF for three times, added piperidine/DMF (1:4, v/v) solution to react for 20 min to remove the protecting group Fmoc. The solution was removed by vacuum filtration, then washed the resin with DMF for six times.
3. Weighed Fmoc-Thr(tBu)-OH and TBTU. Added them into the resin and dissolved with DMF. Added DIEA to react for 30 min, and taken out of the resin to perform Kaiser Test. It was proved that the reaction was complete when the solution became bright yellow and the resin became yellow. The solvent could be removed by vacuum filtration.
4. Repeated steps 2 and 3 could obtain the peptide segment with the protecting group, namely Fmoc-Cys(Trt)-Ser(tBu)-Asn(Trt)-Leu-Ser(tBu)-Thr(tBu)-Cys(Trt)-Gly-Leu-Gly-Lys(Boc)-Leu-Ser(tBu)-Gln(Trt)- Glu(OtBu)-Ala-His(Trt)-Lys(Boc)-Leu-Gln(Trt)-Thr(tBu)-Tyr(tBu)-Pro-Arg(Pbf)-Thr(tBu)-Asn(Trt)-Thr(tBu)-Gly-Ser(tBu)-Gly-Thr(tBu)-Pro-2-Cl-Trt resin. Removed the Fmoc group, washed the resin with DMF, DCM and methanol for three times each, and then drained the resin by vacuum filtration.
5. The resin and the side-chain protecting group were removed by treatment with cleavage reagent (TFA, EDT, TA, phenol and distilled water mixed in certain proportion). Filtered with gravel core, added ether into the filtrate for precipitation, centrifuged and washed the solid for three times, drained by vacuum filtration.
6. Dissolved with H₂O/acetonitrile (9:1, v/v), and the volume was increased to 100 mL. Added dilute ammonia solution to adjust pH to basic (pH≈8), and the sample was taken to test the activity of the thiol group. It indicated the presence of the thiol group when the solution turned yellow. The oxidation was completed (more than 90%) when the solution became clear after addition of 2-3 drops of hydrogen peroxide to react for 5-10 min. Added glacial acetic acid to adjust pH to acidic (pH≈6), and the chemical structure of the peptide was characterized by mass spectrometry. The target peptide of the correct molecular weight was obtained by purification using reversed-phase HPLC on a C18 column.
7. The measured molecular weight of peptide SEQ ID NO: 234 is 3349.00 Da ([M+5H]⁵⁺=670.80).

### VI. Synthesis Method 5

### SEQ ID NO: 235

### (Cys-Ser-Asn-Leu-Ser-Thr-Cys-Gly-Leu-Gly-Lys-Leu-Ser-Gln-Glu-Ala-His-Lys-Leu-Gln-Thr-Tyr-Pro-Arg-Thr-Asn-Thr-Gly-Ser-Gly-Thr-Pro-Arg-Thr-Asn-Thr-Gly-Ser-Gly-Thr-Pro-Gly-Cys-Ala-Arg-Ala-Thr-Lys-Ser-Ile-Pro-Pro-Ile-Cys-Phe)

1. Weighed Fmoc-Phe-Wang resin into the glass reaction column and added DCM to swell for 30 min, the DCM was removed by vacuum filtration.
2. Washed the resin with DMF for three times, added piperidine/DMF (1:4, v/v) solution to react for 20 min to remove the protecting group Fmoc. The solution was removed by vacuum filtration, then washed the resin with DMF for six times.
3. Weighed Fmoc-Cys(Trt)-OH and TBTU. Added them into the resin and dissolved by DMF. Added DIEA to react for 30 min, and took out of the resin to perform Kaiser Test. It was proved that the reaction was completed when the solution became bright yellow and the resin became yellow. The solvent could be extracted by vacuum filtration.
4. Repeat steps 2 and 3 could obtain the peptide segment with the protecting group, namely Fmoc-Cys(Acm)-Ser(tBu)-Asn(Trt)-Leu-Ser(tBu)-Thr(tBu)-Cys(Acm)-Gly-Leu-Gly-Lys(Boc)-Leu-Ser(tBu)-Gln(Trt)-Glu(OtBu)- Ala-His(Trt)-Lys(Boc)-Leu-Gln(Trt)-Thr(tBu)-Tyr(tBu)-Pro-Arg(Pbf)-Thr(tBu)-Asn(Trt)-Thr(tBu)-Gly-Ser(tBu)-Gly-Thr(tBu)-Pro-Arg(Pbf)-Thr(tBu)-Asn(Trt)-Thr(tBu)-Gly-Ser(tBu)-Gly-Thr(tBu)-Pro-Gly-Cys(Trt)-Ala-Arg(Pbf)-Ala-Thr(tBu) -Lys(Boc)-Ser(tBu)-Ile-Pro-Pro-Ile-Cys(Trt)-Phe-Wang resin. Removed the Fmoc, washed the resin with DMF, DCM and methanol for three times each, and then drained the resin by vacuum filtration.
5. The resin and the side-chain protecting group was removed by treatment with cleavage reagent (TFA, EDT, TA, phenol and distilled water mixed in certain proportion). Filtered with gravel core, added ether into the filtrate for precipitation, centrifuged and washed the solid for three times, drained by vacuum filtration.
6. The sample was purified by using reversed-phase HPLC on a C18 column, and the purified chromatographic peak of the first time was collected. Added dilute ammonia solution to adjust pH to basic (pH≈8) and the sample is taken out to test the activity of the thiol group. It indicated the presence of the thiol group when the solution turned yellow. The oxidation was completed (more than 90%) when the solution became clear after addition of 2-3 drops of hydrogen peroxide to react for 5-10 min. Added glacial acetic acid to adjust pH to acidic (pH≈6), purified the sample and collected the chromatographic peak again.
7. Slowly dripped iodine-contained methanol solution (Ig iodine/100 mL methanol) into the solution of the purified chromatographic peak of the second time until the color is constantly dark brown. Observed the reaction until it was complete, and obtained the final target peptide by purification. The chemical structure was characterized by mass spectrometry.
8. The measured molecular weight of peptide SEQ ID NO: 235 is 4792.80 Da ([M+6H]⁶+=799.80).

### SEQ ID NO: 236

### (Cys-Ser-Asn-Leu-Ser-Thr-Cys-Gly-Leu-Gly-Lys-Leu-Ser-Gln-Glu-Ala-His-Lys-Leu-Gln-Thr-Tyr-Pro-Arg-Thr-Asn-Thr-Gly-Ser-Gly-Thr-Pro-Arg-Thr-Asn-Thr-Gly-Ser-Gly-Thr-Pro-Gly-Phe-Cys-Thr-Tyr-Ser-Ile-Pro-Pro-Gln-Cys-Tyr-Gly)

Fmoc-Gly-Wang resin was selected as the starting material of chemical synthesis of peptide SEQ ID NO: 236, which was synthesized according to the method described in chemical synthesis of peptide SEQ ID NO: 235. At first, the amino acids were added successively to synthesize the peptide segment with the protecting groups, namely Fmoc-Cys(Acm)-Ser(tBu)-Asn(Trt)-Leu-Ser(tBu)-Thr(tBu)-Cys(Acm)-Gly-Leu-Gly-Lys(Boc)-Leu-Ser(tBu)-Gln(Trt)-Glu-Ala-His(Trt)-Lys(Boc)-Leu-Gln(Trt)-Thr(tBu)-Tyr(tBu)-Pro-Arg(Pbf)-Thr(tBu)-Asn (Trt)-Thr(tBu)-Gly-Ser(tBu)-Gly-Thr(tBu)-Pro-Arg(Pbf)-Thr(tBu)-Asn(Trt)-Thr(tBu)-Gly-Ser(tBu)-Gly-Thr(tBu)-Pro-Gly-Phe-Cys(Trt)-Thr(tBu)-Tyr(tBu)-Ser(tBu)-Ile-Pro-Pro-Gln(Trt)-Cys(Trt)-Tyr(tBu)-Gly-Wang resin. Then the Fmoc was removed, and the resin and the side-chain protecting groups were also removed by addition of the cleavage buffer, followed by formation of the disulfide bond via oxidation. Finally, the target peptide segment was obtained with the measured molecular weight of 4764.50 Da ([M+5H]⁵⁺=953.90).

### SEQ ID NO: 237

### (Cys-Ser-Asn-Leu-Ser-Thr-Cys-Gly-Leu-Gly-Lys-Leu-Ser-Gln-Glu-Ala-His-Lys-Leu-Gln-Thr-Tyr-Pro-Arg-Thr-Asn-Thr-Gly-Ser-Gly-Thr-Pro-Arg-Thr-Asn-Thr-Gly-Ser-Gly-Thr-Pro-Gly-Ile-Cys-Thr-Ala-Ser-Ile-Pro-Pro-Ile-Cys-Gln)

Fmoc-Gln(Trt)-Wang resin was selected as the starting material of chemical synthesis of peptide SEQ ID NO: 237, which was synthesized according to the method described in chemical synthesis of peptide SEQ ID NO: 235. At first, the amino acids were added successively to synthesize the peptide segment with the protecting groups, namely Fmoc-Cys(Acm)-Ser(tBu)-Asn(Trt)-Leu-Ser(tBu)-Thr(tBu)-Cys(Acm)-Gly-Leu-Gly-Lys(Boc)-Leu-Ser (tBu)-Gln(Trt)-Glu(OtBu)-Ala-His(Trt)-Lys(Boc)-Leu-Gln(Trt)-Thr(tBu)-Tyr(tBu)-Pro-Arg(Pbf)-Thr(tBu)-Asn(Trt)-Thr(tBu)-Gly-Ser(tBu)-Gly-Thr(tBu)-Pro-Arg(Pbf)-Thr(tBu)-Asn(Trt)-Thr(tBu)-Gly-Ser(tBu)-Gly-Thr(tBu)-Pro-Gly-Ile- Cys(Trt)-Thr(tBu)-Ala-Ser(tBu)-Ile-Pro-Pro-Ile-Cys(Trt)-Gln(Trt)-Wang resin. Then the Fmoc was removed, and the resin and the side-chain protecting groups were also removed by addition of cleavage buffer, followed by formation of disulfide bond via oxidation. Finally, the target peptide segment was obtained with the measured molecular weight of 4531.50 Da ([M+5H]⁵+=907.30).

### VII. Synthesis Method 6

### Acetylated and Amidated SEQ ID NO: 194

### (Ac-Gly-Arg-Cys-Thr-Lys-Ser-Ile-Pro-Pro-Ile-Cys-Phe-Pro-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-NH₂)

1. Synthesis of the peptide (SEQ ID NO: 194) of 0.1 mmol was performed using Fmoc-Lys(Boc)-Rink Amide AM resin as the initial material. The peptide is synthesized from C-terminal to N-terminal, the N-terminal Fmoc protective group is removed by piperidine/DMF (1:3, v/v) firstly to make the N-terminal a free amino group, 4-fold equivalent Fmoc-Cys(Trt)-OH is dissolved into HOBt/DIC to graft with the resin, the second amino acid residue of C-terminal (Gly) is introduced to obtain Fmoc-Gly-Lys(Boc)-Rink Amide AM resin. As mentioned above, deprotect firstly and then repeatedly connect each amino acid residue of the polypeptide sequence successively, and remove the Fmoc of the last amino acid residue through the method of HOBt/DIC reaction at the last step of the connection of the peptide chain, use the solution of DMF where in 10 times excessed acetic anhydride and 20 times excessed DIEA dissolved for acetic acid reaction, after the synthesis of the whole peptide, the peptide segment with protective groups is obtained, namely Ac-Gly-Arg(Pbf)-Cys(Trt)-Thr(tBu)-Lys(Boc)-Ser(tBu)-Ile-Pro-Pro-Ile-Cys(Trt)-Phe-Pro-Gly-Gly-Ile-Pro-Ile-Gly-Gly-His(Trt)-Ala-Glu(OtBu)-Gly-Thr(tBu)-Phe-Thr(tBu)-Ser(tBu)- Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Rink-Amide Am resin. DMF and DCM are necessary to be used to wash the resin alternately for more than 6 times after each step of the reaction above, and the reaction is controlled through Kaiser Test test. If the condensation reaction of any one of the amino acid residues was incomplete, the condensation should be repeated once, until the desired target peptide segment is obtained.
2. The peptide was removed of Fmoc group and cleaved from the resin by treatment with cleavage reagent (TFA, EDT, TA, phenol, distilled water, TIPS mixed in certain proportion) at 30° C. for 3 h. After cleavage of the protecting group, the filtrate was added into a large amount of cold ether to precipitate the peptide, and then centrifuged. Washed with ether for several times and lyophilized, the crude peptide was obtained, namely Ac-Gly-Arg-Cys-Thr-Lys-Ser-Ile-Pro-Pro-Ile-Cys-Phe-Pro-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val- Lys-Gly-Arg-Gly-Gly-Lys-NH₂.
3. The crude peptide mentioned above was dissolved into DMSO/H₂O (1:4, v/v) solution at the concentration of 4 mg/mL. The reaction solution was taken out and analyzed by HPLC after 24 h. If the oxidation reaction was complete, then performed the purification directly. If the oxidation was incomplete, then the reaction time should be extended until it was complete.
4. The target peptide was obtained through the purification by reverse-phase HPLC performed on a C18 column, whose chemical structure was characterized by MALDI-TOF-MS. The measured molecular weight of SEQ ID NO: 194 is 5533.01 Da ([M+H]⁺).

### Acetylated and Amidated SEQ ID NO: 196

### (Ac-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Arg-Cys-Thr-Lys-Ser-Ile-Pro-Pro-Ile-Cys-Phe-Pro-NH₂)

Fmoc-Pro-Rink Amide-AM resin was selected as the starting material of chemical synthesis of peptide SEQ ID NO: 196, which was synthesized according to the method described in SEQ ID NO: 194. At first, the amino acids were added successively to synthesize the peptide segment with the protecting groups, namely Ac-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val- Lys(Boc)-Gly-Arg(Pbf)-Gly-Gly-Lys(Boc)-Gly-Arg(Pbf)-Cys(Trt)-Thr(tBu)-Lys(Boc)-Ser(tBu)-Ile-Pro-Pro-Ile-Cys(Trt)-Phe-Pro-Rink Amide-AM resin. Then the Fmoc was removed, and the resin and the side-chain protecting groups were also removed by addition of cleavage buffer, followed by formation of disulfide bond via oxidation. Finally, the target peptide segment is obtained with the measured molecular weight of 5476.14 Da ([M+H]⁺).

### Acetylated and Amidated SEQ ID NO: 198

### (Ac-Cys-Gly-Arg-Ala-Thr-Lys-Ser-Ile-Pro-Pro-Ile-Cys-Phe-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-NH₂)

Fmoc-Lys(Boc)-Rink Amide-AM resin was selected as the starting material of chemical synthesis of peptide SEQ ID NO: 198, which was synthesized according to the method described in chemical synthesis of peptide SEQ ID NO: 194. At first, the amino acids were added successively to synthesize the peptide segment with the protecting groups, namely Ac-Cys(Trt)-Gly-Arg(Pbf)-Ala-Thr(tBu)-Lys(Boc)-Ser(tBu)-Ile-Pro-Pro-Ile-Cys(Trt)-Phe-Gly-Gly-Ile-Pro-Ile-Gly-Gly-His(Trt)-Ala-Glu(OtBu)-Gly-Thr(tBu)-Phe-Thr(tBu)-Ser(tBu)- Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Rink Amide AM resin. Then the Fmoc was removed, and the resin and the side-chain protecting groups were also removed by addition of the cleavage buffer, followed by formation of the disulfide bond via oxidation. Finally, the target peptide segment is obtained with the measured molecular weight of 5506.83 Da ([M+H]⁺).

### Acetylated and Amidated SEQ ID NO: 200

### (Ac-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Cys-Gly-Arg-Ala-Thr-Lys-Ser-Ile-Pro-Pro-Ile-Cys-Phe-NH₂)

Fmoc-Phe-Rink Amide-AM resin was selected as the starting material of chemical synthesis of peptide SEQ ID NO: 198, which was synthesized according to the method described in chemical synthesis of peptide SEQ ID NO: 194. At first, the amino acids were added successively to synthesize the peptide segment with the protecting groups, namely Ac-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)-Glu(OtBu)-Phe- Ile-Ala-Trp(Boc)-Leu-Val-Lys(Boc)-Gly-Arg(Pbf)-Gly-Gly-Lys(Boc)-Gly-Cys(Trt)-Gly-Arg(Pbf)-Ala-Thr(tBu)-Lys(Boc)-Ser(tBu)-Ile-Pro-Pro-Ile-Cys(Trt)-Phe-Rink Amide AM resin. Then the Fmoc was removed, and the resin and the side-chain protecting groups were also removed by addition of the cleavage buffer, followed by formation of the disulfide bond via oxidation. Finally, the target peptide segment is obtained with the measured molecular weight of 5507.42 Da ([M+H]⁺).

### VIII. Synthesis Method 7

### N-Terminal PEGylated SEQ ID NO: 200

### (PEG-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys-Gly-Cys-Gly-Arg-Ala-Thr-Lys-Ser-Ile-Pro-Pro-Ile-Cys-Phe)

1. Weighed Fmoc-Phe-Wang resin into the glass reaction column and add DCM to swell for 30 min, the DCM was removed by vacuum filtration.
2. Washed the resin with DMF for 3 times, added piperidine/DMF (1:4, v/v) solution to react for 20 min to remove the protecting group Fmoc. The solution was removed by vacuum filtration, then washed the resin with DMF for 6 times.
3. Weighed Fmoc-Cys(Trt)-OH and TBTU. Added them into the resin and dissolved by DMF. Added DIEA to react for 30 min, and took out of the resin to perform Kaiser Test. It was proved that the reaction was complete when the solution became bright yellow and the resin became yellow. The solvent could be extracted by vacuum filtration.
4. Repeated steps 2 and 3 until connection of the last material Fmoc-PEG8-CH₂CH₂COOH. After 8 h reaction, Fmoc was removed and obtained the peptide segment with PEG modification of N-terminus and side-chain with the protecting group, namely PEG-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)-Val-Ser(tBu)-Ser(tBu)-Tyr(tBu)-Leu-Glu(OtBu)-Gly-Gln(Trt)-Ala-Ala-Lys(Boc)- Glu(OtBu)-Phe-Ile-Ala-Trp(Boc)-Leu-Val-Lys(Boc)-Gly-Arg(Pbf)-Gly-Gly-Lys(Boc)-Gly-Cys(Trt)-Gly-Arg(Pbf)-Ala-Thr(tBu)-Lys(Boc)-Ser(tBu)-Ile-Pro-Pro-Ile-Cys(Trt)-Phe-Wang resin. Washed the resin with DMF, DCM and methanol for three times each, and drained the resin by vacuum filtration.
5. The resin and the side-chain protecting group was removed by treatment with cleavage reagent (TFA, EDT, TA, phenol and distilled water mixed in certain proportion). Filtered with gravel core, added ether into the filtrate for precipitation, centrifuged and washed the solid for three times, drained by vacuum filtration.
6. Dissolved with H₂O/acetonitrile (9:1, v/v), and the volume was increased to 100 mL. Added dilute ammonia solution to adjust pH to basic (pH≈8) and the sample was taken out to test the activity of the thiol group. It indicated the presence of the thiol group when the solution turned yellow. The oxidation was completed (more than 90%) when the solution became clear after addition of 2-3 drops of hydrogen peroxide to react for 5-10 min. Added glacial acetic acid to adjust pH to acidic (pH≈6), and the chemical structure of the peptide was characterized by mass spectrometry. The target peptide of the correct molecular weight was obtained by purification using reversed-phase HPLC on a C18 column. The measured molecular weight of SEQ ID NO: 200 is 5888.73 Da ([M+H]⁺).

### N-Terminal PEGylated SEQ ID NO: 204

### (PEG-Phe-Cys-Thr-Tyr-Ser-Ile-Pro-Pro-Gln-Cys-Tyr-Gly-Gly-Ile-Pro-Ile-Gly-Gly-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-Gly-Lys)

Fmoc-Lys(Boc)-Wang resin was selected as the starting material of chemical synthesis of peptide SEQ ID NO: 204, which was synthesized according to the method described in chemical synthesis of peptide SEQ ID NO: 200. At first, the amino acids were added successively to synthesize the peptide segment with the protecting groups, namely Fmoc-PEG-Phe-Cys(Trt)-Thr(tBu)-Tyr(tBu)-Ser(tBu)-Ile-Pro-Pro-Gln(Trt)-Cys(Trt)-Tyr(tBu)-Gly-Gly-Ile-Pro-Ile-Gly-Gly-His(Trt)-Ala-Glu(OtBu)-Gly-Thr(tBu)-Phe-Thr(tBu)-Ser(tBu)-Asp(OtBu)- Val-Ser(tBu)-Ser(tBu)-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pbf)-Gly-Gly-Lys(Boc)-Wang resin. Then the Fmoc group was removed, and the resin and the side-chain protecting groups were also removed by addition of the cleavage buffer, followed by formation of the disulfide bond via oxidation. Finally, the target peptide segment is obtained, the chemical structure is Characterized by Mass Spectrometry, the Measured Molecular Weight is 5817.47 Da ([M+H]⁺).

### Example 2: Design and Evolution of Inhibitory Peptides Against Trypsin

### Determination of the Michaelis Constant Kₘ Value:

(1) Added 200 μL of reaction buffer (20 mM CaCl₂ and 50 mM Tris-HCl, pH 7.8) into the 96-well plate, and preheated at 37° C. for 15 min. Then added 5 μL of different concentrations of substrates (p-Nitroanilide, pNA, 0.5% in DMSO), and mixed by centrifugation at 500 rpm for 1 min. Finally, the plate was incubated at 37° C. for 120 min, and the absorbance value at 405 nm was measured. In the 205 μL of reaction system, the final concentrations of pNA were 0, 0.025, 0.05, 0.075, 0.1, 0.125, 0.15, 0.2, and 0.25 mM, respectively. The experiment was performed in triplicate. The standard curve was obtained by plotting the OD_{405 nm} value with pNA concentration.
(2) Added 190 μL of reaction buffer (20 mM CaCl₂ and 50 mM Tris-HCl, pH 7.8) and 10 μL of 1 μM trypsin into the 96-well plate, and preheated at 37° C. for 15 min. Then added 5 μL of different concentrations of substrates (BApNA, 0.5% in DMSO) and mixed by centrifugation at 500 rpm for 1 min. Finally, the plate was incubated at 37° C. for 120 min, and the absorbance value at 405 nm was measured. In the 205 μL of reaction system, the final concentrations of BApNA were 0, 0.125, 0.2, 0.33, 0.5, 0.75, 1.0 and 1.25 mM, respectively. The experiment was performed in triplicate. The corresponding curve was obtained by plotting the OD_{405 nm} value with time. Divided the slope of the curve by the slope of the standard curve and the enzyme concentration to obtain the initial velocity V₀ (mM/(min*mM protein). Plotted the initial velocity V₀ with the concentration of substrate BApNA using Prism software, and the Michaelis constant Kₘ value of trypsin hydrolyzing BApNA was obtained.

### Determination of the Inhibition Constant Kᵢ Value:

(1) Added a total 190 μL of reaction buffer (20 mM CaCl₂ and 50 mM Tris-HCl, pH 7.8) along with different concentrations of anti-trypsin peptides (BTs) into the pre-cooled 96-well plate, followed by preheating at 37° C., and spined down at 500 rpm for 5 min. Next added 10 μL of 1 μM trypsin, incubated at 37° C., and then spined down at 500 rpm for 10 min. Finally, added 5 μL of 50 mM BApNA, and mixed by centrifugation at 500 rpm for 1 min. The plate was incubated at 37° C. for 260 min, and the absorbance value at 405 nm was measured. The experiment was performed in triplicate. The blank control only contained the reaction buffer and the substrate as the minimum absorption value (Min OD_{405 nm}). The negative control only contained the reaction buffer, the enzyme and the substrate as the maximum absorption value (Max OD_{405 nm}).
(2) In the reaction system of 205 μL, the final concentrations of trypsin and BApNA were 50 nM and 1.22 mM, respectively.
(3) Data statistics

Residual activity of the enzyme (%)=(1−(Max OD_{405 nm}−Sample OD_{405 nm})/(Max OD_{405 nm}−Min OD_{405 nm}))*100

Plotted the residual activity of the enzyme with substrate concentration to obtain the half inhibitory concentration (IC₅₀) of BTs scaffold against trypsin. Then, substituted it into the formula Kᵢ=IC₅₀/(1+S/Kₘ) (S, IC₅₀ and Kₘ were substrate concentration, half inhibitory concentration and Michaelis constant, respectively) to obtain the inhibition constant Kᵢ of BTs scaffold against trypsin.

### Results:

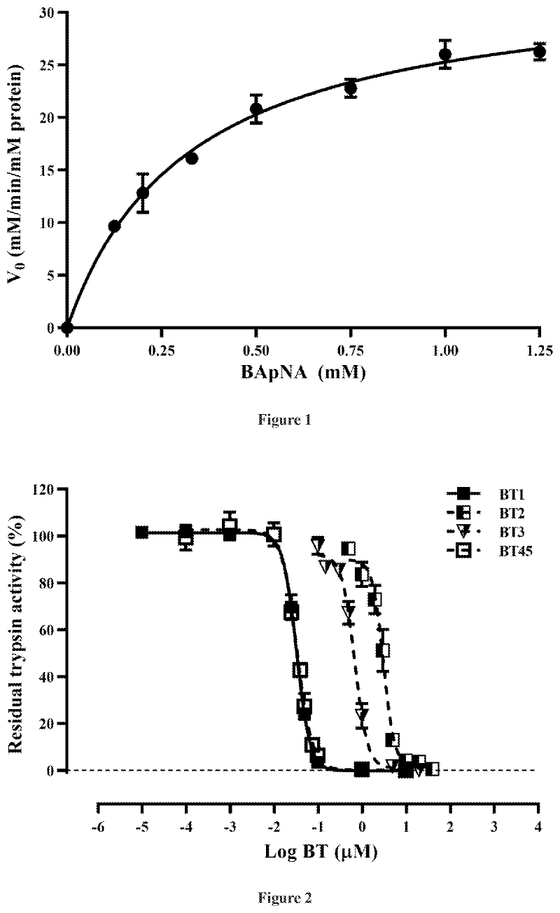

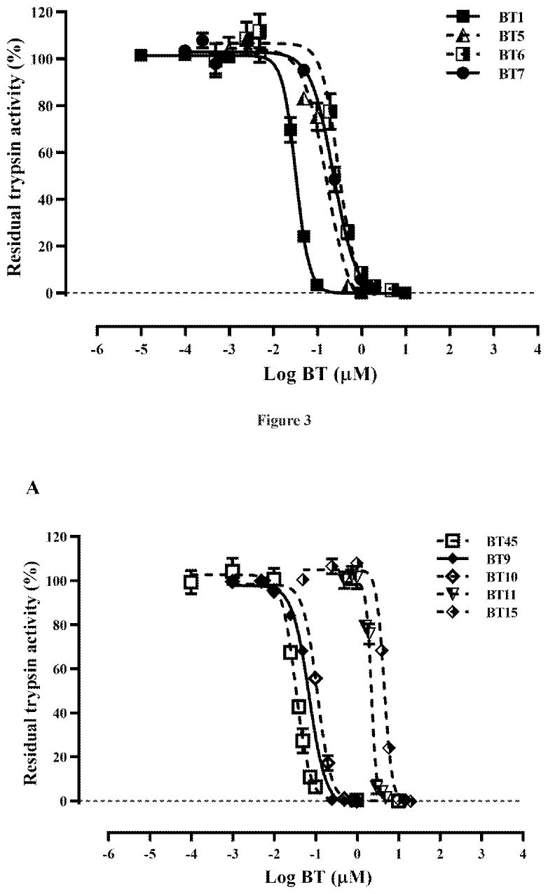

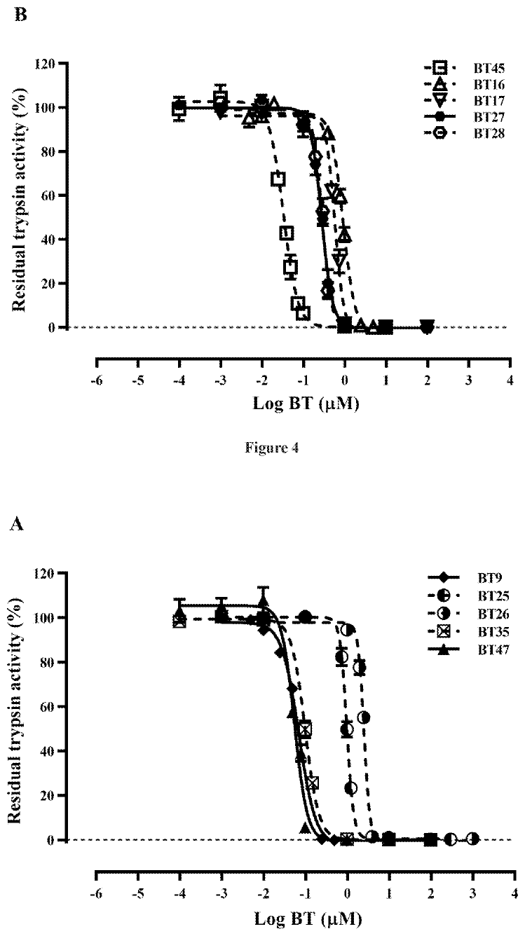

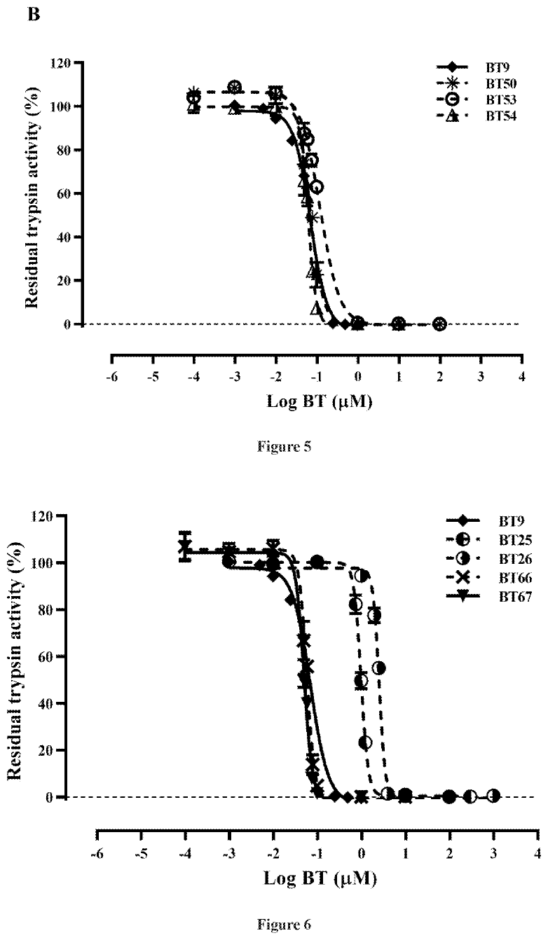

According to the OD_{405 nm} value of pNA produced by trypsin hydrolyzing different concentrations of BApNA in reference to that of the standard curve, plotted the initial velocity V₀ with the concentration of substrate BApNA using Prism software, obtaining Michaelis constant Kₘ value of 0.33 mM (R²=0.9966) (FIG. 1). Using a rational design method, linear and N/C truncated SFTI-1 analogues BT1 and BT45 were designed and synthesized. The inhibition constants (Kᵢ) of them towards trypsin were determined to be both 6.4 nM (FIG. 2 and Table 3), in consistent with the research results disclosed in the literature [Korsinczky M L, Schirra H J, Rosengren K J, West J, Condie B A, Otvos L, et al. Solution structures by 1H NMR of the novel cyclic trypsin inhibitor SFTI-1 from sunflower seeds and an acyclic permutant. J Mol Biol, 2001, 311: 579-591.]. The results confirmed that truncation of 1 (G) and 2 (FD) amino acid residues at the N-terminus and C-terminus of linear SFTI-1 did not affect trypsin inhibitory activity. At the same time, BT2 and BT3 with mutations at P3 site were designed and synthesized, and their inhibition constants (Kᵢ) were determined to be 650 and 140 nM, respectively (FIG. 2 and Table 3). Although the inhibitory activity of these two peptides decreased, it demonstrated that the P3 site of inhibitory activity loop of SFTI-1 was tolerant to mutation, and the peptide segment (loop) between the disulfide bond could be extended. Subsequently, BT5, BT6, and BT7 were synthesized by optimizing the loop between the disulfide bond, and their inhibition constants (Kᵢ) were determined to be 30, 60 and 50 nM, respectively (FIG. 3, Table 2, and Table 4). Then, the simplified structure and the expanded loop with P3 site mutated were combined to design and synthesize a series of mutants (BT8-BT36) for amino acid residue replacement at P1′-P7′ (Table 2). The results of the inhibition constant (Kᵢ) showed that the deletion of phenylalanine at P7′ site (BT8, IC₅₀>50 μM) and substitution of proline at the P3′ site with alanine (BT20, IC₅₀>50 μM) greatly reduced its trypsin inhibitory activity. Among them, BT9 derived from loop expansion of BT45 exhibited good inhibitory activity (Kᵢ=10 nM), while substitutions at other sites exhibited different effects. Among them, BT10 was a mutant with mutation of proline to alanine at P4′ site, whose inhibitory activity (Kᵢ=20 nM) was less affected. Secondly, the P1 site of BT17 with lysine mutated to arginine had a nearly 12-fold decrease in inhibitory activity compared to BT9. Next, other amino acid substitutions of sites P1′ (e.g., mutant BT27, BT22) and P2′ (e.g., mutant BT28, BT16, BT14, BT21) reduced their anti-trypsin activities; and the amino acid substitution of P5′ (e.g., mutant BT15, BT12) and P7′ (e.g., mutant BT12, BT18, BT19, BT24) also showed a significant impact on their anti-trypsisn activities. In addition, further expanding the loop length between the disulfide bond on the basis of BT9 still maintained good inhibitory activity against trypsin (e.g., mutant BT11, BT13, BT32, BT33, BT29) (FIG. 4, Table 2, and Table 5).

Mutation studies at the sites P2 (BT26), P3 (BT35), P4 (BT25), and P5 (BT66) of BT9 have been found that it could be replaced by other amino acid residues, with alanine substitution of the P3 site being replaced by γ-aminobutyric acid exhibited almost equivalent inhibitory activity of peptide BT9 against trypsin, leading to the further synthesis of a series of BT47-BT60 scaffolds targeting the P3 site. Among them, mutants BT47, BT50, BT53, and BT54 exhibited good inhibitory activities against trypsin (FIG. 5, Table 2, and Table 6). Replacing glycine at P5 site to proline which promoting D3-folding formation, scaffold peptides still exhibited good inhibitory activity against trypsin, resulting in the synthesis of scaffolds of BT66-BT80. Among them, scaffold peptides BT66 and BT67 exhibited higher trypsin inhibitory activities against trypsin (FIG. 6, Table 2, and Table 7).

TABLE 2
Molecular structures and inhibitory activities of anti-trypsin peptides

| | | | | | Theoretical |
|---|---|---|---|---|---|
| | | | IC₅₀ | Kᵢ | molecular weight |
| NO. | Scafflods | Amino acid sequences^{a} | (μM) | (μM) | (Da) |
| 1 | BT1 | GRCTKSIPPICFPD | 0.03 | 0.0064 | 1531.82 |
| 2 | BT2 | CGAKGTKSIPPICFPD | 3.03 | 0.65 | 1631.94 |
| 3 | BT3 | CGAKATKSIPPICFPD | 0.68 | 0.14 | 1645.97 |
| 4 | BT4 | CGAKGTKSIPPIGFCD | >50 | N.A. | 1591.88 |
| 5 | BT5 | CGRATKSIPPICFPD | 0.16 | 0.03 | 1602.90 |
| 6 | BT6 | CGSATKSIPPICFPD | 0.29 | 0.06 | 1533.79 |
| 7 | BT7 | CGAATKSIPPICFPD | 0.23 | 0.05 | 1517.79 |
| 8 | BT8 | CGRATKSIPPIC | >50 | N.A. | 1243.52 |
| 9 | BT9 | CGRATKSIPPICF | 0.07 | 0.01 | 1390.70 |
| 10 | BT10 | CGRATKSIPAICF | 0.11 | 0.02 | 1364.66 |
| 11 | BT11 | CGRATKSIPPIAFC | 2.20 | 0.47 | 1461.78 |
| 12 | BT12 | CGRATKSIPPQCY | 8.46 | 1.80 | 1421.67 |
| 13 | BT13 | CGRATKSIPPIAC | 31.49 | 6.70 | 1314.60 |
| 14 | BT14 | CGRATKSLPPACF | 1.65 | 0.35 | 1350.62 |
| 15 | BT15 | CGRATRSIPPACF | 4.49 | 0.96 | 1378.63 |
| 16 | BT16 | CGRATKSLPAICF | 0.91 | 0.19 | 1366.66 |
| 17 | BT17 | CGRATRSIPPICF | 0.58 | 0.12 | 1418.71 |
| 18 | BT18 | CGRATRSIPPICY | 39.35 | 8.38 | 1434.71 |
| 19 | BT19 | CGRATRSIPPICA | 43.43 | 9.25 | 1342.61 |
| 20 | BT20 | CGRATRSIAPICF | >50 | N.A. | 1392.67 |
| 21 | BT21 | CGRATRSAPPICF | 18.06 | 3.85 | 1376.63 |
| 22 | BT22 | CGRATRAIPPICF | 4.68 | 1.00 | 1402.71 |
| 23 | BT23 | GTCTRSIPPICNPN | 1.82 | 0.39 | 1470.70 |
| 24 | BT24 | CGTATKSIPPICN | 33.90 | 7.22 | 1302.54 |
| 25 | BT25 | CGTATKSIPPICF | 0.97 | 0.21 | 1335.61 |
| 26 | BT26 | CGRAAKSIPPICF | 2.50 | 0.53 | 1360.67 |
| 27 | BT27 | CGRATKAIPPICF | 0.28 | 0.06 | 1374.70 |
| 28 | BT28 | CGRATKSNlePPICF | 0.29 | 0.06 | 1390.71 |
| 29 | BT29 | HcyGRATKSIPPIAFHcy | 10.6 | 2.26 | 1489.86 |
| 30 | BT30 | CGRATKSIPPIFC | >50 | N.A. | 1390.70 |
| 31 | BT31 | CGRATKSIPPAFC | >50 | N.A. | 1348.62 |
| 32 | BT32 | CGRATKSIPPIAFGC | 1.54 | 0.33 | 1518.83 |
| 33 | BT33 | HcyGRATKSIPPIAFGHcy, | 5.57 | 1.19 | 1546.91 |
| 34 | BT34 | CGRATKSIPPQARC | >50 | N.A. | 1485.76 |
| 35 | BT35 | CGRAbuTKSIPPICF | 0.06 | 0.01 | 1404.74 |
| 36 | BT36 | CWTKSIPPKPC | 4.65 | 0.99 | 1257.55 |
| 37 | BT37 | CGWTKSIPPKPC | >50 | N.A. | 1314.60 |
| 38 | BT38 | CGRWTKSIPPKPC | >50 | N.A. | 1470.79 |
| 39 | BT39 | CGRWTKSIPPAFC | >50 | N.A. | 1463.75 |
| 40 | BT40 | CGRWTKSIPPIFC | >50 | N.A. | 1505.83 |
| 41 | BT41 | GRCPKILKKCF | >50 | N.A. | 1290.67 |
| 42 | BT42 | CGRAPKILKKCF | >50 | N.A. | 1361.75 |
| 43 | BT43 | GRCPKILQRCF | >50 | N.A. | 1318.64 |
| 44 | BT44 | CGRAPKILQRCF | >50 | N.A. | 1389.72 |
| 45 | BT45 | RCTKSIPPICF | 0.03 | 0.0064 | 1262.57 |
| 46 | BT46 | CHypRATKSIPPICF | 0.07 | 0.01 | 1444.76 |
| 47 | BT47 | CGRSTKSIPPICF | 0.05 | 0.01 | 1406.70 |
| 48 | BT48 | CGRLTKSIPPICF | 0.18 | 0.04 | 1432.78 |
| 49 | BT49 | CGRITKSIPPICF | 0.50 | 0.11 | 1432.78 |
| 50 | BT50 | CGRNleTKSIPPICF | 0.07 | 0.01 | 1432.79 |
| 51 | BT51 | CGRVTKSIPPICF | 0.41 | 0.09 | 1418.75 |
| 52 | BT52 | CGRFTKSIPPICF | 0.13 | 0.03 | 1466.80 |
| 53 | BT53 | CGRYTKSIPPICF | 0.12 | 0.03 | 1482.79 |
| 54 | BT54 | CGRQTKSIPPICF | 0.06 | 0.01 | 1447.75 |
| 55 | BT55 | CGRNTKSIPPICF | 0.30 | 0.06 | 1433.72 |
| 56 | BT56 | CGRHTKSIPPICF | 0.11 | 0.02 | 1456.76 |
| 57 | BT57 | CGRWTKSIPPICF | 0.13 | 0.03 | 1505.83 |
| 58 | BT58 | CGRETKSIPPICF | 0.12 | 0.03 | 1448.73 |
| 59 | BT59 | CGRPTKSIPPICF | 26.27 | 5.59 | 1416.74 |
| 60 | BT60 | CGRGTKSIPPICF | 0.43 | 0.09 | 1376.67 |
| 61 | BT61 | RCTRSIPPHCW | 0.89 | 0.19 | 1353.60 |
| 62 | BT62 | RCTKSIPPHCF | 0.07 | 0.01 | 1286.55 |
| 63 | BT63 | RCTKSIPPQCH | 0.09 | 0.02 | 1267.50 |
| 64 | BT64 | RCTKSNPPQCQ | 3.43 | 0.73 | 1259.44 |
| 65 | BT65 | RCTKSLPPQCS | 0.62 | 0.13 | 1217.44 |
| 66 | BT66 | CPRATKSIPPICF | 0.06 | 0.01 | 1430.76 |
| 67 | BT67 | CARATKSIPPICF | 0.05 | 0.01 | 1404.72 |
| 68 | BT68 | ACTKSNPPQCR | 2.43 | 0.52 | 1202.38 |
| 69 | BT69 | CARATKSIHypPICF | 0.06 | 0.01 | 1420.72 |
| 70 | BT70 | CARATKSIPHypICF | 0.15 | 0.03 | 1420.72 |
| 71 | BT71 | CVRATKSIPPICF | 0.11 | 0.02 | 1432.78 |
| 72 | BT72 | CLRATKSIPPICF | 0.13 | 0.03 | 1446.80 |
| 73 | BT73 | CIRATKSIPPICF | 0.12 | 0.03 | 1446.80 |
| 74 | BT74 | CAbuRATKSIPPICF | 0.09 | 0.02 | 1418.77 |
| 75 | BT75 | CSRATKSIPPICF | 0.04 | 0.0085 | 1420.72 |
| 76 | BT76 | CRRATKSIPPICF | 0.05 | 0.01 | 1489.83 |
| 77 | BT77 | CKRATKSIPPICF | 0.04 | 0.0085 | 1461.82 |
| 78 | BT78 | CERATKSIPPICF | 0.03 | 0.0064 | 1462.76 |
| 79 | BT79 | CQRATKSIPPICF | 0.05 | 0.01 | 1461.78 |
| 80 | BT80 | CNleRATKSIPPICF | 0.10 | 0.02 | 1446.82 |

^{a}: A disulfide bond is formed between two cysteine residues of anti-trypsin peptide
scaffolds.
N.A: Molecules with weak activity are no longer measured for Kᵢ value.

TABLE 3
Determination of the inhibitory activities of anti-trypsin peptides

| | Residual | | Residual | | Residual | | Residual |
|---|---|---|---|---|---|---|---|
| | trypsin | | trypsin | | trypsin | | trypsin |
| BT1 | activity | BT2 | activity | BT3 | activity | BT45 | activity |
| (μM) | (%) | (μM) | (%) | (μM) | (%) | (μM) | (%) |
| 0.0000098 | 101.6 ± 1.0 | 0.488 | 94.6 ± 1.7 | 0.098 | 95.7 ± 3.6 | 0.000098 | 99.3 ± 5.3 |
| 0.000098 | 101.8 ± 2.2 | 0.98 | 83.6 ± 5.2 | 0.1463 | 86.8 ± 2.5 | 0.00098 | 104.3 ± 6.0 |
| 0.00098 | 100.8 ± 1.7 | 1.95 | 72.9 ± 6.1 | 0.293 | 85.6 ± 1.3 | 0.0098 | 100.7 ± 5.0 |
| 0.02439 | 69.6 ± 5.3 | 2.93 | 51.2 ± 9.0 | 0.488 | 67.2 ± 4.8 | 0.02439 | 67.4 ± 2.6 |
| 0.0488 | 24.1± 0.5 | 4.88 | 12.9 ± 1.3 | 0.98 | 23.3 ± 5.3 | 0.0390 | 42.7 ± 2.9 |
| 0.098 | 3.3 ± 0.7 | 9.76 | 4.2 ± 0.4 | 4.88 | 1.8 ± 0.9 | 0.0488 | 27.3 ± 5.5 |
| 0.98 | 0.0 ± 0.1 | 19.51 | 3.7 ± 0.5 | 9.76 | 0.9 ± 0.4 | 0.07317 | 10.8 ± 1.6 |
| 9.76 | 0.0 ± 0.0 | 39.02 | 0.5 ± 0.1 | 19.51 | 0.1 ± 0.0 | 0.098 | 6.3 ± 0.4 |
| | | | | | | 0.98 | 0.4 ± 0.0 |
| | | | | | | 9.76 | 0.0 ± 0.0 |

TABLE 4
Determination of the inhibitory activities of anti-trypsin peptides

| | Residual | | Residual | | Residual | | Residual |
|---|---|---|---|---|---|---|---|
| | trypsin | | trypsin | | trypsin | | trypsin |
| BT1 | activity | BT5 | activity | BT6 | activity | BT7 | activity |
| (μM) | (%) | (μM) | (%) | (μM) | (%) | (pμM) | (%) |
| 0.0000098 | 101.6 ± 1.0 | 0.00098 | 104.0 ± 5.2 | 0.000488 | 99.3 ± 7.2 | 0.000098 | 103.5 ± 1.6 |
| 0.000098 | 101.8 ± 2.2 | 0.002439 | 108.1 ± 2.6 | 0.002439 | 108.6 ± 7.1 | 0.0002439 | 107.8 ± 3.2 |
| 0.00098 | 100.8 ± 1.7 | 0.00488 | 103.7 ± 5.2 | 0.00488 | 111.9 ± 7.2 | 0.000488 | 97.9 ± 4.3 |
| 0.02439 | 69.6 ± 5.3 | 0.0488 | 83.1 ± 0.4 | 0.195 | 77.5 ± 7.6 | 0.0488 | 95.2 ± 1.1 |
| 0.0488 | 24.1 ± 0.5 | 0.098 | 75.2 ± 5.9 | 0.488 | 25.9 ± 2.9 | 0.2439 | 48.5 ± 5.3 |
| 0.098 | 3.3 ± 0.7 | 0.488 | 3.0 ± 0.2 | 0.98 | 8.5 ± 0.9 | 0.98 | 5.7 ± 1.7 |
| 0.98 | 0.0 ± 0.1 | 0.98 | 0.7 ± 0.1 | 1.95 | 2.8 ± 0.0 | 1.463 | 2.9 ± 1.1 |
| 9.76 | 0.0 ± 0.0 | | | 4.88 | 1.2 ± 0.2 | 1.95 | 1.5 ± 0.3 |

TABLE 5
Determination of the inhibitory activities of anti-trypsin peptides

| BT45 (μM) | Residual trypsin activity (%) |
|---|---|
| 0.000098 | 99.3 ± 5.3 |
| 0.00098 | 104.3 ± 6.0 |
| 0.0098 | 100.7 ± 5.0 |
| 0.02439 | 67.4 ± 2.6 |
| 0.0390 | 42.7 ± 2.9 |
| 0.0488 | 27.3 ± 5.5 |
| 0.07317 | 10.8 ± 1.6 |
| 0.098 | 6.3 ± 0.4 |
| 0.98 | 0.4 ± 0.0 |
| 9.76 | 0.0 ± 0.0 |

| | Residual | | Residual | | Residual | | Residual |
|---|---|---|---|---|---|---|---|
| | trypsin | | trypsin | | trypsin | | trypsin |
| BT9 | activity | BT10 | activity | BT11 | activity | BT12 | activity |
| (μM) | (%) | (μM) | (%) | (μM) | (%) | (μM) | (%) |
| 0.00098 | 100.4 ± 0.6 | 0.00098 | 99.7 ± 0.6 | 0.488 | 100.2 ± 3.7 | 0.0488 | 100.5 ± 2.0 |
| 0.00488 | 99.0 ± 0.5 | 0.00488 | 99.8 ± 1.8 | 0.7317 | 102.4 ± 4.2 | 0.2439 | 106.5 ± 3.4 |
| 0.0098 | 94.3 ± 1.3 | 0.0098 | 95.5 ± 1.1 | 0.98 | 101.5 ± 5.1 | 0.98 | 107.9 ± 1.4 |
| 0.02439 | 84.2 ± 1.9 | 0.098 | 55.6 ± 2.3 | 1.561 | 79.3 ± 1.0 | 3.90 | 68.3 ± 0.1 |
| 0.0488 | 68.1 ± 1.4 | 0.195 | 17.2 ± 3.4 | 1.95 | 75.8 ± 4.6 | 5.85 | 24.0 ± 1.2 |
| 0.2439 | 0.3 ± 0.0 | 0.488 | 1.0 ± 0.1 | 2.93 | 6.2 ± 3.1 | 9.76 | 0.7 ± 0.1 |
| 0.488 | −0.2 ± 0.1 | 0.7317 | 0.2 ± 0.1 | 3.90 | 4.1 ± 1.0 | 14.63 | 0.3 ± 0.1 |
| 0.98 | −0.4 ± 0.0 | 0.98 | −0.1 ± 0.1 | 4.88 | 1.4 ± 0.3 | 19.51 | −0.2 ± 0.1 |

| | Residual | | Residual | | Residual | | Residual |
|---|---|---|---|---|---|---|---|
| | trypsin | | trypsin | | trypsin | | trypsin |
| BT16 | activity | BT17 | activity | BT27 | activity | BT28 | activity |
| (μM) | (%) | (μM) | (%) | (μM) | (%) | (μM) | (%) |
| 0.00488 | 95.4 ± 4.3 | 0.00098 | 97.9 ± 0.8 | 0.000098 | 101.0 ± 0.7 | 0.00098 | 102.3 ± 0.7 |
| 0.0098 | 96.1 ± 0.6 | 0.0098 | 97.5 ± 0.7 | 0.00098 | 98.9 ± 1.5 | 0.0098 | 101.8 ± 0.1 |
| 0.0195 | 101.7 ± 1.2 | 0.098 | 93.6 ± 4.5 | 0.0098 | 103.5 ± 2.4 | 0.098 | 92.2 ± 5.5 |
| 0.390 | 88.4 ± 2.0 | 0.488 | 61.9 ± 3.0 | 0.098 | 92.0 ± 1.9 | 0.195 | 77.6 ± 8.3 |
| 0.780 | 59.7 ± 3.2 | 0.7317 | 30.0 ± 5.3 | 0.195 | 74.1 ± 1.3 | 0.293 | 52.7 ± 5.9 |
| 0.98 | 42.2 ± 3.3 | 0.98 | 1.5 ± 0.3 | 0.293 | 49.2 ± 3.0 | 0.390 | 16.7 ± 3.7 |
| 2.439 | 0.8 ± 0.1 | 9.76 | 0.1 ± 0.0 | 0.390 | 19.9 ± 6.3 | 0.98 | 0.7 ± 0.0 |
| 4.88 | 0.0 ± 0.2 | 97.56 | 0.1 ± 0.0 | 0.98 | 1.3 ± 0.3 | 9.76 | 0.1 ± 0.0 |
| | | | | 9.76 | 0.1 ± 0.0 | 97.56 | 0.0 ± 0.0 |
| | | | | 97.56 | 0.1 ± 0.0 | | |

TABLE 6
Determination of the inhibitory activities of anti-trypsin peptides

| | Residual | | Residual | | Residual | | Residual |
|---|---|---|---|---|---|---|---|
| | trypsin | | trypsin | | trypsin | | trypsin |
| BT9 | activity | BT25 | activity | BT26 | activity | BT35 | activity |
| (μM) | (%) | (μM) | (%) | (μM) | (%) | (μM) | (%) |
| 0.00098 | 100.4 ± 0.6 | 0.00098 | 100.6 ± 1.3 | 0.0098 | 99.5 ± 0.9 | 0.000098 | 98.3 ± 1.1 |
| 0.00488 | 99.0 ± 0.5 | 0.0098 | 99.6 ± 1.4 | 0.098 | 100.2 ± 1.4 | 0.00098 | 100.4 ± 0.7 |
| 0.0098 | 94.3 ± 1.3 | 0.098 | 100.4 ± 1.2 | 0.98 | 94.5 ± 0.7 | 0.0098 | 99.0 ± 2.4 |
| 0.02439 | 84.2 ± 1.9 | 0.7317 | 82.3 ± 3.9 | 1.95 | 77.6 ± 3.1 | 0.098 | 49.8 ± 3.7 |
| 0.0488 | 68.1 ± 1.4 | 0.98 | 49.7 ± 3.5 | 2.439 | 55.1 ± 1.3 | 0.1463 | 25.6 ± 2.0 |
| 0.2439 | 0.3 ± 0.0 | 1.2195 | 23.2 ± 2.1 | 3.90 | 1.4 ± 0.1 | 0.98 | 0.3 ± 0.0 |
| 0.488 | −0.2 ± 0.1 | 9.76 | 1.0 ± 0.1 | 9.76 | 0.4 ± 0.1 | 9.76 | 0.1 ± 0.0 |
| 0.98 | −0.4 ± 0.0 | 97.56 | 0.2 ± 0.1 | 97.56 | 0.1 ± 0.0 | 97.56 | 0.0 ± 0.0 |
| | | 292.68 | 0.2 ± 0.2 | 975.61 | 0.5 ± 0.0 | | |

| | Residual | | Residual | | Residual | | Residual |
|---|---|---|---|---|---|---|---|
| | trypsin | | trypsin | | trypsin | | trypsin |
| BT47 | activity | BT50 | activity | BT53 | activity | BT54 | activity |
| (μM) | (%) | (μM) | (%) | (μM) | (%) | (μM) | (%) |
| 0.000098 | 103.0 ± 5.2 | 0.000098 | 106.1 ± 2.6 | 0.000098 | 104.2 ± 0.5 | 0.000098 | 101.0 ± 3.9 |
| 0.00098 | 105.1 ± 3.6 | 0.00098 | 108.5 ± 1.0 | 0.00098 | 108.9 ± 2.0 | 0.00098 | 99.2 ± 1.7 |
| 0.0098 | 107.9 ± 5.8 | 0.0098 | 105.0 ± 3.9 | 0.0098 | 106.0 ± 0.4 | 0.0098 | 99.9 ± 4.1 |
| 0.0488 | 57.4 ± 1.1 | 0.0488 | 73.5 ± 1.6 | 0.0488 | 87.4 ± 4.9 | 0.0488 | 65.8 ± 6.7 |
| 0.0780 | 39.0 ± 3.8 | 0.0780 | 48.9 ± 1.8 | 0.0585 | 84.9 ± 2.2 | 0.0585 | 58.4 ± 4.3 |
| 0.098 | 5.6 ± 0.7 | 0.098 | 22.6 ± 5.7 | 0.07317 | 75.2 ± 2.8 | 0.0780 | 24.3 ± 2.4 |
| 0.98 | 0.2 ± 0.0 | 0.98 | 0.1 ± 0.0 | 0.098 | 63.0 ± 1.9 | 0.098 | 7.2 ± 2.4 |
| 9.76 | 0.0 ± 0.1 | 9.76 | 0.0 ± 0.1 | 0.98 | 0.5 ± 0.2 | 0.98 | 0.1 ± 0.0 |
| 97.56 | 0.1 ± 0.0 | 97.56 | 0.0 ± 0.1 | 9.76 | 0.2 ± 0.0 | 9.76 | −0.1 ± 0.0 |
| | | | | 97.56 | 0.0 ± 0.1 | | |

TABLE 7
Determination of the inhibitory activities of anti-trypsin peptides

| BT9 (μM) | Residual trypsin activity (%) |
|---|---|
| 0.00098 | 100.4 ± 0.6 |
| 0.00488 | 99.0 ± 0.5 |
| 0.0098 | 94.3 ± 1.3 |
| 0.02439 | 84.2 ± 1.9 |
| 0.0488 | 68.1 ± 1.4 |
| 0.2439 | 0.3 ± 0.0 |
| 0.488 | −0.2 ± 0.1 |
| 0.98 | −0.4 ± 0.0 |

| | Residual | | Residual | | Residual | | Residual |
|---|---|---|---|---|---|---|---|
| | trypsin | | trypsin | | trypsin | | trypsin |
| BT25 | activity | BT26 | activity | BT66 | activity | BT67 | activity |
| (μM) | (%) | (μM) | (%) | (μM) | (%) | (μM) | (%) |
| 0.00098 | 100.6 ± 1.3 | 0.0098 | 99.5 ± 0.9 | 0.000098 | 107.0 ± 6.2 | 0.000098 | 107.0 ± 5.5 |
| 0.0098 | 99.6 ± 1.4 | 0.098 | 100.2 ± 1.4 | 0.00098 | 104.7 ± 2.1 | 0.00098 | 104.4 ± 3.9 |
| 0.098 | 100.4 ± 1.2 | 0.98 | 94.5 ± 0.7 | 0.0098 | 106.2 ± 3.4 | 0.0098 | 102.0 ± 2.4 |
| 0.7317 | 82.3 ± 3.9 | 1.95 | 77.6 ± 3.1 | 0.0488 | 66.8 ± 8.2 | 0.0488 | 49.6 ± 2.8 |
| 0.98 | 49.7 ± 3.5 | 2.439 | 55.1 ± 1.3 | 0.0585 | 55.7 ± 2.4 | 0.0585 | 39.9 ± 1.7 |
| 1.2195 | 23.2 ± 2.1 | 3.90 | 1.4 ± 0.1 | 0.0780 | 13.9 ± 4.2 | 0.07317 | 8.1 ± 2.5 |
| 9.76 | 1.0 ± 0.1 | 9.76 | 0.4 ± 0.1 | 0.098 | 4.8 ± 0.7 | 0.098 | 1.5 ± 0.3 |
| 97.56 | 0.2 ± 0.1 | 97.56 | 0.1 ± 0.0 | 0.98 | 0.1 ± 0.0 | 0.98 | 0.1 ± 0.2 |
| 292.68 | 0.2 ± 0.2 | 975.61 | 0.5 ± 0.0 | 9.76 | 0.1 ± 0.0 | 9.76 | 0.1 ± 0.1 |

### Example 3: Design and Inhibitory Activity Evaluation of Peptides Against Chymotrypsin

### Determination of the Michaelis Constant Kₘ Value:

(1) Added a total 190 μL of reaction buffer (20 mM CaCl₂, 50 mM Tris-HCl (pH 7.8)) into the 96 well plate, followed by preheating at 37° C. for 15 min. Then added 2 μL of different concentrations of substrates (p-Nitroanilide, pNA, 0.5% in DMSO), and mixed by centrifugation at 500 rpm for 1 min. Finally, the plate was incubated at 37° C. for 20 min, and the absorbance value at 405 nm was measured. In the 200 μL of reaction system, the final concentrations of pNA were 0, 0.025, 0.05, 0.075, 0.1, 0.125, 0.15, 0.25, and 0.3 mM, respectively. Make three repetitions for each concentration and plot the OD₄₀₅ m value with pNA concentration to obtain the standard curve.
(2) Added a total 190 μL of reaction buffer (20 mM CaCl₂, 50 mM Tris-HCl (pH 7.8)) and 8 μL of 0.75 μM chymotrypsin into the 96 well plate, followed by preheating at 37° C. for 5 min. Then added 2 μL of different concentrations of substrates (AAPFpNA, dissolved in DMSO), and mixed by centrifugation at 500 rpm for 1 min. Finally, the plate was incubated at 37° C. for 20 min, and the absorbance value at 405 nm was measured. In the 200 μL of reaction system, the final concentrations of AAPFpNA were 0, 0.125, 0.25, 0.285, 0.33, 0.4, and 0.5 mM, respectively. Make three repetitions for each concentration and plot the OD_{405 nm} value with time to obtain the corresponding curve. Divide the slope of the curve by the slope of the standard curve and the enzyme concentration to obtain the initial velocity V₀ (mM/(min*mM protein). Plot the initial velocity V₀ with the concentration of substrate AAPFpNA using Prism software, and the Michaelis constant Kₘ value of chymotrypsin hydrolyzing AAPFpNA was obtained.

### Determination of the Inhibition Constant Kᵢ Value:

(1) Added a total 190 μL of reaction buffer (20 mM CaCl₂, 50 mM Tris-HCl (pH 7.8)), different concentrations of anti-chymorypsin peptides (CHs) into the pre-cold 96 well plate, followed by preheating at 37° C. for 5 min and centrifugated at 500 rpm for 1 min and allowed to stand for 4 minutes. Then added 8 μL of 750 nM chymotrypsin and incubated at 37° C. for 10 min, and centrifugated at 500 rpm for 1 min and allowed to stand for 9 minutes. Finally, added 2 μL of 50 mM AAPFpNA, and mixed by centrifugation at 500 rpm for 1 min. The plate was incubated at 37° C. for 90 min, and the absorbance value at 405 nm was measured. Three repetitions were made, and only reaction buffer and substrate were added in the blank control as the minimum absorption value (Min OD_{405 nm}); Only reaction buffer, enzyme and substrate were added in the negative control as the maximum absorption value (Max OD₄₀₅).
(2) In the 200 μL of reaction system, the final concentrations of chymotrypsin and AAPFpNA were 30 nM and 0.5 mM, respectively.
(3) Data statistics

Residual activity of enzyme (%)=(1−(Max OD_{405 nm}−Sample OD_{405 nm})/(Max OD_{405 nm}−Min OD_{405 nm}))*100

Plotted the residual activity of enzyme with substrate concentration to obtain the half inhibitory concentration (IC50) of anti-chymorypsin peptides (CHs). Then, substituted it into the formula Kᵢ=IC50/(1+S/Kₘ) (S, IC50, and Kₘ were substrate concentration, half inhibitory concentration, and Michaelis constant, respectively) to obtain the inhibition constant Kᵢ of anti-chymorypsin peptides (CHs).

### Results:

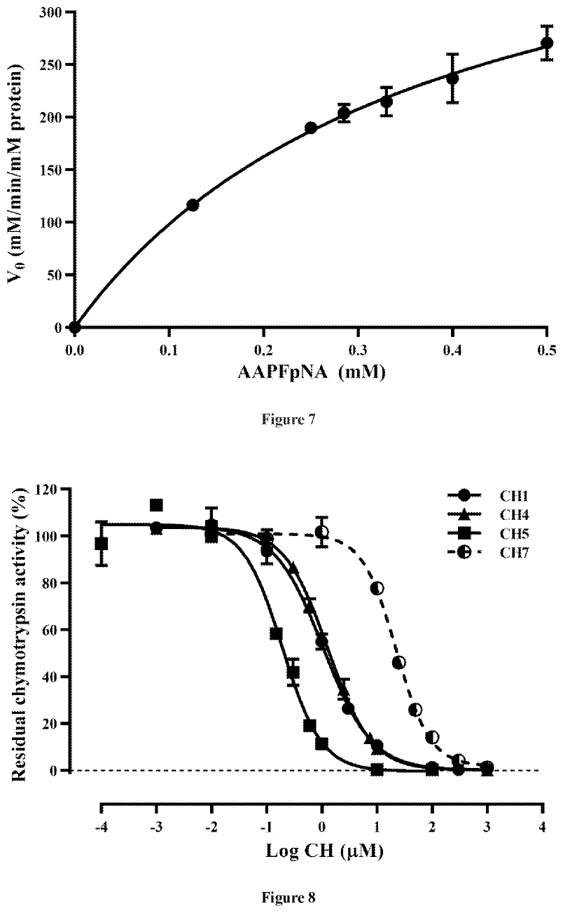

Using a certain concentration of chymotrypsin to catalyze different concentrations of AAPFpNA to produce pNA, and the absorbance value of OD_{405 nm} was measured. Referring to the standard curve, Prism software was used to plot the initial velocity V₀ with the concentration of substrate AAPFpNA, and the Michaelis constant Kₘ value of chymotrypsin hydrolyzing AAPFpNA was obtained to be 0.38 mM (R²=0.9988) (FIG. 7).

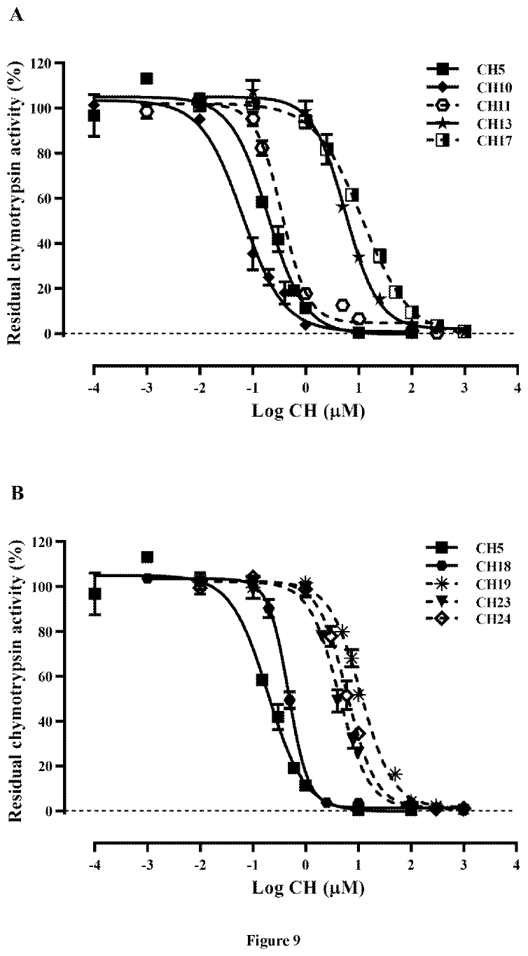

There is limited research on active peptides derived from BBI and SFTI-1 that inhibit chymotrypsin. A literature had reported that peptide analog CH4 derived from SFTI-1 has good inhibitory activity against chymotrypsin [McBride J D, Freeman N, Domingo G J, Leatherbarrow R J. Selection of chymotrypsin inhibitors from a conformationally constrained combinatorial peptide library. J Mol Biol, 1996, 259: 819-827]. The invention combined the specificity of serine protease at P1 site and the results of anti-trypsin peptides to synthesize peptides CH1, CH4, and CH5 with 0.46, 0.55, and 0.08 μM of inhibition constants Kᵢ against chymotrypsin. At the same time, similar peptides CH2, CH3, CH6, CH7, CH8, and CH9 were synthesized based on the characteristics of the ring extension between the disulfide bonds of anti-trypsin peptides. Only CH7 and CH9 had certain inhibitory activity against chymotrypsin, indicating that chymotrypsin may differ structurally from trypsin, and the ring extension structure between disulfide bonds is not suitable for optimizing the structure of anti-chymotrypsin peptides (FIG. 8, Table 8, and Table 9).

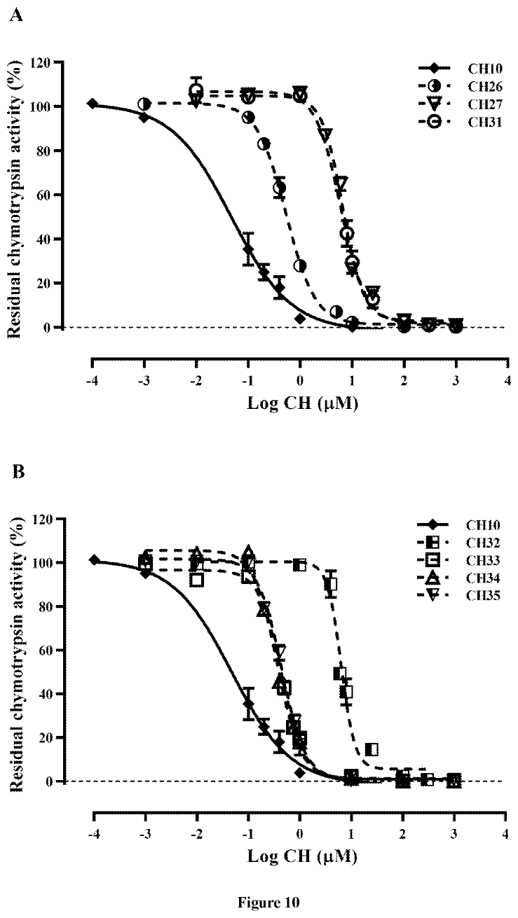

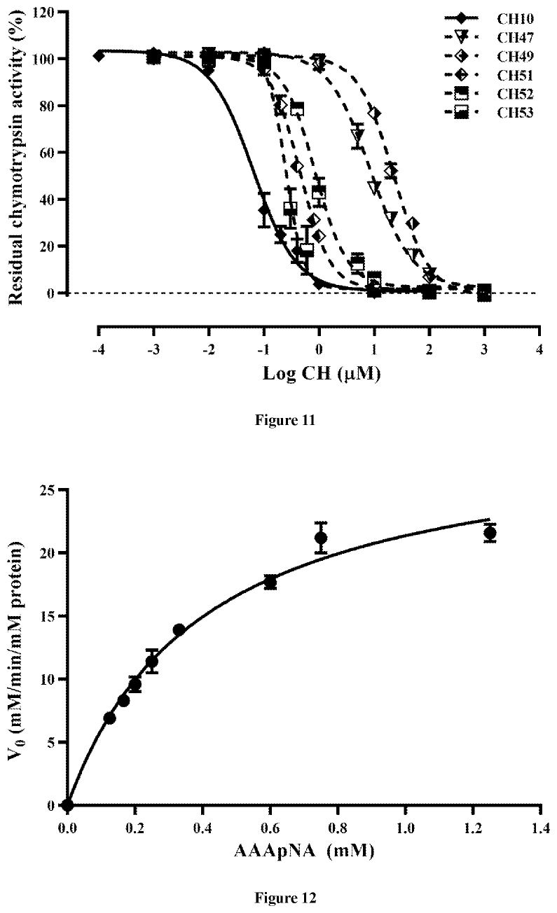

Based on the specificity of chymotrypsin P1 site and the good inhibitory activity of peptide CH5, a series of analogues and ring-expanding analogues of their disulfide bond loop were synthesized for the substitutions of P1 and P4 sites of amino acid residues. Then the chymotrypsin inhibition constant was determined, and the results showed that peptide CH10 had good inhibitory activity (Kᵢ=30 nM). Compared with the inhibitory activity of CH11, CH17, CH18, and CH19, the P1 site was preferably tyrosine, while the P4 site was preferably hydrophobic amino acid residue; The corresponding analogues CH13, CH23, and CH24 of disulfide bond ring expanding also exhibit good inhibitory activity (FIG. 9 and Table 8). Based on the effect of amino acid residues substitution at the P4′, P5′, and P7′ sites on the inhibitory activity against chymotrypsin, peptide analogues CH26-CH35 were synthesized. The determination of inhibition constants showed that the substitution of amino acids at the P4′, P5′, and P7′ sites had a significant impact on its activity. Among them, peptides CH26, CH33, CH34, and CH35 exhibited good inhibitory activity, while peptides CH27, CH31 and CH32 that had disulfide bond ring expansion also showed certain inhibitory activity (FIG. 10, Table 8, and Table 10). In addition, analogues CH36-CH53 with different site substitutions were synthesized, and the determination of inhibition constants showed that peptides CH47, CH49, CH51, CH52, and CH53 exhibited good chymotrypsin inhibitory activity (FIG. 11 and Table 8).

TABLE 8
The structure and activities of peptides against chymotrypsin

| | | | | | Theoretical |
|---|---|---|---|---|---|
| | | | IC₅₀ | Kᵢ | molecular weight |
| NO. | skeletons | Amino acid residue sequenceª | (μM) | (μM) | (Da) |
| 81 | CH1 | LCTFSIPPQCYG | 1.07 | 0.46 | 1326.56 |
| 82 | CH2 | CLAFSIPPQCYG | >50 | N.A. | 1296.54 |
| 83 | CH3 | CGLAFSIPPQCYG | >50 | N.A. | 1353.59 |
| 84 | CH4 | SCTYSIPPQCYG | 1.28 | 0.55 | 1316.48 |
| 85 | CH5 | FCTFSIPPQCYG | 0.19 | 0.08 | 1360.58 |
| 86 | CH6 | CGSGTYSIPPQCYG | >50 | N.A. | 1430.59 |
| 87 | CH7 | CGFGTFSIPPQCYG | 22.34 | 9.65 | 1474.68 |
| 88 | CH8 | CSATYSIPPQCY | >50 | N.A. | 1330.51 |
| 89 | CH9 | CGSATYSIPPQCY | 28.41 | 12.27 | 1387.56 |
| 90 | CH10 | FCTYSIPPQCYG | 0.06 | 0.03 | 1376.58 |
| 91 | CH11 | SCTFSIPPQCYG | 0.35 | 0.15 | 1300.48 |
| 92 | CH12 | CGFATFSIPPQCYG | >50 | N.A. | 1488.71 |
| 93 | CH13 | CGSATFSIPPQCYG | 5.62 | 2.43 | 1428.61 |
| 94 | CH14 | CGFATYSIPPQCYG | >50 | N.A. | 1504.71 |
| 95 | CH15 | CGSATYSIPPQCYG | 4.60 | 1.99 | 1441.61 |
| 96 | CH16 | CSATYSIPPQCYG | >50 | N.A. | 1387.56 |
| 97 | CH17 | ICTFSIPAQCV | 11.79 | 5.09 | 1179.43 |
| 98 | CH18 | ACTYSIPAKCF | 0.47 | 0.20 | 1201.44 |
| 99 | CH19 | FCTLSIPPQCYG | 11.28 | 4.87 | 1326.56 |
| 100 | CH20 | GKCLYSIPPICFPN | 16.45 | 7.10 | 1549.88 |
| 101 | CH21 | CGNATYSIPPQCYG | >50 | N.A. | 1471.64 |
| 102 | CH22 | CGTATYSIPPQCYG | >50 | N.A. | 1458.64 |
| 103 | CH23 | CGSATYSIPAQCVG | 4.31 | 1.86 | 1354.53 |
| 104 | CH24 | CGSATYSIPAKCFG | 6.14 | 2.65 | 1402.62 |
| 105 | CH25 | GTCTFSIPPICNPN | 0.28 | 0.12 | 1461.69 |
| 106 | CH26 | GTCTFSIPPICN | 0.54 | 0.23 | 1250.47 |
| 107 | CH27 | CGTATFSIPPICN | 6.66 | 2.88 | 1321.54 |
| 108 | CH28 | CPGEAMAYIRSCF | >50 | N.A. | 1445.70 |
| 109 | CH29 | CGGSATYSIPPQCY | >50 | N.A. | 1444.61 |
| 110 | CH30 | CGYATYSIPPQCYG | >50 | N.A. | 1520.71 |
| 111 | CH31 | CGAATYSIPAKCF | 6.08 | 2.63 | 1329.57 |
| 112 | CH32 | CGGAATYSIPAKCF | 6.38 | 2.76 | 1386.62 |
| 113 | CH33 | FCTYSIPPQCY | 0.44 | 0.19 | 1319.53 |
| 114 | CH34 | FCTYSIPPQCYA | 0.37 | 0.16 | 1390.61 |
| 115 | CH35 | FCTYSIPPQCR | 0.45 | 0.19 | 1312.54 |
| 116 | CH36 | FCTYSIPAKCY | 3.23 | 1.39 | 1293.53 |
| 117 | CH37 | FCTYSIPAQCY | 1.81 | 0.78 | 1293.49 |
| 118 | CH38 | ICTFSIPAQCI | 4.98 | 2.15 | 1193.46 |
| 119 | CH39 | ICTFSIPAQCV | 3.80 | 1.64 | 1179.43 |
| 120 | CH40 | ICTFSIPAQCF | 2.46 | 1.06 | 1227.47 |
| 121 | CH41 | FCTYSMPPHCV | 2.60 | 1.12 | 1282.53 |
| 122 | CH42 | RCDFSWPPRCL | >50 | N.A. | 1377.62 |
| 123 | CH43 | FCAYSNPPQCQ | 9.20 | 3.97 | 1255.40 |
| 124 | CH44 | FCAYSNPPKCQ | 19.90 | 8.59 | 1255.44 |
| 125 | CH45 | FCAYSYPPKCQ | 19.19 | 8.29 | 1304.52 |
| 126 | CH46 | FCNYSNPPQCQ | >50 | N.A. | 1298.43 |
| 127 | CH47 | ICTYSIPAQCI | 8.52 | 3.68 | 1209.46 |
| 128 | CH48 | VCTFSNPAMCH | >50 | N.A. | 1207.42 |
| 129 | CH49 | MCTFSHPAKCV | 22.71 | 9.81 | 1221.49 |
| 130 | CH50 | MCTFSDPGMCS | >50 | N.A. | 1176.37 |
| 131 | CH51 | PCTYSIPPQCY | 0.46 | 0.20 | 1269.47 |
| 132 | CH52 | FCTYSIPHypQCYG | 0.84 | 0.36 | 1392.58 |
| 133 | CH53 | FCTYSIHypPQCYG | 0.25 | 0.11 | 1392.58 |

^{a}: A disulfide bond is formed between two cysteine residues within the peptide
scaffold against chymotrypsin.
N.A.: Molecules with weak activity are no longer measured for Kᵢ value.

TABLE 9
Determination of the activities of peptides against chymotrypsin

| | Residual | | Residual | | Residual | | Residual |
|---|---|---|---|---|---|---|---|
| | activity of | | activity of | | activity of | | activity of |
| CH1 | chymotrypsin | CH4 | chymotrypsin | CH5 | chymotrypsin | CH7 | chymotrypsin |
| (μM) | (%) | (μM) | (%) | (μM) | (%) | (μM) | (%) |
| 0.001 | 103.5 ± 1.6 | 0.001 | 103.2 ± 0.9 | 0.0001 | 96.7 ± 9.3 | 0.01 | 100.9 ± 1.3 |
| 0.01 | 104.8 ± 7.2 | 0.01 | 101.8 ± 0.8 | 0.001 | 113.2 ± 2.4 | 0.1 | 98.8 ± 3.9 |
| 0.1 | 93.6 ± 5.5 | 0.1 | 100.7 ± 2.0 | 0.01 | 102.5 ± 3.7 | 1 | 101.7 ± 6.3 |
| 1 | 55.0 ± 3.3 | 0.3 | 86.7 ± 1.4 | 0.15 | 58.3 ± 2.3 | 10 | 77.7 ± 2.0 |
| 3 | 26.4 ± 1.1 | 0.6 | 70.4 ± 2.8 | 0.3 | 41.9 ± 5.7 | 25 | 46.0 ± 1.0 |
| 10 | 10.6 ± 0.2 | 2.5 | 34.6 ± 4.3 | 0.6 | 19.0 ± 0.8 | 50 | 25.8 ± 0.9 |
| 100 | 1.2 ± 0.1 | 7.5 | 13.8 ± 0.6 | 1 | 11.3 ± 1.5 | 100 | 14.1 ± 0.9 |
| 300 | 0.3 ± 0.1 | 10 | 9.3 ± 1.1 | 10 | 0.3 ± 0.0 | 300 | 4.1 ± 0.1 |
| 1000 | 0.5 ± 0.1 | 100 | 0.7 ± 0.1 | 100 | 0.3 ± 0.2 | 1000 | 1.2 ± 0.1 |
| | | 1000 | 0.2 ± 0.0 | | | | |

TABLE 10
Determination of the activities of peptides against chymotrypsin

| | Residual | | Residual | | Residual | | Residual |
|---|---|---|---|---|---|---|---|
| | activity of | | activity of | | activity of | | activity of |
| CH10 | chymotrypsin | CH26 | chymotrypsin | CH27 | chymotrypsin | CH31 | chymotrypsin |
| (μM) | (%) | (μM) | %) | (μM) | (%) | (μM) | (%) |
| 0.0001 | 101.3* | 0.001 | 101.1 ± 1.6 | 0.01 | 102.7 ± 2.4 | 0.01 | 107.4 ± 5.6 |
| 0.01 | 95.1 ± 1.2 | 0.1 | 95.1 ± 2.1 | 0.1 | 105.3 ± 0.9 | 0.1 | 104.3 ± 1.7 |
| 0.1 | 35.4 ± 7.2 | 0.2 | 83.1 ± 1.1 | 1 | 106.0 ± 0.7 | 1 | 104.8 ± 2.6 |
| 0.2 | 24.9 ± 3.5 | 0.4 | 63.2 ± 4.5 | 3 | 86.8 ± 1.5 | 8 | 42.5 ± 5.9 |
| 0.4 | 18.0 ± 4.9 | 1 | 27.8 ± 2.5 | 6 | 64.9 ± 3.1 | 10 | 29.5 ± 5.1 |
| 1 | 3.8 ± 0.4 | 5 | 7.0 ± 0.5 | 10 | 26.2 ± 2.4 | 25 | 12.7 ± 4.0 |
| 10 | 0.1 ± 0.1 | 10 | 2.2 ± 0.2 | 25 | 15.5 ± 0.3 | 100 | 2.6 ± 0.3 |
| 100 | −0.1 ± 0.0 | 100 | 0.3 ± 0.1 | 100 | 2.9 ± 0.3 | 300 | 1.0 ± 0.1 |
| | | 1000 | 0.1 ± 0.0 | 300 | 1.2 ± 0.0 | 1000 | 0.4 ± 0.0 |
| | | | | 1000 | 0.7 ± 0.1 | | |

| | Residual | | Residual | | Residual | | Residual |
|---|---|---|---|---|---|---|---|
| | activity of | | activity of | | activity of | | activity of |
| CH32 | chymotrypsin | CH33 | chymotrypsin | CH34 | chymotrypsin | CH35 | chymotrypsin |
| (μM) | (%) | (μM) | (%) | (μM) | (%) | (μM) | (%) |
| 0.01 | 99.5 ± 0.1 | 0.001 | 99.5 ± 1.4 | 0.001 | 102.3 ± 0.8 | 0.001 | 100.8 ± 0.9 |
| 0.1 | 100.2 ± 1.9 | 0.01 | 92.1 ± 2.1 | 0.01 | 104.1 ± 0.5 | 0.01 | 100.7 ± 1.2 |
| 1 | 99.0 ± 2.0 | 0.1 | 93.6 ± 1.6 | 0.1 | 104.8 ± 0.6 | 0.1 | 100.8 ± 2.6 |
| 4 | 90.1 ± 6.1 | 0.5 | 42.7 ± 3.1 | 0.2 | 78.5 ± 2.5 | 0.2 | 78.9 ± 2.9 |
| 6 | 49.2 ± 2.7 | 0.75 | 24.6 ± 2.0 | 0.4 | 45.5 ± 1.5 | 0.4 | 58.8 ± 3.4 |
| 8 | 40.8 ± 6.0 | 1 | 19.8 ± 1.0 | 0.75 | 24.5 ± 0.9 | 0.8 | 27.3 ± 2.9 |
| 25 | 14.4 ± 1.1 | 10 | 2.0 ± 0.2 | 1 | 17.8 ± 0.9 | 1 | 16.6 ± 4.7 |
| 100 | 2.2 ± 0.2 | 100 | 0.1 ± 0.0 | 10 | 1.2 ± 0.1 | 10 | 2.6 ± 0.2 |
| 300 | 0.7 ± 0.1 | 1000 | 0.1 ± 0.0 | 100 | 0.1 ± 0.0 | 100 | 0.0 ± 0.1 |
| | | | | 1000 | 0.1 ± 0.1 | 1000 | 0.9 ± 0.1 |

*CH10 did not inhibit the enzymatic activity at the concentration of 0.0001 μM, and two repetitions were discarded for large sampling error.

### Example 4: Design and Evaluation of Inhibitory Activity of Peptides that Inhibit Pancreatic Elastase

### Determination of the Michaelis Constant Kₘ Value:

(1) Added a total 198 μL of reaction buffer (20 mM CaCl₂, 50 mM Tris-HCl (pH 8.0)) into the 96 well plate, followed by preheating at 37° C. for 15 min. Then added 2 μL of different concentrations of substrates (pNA, dissolved in DMSO), and mixed by centrifugation at 500 rpm for 1 min. Finally, the plate was incubated at 37° C. for 30 min, and the absorbance value at 405 nm was measured. In the 200 μL of reaction system, the final concentrations of pNA were 0, 0.025, 0.05, 0.075, 0.1, 0.125, 0.15, 0.175, and 0.2 mM, respectively. Make three repetitions for each concentration and plot the OD₄₀₅ m value with pNA concentration to obtain the standard curve.
(2) Added a total 190 μL of reaction buffer (20 mM CaCl₂, 50 mM Tris-HCl (pH 8.0)) and 8 μL of 4.375 μM elastase into the 96 well plate, followed by preheating at 37° C. for 5 min. Then added 2 μL of different concentrations of substrates (AAApNA, dissolved in DMSO), and mixed by centrifugation at 500 rpm for 1 min. Finally, the plate was incubated at 37° C. for 30 min, and the absorbance value at 405 nm was measured. In the 200 μL of reaction system, the final concentrations of AAApNA were 0, 0.125, 0.166, 0.2, 0.25, 0.33, 0.6, 0.75, and 1.25 mM, respectively. Make three repetitions for each concentration and plot the OD_{405 nm} value with time to obtain the corresponding curve. Divide the slope of the curve by the slope of the standard curve and the enzyme concentration to obtain the initial velocity V₀ (mM/(min*mM protein). Plot the initial velocity V₀ with the concentration of substrate AAApNA using Prism software, and the Michaelis constant Kₘ value of elastase hydrolyzing AAApNA was obtained.

### Determination of the Inhibition Constant Kᵢ Value:

(1) Added a total 190 μL of different concentrations of peptides (ECs) against elastase and reaction buffer (20 mM CaCl₂, 50 mM Tris-HCl buffer (pH 8.0)) into the pre-cold 96 well plate, followed by preheating at 37° C. for 5 min and centrifugated at 500 rpm for 1 min and allowed to stand for 4 minutes. Then added 8 μL of 12.5 μM elastase and incubated at 37° C. for 10 min and centrifugated at 500 rpm for 1 min and allowed to stand for 9 minutes. Finally, added 2 μL of 100 mM AAApNA, and mixed by centrifugation at 500 rpm for 1 min. The plate was incubated at 37° C. for 60 min, and the absorbance value at 405 nm was measured. Three repetitions were made, and only reaction buffer and substrate were added in the blank control as the minimum absorption value (Min OD_{405 nm}); Only reaction buffer, enzyme and substrate were added in the negative control as the maximum absorption value (Max OD₄₀₅ nm).
(2) In the 200 μL of reaction system, the final concentrations of elastase and AAApNA were 0.5 μM and 1 mM, respectively.
(3) Data statistics

Residual activity of enzyme (%)=(1−(Max OD_{405 nm}−Sample OD_{405 nm})/(Max OD_{405 nm}−Min OD_{405 nm}))*100

Plot the residual activity of enzyme with substrate concentration to obtain the half inhibitory concentration (IC50) of peptide scaffolds ECs against elastase. Then, substitute it into the formula Kᵢ=IC50/(1+S/Kₘ) (S, IC50, and Kₘ are substrate concentration, half inhibitory concentration, and Michaelis constant, respectively) to obtain the inhibition constant Kᵢ of ECs skeleton inhibiting elastase.

### Results:

Using a certain concentration of elastase to catalyze different concentrations of AAApNA to produce pNA, and the absorbance value of OD_{405 nm} was measured. Referring to the standard curve, Prism software was used to plot the initial velocity V₀ with the concentration of substrate AAApNA, and the Michaelis constant Kₘ value of elastase hydrolyzing AAApNA was obtained to be 0.40 mM (R²=0.9885) (FIG. 12).

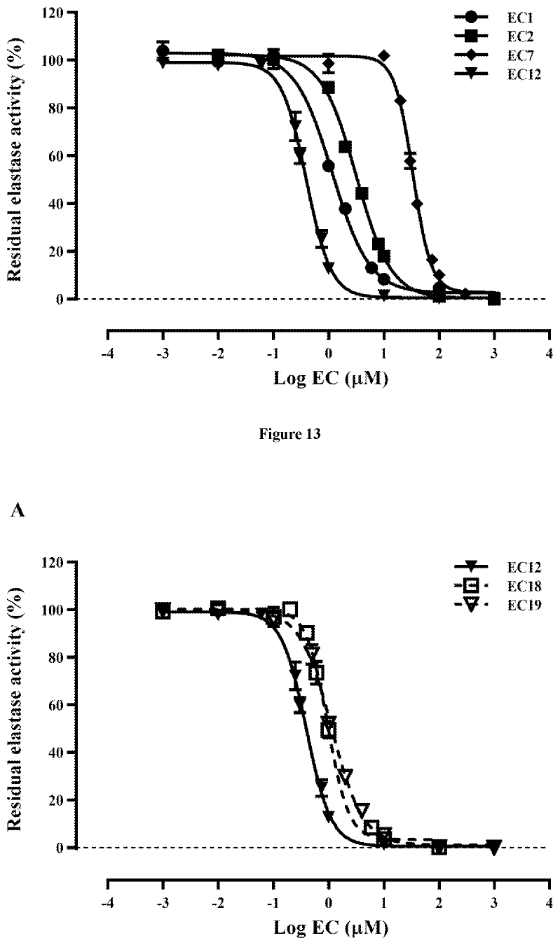

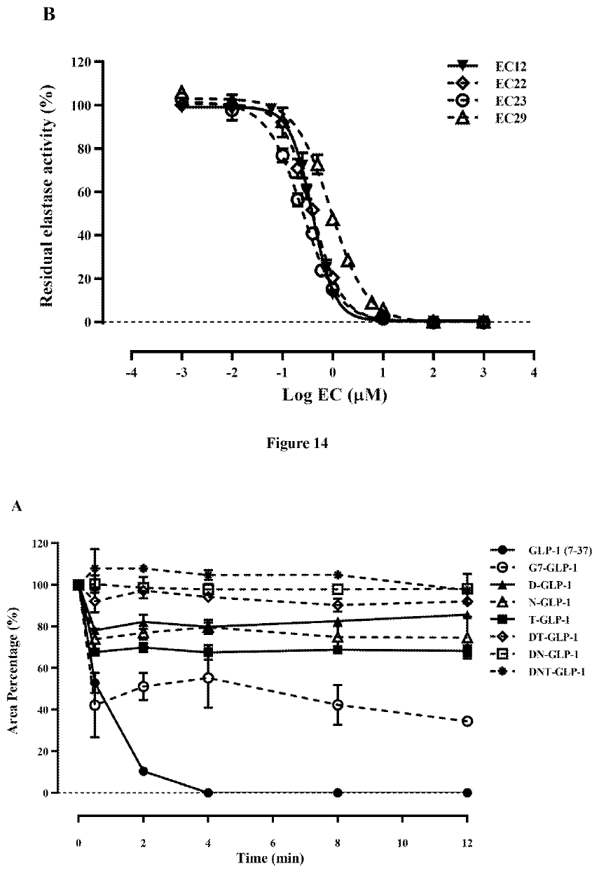

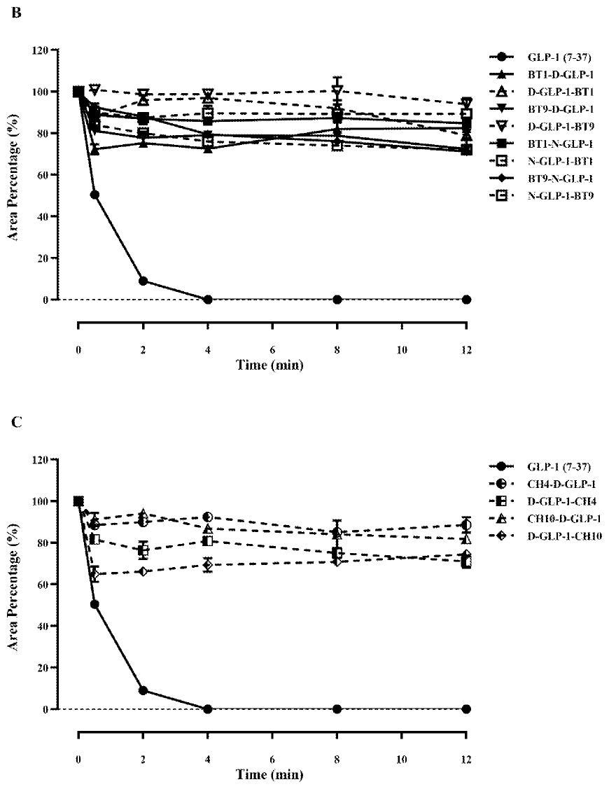

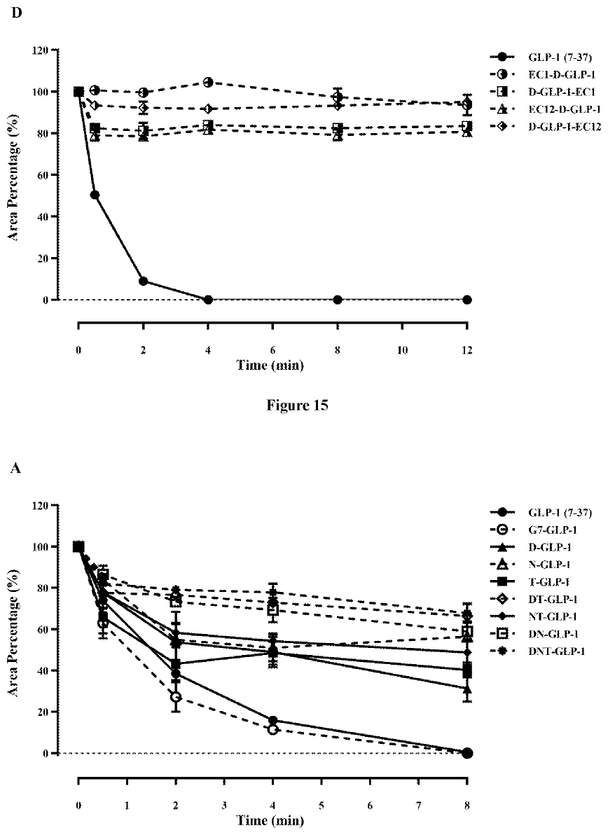

There are few reports on the active peptides of pancreatic elastase, and only the literature reports that the analogues of peptide EC1 have good inhibitory activity against pancreatic elastase [McBride J D, Free H N, Leatherbarrow R J. Selection of human elastase inhibitors from a conformably constrained combinatorial peptide library. Eur J Biochem, 1999, 266: 403-412.]. In this invention, the elastase inhibitory peptides EC1-EC12 based on the specificity of serine protease with P1 site and the results of trypsin and chymotrypsin inhibitory peptides had been synthesized. The results of determining the inhibition constant Kᵢ of Elastase showed that peptides EC1 and EC12 with alanine at P1 site had better inhibitory activity against Elastase, and EC12 had better inhibitory activity than peptides EC1 and EC2, indicating that amino acid substitution at P5′ and P7′ sites had a great impact on its inhibitory activity, while only the analog EC7 with disulfide ring extension, showed weaker inhibitory activity (FIG. 13 and Table 11). Then, analogues EC13-EC29 with different site substitutions were synthesized. The determination of inhibition constants showed that peptide EC23 (Kᵢ=70 nM) had a certain increase of inhibitory activity against elastase compared to peptide EC12 (Kᵢ=110 nM), while the decrease in inhibitory activity of peptides EC25-EC28 indicated that the amino acid substitution at the P1′ position had a significant impact. The substitution of sites P4, P5′, and P7′ had an impact on its inhibitory activity but was less than that at the P1′ position (FIG. 14, Table 11, and Table 12). Subsequently, peptides EC30-EC45 and its hydroxyproline containing analogues EC46-EC48 were synthesized on the basis of EC23.

TABLE 11
The structure and inhibitory activities of peptides against elastase

| | | | | | Theoretical |
|---|---|---|---|---|---|
| | | | IC₅₀ | Kᵢ | molecular weight |
| NO. | Skeletons | Amino acid residue sequence^{a} | (μM) | (μM) | (Da) |
| 134 | EC1 | LCTASIPPQCY | 1.17 | 0.33 | 1193.41 |
| 135 | EC2 | LCTLSIPPQCY | 3.19 | 0.91 | 1235.49 |
| 136 | EC3 | CGLATASIPPQCY | >50 | N.A. | 1321.54 |
| 137 | EC4 | CGLGTASIPPQCY | >50 | N.A. | 1307.52 |
| 138 | EC5 | CLATASIPPQCY | >50 | N.A. | 1264.49 |
| 139 | EC6 | CGLATASIPPICO | >50 | N.A. | 1271.53 |
| 140 | EC7 | CGLATLSIPPICQ | 33.66 | 9.62 | 1313.61 |
| 141 | EC8 | CGRETASIPPICQ | >50 | N.A. | 1372.59 |
| 142 | EC9 | CGRETASIPPQCK | >50 | N.A. | 1387.61 |
| 143 | EC10 | CGRETASIPPQKC | >50 | N.A. | 1387.61 |
| 144 | EC11 | CGRETASIPPQKGC | >50 | N.A. | 1444.66 |
| 145 | EC12 | LCTASIPPICQ | 0.40 | 0.11 | 1143.40 |
| 146 | EC13 | ICTLSIPAQCV | >50 | N.A. | 1145.41 |
| 147 | EC14 | YCTASIPPQCY | 7.14 | 2.04 | 1243.43 |
| 148 | EC15 | CGYATASIPPQCY | >50 | N.A. | 1371.56 |
| 149 | EC16 | CGGLATASIPPICQ | >50 | N.A. | 1328.58 |
| 150 | EC17 | CGGLATLSIPPICQ | >50 | N.A. | 1370.66 |
| 151 | EC18 | LCTASIPPQCQ | 0.96 | 0.27 | 1158.37 |
| 152 | EC19 | ICTASIPPQCQ | 1.14 | 0.33 | 1158.37 |
| 153 | EC20 | LCTASIPPQCR | >50 | N.A. | 1186.43 |
| 154 | EC21 | LCTASIPPICR | 15.28 | 4.37 | 1171.45 |
| 155 | EC22 | VCTASIPPICQ | 0.40 | 0.11 | 1129.37 |
| 156 | EC23 | ICTASIPPICQ | 0.25 | 0.07 | 1143.40 |
| 157 | EC24 | FCTASIPPICQ | 2.23 | 0.64 | 1177.41 |
| 158 | EC25 | LCTASNPPICQ | 1.05 | 0.30 | 1144.34 |
| 159 | EC26 | MCTASMPPQCH | 23.93 | 6.84 | 1203.45 |
| 160 | EC27 | ICTASYPPQCR | 8.53 | 2.44 | 1236.44 |
| 161 | EC28 | LCTASNPPTCR | >50 | N.A. | 1160.34 |
| 162 | EC29 | YCTASIPPICQ | 0.92 | 0.26 | 1193.41 |
| 163 | EC30 | CGIATASIPPICQ | >50 | N.A. | 1271.53 |
| 164 | EC31 | CGIAbuTASIPPICQ | >50 | N.A. | 1285.57 |
| 165 | EC32 | CGINleTASIPPICQ | 21.39 | 6.11 | 1313.62 |
| 166 | EC33 | CGILTASIPPICQ | 26.53 | 7.58 | 1313.61 |
| 167 | EC34 | CGISTASIPPICQ | >50 | N.A. | 1287.53 |
| 168 | EC35 | CGITTASIPPICQ | — | — | 1301.55 |
| 169 | EC36 | CGIFTASIPPICQ | >50 | N.A. | 1347.63 |
| 170 | EC37 | CGIYTASIPPICQ | — | — | 1363.63 |
| 171 | EC38 | CGINTASIPPICQ | 1 | — | 1314.55 |
| 172 | EC39 | CGIQTASIPPICQ | >50 | N.A. | 1328.58 |
| 173 | EC40 | CGIHTASIPPICQ | >50 | N.A. | 1337.59 |
| 174 | EC41 | CGIRTASIPPICQ | — | — | 1356.64 |
| 175 | EC42 | CGIKTASIPPICQ | >50 | N.A. | 1328.62 |
| 176 | EC43 | CGIWTASIPPICQ | — | — | 1386.66 |
| 177 | EC44 | CPIATASIPPICQ | >50 | N.A. | 1311.59 |
| 178 | EC45 | CAIATASIPPICQ | 47.00 | 13.43 | 1285.55 |
| 179 | EC46 | CHypIATASIPPICQ | >50 | N.A. | 1327.59 |
| 180 | EC47 | ICTASIHypPICQ | 0.40 | 0.11 | 1159.40 |
| 181 | EC48 | ICTASIPHypICQ | 0.55 | 0.16 | 1159.40 |
| 182 | EC49 | QGADTPPVGGLCTASIPPQCY | 0.99 | 0.28 | 2073.34 |
| 183 | EC50 | SNGNAVEDGGLCTASIPPQCY | 0.97 | 0.28 | 2094.27 |

ₐ: A disulfide bond is formed between two cysteine residues within the peptide
scaffolds against elastase.
N.A.: Molecules with weak activity are no longer measured for Kᵢ value.

TABLE 12
Determination of the inhibitory activities of peptides against elastase

| EC12 | Residual activity of | EC18 | Residual activity of | EC19 | Residual activity of |
|---|---|---|---|---|---|
| (μM) | elastase (%) | (μM) | elastase (%) | (μM) | elastase (%) |
| 0.001 | 98.8 ± 1.1 | 0.001 | 99.2 ± 2.0 | 0.001 | 99.9 ± 0.5 |
| 0.01 | 97.8 ± 1.0 | 0.01 | 100.6 ± 1.5 | 0.01 | 99.4 ± 0.1 |
| 0.06 | 98.2 ± 2.1 | 0.1 | 97.0 ± 4.0 | 0.1 | 96.1 ± 0.9 |
| 0.25 | 72.2 ± 5.9 | 0.2 | 100.2 ± 2.0 | 0.5 | 81.2 ± 4.1 |
| 0.3 | 59.9 ± 3.2 | 0.4 | 90.3 ± 0.5 | 1 | 52.1 ± 1.8 |
| 0.75 | 25.1 ± 3.6 | 0.6 | 73.5 ± 4.8 | 2 | 29.8 ± 0.8 |
| 1 | 12.7 ± 1.0 | 1 | 49.4 ± 3.3 | 4 | 15.5 ± 0.3 |
| 10 | 1.3 ± 0.1 | 6 | 8.4 ± 0.4 | 10 | 5.4 ± 0.3 |
| 100 | 0.2 ± 0.0 | 10 | 3.6 ± 0.5 | 100 | 0.2 ± 0.1 |
| 1000 | 0.3 ± 0.0 | 100 | 0.1 ± 0.0 | 1000 | 0.0 ± 0.0 |
| EC22 | Residual activity of | EC23 | Residual activity of | EC29 | Residual activity of |
| (μM) | elastase (%) | (μM) | elastase (%) | (μM) | elastase (%) |
| 0.001 | 100.1 ± 1.0 | 0.001 | 103.0 ± 2.2 | 0.001 | 105.9 ± 2.2 |
| 0.01 | 99.8 ± 0.3 | 0.01 | 97.8 ± 4.8 | 0.01 | 102.0 ± 2.9 |
| 0.1 | 92.0 ± 6.8 | 0.1 | 76.8 ± 3.1 | 0.1 | 92.6 ± 2.7 |
| 0.2 | 70.8 ± 4.3 | 0.2 | 56.4 ± 2.9 | 0.5 | 72.7 ± 4.4 |
| 0.4 | 51.6 ± 1.7 | 0.4 | 40.8 ± 2.0 | 1 | 47.5 ± 0.7 |
| 1 | 20.4 ± 1.2 | 0.6 | 23.7 ± 0.2 | 2 | 28.5 ± 0.4 |
| 10 | 2.4 ± 0.2 | 1 | 15.1 ± 0.9 | 6 | 8.9 ± 0.4 |
| 100 | 0.0 ± 0.0 | 10 | 1.5 ± 0.3 | 10 | 5.9 ± 0.4 |
| 1000 | −0.2 ± 0.2 | 100 | −0.2 ± 0.1 | 100 | 0.1 ± 0.1 |
| | | 1000 | −0.2 ± 0.1 | 1000 | 0.1 ± 0.0 |

### Example 5 Improve the Stability of Glucagon Like Peptide-1 (GLP-1) Against Dipeptidyl Peptidase IV (DPP-IV) and Neutral Endopeptidase 24.11 (NEP24.11)

To improve the stability of GLP-1 in blood circulation, GLP-1 analogues (hybrid peptides), which contain DPP-IV inhibitory peptide diprotin A (IPI) and NEP24.11 inhibitory peptide Opiorphin (QRFSR) were designed and synthesized. The structural sequences are shown in Table 13.

### Tolerance of GLP-1 and its Analogues (Hybrid Peptides) to DPP-IV:

To investigate the tolerance of GLP-1 and its analogues to DPP-IV, the following experiments were carried out:

Control test: Take three sterile EP tubes and add 5 μL of 250 μM GLP-1 or GLP-1 analogs, 45 μL of 100 mM Tris-HCl buffer (pH 8.0) and 7.5 μL of 10% TFA. Mix by centrifugation at 8000 rpm for 30 seconds.

Enzymatic hydrolysis kinetics of DPP-IV on GLP-1 and its analogues (hybrid peptides): (1) Take three sterile EP tubes and separately add 30 μL of 250 μM GLP-1 or GLP-1 analogs and 240 μL of 100 mM Tris-HCl buffer (pH 8.0). (2) Arrange a certain volume of 0.005 μg/μL DPP-IV solution in another sterile EP. (3) Incubate the EP tubes containing peptides and enzymes at 37° C. for 5 min, and separately add 30 μL of DPP-IV solution to each EP tube containing peptides and mix well. Start timing and remove 50 μL of reaction solution at 0.5, 2.0, 4.0, 8.0, and 12.0 hours later, respectively. Then add 7.5 μL of 10% TFA to terminate the reaction and mix well by centrifugation at 8000 rpm for 30 seconds. In the 50 μL of reaction system, the final concentrations of GLP-1 or GLP-1 analogues and DPP-IV were 25 μM and 0.5 ng/μL, respectively. There were three replicates at each time point, and the peak area of the peptide at each time point was detected using reverse phase high-performance liquid chromatography (RP-HPLC). The ratio of the remaining peak area of the sample at detection time T (h) to the peak area of the prototype peptide at 0 h was calculated as the remaining percentage (%) of the peptide.

Results: To rule out the influence of trypsin inhibitory peptides on the hydrolysis of GLP-1 and its analogues by DPP-IV, GLP-1 analogues SEQ ID NO: 189, SEQ ID NO: 190, SEQ ID NO: 191, and SEQ ID NO: 193 containing partial peptide segment of BT43 (SEQ ID NO: 43) were synthesized (Table 13). Determine the remaining prototype sample ratio of experimental samples after DPP-IV treatment for different time by HPLC. The results showed that seven glycine residues directly attached at the N-terminus of GLP-1 (G7-GLP-1, SEQ ID NO: 186) can form a protective effect on the tolerance of GLP-1 to DPP-IV degradation. After 12 hours of treatment, G7-GLP-1 still had about 34.5% remaining, while GLP-1 (7-37) had been absolutely degraded after about 4 hours; Introduction of DPP-IV inhibitory peptide diprotin A (IPI) in D-GLP-1 (SEQ ID NO: 187) showed good stability in tolerating DPP-IV degradation, and 85.6% of prototype peptide remained after 12 hours of treatment (FIG. 15A and Table 14). Introduction of inhibitory peptides against trypsin at the N/C terminal of GLP-1 (SEQ ID NO: 194-201) exhibited good stability in tolerating DPP-IV degradation (FIG. 15B and Table 14). Introduction of inhibitory peptides against chymotrypsin at the N/C terminal of GLP-1 (SEQ ID NO: 202-205) also displayed good stability in tolerating DPP-IV degradation (FIG. 15C and Table 14). Similarly, introduction of inhibitory peptides against elastase at the N/C terminal of GLP-1 (SEQ ID NO: 206-209) also showed good stability against DPP-IV degradation (FIG. 15D and Table 14). The results indicated that the introduction of active peptide skeletons D, N, T, BT, CH, and EC that inhibit different metabolic enzymes can enhance the tolerance of GLP-1 to DPP-IV.

TABLE 13
The structures of GLP-1 and its analogs

| | | | Theoretical |
|---|---|---|---|
| | | | molecular |
| | | | weight |
| NO. | peptides | Amino acid sequences^{a} | (Da) |
| 184 | GLP-1 (7-37) | HAEGTFTSDVSSYLEGQAAKEFIAWLVKGRG | 3355.71 |
| 185 | GLP-1(G8) | HGEGTFTSDVSSYLEGQAAKEFIAWLVKGRG | 3341.68 |
| 186 | G7-GLP-1 | GGGGGGGHAEGTFTSDVSSYLEGQAAKEFIAW | 3755.07 |
| | | LVKGRG | |
| 187 | D-GLP-1 | GIPIGGHAEGTFTSDVSSYLEGQAAKEFIAWLV | 4092.58 |
| | | KGRGGK | |
| 188 | N-GLP-1 | 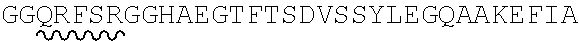 | 4443.91 |
| | | WLVKGRGGK | |
| 189 | T-GLP-1 | 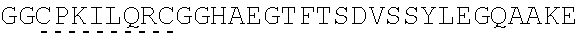 | 4709.35 |
| | | FIAWLVKGRGGK | |
| 190 | DT-GLP-1 | 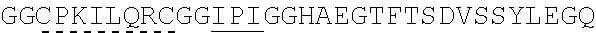 | 5146.89 |
| | | AAKEFIAWLVKGRGGK | |
| 191 | NT-GLP-1 | 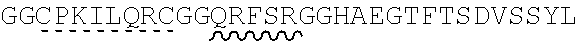 | 5498.21 |
| | | EGQAAKEFIAWLVKGRGGK | |
| 192 | DN-GLP-1 | 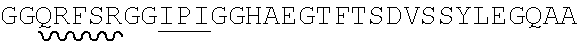 | 4881.44 |
| | | KEFIAWLVKGRGGK | |
| 193 | DNT-GLP-1 | 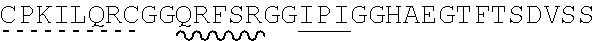 | 5821.65 |
| | | YLEGQAAKEFIAWLVKGRGGK | |
| 194 | BT1-D-GLP-1 | 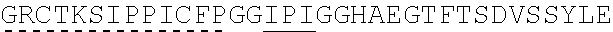 | 5491.30 |
| | | GQAAKEFIAWLVKGRGGK | |
| 195 | BT1-N-GLP-1 | 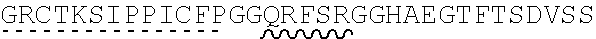 | 5842.63 |
| | | YLEGQAAKEFIAWLVKGRGGK | |
| 196 | D-GLP-1-BT1 | GIPIGGHAEGTFTSDVSSYLEGQAAKEFIAWLVK | 5434.25 |
| | | 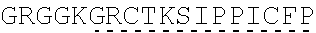 | |
| 197 | N-GLP-1-BT1 | 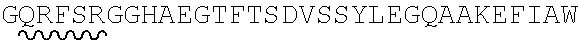 | 5785.57 |
| | | 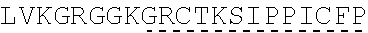 | |
| 198 | BT9-D-GLP-1 | 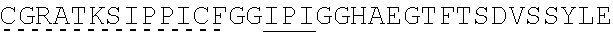 | 5465.26 |
| | | GQAAKEFIAWLVKGRGGK | |
| 199 | BT9-N-GLP-1 | 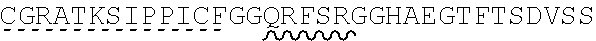 | 5816.59 |
| | | YLEGQAAKEFIAWLVKGRGGK | |
| 200 | D-GLP-1-BT9 | GIPIGGHAEGTFTSDVSSYLEGQAAKEFIAWLVK | 5465.26 |
| | | 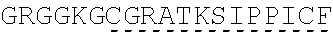 | |
| 201 | N-GLP-1-BT9 | 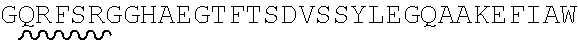 | 5816.59 |
| | | 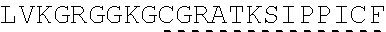 | |
| 202 | CH4-D-GLP-1 | SCTYSIPPQCYGGIPIGGHAEGTFTSDVSSYLEGQ | 5333.99 |
| | | AAKEFIAWLVKGRGGK | |
| 203 | D-GLP-1-CH4 | GIPIGGHAEGTFTSDVSSYLEGQAAKEFIAWLVK | 5391.05 |
| | | GRGGKGSCTYSIPPQCYG | |
| 204 | CH10-D-GLP- | FCTYSIPPQCYGGIPIGGHAEGTFTSDVSSYLEGQ | 5394.09 |
| | 1 | AAKEFIAWLVKGRGGK | |
| 205 | D-GLP-1- | GIPIGGHAEGTFTSDVSSYLEGQAAKEFIAWLVK | 5451.15 |
| | CH10 | GRGGKGFCTYSIPPQCYG | |
| 206 | EC1-D-GLP-1 | LCTASIPPQCYGGIPIGGHAEGTFTSDVSSYLEGQ | 5267.98 |
| | | AAKEFIAWLVKGRGGK | |
| 207 | D-GLP-1-EC1 | GIPIGGHAEGTFTSDVSSYLEGQAAKEFIAWLVK | 5267.98 |
| | | GRGGKGLCTASIPPQCY | |
| 208 | EC12-D-GLP- | LCTASIPPICQGGIPIGGHAEGTFTSDVSSYLEGQ | 5217.96 |
| | 1 | AAKEFIAWLVKGRGGK | |
| 209 | D-GLP-1- | GIPIGGHAEGTFTSDVSSYLEGQAAKEFIAWLVK | 5217.96 |
| | EC12 | GRGGKGLCTASIPPICQ | |

^{a}The peptide scaffolds against DPP-IV, NEP24.11, trypsin, chymotrypsin and elastase are separately named D, N, T, BT, CH and EC, and are marked with straight lines, wavy lines, dotted lines, double straight lines and italics. In addition, disulfide bonds are formed between the two cysteine residues in the peptide scaffolds against trypsin, chymotrypsin and elastase in the polypeptide sequence.

TABLE 14
The stability of GLP-1 and its analogs (SEQ ID NO: 186-189) against DPPIV

| Time | Remaining peak area (%)/DPP-IV | | | | |
|---|---|---|---|---|---|
| (h) | G7-GLP-1 | D-GLP-1 | N-GLP-1 | T-GLP-1 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 42.1 ± 15.5 | 78.1 ± 1.8 | 73.9 ± 5.0 | 67.7 ± 0.8 | 52.9 ± 4.8 |
| 2.0 | 51.1 ± 6.5 | 82.2 ± 3.4 | 76.9 ± 3.0 | 69.9 ± 2.2 | 10.4 ± 0.9 |
| 4.0 | 55.3 ± 14.4 | 79.8 ± 3.4 | 79.6 ± 2.3 | 67.5 ± 3.5 | 0.0 ± 0.0 |
| 8.0 | 42.3 ± 9.6 | 82.6 ± 2.0 | 74.9 ± 5.3 | 68.8 ± 1.5 | 0.0 ± 0.0 |
| 12.0 | 34.5 ± 2.1 | 85.6 ± 2.0 | 74.6 ± 10.1 | 68.2 ± 2.6 | 0.0 ± 0.0 |

The stability of GLP-1 and its analogs (SEQ ID NO: 190-193) against DPPIV

| Time | Remaining peak area (%)/DPP-IV | | | | |
|---|---|---|---|---|---|
| (h) | DT-GLP-1 | NT-GLP-1 | DN-GLP-1 | DNT-GLP-1 | GLP-1 (7-37) |
| 0.0 | 100.0 ± 0.0 | N.A. | 100.0 ± 0.0 | 100.0 ± 0.0 | 100.0 ± 0.0 |
| 0.5 | 92.1 ± 5.4 | N.A. | 100.3 ± 4.2 | 107.9 ± 9.3 | 52.9 ± 4.8 |
| 2.0 | 97.2 ± 1.2 | N.A. | 98.6 ± 5.1 | 107.8 ± 1.7 | 10.4 ± 0.9 |
| 4.0 | 94.1 ± 2.2 | N.A. | 97.8 ± 2.7 | 104.7 ± 2.3 | 0.0 ± 0.0 |
| 8.0 | 90.3 ± 3.2 | N.A. | 97.8 ± 2.2 | 104.8 ± 1.7 | 0.0 ± 0.0 |
| 12.0 | 92.0 ± 1.4 | N.A. | 98.1 ± 7.1 | 97.4 ± 2.1 | 0.0 ± 0.0 |

N.A.: Undetermined.

The stability of GLP-1 and its analogs (SEQ ID NO: 194-197) against DPPIV

| Time | Remaining peak area (%)/DPP-IV | | | | |
|---|---|---|---|---|---|
| (h) | BT1-D-GLP-1 | BT1-N-GLP-1 | D-GLP-1-BT1 | N-GLP-1-BT1 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 72.3 ± 2.4 | 88.9 ± 0.8 | 88.0 ± 6.2 | 84.0 ± 0.1 | 50.5 ± 0.9 |
| 2.0 | 75.3 ± 1.5 | 87.0 ± 2.7 | 95.9 ± 2.3 | 80.1 ± 0.9 | 9.0 ± 0.1 |
| 4.0 | 72.6 ± 2.0 | 85.8 ± 1.8 | 96.9 ± 2.0 | 76.0 ± 1.6 | 0.0 ± 0.0 |
| 8.0 | 82.0 ± 3.2 | 87.3 ± 2.0 | 92.0 ± 3.9 | 74.1 ± 0.5 | 0.0 ± 0.0 |
| 12.0 | 82.5 ± 4.9 | 84.8 ± 1.6 | 79.0 ± 4.6 | 72.1 ± 2.0 | 0.0 ± 0.0 |

The stability of GLP-1 and its analogs (SEQ ID NO: 198-201) against DPPIV

| Time | Remaining peak area (%)/DPP-IV | | | | |
|---|---|---|---|---|---|
| (h) | BT9-D-GLP-1 | BT9-N-GLP-1 | D-GLP-1-BT9 | N-GLP-1-BT9 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 81.1 ± 1.0 | 92.5 ± 1.5 | 100.9 ± 1.2 | 90.0 ± 3.0 | 50.5 ± 0.9 |
| 2.0 | 77.9 ± 2.9 | 88.0 ± 1.8 | 98.7 ± 0.6 | 89.5 ± 1.8 | 9.0 ± 0.1 |
| 4.0 | 79.1 ± 1.3 | 79.4 ± 2.2 | 98.8 ± 2.3 | 89.6 ± 3.6 | 0.0 ± 0.0 |
| 8.0 | 78.8 ± 0.6 | 76.1 ± 1.4 | 100.3 ± 6.5 | 89.2 ± 1.0 | 0.0 ± 0.0 |
| 12.0 | 72.4 ± 1.9 | 71.2 ± 2.1 | 94.0 ± 2.0 | 89.3 ± 7.5 | 0.0 ± 0.0 |

The stability of GLP-1 and its analogs (SEQ ID NO: 202-205) against DPPIV

| Time | Remaining peak area (%)/DPP-IV | | | | |
|---|---|---|---|---|---|
| (h) | CH4-D-GLP-1 | D-GLP-1-CH4 | CH10-D-GLP-1 | D-GLP-1-CH10 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 88.5 ± 0.7 | 81.6 ± 1.9 | 91.2 ± 3.2 | 64.9 ± 3.7 | 50.5 ± 0.9 |
| 2.0 | 90.0 ± 0.9 | 76.4 ± 4.2 | 94.1 ± 2.3 | 66.2 ± 1.0 | 9.0 ± 0.1 |
| 4.0 | 92.4 ± 0.5 | 80.8 ± 1.4 | 86.8 ± 0.8 | 69.3 ± 3.3 | 0.0 ± 0.0 |
| 8.0 | 85.1 ± 0.9 | 75.1 ± 2.4 | 84.1 ± 6.7 | 70.8 ± 0.4 | 0.0 ± 0.0 |
| 12.0 | 88.6 ± 3.7 | 71.0 ± 3.0 | 81.8 ± 0.6 | 74.4 ± 0.3 | 0.0 ± 0.0 |

The stability of GLP-1 and its analogs (SEQ ID NO: 206-209 against DPPIV

| Time | Remaining peak area (%)/DPP-IV | | | | |
|---|---|---|---|---|---|
| (h) | EC1-D-GLP-1 | D-GLP-1-EC1 | EC12-D-GLP-1 | D-GLP-1-EC12 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 100.7 ± 1.7 | 82.5 ± 0.6 | 79.1 ± 2.6 | 93.4 ± 0.8 | 50.5 ± 0.9 |
| 2.0 | 99.6 ± 0.6 | 81.2 ± 3.9 | 78.5 ± 1.1 | 92.3 ± 3.0 | 9.0 ± 0.1 |
| 4.0 | 104.5 ± 0.3 | 84.0 ± 2.2 | 81.7 ± 0.8 | 91.7 ± 0.6 | 0.0 ± 0.0 |
| 8.0 | 97.4 ± 4.2 | 82.5 ± 1.6 | 79.2 ± 2.6 | 93.3 ± 0.2 | 0.0 ± 0.0 |
| 12.0 | 93.6 ± 4.9 | 83.5 ± 0.5 | 80.7 ± 0.3 | 95.1 ± 1.4 | 0.0 ± 0.0 |

### Tolerance of GLP-1 and its Analogues (Hybrid Peptides) to NEP24.11:

Control test: Take three sterile EP tubes and add 6 μL of 250 μM GLP-1 or GLP-1 analogs, and 44 μL of reaction buffer (50 mM HEPES, pH 7.4, 50 mM NaCl), and 7.5 μL of 10% TFA. Mix by centrifugation at 8000 rpm for 30 seconds.

Enzymatic hydrolysis kinetics of NEP24.11 on GLP-1 and its analogues (hybrid peptides): Take three sterile EP tubes and separately add 30 μL of 250 μM GLP-1 or GLP-1 analogs, and 215 μL of reaction buffer (50 mM HEPES, pH 7.4, 50 mM NaCl). Arrange a certain volume of 0.04 μg/μL NEP24.11 enzyme solution in another sterile EP. Incubate the EP tubes containing peptides and enzymes at 37° C. for 5 min, and add 5 μL of NEP24.11 solution to each EP tube containing peptides and mix well. Start timing and remove 50 μL of reaction solution at 0.5, 2.0, 4.0, and 8.0 hours later, respectively. Add 7.5 μL of 10% TFA to terminate the reaction and mix well by centrifugation at 8000 rpm for 30 seconds. In the 50 μL of reaction system, the final concentrations of GLP-1 or GLP-1 analogues and NEP24.11 were 30 μM and 1.0 ng/μL, respectively. There are three replicates at each time point, and the peak area of the peptide at each time point is detected using reverse phase high-performance liquid chromatography (RP-HPLC). The ratio of the remaining peak area of the sample at detection time T (h) to the peak area of the prototype peptide at 0 h is calculated as the remaining percentage (%) of the peptide.

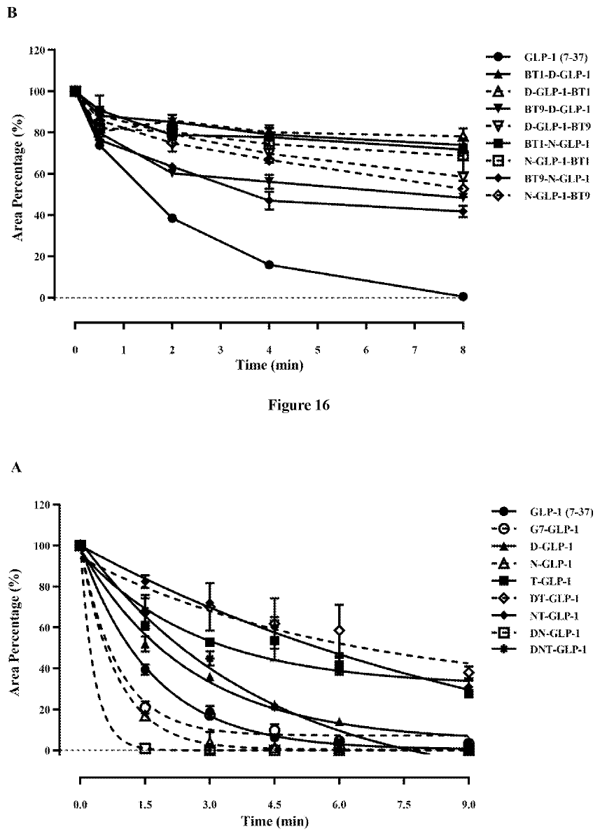

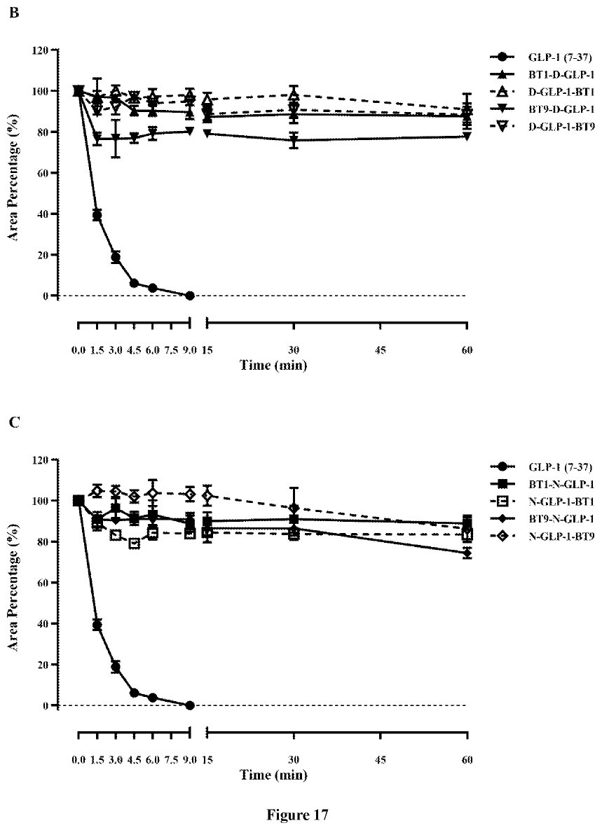

Results: After 8 hours of enzymatic hydrolysis by NEP24.11, GLP-1 (7-37) and G7-GLP-1 were almost completely degraded. The stability of N-GLP-1 (SEQ ID NO: 188) containing the peptide segment of Opiorphin (QRFSR) that inhibits NEP24.11, has been improved the most, with a remaining amount of about 56.4%, indicating that this Opiorphin (QRFSR) peptide segment can indeed exert an inhibitory effect on NEP24.11. Due to the scattered distribution of the cleavage sites of NEP24.11 throughout the GLP-1 molecule, molecules containing two or three inhibitory peptide scaffolds may have varying degrees of tolerance to NEP24.11 due to steric hindrance. The most stable D-GLP-1-BT1 (SEQ ID NO: 196) has a residual amount of nearly 80% after 8 hours of enzyme interaction (Table 15). The kinetic process of NEP24.11 enzymatic hydrolysis of GLP-1 and its analogues is shown in FIG. 16. The results indicate that the introduction of D, N, T, and BT peptide segments that inhibit metabolic enzymes can enhance the tolerance of GLP-1 to NEP24.11.

TABLE 15

| The stability of GLP-1 and its analogs (SEQ ID NO: 186-189) against NEP24.11 | |
|---|---|
| Time | Remaining peak area (%)/NEP24.11 |

| (h) | G7-GLP-1 | D-GLP-1 | N-GLP-1 | T-GLP-1 | GLP-1 (7-37) |
|---|---|---|---|---|---|
| 0.0 | 100.0 ± 0.0 | 100.0 ± 0.0 | 100.0 ± 0.0 | 100.0 ± 0.0 | 100.0 ± 0.0 |
| 0.5 | 63.0 ± 5.1 | 77.8 ± 7.8 | 83.9 ± 2.8 | 65.8 ± 10.3 | 73.9 ± 2.1 |
| 2.0 | 27.2 ± 7.2 | 53.7 ± 8.9 | 54.9 ± 13.6 | 43.3 ± 8.0 | 38.5 ± 0.9 |
| 4.0 | 11.5 ± 0.3 | 49.0 ± 7.3 | 51.0 ± 6.5 | 48.5 ± 5.6 | 15.9 ± 1.6 |
| 8.0 | 0.0 ± 0.0 | 31.3 ± 6.4 | 56.4 ± 15.8 | 40.3 ± 3.6 | 0.5 ± 0.9 |

| The stability of GLP-1 and its analogs (SEQ ID NO: 190-193) against NEP24.11 | |
|---|---|
| Time | Remaining peak area (%)/NEP24.11 |

| (h) | DT-GLP-1 | NT-GLP-1 | DN-GLP-1 | DNT-GLP-1 | GLP-1 (7-37) |
|---|---|---|---|---|---|
| 0.0 | 100.0 ± 0.0 | 100.0 ± 0.0 | 100.0 ± 0.0 | 100.0 ± 0.0 | 100.0 ± 0.0 |
| 0.5 | 77.6 ± 1.7 | 77.9 ± 7.5 | 86.6 ± 4.2 | 82.2 ± 1.3 | 73.9 ± 2.1 |
| 2.0 | 76.7 ± 2.5 | 58.3 ± 4.8 | 73.3 ± 2.1 | 79.1 ± 1.7 | 38.5 ± 0.9 |
| 4.0 | 72.9 ± 1.7 | 54.3 ± 3.7 | 69.3 ± 5.9 | 77.9 ± 4.2 | 15.9 ± 1.6 |
| 8.0 | 66.4 ± 0.0 | 48.8 ± 6.2 | 58.9 ± 4.9 | 67.8 ± 4.7 | 0.5 ± 0.9 |

The stability of GLP-1 and its analogs (SEQ ID NO: 194-197) against NEP24.11

| Time | Remaining peak area (%)/NEP24.11 | | | | |
|---|---|---|---|---|---|
| (h) | BT1-D-GLP-1 | BT1-N-GLP-1 | D-GLP-1-BT1 | N-GLP-1-BT1 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 88.2 ± 2.4 | 90.8 ± 7.1 | 80.3 ± 3.6 | 82.3 ± 8.3 | 73.9 ± 2.1 |
| 2.0 | 85.2 ± 0.5 | 78.8 ± 8.0 | 85.8 ± 2.7 | 80.3 ± 2.0 | 38.5 ± 0.9 |
| 4.0 | 79.1 ± 3.2 | 77.8 ± 4.3 | 80.1 ± 2.1 | 74.4 ± 9.1 | 15.9 ± 1.6 |
| 8.0 | 74.0 ± 1.3 | 71.7 ± 4.9 | 78.2 ± 3.9 | 68.7 ± 8.4 | 0.5 ± 0.9 |

The stability of GLP-1 and its analogs (SEQ ID NO: 198-201) against NEP24.11

| Time | Remaining peak area (%)/NEP24.11 | | | | |
|---|---|---|---|---|---|
| (h) | BT9-D-GLP-1 | BT9-N-GLP-1 | D-GLP-1-BT9 | N-GLP-1-BT9 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 79.8 ± 3.5 | 76.4 ± 1.1 | 88.4 ± 2.3 | 85.9 ± 2.8 | 73.9 ± 2.1 |
| 2.0 | 60.2 ± 1.8 | 63.5 ± 1.9 | 79.3 ± 2.6 | 74.9 ± 1.4 | 38.5 ± 0.9 |
| 4.0 | 56.1 ± 3.4 | 47.0 ± 4.3 | 69.8 ± 2.6 | 66.8 ± 2.1 | 15.9 ± 1.6 |
| 8.0 | 48.4 ± 1.4 | 41.8 ± 2.7 | 58.6 ± 0.7 | 52.7 ± 3.9 | 0.5 ± 0.9 |

### Example 6 Improve the Stability of Glucagon Like Peptide-1 (GLP-1) Against Pancreatic Trypsin, Chymotrypsin and Elastase

### Tolerance of GLP-1 and its Analogues (Hybrid Peptides) Towards Trypsin:

Control test: Take three sterile EP tubes and add 1.5 μL of 1 mM GLP-1 or GLP-1 analogs, 23.5 μL of reaction buffer (20 mM CaCl₂, pH 7.8, 50 mM Tris-HCl), and 3.75 μL of 10% TFA. Mix by centrifugation at 8000 rpm for 30 seconds.

Enzymatic hydrolysis kinetics of trypsin on GLP-1 analogues (SEQ ID NO: 186-193) without peptide scaffolds against trypsin: Take three sterile EP tubes and add 9 μL of 1 mM GLP-1 or GLP-1 analogs, and 135 μL of reaction buffer (20 mM CaCl₂, pH 7.8, 50 mM Tris-HCl). Arrange a certain volume of 0.05 μg/μL trypsin solution in another sterile EP. Incubate the EP tubes containing peptides and enzymes at 37° C. for 5 min, and separately add 6 μL of trypsin solution to each EP tube containing peptides and mix well. Start timing and extract 25 μL of reaction solution at 1.5, 3.0, 4.5, 6.0 and 9.0 min later, respectively. Add 3.75 μL of 10% TFA to terminate the reaction and mix well by centrifugation at 8000 rpm for 30 seconds.

Enzymatic hydrolysis kinetics of trypsin on GLP-1 analogues (SEQ ID NO: 194-201) containing peptide scaffolds against trypsin: Take three sterile EP tubes and add 13.5 μL of 1 mM GLP-1 or GLP-1 analogs and 202.5 μL of reaction buffer (20 mM CaCl₂, pH 7.8, 50 mM Tris-HCl). Arrange a certain volume of 0.05 μg/μL trypsin solution in another sterile EP. Incubate the EP tubes containing peptides and enzymes at 37° C. for 5 min, and add 9 μL of trypsin solution to each EP tube containing peptides and mix well. Start timing and extract 25 μL of reaction solution at 1.5, 3.0, 4.5, 6.0, 9.0, 15.0, 30.0, and 60.0 min later, respectively. Add 3.75 μL of 10% TFA to terminate the reaction and mix well by centrifugation at 8000 rpm for 30 seconds.

In the 25 μL of reaction system of two different sets described above, the final concentrations of GLP-1 or GLP-1 analogues and trypsin were 60 μM and 2.0 ng/μL, respectively. There are three replicates at each time point, and the peak area of the peptide at each time point is detected using reverse phase high-performance liquid chromatography (RP-HPLC). The ratio of the remaining peak area of the sample at detection time T (h) to the peak area of the prototype peptide at 0 h is calculated as the remaining percentage (%) of the peptide.

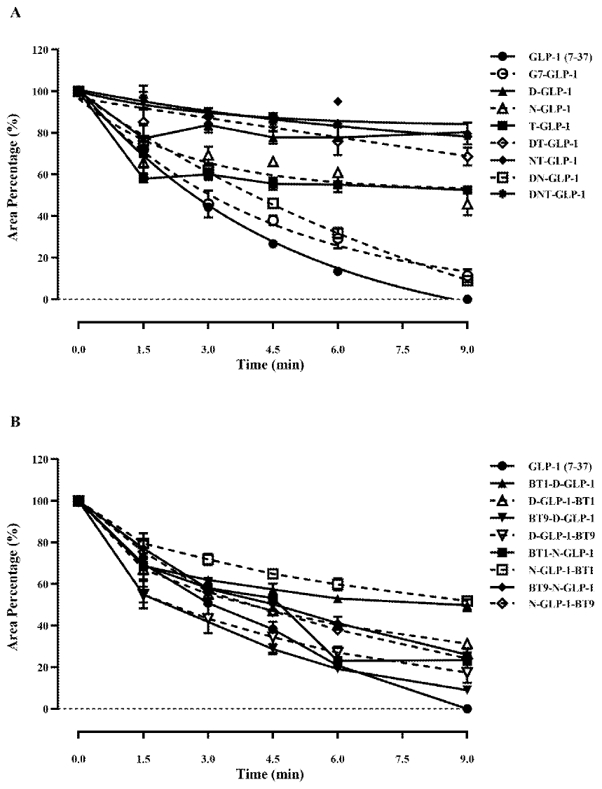

Results: GLP-1 analogs SEQ ID NO: 186-193, which does not contain the inhibitory peptide scaffols against trypsin, has poor tolerance to trypsin hydrolysis and is almost degraded at 9 min; Although the trypsin inhibitory activity of BT43 (SEQ ID NO: 43) is weak, GLP-1 analogues containing a partial inhibitory peptide segment of BT43 (SEQ ID NO: 43) exhibited certain tolerance (FIG. 17A and Table 16). The results indicate that the inhibitory peptide scaffold can to some extent enhance the tolerance of GLP-1 molecules to trypsin, while the introduction of other inhibitory peptide scaffolds is ineffective. DNT-GLP-1 (SEQ ID NO: 193) has also been completely degraded due to significant changes in the secondary structure. The introduction of protease inhibitory scaffolds BT1 and BT9 in GLP-1 analogues SEQ ID NO: 194-201 significantly improved the tolerance of GLP-1 to trypsin, because the remaining amount of the prototype molecule was greater than 75% after 60 minutes of trypsin hydrolysis (FIG. 17B, FIG. 17C, and Table 16).

TABLE 16

| The stability of GLP-1 and its analogs (SEQ ID NO: 186-189) against trypsin | |
|---|---|
| Time | Remaining peak area (%)/trypsin |

| (min) | G7-GLP-1 | D-GLP-1 | N-GLP-1 | T-GLP-1 | GLP-1 (7-37) |
|---|---|---|---|---|---|
| 0.0 | 100.0 ± 0.0 | 100.0 ± 0.0 | 100.0 ± 0.0 | 100.0 ± 0.0 | 100.0 ± 0.0 |
| 1.5 | 21.0 ± 2.9 | 51.9 ± 3.7 | 16.9 ± 1.6 | 60.9 ± 1.1 | 39.4 ± 2.6 |
| 3.0 | 17.2 ± 0.8 | 36.0 ± 1.4 | 3.7 ± 5.3 | 52.8 ± 0.9 | 18.8 ± 2.9 |
| 4.5 | 9.8 ± 2.9 | 22.4 ± 1.4 | 1.2 ± 1.7 | 53.6 ± 9.7 | 6.1 ± 1.9 |
| 6.0 | 4.5 ± 0.8 | 14.1 ± 1.5 | 0.0 ± 0.0 | 41.6 ± 3.6 | 3.8 ± 1.8 |
| 9.0 | 3.4 ± 1.7 | 4.7 ± 0.8 | 0.0 ± 0.0 | 27.6 ± 0.1 | 0.0 ± 0.0 |

| The stability of GLP-1 and its analogs (SEQ ID NO: 190-193) against trypsin | |
|---|---|
| Time | Remaining peak area (%)/trypsin |

| (min) | DT-GLP-1 | NT-GLP-1 | DN-GLP-1 | DNT-GLP-1 | GLP-1 (7-37) |
|---|---|---|---|---|---|
| 0.0 | 100.0 ± 0.0 | 100.0 ± 0.0 | 100.0 ± 0.0 | 100.0 ± 0.0 | 100.0 ± 0.0 |
| 1.5 | 67.5 ± 8.5 | 82.4 ± 3.1 | 1.0 ± 0.1 | 68.3 ± 5.9 | 39.4 ± 2.6 |
| 3.0 | 70.1 ± 11.6 | 72.1 ± 1.8 | 0.0 ± 0.0 | 44.9 ± 3.5 | 18.8 ± 2.9 |
| 4.5 | 61.9 ± 12.3 | 60.2 ± 4.9 | 0.0 ± 0.0 | 21.2 ± 1.1 | 6.1 ± 1.9 |
| 6.0 | 58.5 ± 12.5 | 42.2 ± 5.3 | 0.0 ± 0.0 | 0.0 ± 0.0 | 3.8 ± 1.8 |
| 9.0 | 38.0 2.9 | 31.0 ± 3.1 | 0.0 ± 0.0 | 0.0 ± 0.0 | 0.0 ± 0.0 |

The stability of GLP-1 and its analogs (SEQ ID NO: 194-197) against trypsin

| Time | Remaining peak area (%)/trypsin | | | | |
|---|---|---|---|---|---|
| (min) | BT1-D-GLP-1 | BT1-N-GLP-1 | D-GLP-1-BT1 | N-GLP-1-BT1 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 97.3 ± 8.8 | 90.9 ± 3.6 | 96.9 ± 3.1 | 89.2 ± 3.7 | 39.4 ± 2.6 |
| 3.0 | 96.6 ± 2.0 | 96.4 ± 4.9 | 100.2 ± 2.5 | 83.2 ± 2.3 | 18.8 ± 2.9 |
| 4.5 | 90.4 ± 2.4 | 91.4 ± 3.2 | 96.5 ± 2.1 | 79.1 ± 1.0 | 6.1 ± 1.9 |
| 6.0 | 90.3 ± 2.5 | 93.2 ± 7.0 | 97.3 ± 3.6 | 84.4 ± 3.4 | 3.8 ± 1.8 |
| 9.0 | 89.7 ± 3.4 | 88.8 ± 4.0 | 98.0 ± 3.1 | 83.9 ± 0.5 | 0.0 ± 0.0 |
| 15.0 | 87.3 ± 2.6 | 90.0 ± 4.4 | 95.9 ± 3.2 | 84.5 ± 4.9 | — |
| 30.0 | 88.6 ± 4.4 | 91.0 ± 6.6 | 98.1 ± 4.4 | 83.7 ± 3.0 | — |
| 60.0 | 87.7 ± 6.2 | 88.9 ± 3.0 | 91.0 ± 7.6 | 83.5 ± 1.4 | — |

The stability of GLP-1 and its analogs (SEQ ID NO: 198-201) against trypsin

| Time | Remaining peak area (%)/trypsin | | | | |
|---|---|---|---|---|---|
| (min) | BT9-D-GLP-1 | BT9-N-GLP-1 | D-GLP-1-BT9 | N-GLP-1-BT9 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 76.6 ± 3.0 | 90.8 ± 0.5 | 90.3 ± 1.9 | 104.8 ± 3.0 | 39.4 ± 2.6 |
| 3.0 | 76.7 ± 9.1 | 90.4 ± 2.0 | 92.2 ± 3.7 | 104.5 ± 2.8 | 18.8 ± 2.9 |
| 4.5 | 77.0 ± 2.4 | 91.0 ± 2.1 | 97.3 ± 1.0 | 102.1 ± 3.0 | 6.1 ± 1.9 |
| 6.0 | 79.2 ± 3.1 | 91.1 ± 3.0 | 94.0 ± 1.5 | 103.8 ± 6.4 | 3.8 ± 1.8 |
| 9.0 | 80.2 ± 1.0 | 90.9 ± 3.1 | 94.9 ± 1.8 | 103.2 ± 3.5 | 0.0 ± 0.0 |
| 15.0 | 79.1 ± 2.2 | 86.6 ± 3.5 | 88.7 ± 3.0 | 102.5 ± 4.9 | — |
| 30.0 | 75.8 ± 3.8 | 86.5 ± 3.5 | 90.8 ± 3.6 | 96.4 ± 9.9 | — |
| 60.0 | 77.7 ± 0.5 | 74.4 ± 2.6 | 88.4 ± 3.9 | 86.3 ± 6.4 | — |

### Tolerance of GLP-1 and its Analogues (Hybrid Peptides) to Chymotrypsin:

Control test: Take three sterile EP tubes and add 1.5 μL of 1 mM GLP-1 or GLP-1 analogs, 23.5 μL of reaction buffer (20 mM CaCl₂, pH 7.8, 50 mM Tris-HCl), and 3.75 μL of 10% TFA. Mix by centrifugation at 8000 rpm for 30 seconds.

Enzymatic hydrolysis kinetics of chymotrypsin on GLP-1 analogues (SEQ ID NO: 186-201) without inhibitory peptide scaffolds against chymotrypsin: Take three sterile EP tubes and add 9 μL of 1 mM GLP-1 or GLP-1 analogs and 138 μL of reaction buffer (20 mM CaCl₂, pH 7.8, 50 mM Tris-HCl). Arrange a certain volume of 0.05 μg/μL chymotrypsin solution in another sterile EP. Incubate the EP tubes containing peptides and enzymes at 37° C. for 5 min, and add 3 μL of trypsin solution to each EP tube containing peptides and mix well. Start timing and extract 25 μL of reaction solution at 1.5, 3.0, 4.5, 6.0 and 9.0 min later, respectively. Add 3.75 μL of 10% TFA to terminate the reaction and mix well by centrifugation at 8000 rpm for 30 seconds.

Enzymatic hydrolysis kinetics of chymotrypsin on GLP-1 analogues (SEQ ID NO: 202-205) containing inhibitory peptide scaffolds against chymotrypsin: Take three sterile EP tubes, and add 13.5 μL of 1 mM GLP-1 or GLP-1 analogs and 207 μL of reaction buffer (20 mM CaCl₂, pH 7.8, 50 mM Tris-HCl). Arrange a certain volume of 0.05 μg/μL chymotrypsin solution in another sterile EP. Incubate the EP tubes containing peptides and enzymes at 37° C. for 5 min, and then add 4.5 μL of chymotrypsin solution to each EP tube containing peptides and mix well. Start timing and extract 25 μL of reaction solution at 1.5, 3.0, 4.5, 6.0, 9.0, 15.0, 30.0, and 60.0 min later, respectively. Add 3.75 μL of 10% TFA to terminate the reaction and mix well by centrifugation at 8000 rpm for 30 seconds.

In the 25 μL of reaction system of two different sets described above, the final concentrations of GLP-1 or GLP-1 analogues and chymotrypsin were 60 μM and 1.0 ng/μL, respectively. There are three replicates at each time point, and the peak area of the peptide at each time point is detected using reverse phase high-performance liquid chromatography (RP-HPLC). The ratio of the remaining peak area of the sample at detection time T (h) to the peak area of the prototype peptide at 0 h is calculated as the remaining percentage (%) of the peptide.

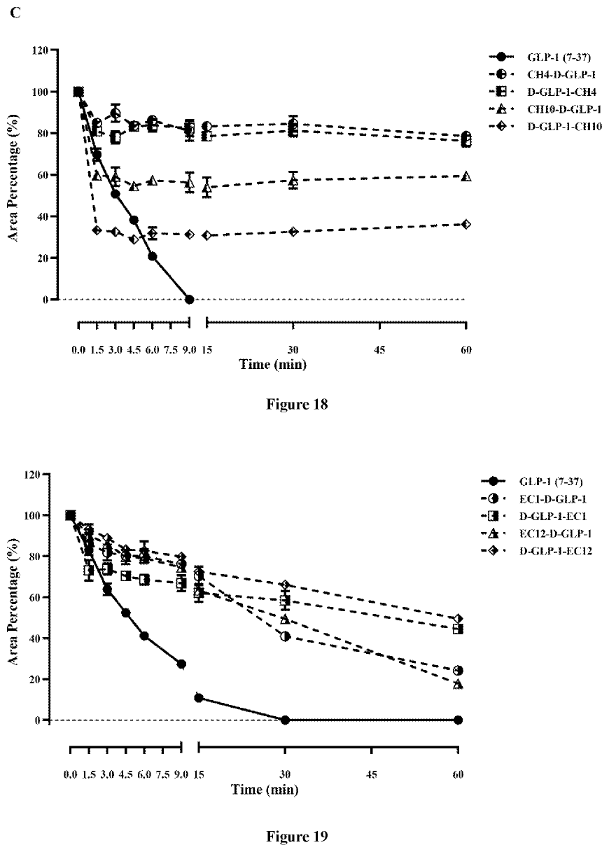

Results: After 9 min of chymotrypsin hydrolysis, GLP-1 was completely degraded, and the results of two experiments were consistent. GLP-1 analogues SEQ ID NO: 186-201 do not contain inhibitory peptide scaffolds against chymotrypsin, and their stability towards chymotrypsin hydrolysis is relatively low. However, GLP-1 analogues SEQ ID NO: 189-191 and SEQ ID NO: 193, which contain a partial inhibitory peptide segment of BT43 (SEQ ID NO: 43), exhibited certain tolerance compared to GLP-1 molecules, with more than 50% of the remaining prototype peptides after 9 min of chymotrypsin hydrolysis (FIGS. 18A&18B and Table 17); The GLP-1 analogs SEQ ID NO: 202-204, which specifically introduced inhibitory peptide scaffolds against chymotrypsin, had more than 60% of the prototype peptide remaining after 60 minutes of chymotrypsin hydrolysis. However, the GLP-1 analog SEQ ID NO: 205 was an exception. The prototype peptide molecule of this hybrid peptide was difficult to achieve baseline separation from the enzymatic hydrolysis product, and calculation errors resulted in a lower residual amount after chymotrypsin hydrolysis (FIG. 18C and Table 17).

TABLE 17
The stability of GLP-1 and its analogs (SEQ ID NO: 186-189) against chymotrypsin

| Time | Remaining peak area (%)/chymotrypsin | | | | |
|---|---|---|---|---|---|
| (min) | G7-GLP-1 | D-GLP-1 | N-GLP-1 | T-GLP-1 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 72.1 ± 11.5 | 77.3 ± 7.0 | 65.8 ± 2.6 | 58.0 ± 1.2 | 72.7 ± 3.3 |
| 3.0 | 45.8 ± 6.5 | 83.8 ± 3.6 | 69.3 ± 4.0 | 60.1 ± 2.7 | 44.3 ± 1.2 |
| 4.5 | 38.0 ± 2.3 | 77.9 ± 3.1 | 66.2 ± 1.9 | 55.6 ± 2.9 | 26.7 ± 2.1 |
| 6.0 | 29.3 ± 4.7 | 77.8 ± 1.0 | 60.9 ± 0.8 | 55.1 ± 3.6 | 13.5 ± 2.2 |
| 9.0 | 11.3 ± 3.2 | 77.4 ± 1.4 | 45.8 ± 5.4 | 52.6 ± 1.7 | 0.0 ± 0.0 |

The stability of GLP-1 and its analogs (SEQ ID NO: 190-193) against chymotrypsin

| Time | Remaining peak area (%)/chymotrypsin | | | | |
|---|---|---|---|---|---|
| (min) | DT-GLP-1 | NT-GLP-1 | DN-GLP-1 | DNT-GLP-1 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 85.2 ± 7.0 | 95.4 ± 4.3 | 76.3 ± 2.0 | 97.0 ± 5.7 | 72.7 ± 3.3 |
| 3.0 | 87.9 ± 3.9 | 84.6 ± 3.2 | 62.0 ± 1.7 | 89.8 ± 0.9 | 44.3 ± 1.2 |
| 4.5 | 87.3 ± 1.2 | 85.5 ± 2.8 | 46.1 ± 0.9 | 85.1 ± 4.4 | 26.7 ± 2.1 |
| 6.0 | 76.0 ± 6.7 | 95.1 ± 0.9 | 31.9 ± 2.5 | 84.3 ± 2.0 | 13.5 ± 2.2 |
| 9.0 | 68.6 ± 4.3 | 79.7 ± 5.2 | 9.0 ± 0.8 | 78.2 ± 2.1 | 0.0 ± 0.0 |

The stability of GLP-1 and its analogs (SEQ ID NO: 194-197) against chymotrypsin

| Time | Remaining peak area (%)/chymotrypsin | | | | |
|---|---|---|---|---|---|
| (min) | BT1-D-GLP-1 | BT1-N-GLP-1 | D-GLP-1-BT1 | N-GLP-1-BT1 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 68.7 ± 1.6 | 69.0 ± 4.4 | 67.1 ± 4.9 | 79.5 ± 4.9 | 69.7 ± 3.0 |
| 3.0 | 61.9 ± 1.5 | 58.1 ± 4.9 | 55.2 ± 0.6 | 71.8 ± 2.7 | 50.8 ± 1.6 |
| 4.5 | 57.4 ± 2.7 | 53.2* | 47.1 ± 1.4 | 64.9 ± 1.0 | 38.3 ± 1.8 |
| 6.0 | 53.1 ± 0.5 | 23.1 ± 0.9 | 40.4 ± 1.0 | 59.8 ± 2.9 | 20.9 ± 1.3 |
| 9.0 | 49.8 ± 2.8 | 23.5 ± 0.2 | 31.3 ± 0.7 | 51.9 ± 1.0 | 0.0 ± 0.0 |

*Baseline separation was not achieved, peak area did not match the actual situation, and
statistics was not conducted.
The stability of GLP-1 and its analogs (SEQ ID NO: 198-201) against chymotrypsin

| Time | Remaining peak area (%)/chymotrypsin | | | | |
|---|---|---|---|---|---|
| (min) | BT9-D-GLP-1 | BT9-N-GLP-1 | D-GLP-1-BT9 | N-GLP-1-BT9 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 54.9 ± 6.6 | 77.1 ± 1.8 | 54.9 ± 3.8 | 75.5 ± 5.3 | 69.7 ± 3.0 |
| 3.0 | — | 57.7 ± 2.0 | 43.3 ± 7.0 | 56.3 ± 2.1 | 50.8 ± 1.6 |
| 4.5 | 28.7 ± 2.4 | 50.4 ± 3.2 | 34.5 ± 7.4 | 47.0 ± 1.6 | 38.3 ± 1.8 |
| 6.0 | 19.2 ± 1.3 | 41.2 ± 3.1 | 27.1 ± 2.9 | 38.1 ± 2.2 | 20.9 ± 1.3 |
| 9.0 | 9.0 ± 0.9 | 26.0 ± 2.9 | 17.4 ± 4.9 | 24.2 ± 3.0 | 0.0 ± 0.0 |

—Not integrated.

The stability of GLP-1 and its analogs (SEQ ID NO: 202-205) against chymotrypsin

| Time | Remaining peak area (%)/chymotrypsin | | | | |
|---|---|---|---|---|---|
| (min) | CH4-D-GLP-1 | D-GLP-1-CH4 | CH10-D-GLP-1 | D-GLP-1-CH10 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 84.9 ± 1.6 | 80.9 ± 1.7 | 59.7 ± 2.1 | 33.4 ± 1.7* | 69.7 ± 3.0 |
| 3.0 | 89.6 ± 4.2 | 78.0 ± 2.9 | 59.1 ± 4.5 | 32.6 ± 0.7 | 50.8 ± 1.6 |
| 4.5 | 83.6 ± 0.5 | 83.2 ± 0.3 | 54.5 ± 1.2 | 28.9 ± 1.1 | 38.3 ± 1.8 |
| 6.0 | 86.2 ± 0.7 | 83.9 ± 3.0 | 57.3 ± 1.5 | 31.9 ± 2.9 | 20.9 ± 1.3 |
| 9.0 | 81.1 ± 4.7 | 82.4 ± 3.9 | 56.3 ± 4.7 | 31.3 ± 0.8 | 0.0 ± 0.0 |
| 15.0 | 83.3 ± 1.4 | 78.5 ± 2.1 | 54.0 ± 4.7 | 30.9 ± 1.0 | — |
| 30.0 | 84.5 ± 3.7 | 81.3 ± 2.8 | 57.4 ± 4.0 | 32.6 ± 0.6 | — |
| 60.0 | 78.6 ± 2.0 | 76.3 ± 2.7 | 59.4 ± 2.1 | 36.2 ± 1.4 | — |

*Baseline separation not achieved.

### Tolerance of GLP-1 and its Analogues (Hybrid Peptides) to Elastase:

Control test: Take three sterile EP tubes and add 1.5 μL of 1 mM GLP-1 or GLP-1 analogs, 23.5 μL of reaction buffer (50 mM Tris-HCl, pH 8.0), and 3.75 μL of 10% TFA. Mix by centrifugation at 8000 rpm for 30 seconds.

Enzymatic hydrolysis kinetics of elastase on GLP-1 analogues (SEQ ID NO: 206-209) containing inhibitory peptide scaffolds against elastase: Take three sterile EP tubes, and add 13.5 μL of 1 mM GLP-1 or GLP-1 analogs and 207 μL of reaction buffer (50 mM Tris-HCl, pH 8.0). Arrange a certain volume of 0.5 μg/μL elastase solution in another sterile EP. Incubate the EP tubes containing peptides and enzymes at 37° C. for 5 min, and then add 4.5 μL of elastase solution to each EP tube containing peptides and mix well. Start timing and remove 25 μL of reaction solution at 1.5, 3.0, 4.5, 6.0, 9.0, 15.0, 30.0, and 60.0 min later, respectively. Add 3.75 μL of 10% TFA to terminate the reaction and mix well by centrifugation at 8000 rpm for 30 seconds. In the 25 μL of reaction system, the final concentrations of GLP-1 or GLP-1 analogues and elastase were 60 μM and 10 ng/μL, respectively. There are three replicates at each time point, and the peak area of the peptide at each time point is detected using reverse phase high-performance liquid chromatography (RP-HPLC). The ratio of the remaining peak area of the sample at detection time T (h) to the peak area of the prototype peptide at 0 h is calculated as the remaining percentage (%) of the peptide.

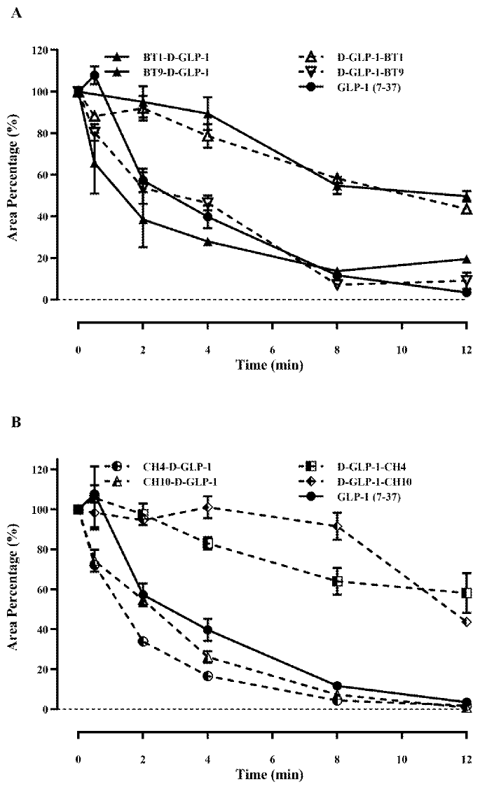

Results: Based on the fact that GLP-1 analogs containing the inhibitory peptide scaffolds against trypsin and chymotrypsin have the tolerance to hydrolysis of metabolic enzymes, in this experimental only the tolerance of GLP-1 analogs containing elastase inhibitory peptides (SEQ ID Nos: 206-209) to degradation of elastase were evaluated. The results showed that about 10% of GLP-1 was left after 15 min of enzymatic hydrolysis, and the remaining of GLP-1 analogues containing inhibitory peptides against elastase was more than 50%. After 30 minutes of enzymatic hydrolysis, no GLP-1 prototype molecule was detected. After 60 min of enzymatic hydrolysis, about 20% of GLP-1 analogs (SEQ ID NO: 206, SEQ ID NO: 208) fused with inhibitory peptide against elastase at the N-terminus remained; However, about 45% of GLP-1 analogs (SEQ ID NO: 207, SEQ ID NO: 209) fused with inhibitory peptide against elastase at the C-terminus remained, indicating that the introduction of EC inhibitory peptide molecules into the C-terminal of GLP-1 molecule can better improve its stability of enzymatic hydrolysis of elastase (FIG. 19 and Table 18).

TABLE 18
The stability of GLP-1 and its analogs (SEQ ID NO: 206-209) against elastase

| Time | Remaining peak area (%)/elastase | | | | |
|---|---|---|---|---|---|
| (min) | EC1-D-GLP-1 | D-GLP-1-EC1 | EC12-D-GLP-1 | D-GLP-1-EC12 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 1.5 | 86.0 ± 5.4 | 73.1 ± 5.0 | 90.2 ± 3.0 | 93.2 ± 2.4 | 82.9 ± 0.7 |
| 3.0 | 81.9 ± 3.9 | 73.8 ± 2.8 | 86.0 ± 1.7 | 88.9 ± 1.6 | 63.9 ± 2.7 |
| 4.5 | 80.3 ± 2.0 | 70.4 ± 2.0 | 79.7 ± 3.5 | 83.3 ± 1.0 | 52.4 ± 0.7 |
| 6.0 | 80.1 ± 3.4 | 68.6 ± 2.4 | 79.1 ± 2.2 | 82.7 ± 4.6 | 41.1 ± 1.5 |
| 9.0 | 76.2 ± 1.6 | 66.9 ± 3.9 | 74.5 ± 2.2 | 79.8 ± 2.2 | 27.4 ± 0.9 |
| 15.0 | 70.4 ± 4.5 | 62.1 ± 4.3 | 63.3 ± 0.7 | 72.5 ± 1.6 | 10.8 ± 0.2 |
| 30.0 | 40.9 ± 1.1 | 58.4 ± 4.6 | 49.4 ± 0.9 | 66.1 ± 2.1 | 0.0 ± 0.0 |
| 60.0 | 24.2 ± 0.6 | 44.5 ± 0.9 | 17.8 ± 2.0 | 49.5 ± 1.1 | 0.0 ± 0.0 |

### Example 7 Serum Stability of Glucagon Like Peptide-1 (GLP-1) Analogue (Hybrid Peptide)

Control test: Take three sterile EP tubes and sequentially add 3 μL of 1 mM GLP-1 or GLP-1 analogs, 25 μL of human serum (obtained from SenBeiJia Biological Technology Co., Ltd.), 72 μL of reaction buffer (50 mM Tris-HCl, pH7.0), and 300 μL of pre-cold absolute methanol. Invert and mix thoroughly, then leave at −20° C. overnight. At the same time, take three sterile EP tubes and add 25 μL of human serum, 75 μL of 50 mM Tris-HCl buffer (pH7.0), and 300 μL of pre-cold absolute methanol. Invert and mix thoroughly, then leave at −20° C. overnight as a negative control, so as to eliminate the interference of proteins or peptides contained in human serum at the peak time of the target peptide after methanol precipitation.

Serum stability experiment: Take three sterile EP tubes and sequentially add 16.5 μL of 1 mM GLP-1 or GLP-1 analogs, and 396 μL of reaction buffer (50 mM Tris-HCl, pH 8.0). Arrange a certain volume of human serum in another sterile EP. Incubate the EP tubes containing peptides and serum at 37° C. for 10 min, and add 137.5 μL of human serum to each EP tube containing peptides and mix well. The final concentrations of GLP-1 or GLP-1 analogues and human serum were 0.03 mM and 25% (v/v), respectively. Start timing and remove 100 μL of reaction solution at 0.5, 2.0, 4.0, 8.0, and 12.0 h later, respectively. Add 300 μL of pre-cold absolute methanol. Invert and mix thoroughly, then leave at −20° C. overnight. All samples were centrifuged at 4° C. for 10 min at 18000 g, and the supernatant was taken and subjected to drain organic solvent using a suction bottle and freeze-dried. Add 60 μL of 50% (v/v) methanol/water solution to dissolve the sample, centrifuge at 4° C. for 5 min at 18000 g, and take the supernatant for RP-HPLC analysis. There are three replicates at each time point, and the peak area of the peptide at each time point is detected using reverse phase high-performance liquid chromatography (RP-HPLC). The ratio of the remaining peak area of the sample at detection time T (h) to the peak area of the prototype peptide at 0 h is calculated as the remaining percentage (%) of the peptide. The negative control shows that the proteins or peptides contained in human serum do not interfere with the detection of the target peptide under this treatment method.

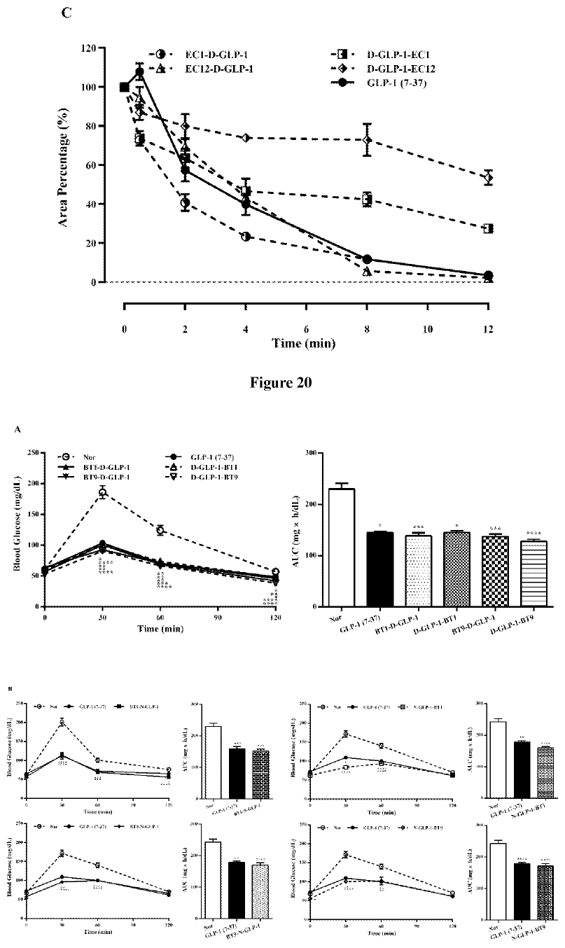

Results: After incubation with human serum for 12 hours, GLP-1 and its analogues remained about 3.5%, which was inconsistent with the reported plasma half-life of only 1-2 minutes. The reason was that GLP-1 was mainly metabolized by metabolic enzymes DPP-IV and NEP24.11 in the systemic circulation, which were membrane proteins and extremely low in normal serum, especially NEP24.11 released in the blood circulation could be used as a biomarker of many physiological and pathological processes. The GLP-1 analogs containing inhibitory peptide scaffolds against trypsin, chymotrypsin and elastase showed high serum stability, and the GLP-1 analogs containing inhibitory peptide scaffolds against trypsin fused both at N-terminus (SEQ ID NO: 194 and SEQ ID NO: 198) and C-terminus (SEQ ID NO: 196 and SEQ ID NO: 200) showed good serum stability; In addition, GLP-1 analogs containing inhibitory peptide scaffolds against chymotrypsin and elastase at C-terminal (SEQ ID NO: 203, SEQ ID NO: 205, SEQ ID NO: 207, SEQ ID NO: 209) are more stable in serum than GLP-1 analogs containing inhibitory peptide scaffolds against chymotrypsin and elastase at N-terminal (SEQ ID NO: 202, SEQ ID NO: 204, SEQ ID NO: 206, SEQ ID NO: 208) (FIG. 20 and Table 19). The results indicate that GLP-1 analogues fused with inhibitory peptide scaffolds that inhibit serine proteases have inhibitory effects on not only DPP-IV and NEP24.11, but also other serine metabolic enzymes in serum, so as to improve the stability in the serum.

TABLE 19
The stability of GLP-1 and its analogues in human serum

| Time | Remaining peak area (%)/human serum | | | | |
|---|---|---|---|---|---|
| (h) | BT1-D-GLP-1 | D-GLP-1-BT1 | BT9-D-GLP-1 | D-GLP-1-BT9 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 68.9 ± 7.1^{a} | 88.2^{b} | 65.6 ± 14.7 | 80.4 ± 4.0 | 107.8 ± 4.3 |
| 2.0 | 95.1 ± 7.5 | 92.0 ± 5.9 | 38.5 ± 13.3 | 53.6 ± 7.6 | 57.3 ± 5.7 |
| 4.0 | 89.4 ± 7.9 | 78.7 ± 5.7 | 28.0^{b} | 46.5 ± 3.5 | 39.8 ± 5.5 |
| 8.0 | 54.8 ± 4.1 | 58.2^{b} | 13.7^{b} | 7.2 ± 1.3 | 11.7 ± 1.0 |
| 12.0 | 49.7 ± 2.5 | 43.6 ± 0.3 | 19.6 ± 1.2 | 9.1 ± 3.9 | 3.5 ± 0.5 |

^{a}The remaining amount of the sample at this time point did not comply with the degradation
law, so no statistics was made.
^{b}Two samples at this time point did not achieve baseline separation.

The stability of GLP-1 and its analogues in human serum

| Time | Remaining peak area (%)/human serum | | | | |
|---|---|---|---|---|---|
| (h) | CH4-D-GLP-1 | D-GLP-1-CH4 | CH10-D-GLP-1 | D-GLP-1-CH10 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 72.3 ± 0.5 | 105.8 ± 15.8 | 74.4 ± 5.5 | 98.4 ± 7.3 | 107.8 ± 4.3 |
| 2.0 | 34.0 ± 0.2 | 97.6 ± 5.4 | 54.8 ± 2.1 | 94.6 ± 2.1 | 57.3 ± 5.7 |
| 4.0 | 16.6 ± 0.8 | 83.0 ± 3.1 | 26.2 ± 2.9 | 101.1 ± 5.5 | 39.8 ± 5.5 |
| 8.0 | 4.4 ± 0.2 | 64.0 ± 6.8 | 7.3 ± 1.0 | 91.6 ± 6.8 | 11.7 ± 1.0 |
| 12.0 | 1.8 ± 0.1 | 58.2 ± 10.0 | 0.8 ± 0.0 | 43.7 ± 2.1 | 3.5 ± 0.5 |

The stability of GLP-1 and its analogues in human serum

| Time | Remaining peak area (%)/human serum | | | | |
|---|---|---|---|---|---|
| (h) | EC1-D-GLP-1 | D-GLP-1-EC1 | EC12-D-GLP-1 | D-GLP-1-EC12 | GLP-1 (7-37) |
| 0.0 | 100.0 ± 0.0 | 100.0 ± 0.0 | 100.0 ± 0.0 | 100.0 ± 0.0 | 100.0 ± 0.0 |
| 0.5 | 72.8 ± 0.8 | 73.7 ± 3.7 | 94.6 ± 5.3 | 86.9 ± 3.8 | 107.8 ± 4.3 |
| 2.0 | 40.8 ± 4.3 | 63.5 ± 4.8 | 69.3 ± 4.0 | 79.9 ± 6.2 | 57.3 ± 5.7 |
| 4.0 | 23.4 ± 1.9 | 46.6 ± 6.4 | 43.4 ± 1.7 | 73.9 ± 1.6 | 39.8 ± 5.5 |
| 8.0 | 11.7 ± 2.0 | 42.4 ± 3.5 | 5.7 ± 0.1 | 72.9 ± 8.2 | 11.7 ± 1.0 |
| 12.0 | 3.4 ± 0.4 | 27.4 ± 0.3 | 2.2 ± 0.1 | 53.5 ± 3.7 | 3.5 ± 0.5 |

### Example 8. In Vivo Hypoglycemic Activity of GLP-1 Analogues (Hybrid Peptides) in Normal ICR Mice

### Subcutaneous Administration:

The day before the experiment, all of the animals were fasted for 15-16 hours with water ad libitum. On the day of the experiment, the animals were randomly divided according to body weight (n=10). Firstly, the blood was collected from the tail tip at 0 hour, and then the animals were administered subcutaneously with GLP-1 analog (SEQ ID NOs: 194-201, 1 μmol/kg) or saline. After 30 minutes, the animals were administered by gavage with glucose solution (2 g/kg), and the blood was collected from the tail tip at 30, 60, and 120 minutes later. Blood glucose was measured using glucose oxidase method. The blood glucose and area under the blood glucose-time curve (AUC) were calculated.

AUC (mg×h/dL)=(BG₀+BG₃₀)×30/60+(BG₃₀+BG₆₀)×30/60+(BG₆₀+BG₁₂₀)×60/60. BG0, BG30, BG60, and BG120 represent blood glucose levels at 0, 30, 60, and 120 minutes after glucose loading, respectively.

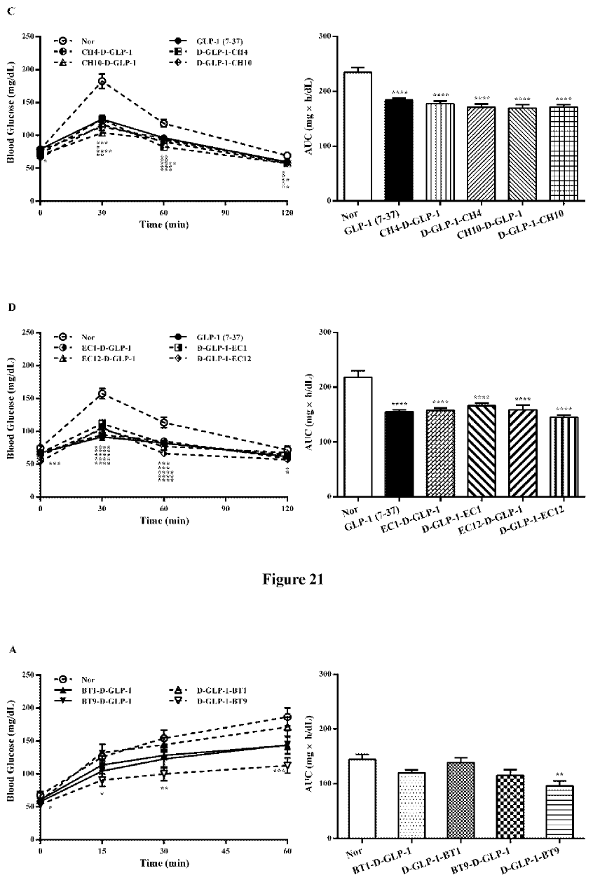

Results: Subcutaneous injection of GLP-1 analogues (SEQ ID NO: 194, SEQ ID NO: 196, SEQ ID NO: 198, and SEQ ID NO: 200) containing both inhibitory peptide scaffolds BT9 (SEQ ID NO: 9) and BT45 (SEQ ID NO: 45) against trypsin, and anti-DPP-IV diprotin A (IPI) peptide segments significantly reduced the blood glucose levels at 30, 60, and 120 minutes after oral glucose loading and the AUC in normal ICR mice (FIG. 21A and Table 20). Subcutaneous injection of GLP-1 analogues (SEQ ID NO: 195, SEQ ID NO: 197, SEQ ID NO: 199, SEQ ID NO: 201) containing both anti-trypsin peptide scaffolds BT9 (SEQ ID NO: 9) and BT45 (SEQ ID NO: 45), and the anti-NEP24.11 Opiorphin (QRFSR) peptide segment significantly reduced the blood glucose levels at 30 and 60 minutes after oral glucose loading and the AUC in normal ICR mice. These results indicated that the introduction of inhibitory peptide scaffolds against trypsin does not disrupt the binding of GLP-1 to receptors.

Subcutaneous injection of GLP-1 analogs (SEQ ID NOs: 202-205) containing both anti-chymotrypsin peptide scaffolds CH4 (SEQ ID NO: 84) and CH10 (SEQ ID NO: 90), and the anti-DPP-IV diprotin A (IPI) peptide segments can also significantly reduce the blood glucose levels at 30, 60, and 120 minutes after oral glucose loading and the AUC in normal ICR mice (FIG. 21C and Table 20), indicating that the introduction of inhibitory peptide scaffolds chymotrypsin does not affect the binding of GLP-1 to receptors.

Subcutaneous injection of GLP-1 analogues (SEQ ID NOs: 206-209) containing both anti-elastase peptide scaffolds EC1 (SEQ ID NO: 134) and EC12 (SEQ ID NO: 145), and the anti-DPP-IV diprotin A (IPI) peptide segments also significantly reduced the blood glucose levels at 30 and 60 minutes after oral glucose loading and the AUC in normal ICR mice (FIG. 21D and Table 20). These results indicated that the introduction of anti-elastase peptide scaffolds does not affect the binding of GLP-1 to receptors.

Subcutaneous injection of acetylated and amidated GLP-1 analogues (SEQ ID NO: 194, SEQ ID NO: 196, SEQ ID NO: 198, and SEQ ID NO: 200), as well as N-terminal PEG modified GLP-1 analogs (SEQ ID NO: 200 and SEQ ID NO: 204) did not show any significant differences in their hypoglycemic activity compared to the unmodified molecules.

TABLE 20
The blood glucose lowering activity of GLP-1 analogs
administered by suncutaneous injection

| | Dose | | | | | | AUC |
|---|---|---|---|---|---|---|---|
| | (μmol/ | | Blood glucose (mg/dL, mean ± SEM) | | | | (mean ± |
| Group | kg) | n | 0 min | 30 min | 60 min | 120 min | SEM) |
| Nor | — | 10 | 60.7 ± | 186.3 ± | 124.2 ± | 57.1 ± | 230.0 ± |
| | | | 2.6 | 10.7 | 7.9 | 2.4 | 11.4 |
| GLP-1 | 1 | 10 | 62.1 ± | 103.7 ± | 70.9 ± | 48.6 ± | 144.9 ± |
| | | | 3.3 | 2.6* | 2.1** | 1.2* | 2.6* |
| BT1-D- | 1 | 10 | 64.2 ± | 92.8 ± | 70.9 ± | 46.1 ± | 138.7 ± |
| GLP-1 | | | 2.2 | 4.5**** | 4.2** | 2.8** | 6.0*** |
| D-GLP- | 1 | 10 | 59.5 ± | 101.4 ± | 73.9 | 48.1 ± | 145.1 ± |
| 1-BT1 | | | 3.7 | 3.1** | 3.0** | 1.9* | 3.5* |
| BT9-D- | 1 | 10 | 57.1 ± | 100.6 ± | 68.8 ± | 42.2 + | 137.3 ± |
| GLP-1 | | | 2.8 | 4.4** | 3.6*** | 1.7**** | 4.9*** |
| D-GLP- | 1 | 10 | 53.5 ± | 90.7 ± | 66.6 ± | 38.6 ± | 128.0 ± |
| 1-BT9 | | | 2.8 | 3.7**** | 2.5**** | 2.1**** | 4.0**** |
| Nor | — | 10 | 63.0 ± | 201.0 ± | 101.0 ± | 76.6 ± | 230.3 ± |
| | | | 2.3 | 10.4 | 6.1 | 3.5 | 9.2 |
| GLP-1 | 1 | 10 | 64.7 ± | 112.4 ± | 72.2 ± | 65.0 ± | 159.1 ± |
| | | | 3.2 | 8.6**** | 5.2*** | 3.0* | 6.8*** |
| BT1-N- | 1 | 10 | 57.3 ± | 114.3 ± | 69.1 ± | 56.1 ± | 151.4 ± |
| GLP-1 | | | 1.8 | 6.4**** | 3.5*** | 2.2**** | 6.8*** |
| Nor | — | 10 | 67.6 ± | 171.1 ± | 140.0 ± | 70.5 ± | 242.7 ± |
| | | | 3.1 | 8.3 | 6.2 | 3.8 | 9.6 |
| GLP-1 | 1 | 10 | 72.4 ± | 109.5 ± | 100.0 ± | 61.7 ± | 178.7 ± |
| | | | 3.8 | 4.1* | 4.0**** | 2.3 | 3.9** |
| N-GLP- | 1 | 10 | 62.4 ± | 83.7 ± | 93.5 ± | 63.2 ± | 159.2 ± |
| 1-BT1 | | | 4.3 | 2.9**** | 3.6**** | 2.3 | 4.4**** |
| Nor | — | 10 | 67.6 ± | 171.1 ± | 140.0 ± | 70.5 ± | 242.7 ± |
| | | | 3.1 | 8.3 | 6.2 | 3.8 | 9.6 |
| GLP-1 | 1 | 10 | 72.4 ± | 109.5 ± | 100.0 ± | 61.7 ± | 178.7 ± |
| | | | 3.8 | 4.1** | 4.0**** | 2.3 | 3.9** |
| BT9-N- | 1 | 10 | 57.9 ± | 96.0 ± | 99.7 ± | 66.1 ± | 170.3 ± |
| GLP-1 | | | 5.8 | 3.9*** | 4.5**** | 6.2 | 6.5**** |
| Nor | — | 10 | 67.6 ± | 171.1 ± | 140.0 ± | 70.5 ± | 242.7 ± |
| | | | 3.1 | 8.3 | 6.2 | 3.8 | 9.6 |
| GLP-1 | 1 | 10 | 72.4 ± | 109.5 ± | 100.0 ± | 61.7 ± | 178.7 ± |
| | | | 3.8 | 4.1** | 4.0** | 2.3 | 3.9*** |
| N-GLP- | 1 | 10 | 55.8 ± | 100.6 ± | 101.7 ± | 61.5 ± | 170.8 ± |
| 1-BT9 | | | 2.6* | 4.1**** | 10 1** | 2.6 | 8.3**** |
| Nor | — | 10 | 78.9 ± | 182.4 ± | 118.3 ± | 69.3 ± | 234.4 ± |
| | | | 3.0 | 11.1 | 5.8 | 3.1 | 9.2 |
| GLP-1 | 1 | 10 | 79.1 ± | 124.6 ± | 96.3 ± | 59.5 ± | 184.1 ± |
| | | | 3.2 | 2.1 | 3.2** | 2.3* | 3.3**** |
| CH4-D- | 1 | 10 | 77.7 ± | 113.2 ± | 95.0 ± | 59.6 ± | 177.0 ± |
| GLP-1 | | | 3.6 | 5.9*** | 3.5** | 1.4* | 5.1**** |
| D-GLP- | 1 | 10 | 72.1 ± | 124.2 ± | 82.7 ± | 57.4 ± | 170.8 ± |
| 1-CH4 | | | 3.7 | 6.4* | 5.0**** | 2.3** | 6.1**** |
| CH10-D- | 1 | 10 | 70.0 ± | 104.1 ± | 93.3 ± | 59.8 ± | 169.4 ± |
| GLP-1 | | | 3.0 | 2.5**** | 4.8*** | 2.2* | 6.0**** |
| D-GLP- | 1 | 10 | 65.9 ± | 116.3 ± | 90.3 ± | 57.7 ± | 171.2 ± |
| 1-CH10 | | | 2.2* | 5.0** | 3.3*** | 1.7** | 4.4**** |
| Nor | — | 10 | 74.9 ± | 157.3 ± | 113.4 ± | 72.0 ± | 218.4 ± |
| | | | 4.5 | 7.7 | 8.0 | 5.4 | 11.7 |
| GLP-1 | 1 | 10 | 67.4 ± | 91.8 ± | 82.2 ± | 61.7 ± | 155.2 ± |
| | | | 2.1 | 5.4**** | 3.7*** | 2.5 | 3.8**** |
| EC1-D- | 1 | 10 | 67.5 ± | 94.8 ± | 85.2 ± | 59.1 ± | 157.7 ± |
| GLP-1 | | | 3.0 | 3.7**** | 3.3*** | 2.1* | 4.5**** |
| D-GLP- | 1 | 10 | 67.3 ± | 111.8 ± | 81.8 ± | 64.5 ± | 166.4 ± |
| 1-EC1 | | | 2.3 | 5.4**** | 2.8**** | 2.2 | 4.5**** |
| EC12-D- | 1 | 10 | 63.3 ± | 103.3 ± | 77.2 ± | 67.6 ± | 159.1 ± |
| GLP-1 | | | 3.5 | 6.2**** | 5.5**** | 2.3 | 8.1**** |
| D-GLP- | 1 | 10 | 54.3 ± | 105.8 ± | 66.3 ± | 57.1 ± | 144.8 ± |
| 1-EC12 | | | 3.1*** | 5.1**** | 3.0**** | 2.9* | 4.6**** |

****p < 0.0001,
***p < 0.001,
**p < 0.01,
*p < 0.05 v.s. Nor.

### Duodenal Administration

Drug delivery technology can use enteric coating technology to achieve oral administration targeting the small intestine. In order to test the feasibility of direct small intestinal administration of GLP-1, the present invention designs duodenal administration. The experimental process is as follows: The day before the experiment, all of the animals were fasted for 15-16 hours with water ad libitum. On the day of the experiment, the animals were randomly divided according to body weight (n=9-11 or 14-15 for combined administration). Firstly, the blood was collected from the tail tip at 0 hours, and then the animals were anesthetized by inhaling ether. A small incision was made near the underside of the stomach using surgical scissors, and the duodenum was carefully removed and administered with GLP-1 analogs (SEQ ID NOs: 194-209, 10 μmol/kg) or saline. Finally, the wound was sutured. After 15 minutes, the glucose solution (2 g/kg) was administered by gavage, and the blood were collected from the tail tips at 15, 30, and 60 minutes after glucose administration. Blood glucose was measured using the glucose oxidase method. The blood glucose at each moment and area under the blood glucose-time curve (AUC) were calculated.

AUC mg×h/dL)=(BG₀+BG₁₅)×15/60+(BG₁₅+BG₃₀)×15/60+(BG₃₀+BG₆₀)×30/60. BG0, BG15, BG30, and BG60 represent blood glucose levels at 0, 15, 30, and 60 minutes after glucose loading, respectively.

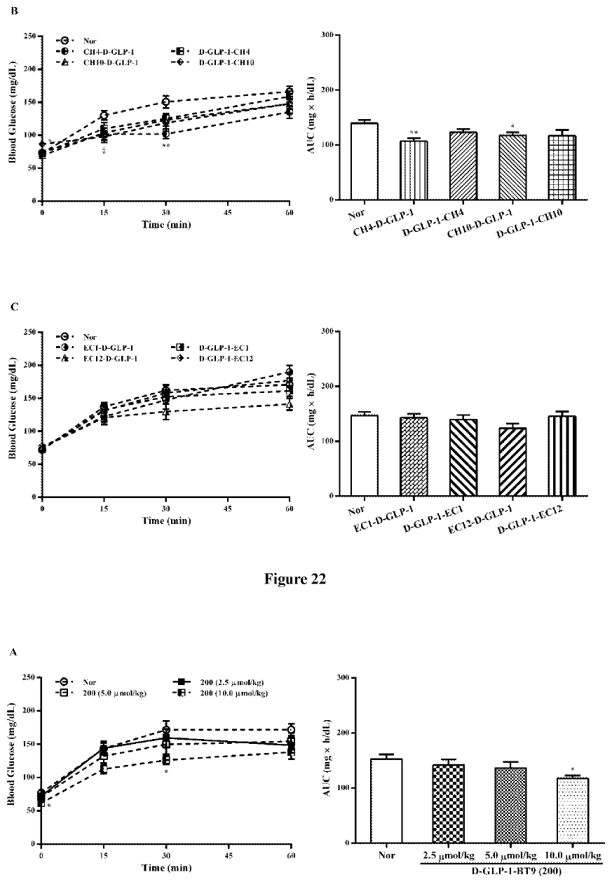

Results: Duodenal administration of GLP-1 analogues (SEQ ID NO: 194, SEQ ID NO: 196, SEQ ID NO: 198, SEQ ID NO: 200) containing both anti-trypsin peptide scaffolds BT9 (SEQ ID NO: 9) and BT45 (SEQ ID NO: 45), and anti-DPP-IV diprotin A (IPI) peptide segments in normal ICR mice displayed different results. Compared with the Nor group, duodenal administration of GLP-1 analog D-GLP-1-BT9 (SEQ ID NO: 200) significantly reduced the blood glucose level at 15, 30, and 60 minutes after oral glucose loading and the AUC; Duodenal administration of GLP-1 analog BT1-D-GLP-1 (SEQ ID NO: 194) reduced the blood glucose by 23.2% at 60 minutes without statistical significance; Duodenal administration of GLP-1 analog BT9-D-GLP-1 (SEQ ID NO: 198) reduced the blood glucose at 60 minutes and the AUC by 22.7% and 20.1%, respectively, but also failed statistical tests (FIG. 22A and Table 21). These results suggested that simultaneous introduction of the anti-trypsin peptide BT9 and the anti-DPP-IV diprotin A (IPI) peptide fragment significantly improved the enzymatic stability of GLP-1 analogues and enable them to exert hypoglycemic activity during duodenal administration. Moreover, the inhibitory peptide BT9 was directly connected to the C-terminal of GLP-1 and exerted stronger activity. Duodenal administration of BT1-D-GLP-1 and BT9-D-GLP-1 also showed a certain hypoglycemic effect in mice.

Duodenal administration of GLP-1 analogues (SEQ ID NO: 195, SEQ ID NO: 197, SEQ ID NO: 199, and SEQ ID NO: 201) containing both anti-trypsin peptide scaffolds BT9 (SEQ ID NO: 9) and BT45 (SEQ ID NO: 45), and the anti-NEP24.11 Opiorphin (QRFSR) peptide segment did not influence the blood glucose levels in normal ICR mice after oral glucose loading.

Duodenal administration of GLP-1 analogues (SEQ ID NOs: 202-205) containing both anti-chymotrypsin peptide scaffolds CH4 (SEQ ID NO: 84) and CH10 (SEQ ID NO: 90), and anti-DPP-IV diprotin A (IPI) peptide segments also displayed different results. Compared to Nor, duodenal administration of GLP-1 analogue CH4-D-GLP-1 (SEQ ID NO: 202) significantly reduced the blood glucose at 30 minutes and the AUC in normal ICR mice after oral glucose loading, with a reduction of 32.3% and 23.6%, respectively; Duodenal administration of GLP-1 analogue CH10-D-GLP-1 (SEQ ID NO: 204) significantly reduced the blood glucose at 15 minutes and the AUC in normal ICR mice after oral glucose loading, with a reduction of 20.4% and 15.8%, respectively; Duodenal administration of GLP-1 analogue D-GLP-1-CH10 (SEQ ID NO: 205) also significantly reduced the blood glucose at 15 minutes in normal ICR mice after oral glucose loading, with a reduction of 24.8% (FIG. 22B and Table 21). These results showed that the introduction of anti-chymotrypsin peptide scaffolds CH4, CH10, and anti-DPP-IV diprotin A (IPI) peptide segments can enhance the enzymatic stability of GLP-1 analogues and enable their duodenal administration to be effectively absorbed into the blood circulation to exert efficacy.

Duodenal administration of GLP-1 analogues (SEQ ID NOs: 206-209) containing both anti-elastase peptide scaffolds EC1 (SEQ ID NO: 134) and EC12 (SEQ ID NO: 145) and anti-DPP-IV diprotin A (IPI) peptide segment did not reduce the blood glucose or AUC in normal ICR mice after oral glucose loading, indicating that the stability of these GLP-1 analogues to withstand elastase enzymatic hydrolysis increased, but still can't resist the degradation of trypsin and chymotrypsin. However, compared with Nor, duodenal administration of GLP-1 analogue EC12-D-GLP-1 (SEQ ID NO: 208) reduced the blood glucose at 15, 30, and 60 minutes by 11.9%, 19.9% and 17.4%, respectively (FIG. 22C and Table 21), displaying a certain hypoglycemic effect, but was not statistically significant.

TABLE 21
The blood glucose lowering activity of GLP-1
analogs administered by duodenal injection

| | dose | | | | | | AUC |
|---|---|---|---|---|---|---|---|
| | (μmol/ | | Blood glucose (mg/dL, mean ± SEM) | | | | (mean ± |
| Group | kg) | n | 0 min | 15 min | 30 min | 60 min | SEM) |
| Nor | — | 11 | 67.8 ± | 125.7 ± | 154.1 ± | 186.5 ± | 144.3 ± |
| | | | 5.4 | 9.8 | 12.2 | 13.5 | 9.2 |
| BT1-D- | 10 | 10 | 60.7 ± | 113.5 ± | 128.0 ± | 143.2 ± | 119.8 ± |
| GLP-1 | | | 3.9 | 8.8 | 6.0 | 6.4 | 5.0 |
| D-GLP- | 10 | 9 | 59.5 ± | 132.1 ± | 144.2 ± | 171.1 ± | 138.4 ± |
| 1-BT1 | | | 3.6 | 12.7 | 11.0 | 15.1 | 9.0 |
| BT9-D- | 10 | 11 | 57.9 ± | 104.3 ± | 122.7 ± | 144.1 ± | 115.3 ± |
| GLP-1 | | | 2.9 | 8.7 | 13.9 | 13.5 | 10.5 |
| D-GLP- | 10 | 9 | 53.7 ± | 90.5 ± | 99.9 ± | 112.6 ± | 96.3 ± |
| 1-BT9 | | | 2.4* | 9.6* | 10.1** | 11.5*** | 8.7** |
| Nor | — | 10 | 72.7 ± | 130.2 ± | 150.7 ± | 166.2 ± | 139.7 ± |
| | | | 1.9 | 6.9 | 9.4 | 8.0 | 6.0 |
| CH4-D- | 10 | 10 | 73.8 ± | 101.9 ± | 102.0 ± | 135.2 ± | 106.8 ± |
| GLP-1 | | | 3.3 | 9.1 | 7.4** | 9.5 | 5.9** |
| D-GLP- | 10 | 10 | 72.5 ± | 110.2 ± | 125.2 ± | 158.8 ± | 123.2 ± |
| 1-CH4 | | | 2.3 | 8.9 | 7.3 | 8.4 | 5.9 |
| CH10-D- | 10 | 10 | 69.2 ± | 103.7 ± | 123.1 ± | 147.6 ± | 117.6 ± |
| GLP-1 | | | 3.5 | 6.6* | 9.0 | 7.8 | 5.5* |
| D-GLP- | 10 | 9 | 86.5 ± | 97.9 ± | 118.7 ± | 148.1 ± | 116.8 ± |
| 1-CH10 | | | 4.5* | 9.0* | 12.4 | 14.8 | 10.5 |
| Nor | — | 10 | 72.0 ± | 137.2 ± | 162.2 ± | 170.8 ± | 146.8 ± |
| | | | 2.2 | 6.8 | 8.6 | 9.8 | 7.1 |
| EC1-D- | 10 | 9 | 76.4 ± | 122.4 ± | 147.3 ± | 190.1 ± | 142.9 ± |
| GLP-1 | | | 3.5 | 8.4 | 13.2 | 9.6 | 6.9 |
| D-GLP- | 10 | 10 | 73.2 ± | 132.4 ± | 152.3 ± | 161.3 ± | 139.7 ± |
| 1-EC1 | | | 2.1 | 9.2 | 10.6 | 8.1 | 7.7 |
| EC12-D- | 10 | 10 | 75.6 ± | 120.8 ± | 129.8 ± | 141.1 ± | 123.6 ± |
| GLP-1 | | | 3.2 | 10.4 | 11.8 | 9.1 | 8.7 |
| D-GLP- | 10 | 9 | 72.9 ± | 131.2 ± | 158.2 ± | 177.1 ± | 145.5 ± |
| 1-EC12 | | | 3.4 | 7.9 | 11.7 | 9.7 | 8.6 |

****p < 0.0001,
***p < 0.001,
**p < 0.01,
*p < 0.05 v.s. Nor.

### Dose-Effect Relationship of GLP-1 Analog Composition Administered by Duodenal Injection

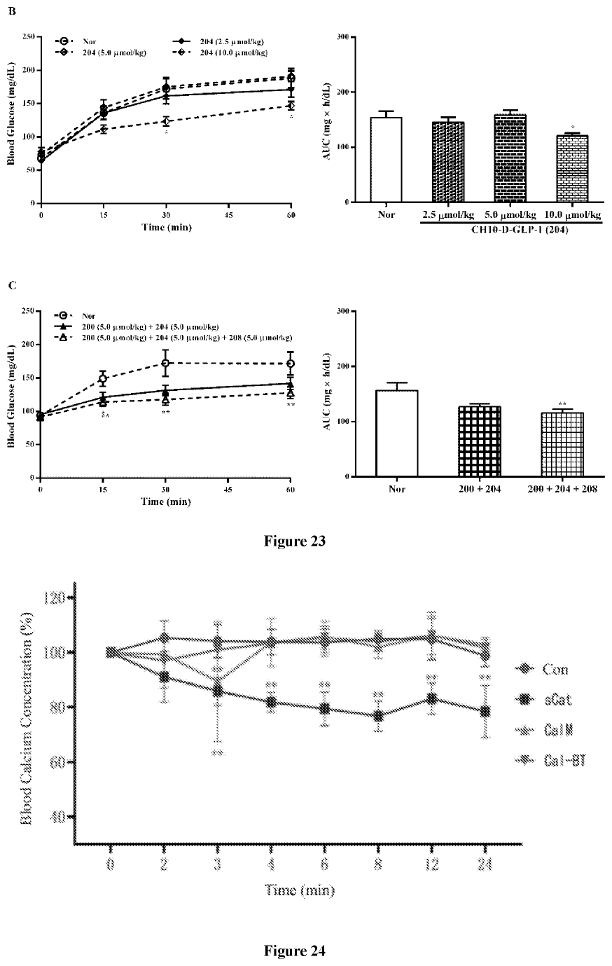

The proteases in the small intestine secreted by the pancreas mainly include trypsin (19% of total protein), chymotrypsin (9% of total protein), and elastase [Whitcomb D C, Low M E. Human pancreatic digestive enzymes. Dig Dis Sci. 2007, 52, 1-17]. In order to detect whether GLP-1 analogues containing different inhibitory peptide scaffolds against serine proteases have combined effects, the effective GLP-1 analogues D-GLP1-BT9 (SEQ ID NO: 200) and CH10-D-GLP-1 (SEQ ID NO: 204) at 10 μmol/kg were selected to perform a dose-effect relationship study. The results showed that duodenal injection of D-GLP1-BT9 and CH10-D-GLP-1 at 2.5 and 5.0 μmol/kg had no significant influence on the blood glucose in normal ICR mice. Then duodenal injection of D-GLP1-BT9 in combination with CH10-D-GLP-1 at 5.0 μmol/kg, as well as combination with CH10-D-GLP-1 and EC12-D-GLP-1 (SEQ ID NO: 208) at 5.0 μmol/kg were performed. The results showed that combination of D-GLP1-BT9 and CH10-D-GLP-1 significantly decreased the blood glucose at 15 minutes after oral glucose loading (p=0.0319) and had a trend of reducing the blood glucose at 30 and 60 minutes after oral glucose loading (p>0.05). To our surprise, combination of D-GLP1-BT9, CH10-D-GLP-1 and EC12-D-GLP-1 significantly reduced the blood glucose at 15, 30 and 60 minutes after oral glucose loading and the AUC (p<0.05) (FIG. 23 and Table 22). These results indicated that GLP-1 analogues containing different inhibitory peptide molecules of serine proteases have a combined effect, and also suggested that oral administration of polypeptides/proteins requires multiple serine protease inhibitors to inhibit the degradation of metabolic enzymes, thereby promoting the effective absorption of polypeptides/proteins in the intestinal epithelium.

TABLE 22
The blood glucose lowering activity of GLP-1
analogs administered by duodenal injection

| | | | | | | | AUC |
|---|---|---|---|---|---|---|---|
| | dose | | Blood glucose (mg/dL, mean ± SEM) | | | | (mean ± |
| Group | (μmol/kg) | n | 0 min | 15 min | 30 min | 60 min | SEM) |
| Nor | — | 9 | 77.0 ± | 143.4 ± | 171.5 ± | 171.7 ± | 152.7 ± |
| | | | 1.7 | 8.3 | 13.5 | 8.8 | 8.3 |
| D-GLP-1-BT9 | 2.5 | 9 | 72.2 ± | 144.1 ± | 159.4 ± | 148.4 ± | 141.9 ± |
| (200) | | | 2.7 | 10.2 | 13.4 | 11.5 | 9.9 |
| D-GLP-1-BT9 | 5.0 | 10 | 70.9 ± | 132.2 ± | 149.6 ± | 154.2 ± | 136.6 ± |
| (200) | | | 3.6 | 10.3 | 14.6 | 12.4 | 10.7 |
| D-GLP-1-BT9 | 10.0 | 11 | 61.5 ± | 112.5 ± | 126.2 ± | 138.1 ± | 117.6 ± |
| (200) | | | 4.9* | 7.0 | 6.5* | 10.5 | 5.2* |
| Nor | — | 9 | 68.1 ± | 136.2 ± | 172.0 ± | 188.1 ± | 154.1 ± |
| | | | 3.9 | 10.4 | 14.9 | 14.3 | 11.4 |
| CH10-D-GLP- | 2.5 | 10 | 63.8 ± | 136.0 ± | 161.6 ± | 171.2 ± | 145.4 ± |
| 1 (204) | | | 2.8 | 9.3 | 11.9 | 11.8 | 9.0 |
| CH10-D-GLP- | 5.0 | 10 | 73.7 ± | 143.8 ± | 175.2 ± | 190.9 ± | 158.6 ± |
| 1 (204) | | | 2.8 | 11.9 | 13.9 | 8.2 | 8.7 |
| CH10-D-GLP- | 10.0 | 10 | 77.9 ± | 111.5 ± | 123.4 ± | 146.8 ± | 120.6 ± |
| 1 (204) | | | 6.2 | 6.1 | 6.9* | 6.2* | 5.2* |
| Nor | — | 15 | 93.7 ± | 148.8 ± | 172.2 ± | 171.6 ± | 156.4 ± |
| | | | 2.3 | 11.2 | 19.9 | 17.2 | 14.2 |
| 200 + 204 | 5 + 5 | 14 | 95.0 ± | 120.9 ± | 131.3 ± | 141.7 ± | 126.8 ± |
| | | | 2.9 | 7.4* | 7.7 | 9.0 | 5.6 |
| 200 + 204 + | 5 + 5 + 5 | 14 | 91.4 ± | 113.9 ± | 117.5 ± | 127.7 ± | 115.9 ± |
| 208 | | | 2.9 | 6.5** | 8.4** | 8.8** | 6.6** |

****p < 0.0001,
***p < 0.001,
**p < 0.01,
*p < 0.05 v.s. Nor.

### Example 9. The Inhibitory Peptide Scaffolds Against Serine Protease Enhances the In Vivo Activity of PCSK9-Targeted Inhibitory Peptides

Based on the in vivo activity study of GLP-1 analogues containing inhibitory peptide scaffolds against serine protease, in order to further study whether these peptide scaffolds can be widely used to improve the efficacy of other therapeutic peptides, a series of polypeptides were designed and synthesized, so as to reach the ability of Pep2-8 (PCSK9_1, SEQ ID NO: 210) to inhibit PCSK9-LDLR interactions (Table 23).

### In Vitro Inhibitory Activity:

Polypeptide PCSK9_1-14 (SEQ ID NOs: 210-223) is dissolved in pure water or DMSO. 85 μL reaction buffer, 5 μL 1 mM polypeptide sample and 10 μL 750 ng/mL PCSK9 protein was pre-incubated at room temperature for 20 minutes before being added to a 96 well plate. The OD450/540 nm value was measured according to the instructions of the PCSK9-LDLR in Vitro Binding Assay Kit (CY-8150, MBL Company, Beijing, China). Solvent control: replace polypeptide with 5 μL solvent. In 100 μL reaction system, the final concentration of the polypeptide and PCSK9 is 50 μM and 75 ng/mL, respectively.

Inhibition rate of polypeptide (%)=(OD450/540 nm_{(solvent control)}−OD450/540 nm (sample))/OD450/540 nm_{(solvent control)}×100

Results: At a final concentration of 50 μM, the polypeptides PCSK9_2, PCSK9_3, PCSK9_5, PCSK9_6, PCSK9_7 and PCSK9_8 that contain the anti-trypsin peptide scaffold BT9 and the peptide PCSK9_9 that contains the anti-trypsin peptide scaffold BT45 have good inhibitory activity on the interaction between PCSK9 and LDLR in comparison to the reported PCSK9_1. The peptides PCSK9_2CH, PCSK9_2EC, PCSK9_3CH, PCSK9_3EC, PCSK9_5CH, PCSK9_5EC, PCSK9_6CH, PCSK9_6EC, PCSK9_9CH and PCSK9_9EC that contains the anti-chymotrypsin and anti-elastase peptide scaffolds CH10 and EC12 also displayed good inhibitory activity on the interaction between PCSK9 and LDLR (Table 24). These results showed that the inhibitory peptide scaffolds against trypsin, chymotrypsin and elastase (BT9, BT45, CH10, and EC12) increased the inhibitory effect of peptide Pep2-8 (PCSK9_1) on the interaction between PCSK9 and LDLR by 2-3 times; Especially the high similarity between the anti-trypsin peptide scaffolds and the anti-chymotrypsin and anti-elastase peptide scaffolds in PCSK9_9 indicated that the inhibitory peptide scaffolds against chymotrypsin and elastase can also enhance the activity of Pep2-8 (PCSK9_1) to inhibit the interaction between PCSK9 and LDLR.

TABLE 23
The amino acid sequences of Pep2-8 and its analogues

| | | | Theoretical |
|---|---|---|---|
| | | | molecular |
| Sequence | | | weight |
| NO. | Peptide | Sequence of amino acidª | (Da) |
| 210 | PCSK9_1 | TVFTSWEEYLDWV | 1582.73 |
| 211 | PCSK9_2 | 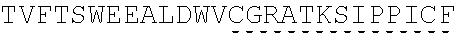 | 2955.41 |
| 212 | PCSK9_3 | 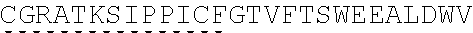 | 3012.46 |
| 213 | PCSK9_4 | 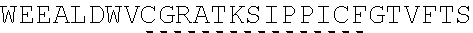 | 2955.41 |
| 214 | PCSK9_5 | 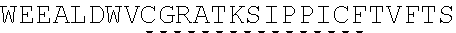 | 3012.46 |
| 215 | PCSK9_6 | 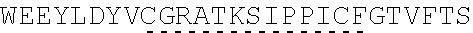 | 3081.52 |
| 216 | PCSK9_7 | 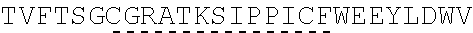 | 3104.56 |
| 217 | PCSK9_8 | 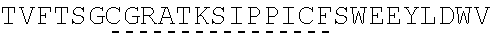 | 3191.64 |
| 218 | PCSK9_9 | 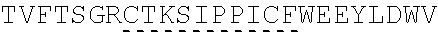 | 2976.43 |
| 219 | PCSK9_10 | 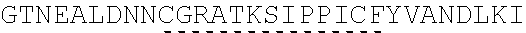 | 3236.68 |
| 220 | PCSK9_11 | 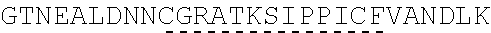 | 2960.35 |
| 221 | PCSK9_12 | 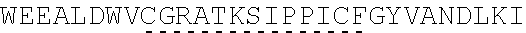 | 3393.94 |
| 222 | PCSK9_13 | 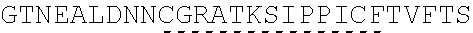 | 2855.21 |
| 223 | PCSK9_14 | 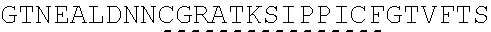 | 2912.26 |
| 224 | PCSK9_2CH | TVFTSWEEALDWVGFCTYSIPPQCYG | 2998.35 |
| 225 | PCSK9_2EC | TVFTSWEEALDWVGICTASIPPICQ | 2765.16 |
| 226 | PCSK9_3CH | FCTYSIPPQCYGGTVFTSWEEALDWV | 2998.35 |
| 227 | PCSK9_3EC | ICTASIPPICQGTVFTSWEEALDWV | 2765.16 |
| 228 | PCSK9_5CH | WEEALDWVGFCTYSIPPQCYGTVFTS | 2998.35 |
| 229 | PCSK9_5EC | WEEALDWVGICTASIPPICQGTVFTS | 2822.22 |
| 230 | PCSK9_6CH | WEEYLDYVGFCTYSIPPQCYGTVFTS | 3067.41 |
| 231 | PCSK9_6EC | WEEYLDYVGICTASIPPICQGTVFTS | 2891.28 |
| 232 | PCSK9_9CH | TVFTSGFCTYSIPPQCYGWEEYLDWV | 3090.44 |
| 233 | PCSK9_9EC | TVFTSGICTASIPPICQWEEYLDWV | 2857.26 |

^{a}In the table, the scaffolds of anti-trypsin, anti-chymotrypsin, and anti-elastase are named BT, CH, and EC, respectively, and are marked with dashed lines, double lines, and italics, respectively. In addition, disulfide bonds are formed between two cysteine residues of the three scaffolds in the polypeptide sequence.

TABLE 24
Inhibitory activity of Pep2-8 analogues
on the interaction of PCSK9-LDLR

| Sample | Inhibitory rate |
|---|---|
| (50 μM) | (%) |
| H₂O | 0 |
| DMSO | 0 |
| PCSK9_1 | 26.0 ± 10.0 |
| PCSK9_2 | 89.9 ± 0.8 |
| PCSK9_3 | 89.9 ± 0.9 |
| PCSK9_4 | 41.4 ± 6.0 |
| PCSK9_5 | 77.9 ± 2.4 |
| PCSK9_6 | 87.0 ± 1.1 |
| PCSK9_7 | 80.7 ± 0.5 |
| PCSK9_8 | 75.7 ± 1.2 |
| PCSK9_9 | 93.9 ± 0.4 |
| PCSK9_10 | −18.5 ± 6.4 |
| PCSK9_11 | 3.6 ± 8.0 |
| PCSK9_12 | 44.7 ± 4.4 |
| PCSK9_13 | 15.8 ± 10.1 |
| PCSK9_14 | −8.7 ± 12.7 |
| PCSK9_2CH | 100.3 ± 1.5 |
| PCSK9_2EC | 73.1 ± 3.2 (10 μM)^{a} |
| | 100.4 ± 0.4 (100 μM) |
| PCSK9_3CH | 99.2 ± 0.7 |
| PCSK9_3EC | 99.6 ± 3.8 |
| PCSK9_5CH | 99.4 ± 0.5 (10 μM) |
| PCSK9_5EC | 85.2 ± 1.7 (10 μM) |
| | 95.3 ± 0.1 (100 μM) |
| PCSK9_6CH | 75.3 ± 4.5 (10 μM) |
| | 93.7 ± 0.7 (100 μM) |
| PCSK9_6EC | 96.7 ± 0.3 (10 μM) |
| | 99.4 ± 0.2 (100 μM) |
| PCSK9_9CH | 98.4 ± 1.7 (10 μM) |
| | 101.6 ± 0.3 (100 μM) |
| PCSK9_9EC | 94.3 ± 1.2 (10 μM) |
| | 101.4 ± 0.2 (100 μM) |

^{a}The concentration of samples in the table was 50 μM. When measuring the IC₅₀ value, the sample with strong inhibitory activity was directly measured at 10 and 100 μM.

### In Vivo Lipid-Lowering Activity:

Model preparation and validation: Normal ICR mice were fasted overnight with water ad libitum. The poloxamer 407 (P407, 500 mg/kg) was intraperitoneally injected on the next day. After 24 hours, serum total cholesterol (TC) and low-density lipoprotein cholesterol (LDL-C) levels were significantly increased. The clinical drug Repatha was injected subcutaneously at a dose of 40 mg/kg for 24 hours, followed by intraperitoneal injection of P407. The serum TC and LDL-C levels were measured 24 hours after injection of P407 (Table 25). The results showed that intraperitoneal injection of P407 significantly increased the serum TC and LDL-C levels in ICR mice, and subcutaneous injection of Repatha (40 mg/kg) significantly reduced the serum TC and LDL-C levels.

TABLE 25
Effects of the marketed drug Repatha on serum TC and
LDL-C levels in P407-induced hyperlipidemia mice

| | | | LDL-C |
|---|---|---|---|
| Group | n | TC (mg/dL) | (mmol/L) |
| Nor | 5 | 99.7 ± 11.6** | 0.218 ± 0.036*** |
| Con | 9 | 233.4 ± 51.0 | 0.799 ± 0.146 |
| Repatha | 8 | 190.0 ± 36.6 | 0.364 ± 0.097*** |

***p < 0.001,
** p < 0.01 vs Con.

### Hypolipidemic Effect of PCSK9 Inhibitory Peptide by Subcutaneous Injection:

The experimental peptide sample was prepared using PEG400 with a final concentration of 2 mol/kg, and the final concentration of PEG400 is 20% (w/v). Normal ICR mice were fasted overnight with water ad libitum. The next day, all of the mice were randomly divided into model control group (Con) and polypeptide administration group (2 μmol/kg) according to body weight. Then, all of the mice were intraperitoneally injected with P407 (500 mg/kg) and fed with feed 2 hours later. Six mice were taken as a normal control group (Nor). After 24 hours, the mice in Con were subcutaneously injected with saline containing 20% PEG400, and the mice in the treatment group were given peptides. Then, the blood was taken at different time points after administration to determine the serum total cholesterol (TC) level. In consideration of the complexity of factors affecting serum LDL-C levels, especially the differences between single and long-term administration, the in vivo activity of PCSK9 inhibitory peptide is mainly observed for changes in serum TC levels.

The results showed that the serum TC level of mice in Con group was significantly increased in comparison to Nor, indicating that the model was successful. In comparison to Con, single subcutaneous injection of PCSK9_5, PCSK9_6, PCSK9_9, PCSK9_5EC, PCSK9_6CH and PCSK9_6EC had a reduced serum TC level (Table 26).

TABLE 26
Effects of subcutaneous injection of PCSK9 inhibitory peptide on serum
total cholesterol levels in P407-induced hyperlipidemia mice

| | dose | | TC (mg/dL) (mean ± sd) | | | |
|---|---|---|---|---|---|---|
| Group | (μmol/kg) | n | 30 min | 60 min | 90 min | 120 min |
| Nor | — | 6 | 106.1 ± 14.6*** | 94.0 ± 15.5*** | 97.5 ± 11.7*** | 97.8 ± 13.9*** |
| Con | — | 9 | 401.1 ± 94.3 | 337.8 ± 71.5 | 374.7 ± 85.3 | 365.8 ± 90.1 |
| PCSK9_1 | 2 | 9 | 392.5 ± 133.0 | 335.0 ± 111.7 | 383.2 ± 125.9 | 370.4 ± 122.3 |
| PCSK9_2 | 2 | 10 | 307.9 ± 108.1 | 317.8 ± 103.9 | 307.8 ± 104.4 | 295.5 ± 95.4 |
| PCSK9_7 | 2 | 10 | 339.8 ± 99.0 | 361.8 ± 95.4 | 346.1 ± 93.7 | 337.6 ± 95.8 |
| PCSK9_9 | 2 | 10 | 314.3 ± 84.2* | 351.6 ± 91.4 | 340.9 ± 97.0 | 327.4 ± 90.4 |

| | dose | | TC (mg/dL) (mean ± sd) | | | |
|---|---|---|---|---|---|---|
| Group | (μmol/kg) | n | 15 min | 30 min | 60 min | 90 min |
| Nor | — | 6 | 115.3 ± 16.2*** | 103.7 ± 12.2*** | 106.6 ± 16.0*** | 103.0 ± 13.9*** |
| Con | — | 10 | 371.3 ± 55.9 | 339.5 ± 48.5 | 352.4 ± 46.1 | 336.5 ± 55.4 |
| PCSK9_3 | 2 | 10 | 346.6 ± 66.2 | 349.0 ± 74.1 | 329.4 ± 64.9 | 326.5 ± 67.8 |
| PCSK9_5 | 2 | 9 | 305.0 ± 52.4* | 323.6 ± 54.2 | 308.9 ± 52.3 | 298.0 ± 59.1 |
| PCSK9_6 | 2 | 8 | 306.2 ± 37.6* | 320.0 ± 37.3 | 306.8 ± 47.2 | 293.4 ± 50.6 |
| Nor | — | 6 | 70.8 ± 10.7*** | 70.2 ± 7.5*** | 55.5 ± 8.5*** | 64.5 ± 7.7*** |
| Con | — | 10 | 217.6 ± 63.2 | 219.5 ± 55.6 | 203.9 ± 66.2 | 194.3 ± 54.6 |
| PCSK9_8 | 2 | 10 | 226.9 ± 48.9 | 221.6 ± 46.5 | 207.4 ± 47.0 | 203.7 ± 50.9 |
| Nor | — | 6 | 79.8 ± 7.0*** | 85.2 ± 7.5** | 79.2 ± 9.7*** | 83.5 ± 7.4** |
| Con | — | 9 | 292.2 ± 110.2 | 283.1 ± 104.6 | 286.0 ± 101.3 | 286.4 ± 116.9 |
| PCSK9_2CH | 2 | 10 | 277.2 ± 76.2 | 258.6 ± 70.0 | 273.7 ± 76.2 | 248.9 ± 76.0 |
| PCSK9_5CH | 2 | 10 | 300.2 ± 71.6 | 306.7 ± 68.5 | 302.3 ± 103.5 | 270.5 ± 74.1 |
| Nor | — | 8 | 105.8 ± 22.9*** | 114.3 ± 20.5*** | 107.4 ± 21.0*** | 96.6 ± 20.7*** |
| Con | — | 10 | 398.1 ± 97.2 | 372.0 ± 54.9 | 361.9 ± 74.1 | 342.5 ± 57.4 |
| PCSK9_3CH | 2 | 10 | 350.9 ± 103.1 | 382.8 ± 77.4 | 393.0 ± 150.9 | 343.5 ± 83.8 |
| PCSK9_6CH | 2 | 10 | 320.1 ± 138.9 | 336.3 ± 86.5 | 269.1 ± 97.9* | 265.5 ± 95.4* |
| PCSK9_9CH | 2 | 10 | 372.7 ± 115.4 | 421.2 ± 75.3 | 365.8 ± 66.4 | 289.1 ± 71.0 |
| Nor | — | 6 | 75.0 ± 5.9*** | 71.0 ± 5.1*** | 68.1 ± 3.7*** | 67.3 ± 4.9*** |
| Con | — | 10 | 305.7 ± 49.5 | 301.5 ± 50.5 | 295.3 ± 53.5 | 277.2 ± 53.6 |
| PCSK9_2EC | 2 | 10 | 295.4 ± 45.5 | 285.9 ± 53.5 | 285.3 ± 48.7 | 275.7 ± 47.5 |
| PCSK9_3EC | | 10 | 288.5 ± 76.9 | 267.3 ± 72.4 | 269.5 ± 69.9 | 260.5 ± 75.9 |
| PCSK9_5EC | 2 | 10 | 266.9 ± 52.0 | 223.1 ± 79.5* | 257.7 ± 46.6 | 253.2 ± 42.5 |
| PCSK9_6EC | | 10 | 243.8 ± 61.0* | 236.9 ± 57.4* | 234.5 ± 63.8* | 227.4 ± 58.6 |
| PCSK9_9EC | 2 | 10 | 278.9 ± 79.7 | 274.4 ± 90.9 | 267.6 ± 78.3 | 265.7 ± 83.8 |

***p < 0.001,
**p < 0.01,
*p < 0.05 vs Con.

### Lipid-Lowering Effect of Inhibitory Peptide Targeting PCSK9 by Duodenal Administration:

Enteric coating technology can be used to achieve oral delivery of drugs targeted small intestine. Considering factors such as gastric emptying and physical barriers to the stomach, in order to accurately detect the feasibility of direct delivery of targeted PCSK9 inhibitory peptide to the small intestine, duodenal delivery is designed. The experimental process is as follows:

The experimental polypeptide sample was prepared using PEG400 with a final concentration of 20 μmol/kg and the final concentration of PEG400 is 50% (w/v). The control group was saline containing PEG400 (50%).

Normal ICR mice were fasted overnight with water ad libitum. The next day, all of the mice were intraperitoneally injected with poloxamer 407 (P407, 500 mg/kg) to establish a model of lipid metabolism disorder. Six mice were intraperitoneally injected with saline as a normal control (Nor). Normal feeding resumed after 2 hours. The model animals were randomly divided into model group (Con) and polypeptide groups according to body weight, and the blood was collected from tail tip (0 min). Then, the animals were anesthetized with ether for duodenal exposure surgery. At the same time, a sample or saline containing PEG400 was injected through the duodenum. Finally, the wound was sutured. The blood was collected from tail tip at 15, 30, 60, and 90 minutes after administration to determine the serum total cholesterol level.

Results: Based on the in vivo activity of subcutaneous injection of the inhibitory peptide targeting PCSK9, PCSK9_6, PCSK9_6CH and PCSK9_6EC was used as test peptides in the duodenal administration experiment. The results showed that peptides PCSK9_6, PCSK9_6CH and PCSK9_6EC at the dose of 20 mol/kg did not show hypolipidemic activity (Table 27). The reason may be that the purity of the synthesized sample is poor, and the prototype polypeptide PCSK9_1 (Pep2-8) itself has shown little effect on lowering total cholesterol during subcutaneous injection experiments.

TABLE 27
Effects of duodenal injection of PCSK9 inhibitory peptide on serum
total cholesterol levels in P407 induced hyperlipidemia mice

| | dose | | TC (mg/dL) (mean ± sd) | | | |
|---|---|---|---|---|---|---|
| Group | (μmol/kg) | n | 15 min | 30 min | 60 min | 90 min |
| Nor | — | 3 | 96.6 ± 6.9*** | 97.3 ± 6.4*** | 95.1 ± 5.3*** | 86.9 ± 9.0*** |
| Con | — | 11 | 298.0 ± 60.8 | 304.2 ± 64.1 | 333.5 ± 59.2 | 294.6 ± 63.6 |
| PCSK9_6 | 20 | 9 | 296.8 ± 66.1 | 309.6 ± 80.7 | 315.3 ± 83.3 | 294.8 ± 87.0 |
| PCSK9_6CH | 20 | 8 | 274.2 ± 65.1 | 307.1 ± 59.0 | 312.7 ± 56.7 | 298.6 ± 65.0 |
| PCSK9_6EC | 20 | 8 | 316.6 ± 34.3 | 293.1 ± 67.3 | 323.9 ± 51.5 | 317.7 ± 44.3 |

***p < 0.001,
**p < 0.01,
*p < 0.05 vs Con.

### Stability Analysis of PCSK9 Inhibitory Peptide Against Trypsin, Chymotrypsin, and Elastase

Referring to the experimental method in Example 5, the enzymatic stability of the PCSK9 inhibitory peptide that was effective in vivo after subcutaneous injection was evaluated in vitro.

Results: PCSK9_1 was easily degraded by chymotrypsin and elastase but was stable towards trypsin for deficiency of basic amino acids in the molecule. PCSK9_6 that displayed lipid-lowering activity in vivo was selected as a representative to analyze its stability against chymotrypsin and elastase. The results showed that although it only contains inhibitory peptide scaffolds against trypsin, it also had certain inhibitory effects on the other two proteases chymotrypsin and elastase (Tables 28 and 29). It also indicates that the promiscuity activity of peptide scaffolds exerts the cross-inhibitory reactivity against chymotrypsin and elastase.

TABLE 28
Stability analysis of PCSK9_1 and its analogues
(SEQ ID NO: 215) against chymotrypsin

| | |
|---|---|
| Time | Residual peak area (%)/chymotrypsin |

| (min) | PCSK9_1 | PCSK9_6 |
|---|---|---|
| 0.0 | 100.0 ± 0.0 | 100.0 ± 0.0 |
| 1.5 | 82.3 ± 5.0 | 59.9 ± 8.2 |
| 3.0 | 80.7 ± 3.3 | 47.1 ± 8.0 |
| 4.5 | 80.1 ± 2.0 | 33.6 ± 3.0 |
| 6.0 | 79.1 ± 5.6 | 33.9 ± 3.2 |
| 9.0 | 75.5 ± 1.6 | 25.9 ± 3.3 |
| 15.0 | 71.2 ± 2.4 | 14.5 ± 0.8 |
| 30.0 | 62.7 ± 2.6 | 2.2 ± 0.1 |
| 60.0 | 47.7 ± 2.0 | 1.0 ± 0.9 |

TABLE 29
Stability analysis of PCSK9_1 and its
analogues (SEQ ID NO: 215) against elastase

| | |
|---|---|
| Time | Residual peak area (%)/elastase |

| (min) | PCSK9_1 | PCSK9_6 |
|---|---|---|
| 0.0 | 100.0 ± 0.0 | 100.0 ± 0.0 |
| 1.5 | 95.4 ± 0.9 | 62.9 ± 4.8 |
| 3.0 | 90.6 ± 2.4 | 59.3 ± 10.9 |
| 4.5 | 83.3 ± 1.8 | 56.4 ± 5.3 |
| 6.0 | 85.5 ± 2.7 | 55.3 ± 6.8 |
| 9.0 | 79.6 ± 0.8 | 67.8 ± 9.3 |
| 15.0 | 66.8 ± 0.8 | 48.9 ± 9.4 |
| 30.0 | 52.9 ± 1.5 | 39.6 ± 1.2 |
| 60.0 | 28.9 ± 0.7 | 48.3 ± 9.4 |

### Example 10 the Inhibitory Peptide Scaffolds Against Serine Protease Enhances the In Vivo Activity of Oral Salmon Calcitonin Analogues

Salmon Calcitonin is a peptide drug for the treatment of senile osteoporosis and osteoarthritis, and the effect is relatively definite. The clinical dosage forms are injection and nasal spray. In order to confirm whether the inhibitory peptide scaffolds against serine protease can improve the efficacy of salmon Calcitonin after oral administration, its analogs containing different inhibitory peptide scaffolds were designed and synthesized (Table 30).

TABLE 30
The amino acid sequences of salmon
Calcitonin analogs

| | | | Theoretical |
|---|---|---|---|
| | | | molecular |
| | | | weight |
| No. | Peptide | amino acid sequencesª | (Da) |
| 234 | CalM | CSNLSTCGLGKLSQEAHKLQT | 3348.73 |
| | | YPRTNTGSGTP | |
| 235 | Cal-BT | CSNLSTCGLGKLSQEAHKLQT | 4792.49 |
| | | 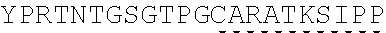 | |
| | | 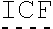 | |
| 236 | Cal-CH | CSNLSTCGLGKLSQEAHKLQT | 4764.34 |
| | | YPRTNTGSGTPGFCTYSIPPQC | |
| | | YG | |
| 237 | Cal-EC | CSNLSTCGLGKLSQEAHKLQT | 4531.16 |
| | | YPRTNTGSGTPGICTASIPPICQ | |

^{a}The skeletons of anti-trypsin, anti-chymotrypsin and anti-elastase are named BT, CH and EC, respectively, which are marked with dotted lines, double lines and italics. In addition, disulfide bonds are formed between the two cysteine residues in these three skeletons of the peptide sequences.

### Stability Analysis of Salmon Calcitonin Analogues Towards Trypsin, Chymotrypsin and Elastase

Referring to the experimental method in Example 5, in vitro stability of salmon Calcitonin analogs with oral administration activity to the enzymatic hydrolysis by trypsin, chymotrypsin and elastase were performed.

Results: Salmon Calcitonin analogues (CalM) were extremely unstable towards trypsin, and most of them were degraded after 3 min of co-incubation; CalM exhibited certain stability towards chymotrypsin, with approximately 4.9% of the prototype peptide remaining after 60 minutes of co-incubation. Salmon Calcitonin analogs Cal-BT, Cal-CH and Cal-EC containing inhibitory peptide scaffolds against serine proteases were resistant to the corresponding protease degradation, respectively. Cal-BT not only tolerated the degradation of trypsin, but also had high tolerance to chymotrypsin; Cal-EC had a certain tolerance to chymotrypsin (Tables 31 and 32).

TABLE 31
Stability analysis of Salmon Calcitonin and its Analogues (SEQ ID NO: 234-237) towards trypsin

| Time | Remaining peak area (%)/Trypsin | | | |
|---|---|---|---|---|
| (min) | CalM | Cal-BT | Cal-CH | Cal-EC |
| 0.0 | 100.0 ± 0.0 | 100.0 ± 0.0 | 100.0 ± 0.0 | — |
| 1.5 | 9.0 ± 2.3 | 84.2 ± 5.6 | 13.4 ± 2.3 | — |
| 3.0 | 0.0 ± 0.0 | 87.8 ± 1.0 | 0.0 ± 0.0 | — |
| 4.5 | 0.0 ± 0.0 | 71.4 ± 0.9 | 0.0 ± 0.0 | — |
| 6.0 | 0.0 ± 0.0 | 74.3 ± 3.4 | 0.0 ± 0.0 | — |
| 9.0 | 0.0 ± 0.0 | 77.1 ± 8.4 | 0.0 ± 0.0 | — |
| 15.0 | — | 69.4 ± 6.5 | — | — |
| 30.0 | — | 69.5 ± 6.6 | — | — |
| 60.0 | — | 68.2 ± 8.4 | — | — |

Stability analysis of Salmon Calcitonin and its Analogues (SEQ ID NO: 234-237) towards chymotrypsin

| Time | Remaining peak area (%)/chymotrypsin | | | |
|---|---|---|---|---|
| (min) | Calcitonin | Cal-BT | Cal-CH | Cal-EC |
| 0.0 | 100.0 ± 0.0 | 100.0 ± 0.0 | 100.0 ± 0.0 | 100.0 ± 0.0 |
| 1.5 | 88.6 ± 1.1 | 86.3 ± 9.0 | 89.3 ± 2.9 | 95.4* |
| 3.0 | 83.5 ± 3.6 | 83.9* | 90.9 ± 4.7 | 31.3* |
| 4.5 | 82.6 ± 5.2 | 73.1 ± 1.7 | 83.8 ± 0.4 | * |
| 6.0 | 84.2 ± 5.5 | 71.8 ± 1.1 | 82.2 ± 4.1 | 29.3 ± 1.4 |
| 9.0 | 75.5 ± 2.3 | 70.0 ± 3.0 | 52.4* | 27.8 ± 0.2 |
| 15.0 | 67.8 ± 1.5 | 61.3* | 51.8 ± 0.4 | 22.3* |
| 30.0 | 48.9 ± 3.4 | 58.9 ± 2.3 | 51.2 ± 1.8 | 21.4* |
| 60.0 | 4.9 ± 0.3 | 46.0 ± 1.3 | 24.6 ± 0.5 | 19.3* |

*Baseline separation not achieved.

### Hypo-Calcifying Effect of Salmon Calcitonin by Subcutaneous Injection

Rats were fasted for 12 hours with water ad libitum before the experiment. The animals were randomly divided into 4 groups (5 animals in each group). The normal control group was injected with normal saline solution, and the commercially available salmon Calcitonin (sCat) and synthetic Calcitonin analog (CalM) were injected subcutaneously. The capsule form Cal BT (1 umol/kg, p.o.) was administered by gavage.

Take blood from the inner canthus of rats at the predetermined time points: 0, 2, 3, 4, 6, 8, 12, and 24 h later, with at least 0.2 mL of blood taken each time. The blood sample was placed at 4° C. for stratification and centrifuged at 3000 rpm for 10 minutes. The serum was separated and measured for serum calcium ion concentration.

The serum calcium ion concentration at 0 h was considered as baseline, the blood calcium concentration at other time was converted into the percentage ratio of baseline. The blood calcium curve was drawn with time as the X axis and the percentage of blood calcium concentration (%) as the Y axis.

Results: The changes of body weight were shown in Table 33; Using the decrease in blood calcium concentration at different times as the evaluation criteria, the results showed that the commercially available salmon Calcitonin (sCat) could effectively reduce the concentration of calcium ions in rats 3, 4, 6, 8, 12 and 24 hours after administration. Salmon Calcitonin analogue (CalM) can effectively reduce the concentration of calcium ions in rats 3 hours after administration, but the capsule form Cal BT did not effectively reduce the concentration of calcium ions in rats (FIG. 24).

TABLE 33
Changes of body weight after administration in SD rats

| | n | Body weight (g) X ± SD | | Changes of body |
|---|---|---|---|---|
| Group | End/Start | Start | End | weight (%) |
| Con | 5/5 | 177.4 ± 0.9 | 160.7 ± 2.4 | −9.42 |
| sCat | 3/5 | 178.5 ± 8.1 | 163.5 ± 8.9 | −8.43 |
| CalM | 5/5 | 180.2 ± 6.8 | 160.9 ± 4.7 | −10.71 |
| Cal-BT | 5/5 | 176.1 ± 5.1 | 158.9 ± 3.2 | −9.80 |

### Example 11 the Inhibitory Peptide Scaffolds Against Serine Proteases Enhances the In Vivo Activity of Interleukin-17A (IL-17A) Targeted Inhibitory Peptides

Based on the in vivo activity study of GLP-1 analogues containing inhibitory peptide scaffolds against serine proteases, in order to further investigate whether these peptide inhibitors can be widely used to improve the efficacy of other therapeutic peptides, a series of inhibitory peptides targeting IL-17A (Table 34) were designed and synthesized with the research goal of 17A (SEQ ID NO: 238), which has inhibitory effects on IL-17A.

TABLE 34
Amino acid sequence of IL-17A inhibitory peptides

| | | | Theoretical |
|---|---|---|---|
| | | | molecular |
| | | | weight |
| No. | Peptide | amino acid sequences^{a} | (Da) |
| 238 | 17A | IHVTIPADLWDWIN | 1692.93 |
| 239 | 17A-BT | 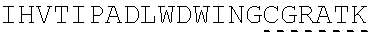 | 3122.67 |
| | | 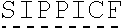 | |
| 240 | 17A-CH | IHVTIPADLWDWINGFCTYSIP | 3108.55 |
| | | PQCYG | |
| 241 | 17A-EC | IHVTIPADLWDWINGICTASIPP | 2875.37 |
| | | ICQ | |

^{a}The skeletons of anti-trypsin, anti-chymotrypsin and anti-elastase are named BT, CH and EC, respectively, which are marked with dotted lines, double lines and italics. In addition, disulfide bonds are formed between the two cysteine residues in these three skeletons of the peptide sequences.

### Stability Analysis of IL-17A Inhibitory Peptides Towards Trypsin, Chymotrypsin and Elastase

Referring to the experimental method in Example 5, in vitro stability of IL-17A inhibitory peptides towards the enzymatic hydrolysis by trypsin, chymotrypsin and elastase were performed.

Results: Peptide 17A is unstable towards chymotrypsin and elastase, but very stable towards trypsin, because there are no basic amino acids in the molecule; peptides 17A-BT, 17A-CH, and 17A-EC with the inhibitory peptide scaffolds against serine proteases separately tolerated to the degradation of corresponding serine proteases, and also exhibited certain inhibitory effects on the other two serine proteases (Table 35).

TABLE 35
Stability analysis of 17A and its Analogues
(SEQ ID NO: 238-241) towards chymotrypsin

| Time | Remaining peak area (%)/chymotrypsin | | | |
|---|---|---|---|---|
| (min) | 17A | 17A-BT | 17A-CH | 17A-EC |
| 0.0 | 100.0 ± 0.0 | 100.0 ± 0.0 | 100.0 ± 0.0 | 100.0 ± 0.0 |
| 1.5 | 68.8 ± 5.5 | 69.6 ± 3.0 | 41.6 ± 3.0 | 85.5 |
| 3.0 | 59.2 ± 6.3 | 68.9 ± 4.8 | 41.7 ± 6.0 | 93.8 ± 2.4 |
| 4.5 | 46.2 ± 5.2 | 48.1 ± 4.3 | 38.8 ± 3.4 | 89.6* |
| 6.0 | 41.4 ± 4.5 | 45.2 ± 1.3 | 45.1 ± 8.5 | 91.6 ± 5.3 |
| 9.0 | 24.3 ± 1.2 | 42.9 ± 3.0 | 44.3 ± 7.6 | * |
| 15.0 | — | 37.0 ± 1.7 | 35.2 ± 3.8 | 79.8 ± 2.6 |
| 30.0 | — | 26.4 ± 0.4 | 30.5 ± 2.5 | 80.9* |
| 60.0 | — | 9.6 ± 0.8 | 35.7 ± 3.0 | 68.0* |

*Baseline separation not achieved.

### The Anti-Inflammatory Activity of IL-17A Inhibitory Peptide:

IL-17A is an inflammatory factor in many chronic inflammatory reactions. In order to quickly evaluate and analyze its anti-inflammatory effects, a mouse ear swelling model was first used for preliminary screening of anti-inflammatory activities. The experimental process is as follows. Kunming male mice (18-20 g, n=10) were labeled with picric acid. All mice in each group were coated with 10 μL croton oil on both sides of the right ear. Immediately after modeling, the positive drugs Secukinumab group (5 mg/kg), inhibitory peptides 17A, 17A-BT, 17A-CH, and 17A-EC (30 mg/kg) were subcutaneously injected. The model control group (Con) was injected with a corresponding volume of physiological saline. After causing inflammation for 4 hours, the mice in each group were killed for cervical dislocation, and then the ear pieces were punched in the symmetrical parts of the left and right ears with a hole punch. The weight was weighed and recorded. The swelling degree and swelling rate were calculated:

Swelling rate=((right ear mass−left ear mass)/left ear mass)*100%

Results: Targeted IL-17A inhibitory peptides 17A-BT and 17A-CH administered (30 mg/kg) subcutaneously can significantly inhibit the inflammatory response to ear swelling induced by croton oil, while 17A and 17A-EC have no inhibitory effect, indicating that the inhibitory peptide scaffolds against serine protease can effectively improve the stability of the IL-17A inhibitory peptide in the blood circulation, thereby improving its efficacy in vivo (Table 36).

TABLE 36
Inhibitory activity of IL-17A inhibitory peptide
administered subcutaneously on mouse ear swelling

| | | | Swelling |
|---|---|---|---|
| | n | | inhibitory |
| Group | final/beginning | Swelling (%) | (%) |
| model control group | 10/10 | 115.80 ± 44.05 | — |
| (Con) | | | |
| Secukinumab | 10/10 | 72.54 ± 25.32* | 37.36 |
| 17A | 10/10 | 92.87 ± 27.01 | 19.80 |
| 17A-BT | 10/10 | 74.70 ± 32.96* | 35.49 |
| 17A-CH | 10/10 | 82.48 ± 19.04* | 28.77 |
| 17A-EC | 10/10 | 90.05 ± 25.22 | 22.24 |

***p < 0.001,
**p < 0.01,
*p < 0.05 vs Con.

### The Anti-Inflammatory Activity of IL-17A Inhibitory Peptide Administered Via Duodenum:

Enteric coating technology can be used to achieve oral administration of targeted small intestine drugs. Considering factors such as gastric emptying and physical barriers in the stomach, in order to accurately detect the feasibility of direct intestinal administration of targeted IL-17A inhibitory peptide, duodenal administration is designed. Eight mice in each group are subjected to surgical exposure of the duodenum under ether anesthesia, and the drug is injected according to different grouping schemes.

The model control group (Con) is given PEG400 (50%, w/v)/physiological saline. The administration group was given different polypeptide samples (300 mg/kg), while the positive control group was given dexamethasone (1 mg/mL, 10 mL/kg), and then the muscular layer and cortex were sutured. Ear swelling model was established 6 minutes after suture. All mice in each group were coated with 10 μL croton oil on both sides of the right ear. After 4 hours of inflammation, the mice in each group were killed for cervical dislocation. After that, ear pieces were punched in the symmetrical parts of the left and right ears with a hole punch and weighed. Their mass was recorded. The swelling degree and swelling rate were calculated:

Swelling rate=((right ear mass−left ear mass)/left ear mass)*100%

Results: Compared with the model control group, the inhibitory effects of the inhibitory peptides 17A-BT (P<0.01) and 17A-CH (P<0.05) targeting IL-17A on mouse ear swelling via duodenal administration were statistically significant (Table 37).

TABLE 37
Inhibitory activity of 17A-BT and 17A-CH administered
via duodenum on mouse ear swelling

| | | | Swelling |
|---|---|---|---|
| | n | | inhibitory |
| Group | final/beginning | Swelling (%) | (%) |
| model control group | 8/8 | 96.15 ± 13.50 | — |
| (Con) | | | |
| 17A-BT | 8/8 | 53.83 ± 14.17** | 44.01 |
| dexamethasone | 8/8 | 58.11 ± 18.81** | 39.57 |
| (Dex) | | | |
| model control group | 8/8 | 92.28 ± 17.42 | — |
| (Model) | | | |
| 17A-CH | 8/8 | 68.93 ± 16.03* | 25.31 |
| dexamethasone | 8/8 | 65.67 ± 15.52* | 28.84 |
| (Dex) | | | |

***p < 0.001,
**p < 0.01,
*p < 0.05 vs Con.

### Example 12 Coating Enteric Coated Capsules Using Dip Coating Method

Fill the capsules (size M, Torpac) with bromophenol blue powder as a tracer for enteric coating. Soak 2/3 of the capsule surface in the coating material Eudragit L100-55 mixture (Eudragit L100-55/0.9 g, PEG 400/0.14 g, Tween 80/0.01 g, acetone/3.8 mL, isopropanol/5.7 mL, water/0.5 mL) for 15 seconds and dry for 30 minutes. Turn it upside down again, operate the remaining 1/3 of the capsule surface in the same way, repeat soaking 3 times, and dry at room temperature in a fume hood for 72 hours. Then immerse the capsule coated with enteric coating in simulated gastric fluid (pH 1.6) for 2 hours, or in simulated intestinal fluid (pH 6.5) for 1 hour. Monitor the amount of bromophenol blue released by capsule disintegration as the immersion time increases, i.e., measure the absorption at 422 nM to determine the effectiveness of capsule encapsulation. The results indicated that the thickness of the capsule coating is 0.16+0.05 nm; The release of bromophenol blue was 2.8˜6.5% (92˜97% remained intact) after 2 hours of incubation in simulated gastric juice, and 44˜51.3% after 1 hour of incubation in simulated intestinal juice.

1. A peptide having a general formula (M), or its analog having N-terminal, C-terminal, or side chain modified by PEGylation, phosphorylation, amidation, or acylation, or a pharmaceutically acceptable salt thereof,
Xaa6-Xaa5-Xaa4-Xaa3-Xaa2-Xaa1-Xaa1′-Xaa2′-Xaa3′-Xaa4′-Xaa5′-Cys6′-Xaa7′-Xaa8′ (M);
wherein:
Xaa1 is selected from the group consisting of Lys, Arg, Tyr, Phe, Ala, and Leu;
Xaa2 is selected from the group consisting of Thr and Ala;
Xaa3 is selected from the group consisting of Ala, Abu, Tyr, Nle, Ser, Gln, Leu, Ile, Val, Phe, Asn, His, Trp, Glu, Pro, Hyp, Gly, Thr, Arg, Cys, and Hcy;
Xaa4 is selected from the group consisting of Lys, Ser, Ala, Thr, Tyr, Leu, Ile, Val, Met, and Arg;
Xaa5 is selected from the group consisting of Gly, Pro, Ala, Hyp, Val, Leu, Ile, Abu, Ser, Arg, Lys, Glu, Gin, and Nle, or absent;
Xaa6 is selected from the group consisting of Cys and Hcy, or absent;
Xaa1′ is selected from the group consisting of Ser and Ala;
Xaa2′ is selected from the group consisting of Ile, Leu, Nle, Arg, Phe, Tyr, Asn, Val, Met, Thr, His, Lys, Ser, Ala, Met, Asp, Trp, and Glu;
Xaa3′ is selected from the group consisting of Pro and Hyp;
Xaa4′ is selected from the group consisting of Pro, Ala, Gly, and Hyp;
Xaa5′ is selected from the group consisting of Ile, Leu, Ala, Gln, Met, Phe, Asp, Glu, His, Tyr, Ser, Thr, Val, Asn, Lys, Arg, Gly, and Trp;
Cys6′ is selected from the group consisting of Cys and Hcy;
Xaa7′ is selected from the group consisting of Phe, Tyr, Asn, Ala, Trp, His, Gln, Ser, Hyp, Val, Arg, and Ile;
Xaa8′ is selected from the group consisting of Gly and Ala, or absent;
wherein, one and only one of Xaa3 and Xaa6 must be Cys, or Hcy,
when Xaa3 is Cys or Hcy, both Xaa5 and Xaa6 are absent, and the peptide is cyclized via a disulfide bond between Xaa3 and Cys6′;
when Xaa6 is Cys or Hcy, the peptide is cyclized via a disulfide bond between Xaa6 and Cys6′.

2. The peptide, or its analog having N-terminal, C-terminal, or side chain modified by PEGylation, phosphorylation, amidation, or acylation, or a pharmaceutically acceptable salt thereof according to claim 1, wherein the peptide has a general formula (I):
Cys6-Xaa5-Xaa4-Xaa3-Xaa2-Xaa1-Xaa1′-Xaa2′-Xaa3′-Xaa4′-Xaa5′-Cys6′-Xaa7′ (I);
wherein, Cys6 and Cys6′ are independently selected from Cys or Hcy, respectively; the peptide is cyclized via a disulfide bond between Cys6 and Cys6′;
wherein with the proviso that
if Xaa1 is Lys or Arg, then
Xaa2 is selected from the group consisting of Thr and Ala;
Xaa3 is selected from the group consisting of Ala, Abu, Tyr, Nle, Ser, Gln, Leu, Ile, Val, Phe, Asn, His, Trp, Glu, Pro, Hyp, and Gly;
Xaa4 is selected from the group consisting of Arg, Lys, Ser, Ala, and Thr;
Xaa5 is selected from the group consisting of Gly, Pro, Ala, Hyp, Val, Leu, Ile, Abu, Ser, Arg, Lys, Glu, Gin, and Nle;
Xaa1′ is selected from the group consisting of Ser and Ala;
Xaa2′ is selected from the group consisting of Ile, Leu, Nle, Arg, Phe, Tyr, Asn, Val, Met, Thr, His, Lys, Ser, Ala, and Met;
Xaa3′ is selected from the group consisting of Pro and Hyp;
Xaa4′ is selected from the group consisting of Pro, Ala, and Hyp;
Xaa5′ is selected from the group consisting of Ile, Leu, Ala, Gln, Met, Phe, Asp, Glu, His, Tyr, Ser, Thr, Val, Asn, Lys, Arg, and Gly;
Xaa7′ is selected from the group consisting of Phe, Tyr, Asn, Ala, Trp, His, Gln, Ser, and Hyp;
wherein with the proviso that
if Xaa1 is Tyr, or Phe, then
Xaa2 is selected from the group consisting of Thr and Ala;
Xaa3 is selected from the group consisting of Ala, Abu, Gly, Tyr, Nle, Ser, Gln, Leu, Ile, Val, Phe, Asn, His, Trp, Glu, Pro, and Arg;
Xaa4 is selected from the group consisting of Ser, Ala, Phe, Thr, Lys, Tyr, Leu, Ile, Val, Met, and Arg;
Xaa5 is selected from the group consisting of Gly, Pro, Hyp, and Ala;
Xaa1′ is selected from the group consisting of Ser and Ala;
Xaa2′ is selected from the group consisting of Ile, Phe, Leu, Ala, Met, Asn, His, Asp, Tyr, Trp, and Glu;
Xaa3′ is selected from the group consisting of Pro and Hyp;
Xaa4′ is selected from the group consisting of Pro, Ala, Gly and Hyp;
Xaa5′ is selected from the group consisting of Ile, Leu, Gln, Met, Arg, Phe, His, Lys, Arg, Trp, Tyr, Ala, Ser, Thr, Val, Asp, Asn, Glu, and Gly;
Xaa7′ is selected from the group consisting of Tyr, Phe, Asn, Val, Arg, Ile, Gln, Ser, and His;
wherein with the proviso that
if Xaa1 is Ala, or Leu, then
Xaa2 is selected from the group consisting of Thr and Ala;
Xaa3 is selected from the group consisting of Ala, Abu, Gly, Tyr, Nle, Ser, Gln, Leu, Ile, Val, Phe, Asn, His, Trp, Glu, Pro, and Arg;
Xaa4 is selected from the group consisting of Ile, Leu, Val, Ala, and Tyr;
Xaa5 is selected from the group consisting of Gly, Pro, Hyp, and Ala;
Xaa1′ is selected from the group consisting of Ser and Ala;
Xaa2′ is selected from the group consisting of Ile, Asn, Tyr, and Ala;
Xaa3′ is selected from the group consisting of Pro and Hyp;
Xaa4′ is selected from the group consisting of Pro, Hyp and Ala;
Xaa5′ is selected from the group consisting of Ile and Gln; and
Xaa7′ is selected from the group consisting of Gln, Tyr, Arg, His and Asn.

3. The peptide, or its analog having N-terminal, C-terminal, or side chain modified by PEGylation, phosphorylation, amidation, or acylation, or a pharmaceutically acceptable salt thereof according to claim 2, wherein
Xaa1 is selected from the group consisting of Lys and Arg;
Xaa2 is selected from the group consisting of Thr and Ala;
Xaa3 is selected from the group consisting of Ala, Abu, Tyr, Gly, Nle, Ser, Thr, and Gln;
Xaa4 is selected from the group consisting of Arg, Lys, Ser, Ala, and Thr;
Xaa5 is selected from the group consisting of Ala, Gly, and Pro;
Xaa1′ is selected from the group consisting of Ser and Ala;
Xaa2′ is selected from the group consisting of Ile, Leu, Nle and Ala;
Xaa3′ is selected from the group consisting of Pro and Hyp;
Xaa4′ is selected from the group consisting of Pro and Ala;
Xaa5′ is selected from the group consisting of Ile, Ala, and Gln; and
Xaa7′ is selected from the group consisting of Phe and Tyr.

4. The peptide, or its analog having N-terminal, C-terminal, or side chain modified by PEGylation, phosphorylation, amidation, or acylation, or a pharmaceutically acceptable salt thereof according to claim 3, wherein the peptide is selected from the group consisting of the following sequences: SEQ ID NO: 9, SEQ ID NO: 10, SEQ ID NO: 16, SEQ ID NO: 17, SEQ ID NO: 25, SEQ ID NO: 27, SEQ ID NO: 28, SEQ ID NO: 35, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 60, SEQ ID NO: 66, SEQ ID NO: 67, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 80, and SEQ ID NO: 79.

5. The peptide, or its analog having N-terminal, C-terminal, or side chain modified by PEGylation, phosphorylation, amidation, or acylation, or a pharmaceutically acceptable salt thereof according to claim 2, wherein
Xaa1 is selected from the group consisting of Tyr and Phe;
Xaa2 is selected from the group consisting of Thr and Ala;
Xaa3 is selected from the group consisting of Ala and Abu;
Xaa4 is selected from the group consisting of Ser, Ala, Phe, and Thr;
Xaa5 is selected from the group consisting of Ala, Gly, and Pro;
Xaa1′ is Ser;
Xaa2′ is selected from the group consisting of Ile, Ala, and Asn;
Xaa3′ is selected from the group consisting of Pro and Hyp;
Xaa4′ is selected from the group consisting of Pro, Ala, and Hyp;
Xaa5′ is selected from the group consisting of Ile and Gln; and
Xaa7′ is selected from the group consisting of Tyr, Phe, Asn, Gln, and His.

6. The peptide, or its analog having N-terminal, C-terminal, or side chain modified by PEGylation, phosphorylation, amidation, or acylation, or a pharmaceutically acceptable salt thereof according to claim 2, wherein
Xaa1 is selected from the group consisting of Ala and Leu;
Xaa2 is selected from the group consisting of Thr and Ala;
Xaa3 is selected from the group consisting of Ala, Abu, Gly, Tyr, Nle, Ser, Gln, Leu, Ile, Val, Phe, Asn, His, Trp, Glu, Pro, and Arg;
Xaa4 is selected from the group consisting of Ile, Leu, Val, Ala, and Tyr;
Xaa5 is selected from the group consisting of Gly, Pro, Ala, and Hyp;
Xaa1′ is selected from the group consisting of Ser and Ala;
Xaa2′ is selected from the group consisting of Ile and Asn;
Xaa3′ is selected from the group consisting of Pro and Hyp;
Xaa4′ is selected from the group consisting of Pro and Hyp;
Xaa5′ is selected from the group consisting of Ile and Gln; and
Xaa7′ is selected from the group consisting of Gln and Tyr.

7. The peptide, or its analog having N-terminal, C-terminal, or side chain modified by PEGylation, phosphorylation, amidation, or acylation, or a pharmaceutically acceptable salt thereof according to claim 1, wherein the peptide has a general formula (II):
Xaa4-Cys3-Xaa2-Xaa1-Xaa1′-Xaa2′-Xaa3′-Xaa4′-Xaa5′-Cys6′-Xaa7′-Xaa8′ (II);
wherein, Cys3 and Cys6′ are independently selected from Cys or Hcy, respectively; the peptide is cyclized via a disulfide bond between Cys3 and Cys6′;
wherein with the proviso that
if Xaa1 is Lys or Arg, then
Xaa2 is selected from the group consisting of Thr and Ala;
Xaa4 is selected from the group consisting of Arg, Lys, Ser, Ala, and Thr;
Xaa1′ is selected from the group consisting of Ser and Ala;
Xaa2′ is selected from the group consisting of Ile, Leu, Nle, Arg, Phe, Tyr, Asn, Val, Met, Thr, His, Lys, Ser, Ala and Met;
Xaa3′ is selected from the group consisting of Pro and Hyp;
Xaa4′ is selected from the group consisting of Pro, Ala and Hyp;
Xaa5′ is selected from the group consisting of Ile, Leu, Ala, Gln, Met, Phe, Asp, Glu, His, Tyr, Ser, Thr, Val, Asn, Lys, Arg and Gly;
Xaa7′ is selected from the group consisting of Phe, Tyr, Asn, Ala, Trp, His, Gln, Ser and Hyp;
Xaa8′ is absent;
wherein with the proviso that
if Xaa1 is Tyr or Phe, then
Xaa2 is selected from the group consisting of Thr and Ala;
Xaa4 is selected from the group consisting of Ser, Ala, Phe, Thr, Lys, Tyr, Leu, Ile, Val, Met and Arg;
Xaa1′ is selected from the group consisting of Ser and Ala;
Xaa2′ is selected from the group consisting of Ile, Phe, Leu, Ala, Met, Asn, His, Asp, Tyr, Trp and Glu;
Xaa3′ is selected from the group consisting of Pro and Hyp;
Xaa4′ is selected from the group consisting of Pro, Ala, Gly and Hyp;
Xaa5′ is selected from the group consisting of Ile, Leu, Gln, Met, Arg, Phe, His, Lys, Arg, Trp, Tyr, Ala, Ser, Thr, Val, Asp, Asn, Glu and Gly;
Xaa7′ is selected from the group consisting of Tyr, Phe, Asn, Val, Arg, Ile, Gln, Ser and His;
Xaa8′ is selected from the group consisting of Gly and Ala, or absent;
wherein with the proviso that
If Xaa1 is Ala, or Leu, then
Xaa2 is selected from the group consisting of Thr and Ala;
Xaa4 is selected from the group consisting of Ile, Leu, Val, Ala and Tyr;
Xaa1′ is selected from the group consisting of Ser and Ala;
Xaa2′ is selected from the group consisting of Ile, Asn, Tyr and Ala;
Xaa3′ is selected from the group consisting of Pro and Hyp;
Xaa4′ is selected from the group consisting of Pro, Hyp and Ala;
Xaa5′ is selected from the group consisting of Ile and Gln;
Xaa7′ is selected from the group consisting of Gln, Tyr, Arg, His and Asn; and
Xaa8′ is absent.

8. The peptide, or its analog having N-terminal, C-terminal, or side chain modified by PEGylation, phosphorylation, amidation, or acylation, or a pharmaceutically acceptable salt thereof according to claim 7, wherein
Xaa1 is selected from the group consisting of Lys and Arg;
Xaa2 is selected from the group consisting of Thr and Ala;
Xaa4 is selected from the group consisting of Arg, Lys, Ser, Ala and Thr;
Xaa1′ is selected from the group consisting of Ser and Ala;
Xaa2′ is selected from the group consisting of Ile, Leu, Nle and Ala;
Xaa3′ is selected from the group consisting of Pro and Hyp;
Xaa4′ is selected from the group consisting of Pro and Ala;
Xaa5′ is selected from the group consisting of Ile, Ala and Gln;
Xaa7′ is selected from the group consisting of Phe and Tyr; and
Xaa8′ is absent.

9. The peptide, or its analog having N-terminal, C-terminal, or side chain modified by PEGylation, phosphorylation, amidation or acylation, or a pharmaceutically acceptable salt thereof according to claim 8, wherein the peptide is selected from the group consisting of the following sequences: SEQ ID NO: 45, SEQ ID NO: 61, SEQ ID NO: 62, SEQ ID NO: 63, SEQ ID NO: 65, and SEQ ID NO: 68.

10. The peptide, or its analog having N-terminal, C-terminal, or side chain modified by PEGylation, phosphorylation, amidation or acylation, or a pharmaceutically acceptable salt thereof according to claim 7, wherein
Xaa1 is selected from the group consisting of Tyr and Phe;
Xaa2 is selected from the group consisting of Thr and Ala;
Xaa4 is selected from the group consisting of Ser, Ala, Phe and Thr;
Xaa1′ is Ser;
Xaa2′ is selected from the group consisting of Ile, Ala and Asn;
Xaa3′ is selected from the group consisting of Pro and Hyp;
Xaa4′ is selected from the group consisting of Pro, Ala and Hyp;
Xaa5′ is selected from the group consisting of Ile and Gln;
Xaa7′ is selected from the group consisting of Tyr, Phe, Asn, Gln and His; and
Xaa8′ is Gly, or absent.

11. The peptide, or its analog having N-terminal, C-terminal, or side chain modified by PEGylation, phosphorylation, amidation or acylation, or a pharmaceutically acceptable salt thereof according to claim 10, wherein a peptide is selected from the group consisting of the following sequences: SEQ ID NO: 85, SEQ ID NO: 90, SEQ ID NO: 91, SEQ ID NO: 98, SEQ ID NO: 105, SEQ ID NO: 106, SEQ ID NO: 113, SEQ ID NO: 114, SEQ ID NO: 115, SEQ ID NO: 131, SEQ ID NO: 132, and SEQ ID NO: 133.

12. The peptide, or its analog having N-terminal, C-terminal, or side chain modified by PEGylation, phosphorylation, amidation or acylation, or a pharmaceutically acceptable salt thereof according to claim 7, wherein
Xaa1 is selected from the group consisting of Ala and Leu;
Xaa2 is selected from the group consisting of Thr and Ala;
Xaa4 is selected from the group consisting of Ile, Leu, Val, Ala and Tyr;
Xaa1′ is selected from the group consisting of Ser and Ala;
Xaa2′ is selected from the group consisting of Ile and Asn;
Xaa3′ is selected from the group consisting of Pro and Hyp;
Xaa4′ is selected from the group consisting of Pro and Hyp;
Xaa5′ is selected from the group consisting of Ile and Gln;
Xaa7′ is selected from the group consisting of Gln and Tyr; and
Xaa8′ is absent.

13. The peptide, or its analog having N-terminal, C-terminal, or side chain modified by PEGylation, phosphorylation, amidation or acylation, or a pharmaceutically acceptable salt thereof according to claim 12, wherein a peptide is selected from the group consisting of the following sequences: SEQ ID NO: 134, SEQ ID NO: 145, SEQ ID NO: 151, SEQ ID NO: 155, SEQ ID NO: 156, SEQ ID NO: 158, SEQ ID NO: 181, and SEQ ID NO: 162.

14. A method for inhibiting trypsin, chymotrypsin or elastase of the serine protease family, comprising administering the peptide, or its analog having N-terminal, C-terminal, or side chain modified by PEGylation, phosphorylation, amidation or acylation, or a pharmaceutically acceptable salt thereof as defined in claim 1, to trypsin, chymotrypsin or elastase of the serine protease family.

15. A hybrid peptide having a structure of Formula (III), Formula (IV) or Formula (V):
B-L-A (III);
A-L-B (IV);
A1-L1-B-L2-A2 (V);
wherein:
the molecular mass of the hybrid peptide is 1.5 kDa to 30 kDa;
B is the peptide, or its analog having N-terminal, C-terminal, or side chain modified by PEGylation, phosphorylation, amidation or acylation, or a pharmaceutically acceptable salt thereof as defined in claim 1;
L is a linker which optionally has 1, 2, 3, 4 or 5 glycine or proline residues;
A is a bioactive oligopeptide, which is selected from the group consisting of therapeutic proteins, peptides and glycoproteins;
A1 and A2 are peptide segments of N-terminal and C-terminal of bioactive oligopeptide A, respectively;
L1 and L2 are linkers which optionally have 1, 2, 3, 4 or 5 glycine or proline residues, or absent.

16. The hybrid peptide according to claim 15, wherein the bioactive oligopeptide is selected from glucagon-like peptide-1, its analogues, or its peptide segments.

17. The hybrid peptide according to claim 16, wherein the hybrid peptide is selected from the group consisting of the following sequences: SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208 and SEQ ID NO: 209.

18. A method for treating type II diabetes and/or obesity, comprising administering the hybrid peptide as defined in claim 16 to the subject in need thereof.

19. The hybrid peptide according to claim 15, wherein the bioactive oligopeptide is selected from the peptide of sequence SEQ ID NO: 210, or its mutants.

20. The hybrid peptide according to claim 19, wherein the peptide is selected from the group consisting of the following sequences: SEQ ID NO: 211, SEQ ID NO: 212, SEQ ID NO: 214, SEQ ID NO: 215, SEQ ID NO: 216, SEQ ID NO: 217, SEQ ID NO: 218, SEQ ID NO:224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232 and SEQ ID NO: 233.

21. A method for treating familial hypercholesterolemia, comprising administering the hybrid peptide as define in claim 19 to the subject in need thereof.

22. The hybrid peptide according to claim 15, wherein the bioactive oligopeptide is selected from salmon calcitonin, its analogues or its mutants, and the salmon calcitonin is selected from the sequence of SEQ ID NO: 234.

23. The hybrid peptide according to claim 22, wherein the peptide is selected from the group consisting of the following sequences: SEQ ID NO: 235, SEQ ID NO: 236 and SEQ ID NO: 237.

24. A method for treating osteoporosis and/or osteoarthritis, comprising administering the hybrid peptide as defined in claim 22 to the subject in need thereof.

25. The hybrid peptide according to claim 15, wherein the bioactive oligopeptide is selected from the sequence of SEQ ID NO: 238, its analogues or its mutants.

26. The peptide according to claim 25, wherein the peptide is selected from the group consisting of the following sequence: SEQ ID NO: 239, SEQ ID NO: 240 and SEQ ID NO: 241.

27. A method for treating inflammatory lung disease, asthma, chronic obstructive pulmonary disease, inflammatory bowel disease, arthritis, autoimmune disease, rheumatoid arthritis, psoriasis, and systemic sclerosis, comprising administering they hybrid peptide as defined in claim 25 to the subject in need thereof.

28. A pharmaceutical composition, wherein the composition comprises a hybrid peptide as defined in claim 15 and a pharmaceutically acceptable carrier.
